(19) United States
(12) Patent Application Publication
(10) Pub. No.: US 2024/0376154 A1
(43) Pub. Date: Nov. 14, 2024

(54) BIS-INTERCALATING PEPTIDES, CONJUGATES AND METHODS OF USE

(71) Applicant: ZYMEWORKS BC INC., Vancouver (CA)

(72) Inventors: Mark Edmund Petersen, Vancouver (CA); Geoffrey C. Winters, Vancouver (CA); Manuel Michael Auguste Lasalle, Vancouver (CA); Micheal G. Brant, Vancouver (CA); Vincent K.C. Fung, Vancouver (CA)

(21) Appl. No.: 18/645,956

(22) Filed: Apr. 25, 2024

Related U.S. Application Data
(60) Provisional application No. 63/461,811, filed on Apr. 25, 2023.

(51) Int. Cl. C07K 7/54; A61K 38/00; A61K 47/68; A61P 35/00

(52) U.S. Cl. CPC C07K 7/54; A61K 47/6811; A61P 35/00; A61K 38/00

(57) ABSTRACT

Bis-intercalating peptide compounds having Formula I, which are capable of binding DNA and exerting a cytotoxic effect on cells, such as microbial, viral or tumour cells, and conjugates comprising the compounds linked to a targeting moiety, such as an antibody. The compounds and conjugates may be used in the treatment of cancer or a microbial or viral infection, or as diagnostic agents or labelling agents.

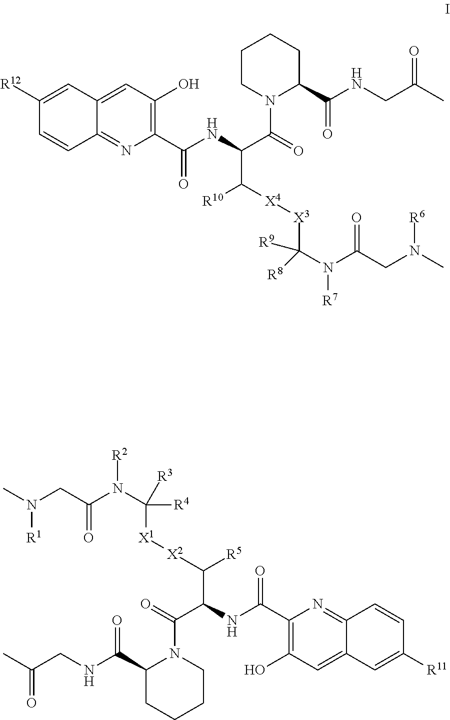

### FIELD

The present disclosure relates to the field of therapeutics and, in particular, to bis-intercalating peptides, conjugates comprising these compounds and methods of preparing and using same to treat cancer or a microbial infection.

### BACKGROUND

The bis-intercalator natural products are a family of non-ribosomal peptides possessing a range of biological properties that include antiviral, antibiotic and anticancer activities. These compounds have a peptidic core structure that is decorated with two planar heteroaromatic units, or “chromophores,” which bind to duplex DNA by bis-intercalation and thus are essential to the biological activity of these compounds.

The bis-intercalators can be divided into two classes, bicyclic and cyclic, based on their peptidic core structures. Examples of bicyclic bis-intercalators include echinomycin, thiocoraline, quinomycin (B and C) and triostin A. Examples of cyclic bis-intercalators include the luzopeptins, sandramycin, the quinoxapeptins and quinaldopeptin. Within the class of cyclic bis-intercalators, the luzopeptins, sandramycin and the quinoxapeptins are depsipeptides, whereas the peptidic core of quinaldopeptin is linked only by peptide bonds and thus differs from the other members of this class due to the lack of ester linkage.

Total synthetic methods for a number of members of the cyclic class of bis-intercalators have been described (Boger, et al., 1996, J. Am. Chem. Soc., 118:1629-1644; Boger, et al., 1999, J. Am. Chem. Soc., 121:11375-11383; Ciufolini, et al., 2000, Angew. Chem. Int. Ed., 39:2493-2495; Valognes, et al., 2001, Tet. Lett., 42:1907-1909; Ichikawa, et al., 2013, J. Org. Chem., 78:12662-12670, and Katayama, et al., 2014, Org. Lett., 16:428-431). U.S. Pat. No. 6,329,497 describes various analogues of sandramycin.

Site-specific conjugation of sandramycin to an anti-CD70 antibody at a drug-to-antibody ratio of approximately 2 has been described (Sussman, et al., 2018, Protein Eng. Des. Select., 31:47-54). Conjugation of sandramycin, luzopeptin A and SW-163D (a bicyclic bis-intercalator) to the anti-HER2 antibody, trastuzumab, at DAR 4 or DAR 2 has also been described (Ratnayake, et al., 2019, Bioconj. Chem., 30:200-209).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are bis-intercalating peptides, conjugates and methods of using same. One aspect of the present disclosure relates to a compound having Formula I:

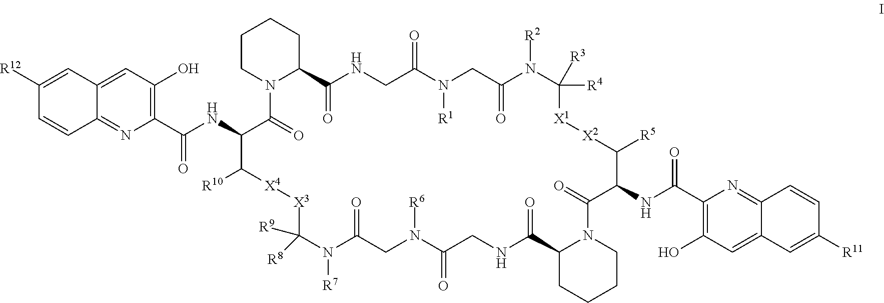

or a pharmaceutically acceptable salt thereof,
wherein:
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—, and
X² and X⁴ are each independently —NR¹⁴— or —O—, and R¹⁴ is H or C₁-C₂ alkyl, with the proviso that the compound is other than:

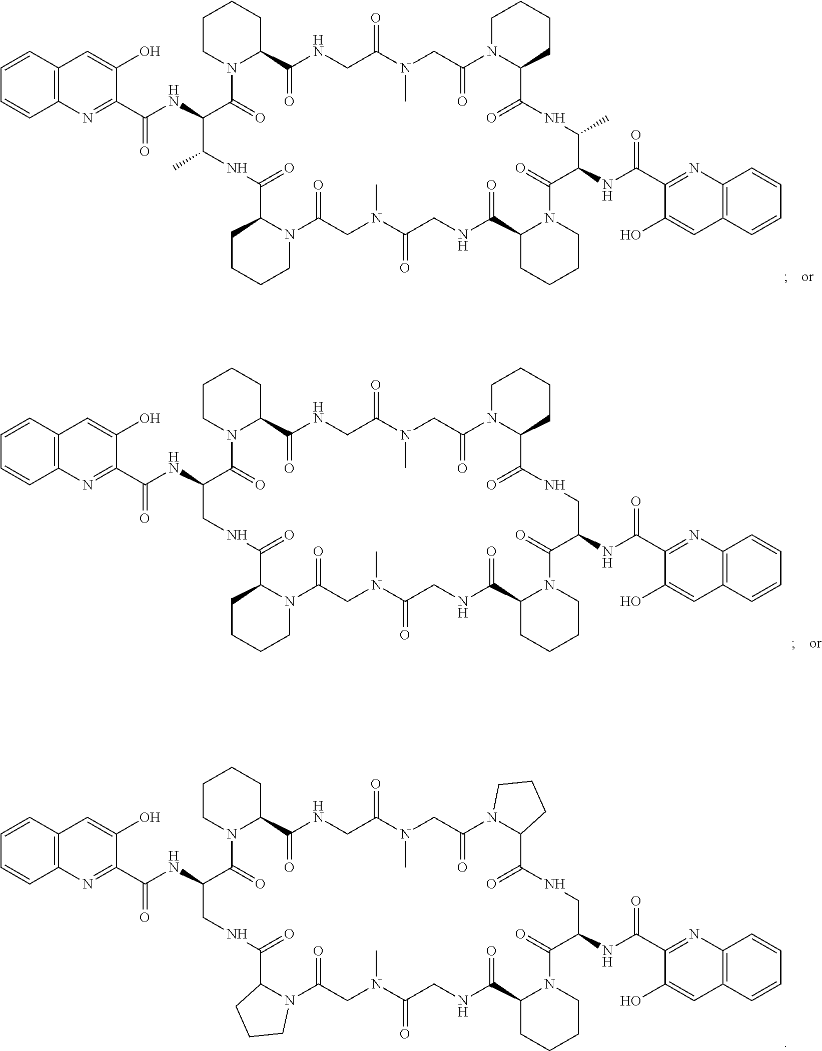

Another aspect of the present disclosure relates to a composition comprising a compound of Formula I and a pharmaceutically acceptable carrier, diluent or excipient.

Another aspect of the present disclosure relates to a method of inhibiting the proliferation of tumour cells, microbial cells or viral cells comprising contacting the cells with a compound of Formula I.

Another aspect of the present disclosure relates to a method of treating a microbial infection in a subject in need thereof, comprising administering to the subject an effective amount of a compound of Formula I.

Another aspect of the present disclosure relates to a method of treating a cancer in a subject in need thereof, comprising administering to the subject an effective amount of a compound of Formula I.

Another aspect of the present disclosure relates to a conjugate having Formula X:

T-[L-(D)ₘ]ₙ (X)
wherein:
T is a targeting moiety;
L is a linker;
D is a compound of Formula I;
m is between 1 and about 10, and
n is between 1 and about 12.

Another aspect of the present disclosure relates to a composition comprising a conjugate of Formula X and a pharmaceutically acceptable carrier, diluent or excipient.

Another aspect of the present disclosure relates to a method of inhibiting the proliferation of tumour cells, microbial cells or viral cells comprising contacting the cells with a conjugate of Formula X.

Another aspect of the present disclosure relates to amethod of treating a microbial infection in a subject in need thereof, comprising administering to the subject an effective amount of a conjugate of Formula X.

Another aspect of the present disclosure relates to a method of treating a cancer in a subject in need thereof, comprising administering to the subject an effective amount of a conjugate of Formula X.

Another aspect of the present disclosure relates to a drug-linker having Formula XI:

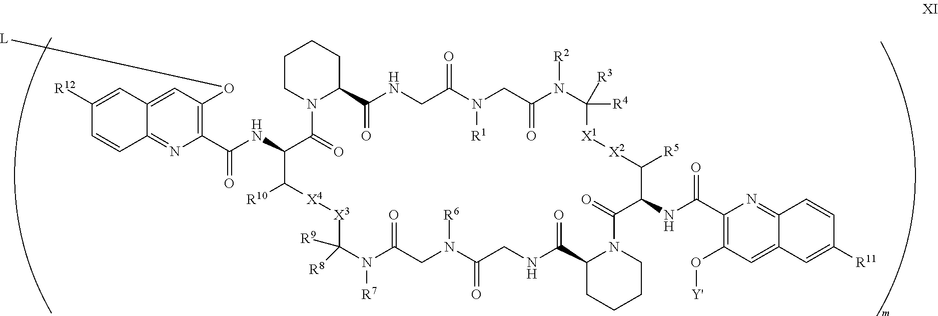

wherein:
L is a linker;
m is between 1 and about 10;
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—;
X² and X⁴ are each independently —NR¹⁴— or —O—, and R¹⁴ is H or C₁-C₂ alkyl, and
Y′ is H or (L^{A})ₚ-HM, where L^{A} is a self-immolative moiety, p is 0, 1 or 2, and HM is a hydrophilic moiety.

### BRIEF DESCRIPTION OF THE DRAWINGS

FIG. 1A, FIG. 1B, and FIG. 1C presents general synthetic schemes that may be employed in the preparation of compounds of Formula I.

FIG. 2 presents a general synthetic scheme that may be employed in the preparation of the compounds of Formula I.

FIG. 3 presents a general synthetic scheme that may be employed in the preparation of compounds of Formula I.

FIG. 4 presents a general synthetic scheme that may be employed in the preparation of drug-linker constructs comprising compounds of Formula I.

FIG. 5 shows the stability of exemplary compounds of Formula I in female mouse plasma compared to sandramycin (Compound 5) and quinaldopeptin.

FIG. 6 shows the effect of the mirvetuximab ADCs, Mi-1 (unmasked) and Mi-2 (comprising a glucuronide mask), on tumour volume in mice subcutaneously implanted with OVCAR-3 ovarian cancer cells.

FIG. 7 shows the effect of the telisotuzumab site-specific ADCs, Te-5 (unmasked) and Te-6 (comprising a glucuronide mask), on tumour volume in mice subcutaneously implanted with EBC-1 lung cancer cells.

### DETAILED DESCRIPTION

The present disclosure relates to bis-intercalating peptide compounds comprising a cyclic peptidic scaffold and two chromophores. The compounds are capable of binding DNA and exerting a cytotoxic effect on cells, such as microbial, viral or tumour cells. The present disclosure also relates to conjugates comprising the compounds described herein linked to a targeting moiety, such as an antibody.

The compounds and conjugates described herein find use in methods of inhibiting the proliferation of cells and/or killing cells, such as microbial, viral or tumour cells. Certain embodiments of the present disclosure thus relate to methods of using the compounds and conjugates described herein in the treatment of cancer or a microbial or viral infection. The compounds and conjugates described herein may also find use as diagnostic agents or labelling agents.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

If a value of a variable that is necessarily an integer is described herein as a range, it is to be understood that the value can be any integer between the stated upper and lower limits of the range and is inclusive of the upper and lower limit values, with each of these values forming an embodiment of the present disclosure. For example, if a variable is described as being from about 1 to about 4, the value can be any integer between 1 and 4 inclusive, i.e. 1, 2, 3 or 4, and each of these values form embodiments of the present disclosure. In addition, each of these values may represent the upper or lower limit of narrower ranges included within the stated range and each of such narrower ranges also form embodiments of the present disclosure, subject to any specifically excluded limits in the stated range.

An “effective amount” of a compound described herein in respect of a particular result to be achieved is an amount sufficient to achieve the desired result. For example, an “effective amount” of a compound when referred to in respect of the killing of cancer cells, refers to an amount of compound sufficient to produce a killing effect.

The term “C₁-C₄ alkyl,” as used herein, refers to a straight chain or branched alkyl group of one to four carbon atoms. Examples include methyl, ethyl, propyl, isopropyl, butyl, isobutyl, secbutyl and tert-butyl (t-butyl).

The term “C₁-C₄ alkoxy,” as used herein, refers to the group-OR, where R is C₁-C₄ alkyl as defined above.

The term “cycloalkyl,” as used herein, refers to a cyclic alkyl group containing the stipulated number of carbon atoms. For example, a C₃-C₆ cycloalkyl is a cyclic alkyl group containing between 3 and 6 carbon atoms. As a further example, a 3-membered cycloalkyl is a cyclic alkyl group containing 3 carbon atoms.

The term “heterocycloalkyl,” as used herein, refers to a cyclic alkyl group in which there is one heteroatom and the remaining number of stipulated atoms are carbon atoms. For example, a 5-membered heterocycloalkyl is a cyclic alkyl group containing one heteroatom and 4 carbon atoms.

The terms “halogen” and “halo,” as used herein, refer to fluorine, bromine, chlorine, and iodine atoms.

It is contemplated that any embodiment discussed herein can be implemented with respect to any method, use or composition disclosed herein, and vice versa.

It is also to be understood that the positive recitation of a feature in one embodiment serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

### Bis-Intercalating Peptides

One aspect of the present disclosure relates to bis-intercalating peptide compounds having Formula I:

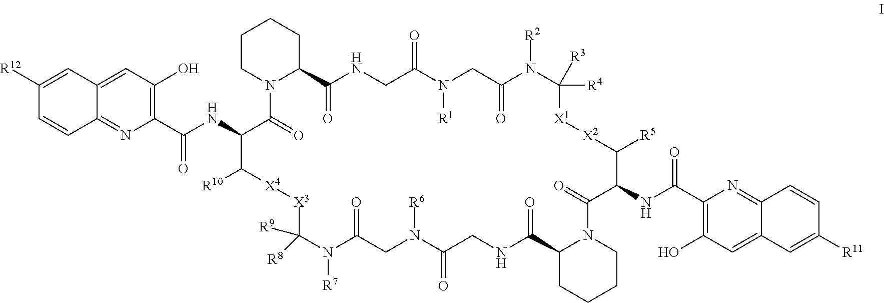

wherein:
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—, and
X² and X⁴ are each independently —NR¹⁴— or —O—, and R¹⁴ is H or C₁-C₂ alkyl, with the proviso that the compound is other than:

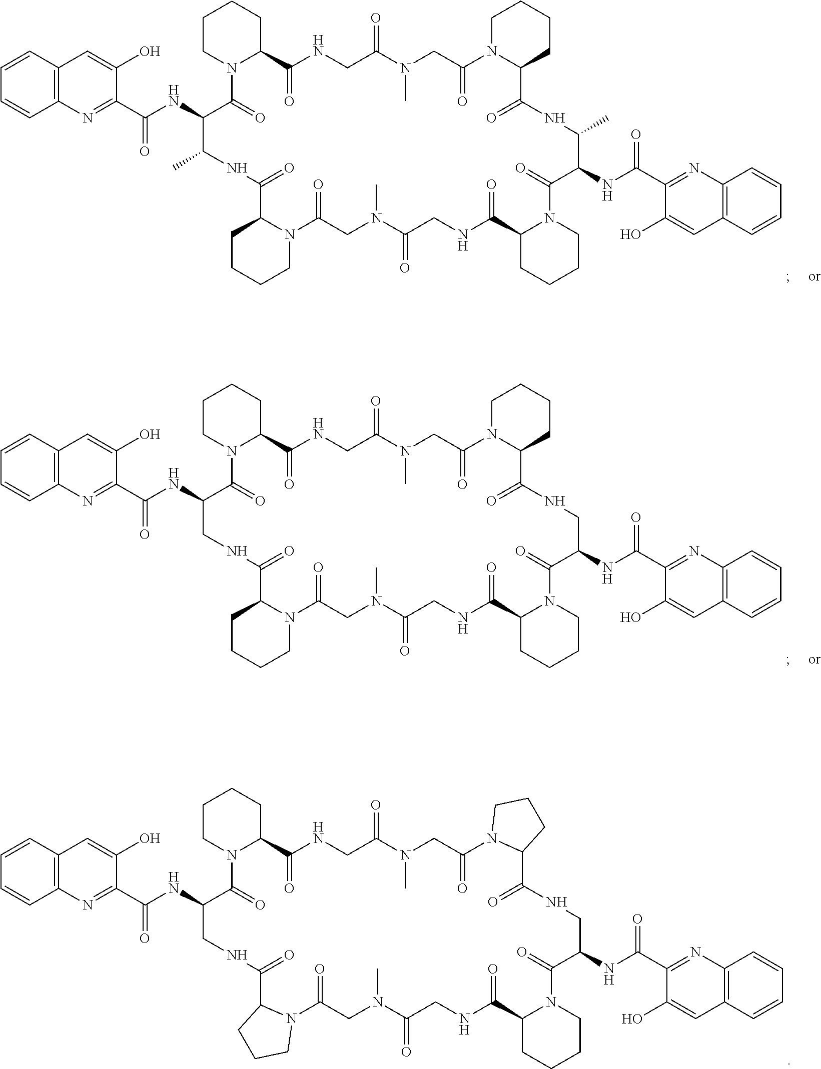

In certain embodiments, in compounds of Formula I, when R² and R³ together with the N and C atoms to which they are bonded form a 6-membered heterocycloalkyl, and R⁷ and R⁸ together with the N and C atoms to which they are bonded form a 6-membered heterocycloalkyl, and X¹ and X³ are each —C(O)—, then one of X² and X⁴ is other than —NH—.

In certain embodiments, in compounds of Formula I, when R² and R³ together with the N and C atoms to which they are bonded form a 5-membered heterocycloalkyl, and R⁷ and R⁸ together with the N and C atoms to which they are bonded form a 5-membered heterocycloalkyl, and X¹ and X³ are each —C(O)—, then one of X² and X⁴ is other than —NH—.

In certain embodiments, in compounds of Formula I:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 7-membered heterocycloalkyl, and R⁴ is H.

In certain embodiments, in compounds of Formula I:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl.

In certain embodiments, in compounds of Formula I:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 7-membered heterocycloalkyl, and R⁹ is H.

In certain embodiments, in compounds of Formula I:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl.

In certain embodiments, in compounds of Formula I, R⁵ and R¹⁰ are each H.

In certain embodiments, in compounds of Formula I, R¹¹ and R¹² are each H.

In certain embodiments, in compounds of Formula I, when X¹ is —CH₂—, X² is —O—, and when X³ is —CH₂—, X⁴ is —O—.

In certain embodiments, in compounds of Formula I, X¹ is —C(O)— and X² is —NR¹⁴— or —O—, or X³ is —C(O)— and X⁴ is —NR¹⁴— or —O—. In some embodiments, in compounds of Formula I, X¹ and X³ are each —C(O)—, and X² and X⁴ are each independently —NR¹⁴— or —O—.

In certain embodiments, in compounds of Formula I, X¹ is —C(O)— and X² is —NR¹⁴—, or X³ is —C(O)— and X⁴ is —NR¹⁴—. In certain embodiments, in compounds of Formula I, X¹ and X³ are each —C(O)—, and X² and X⁴ are each —NR¹⁴—.

Combinations of any of the foregoing embodiments for compounds of Formula I are also contemplated and each combination forms a separate embodiment for the purposes of the present disclosure.

Certain embodiments of the present disclosure relate to bis-intercalating peptide compounds having Formula II:

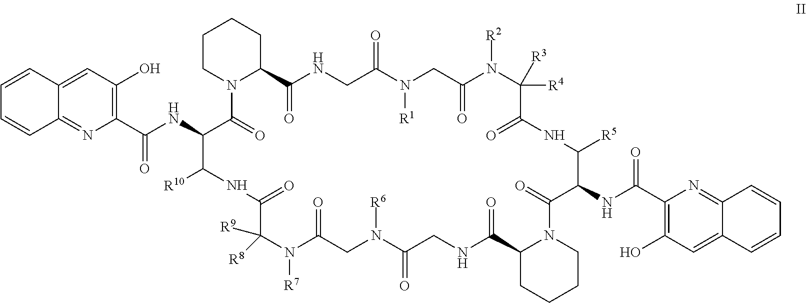

wherein:
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl, and
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
with the proviso that the compound is other than:

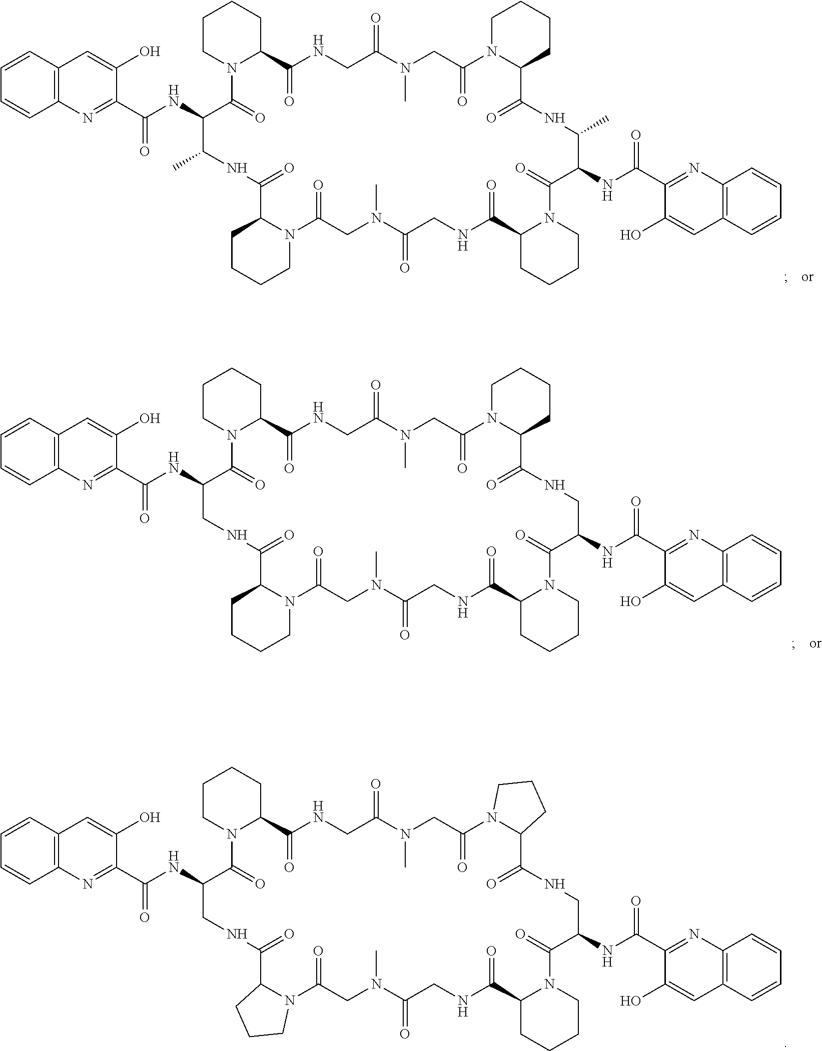

In certain embodiments, in compounds of Formula II:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 7-membered heterocycloalkyl, and R⁴ is H.

In certain embodiments, in compounds of Formula II:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl.

In certain embodiments, in compounds of Formula II:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 7-membered heterocycloalkyl, and R⁹ is H.

In certain embodiments, in compounds of Formula II:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl.

In certain embodiments, in compounds of Formula II, R⁵ and R¹⁰ are each H.

Combinations of any of the foregoing embodiments for compounds of Formula II are also contemplated and each combination forms a separate embodiment for the purposes of the present disclosure.

In certain embodiments of the present disclosure, compounds of Formula I or Formula II further comprise one or more hydrophilic moieties (HM) covalently linked to the compound. In some embodiments, the one or more hydrophilic moieties, HM, may be enzymatically cleavable from the compound. In some embodiments, the one or more hydrophilic moieties, HM, are enzymatically cleavable and linked to the compound via one or more self-immolative moieties.

Examples of hydrophilic moieties include, but are not limited to, various lengths of PEG or mPEG, polysarcosine, various saccharides (including β-glucuronide), sulfonate groups, sulfonamide groups, carboxylate groups and pyrophosphate diesters. Examples of self-immolative (or self-elimination) moities include, but are not limited to, p-aminobenzyl (PAB) groups, p-aminobenzyloxycarbonyl (PABC) groups, methylated ethylene diamine (MED), and aromatic compounds that are electronically similar to a PAB or PABC group (such as heterocyclic derivatives, for example, 2-aminoimidazol-5-methanol derivatives as described in U.S. Pat. No. 7,375,078).

Certain embodiments of the present disclosure thus relate to compounds of Formula III:

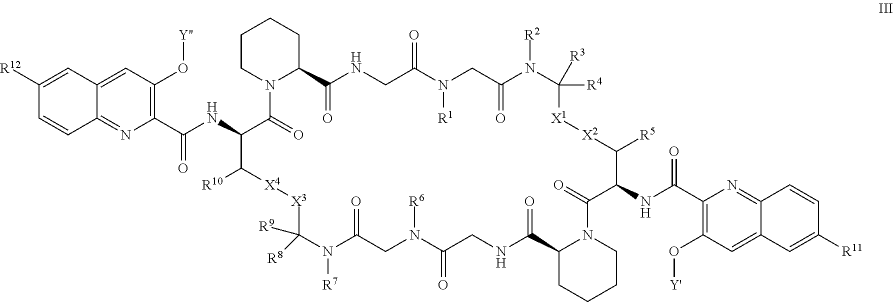

wherein:
R¹, R², R³, R⁴, R⁵, R⁶, R⁷, R⁸, R⁹, R¹⁰, R¹¹, R¹², X¹, X², X³ and X⁴ are as defined for Formula I, and
Y′ and Y″ are each independently H or (L^{A})ₚ-HM, where L^{A} is a self-immolative moiety, p is 0, 1 or 2, and HM is a hydrophilic moiety, and where at least one of Y′ and Y″ is (L^{A})ₚ-HM.

In certain embodiments, in compounds Formula III, R¹¹ and R¹² are each H.

In certain embodiments, in compounds Formula III, when X¹ is —CH₂—, X² is —O—, or when X³ is —CH₂—, X⁴ is —O—.

In certain embodiments, in compounds Formula III, X¹ is —C(O)— and X² is —NR¹⁴— or —O—, or X³ is —C(O)— and X⁴ is —NR¹⁴— or —O—. In some embodiments, in compounds Formula III, X¹ and X³ are each —C(O)—, and X² and X⁴ are each independently —NR¹⁴— or —O—.

In certain embodiments, in compounds of Formula III, X¹ is —C(O)— and X² is —NR¹⁴—, or X³ is —C(O)— and X⁴ is —NR¹⁴—. In certain embodiments, in compounds Formula III, X¹ and X³ are each —C(O)—, and X² and X⁴ are each —NR¹⁴.

In some embodiments, in compounds of Formula III, one of Y′ and Y″ is (L^{A})ₚ-HM, and the other of Y′ and Y″ is H. In some embodiments, in compounds of Formula III, HM is an enzymatically cleavable hydrophilic moiety and p is 1. In some embodiments, in compounds of Formula III, HM is an enzymatically cleavable hydrophilic moiety and p is 0.

Combinations of any of the foregoing embodiments for compounds of Formula III are also contemplated and each combination forms a separate embodiment for the purposes of the present disclosure.

Certain embodiments of the present disclosure relate to bis-intercalating peptide compounds selected from the compounds set forth in Tables 1 and 2 below.

TABLE 1
Exemplary Compounds of Formula I

| | |
|---|---|
| Chemical Structure | Chemical Name |

Lactone-containing compounds

| | |
|---|---|
| 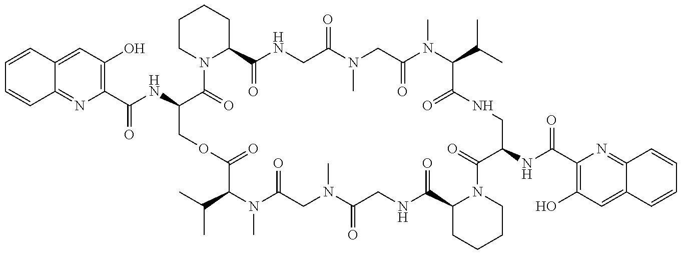 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl-12,15,31,34- tetramethyl- 6,10,13,16,19,25,29,32,35,38-deca- oxotetratriacontahydro-2H,6H,10H- dipyrido[2,1-b₁:2′,1′-l][1] oxa[4,7,10,13,17,20,23,26,29] nonaazacyclodotriacontine- 7,26-diyl)bis(3-hydroxyquinoline- 2-carboxamide), (Compound 14; FD-2) |
| 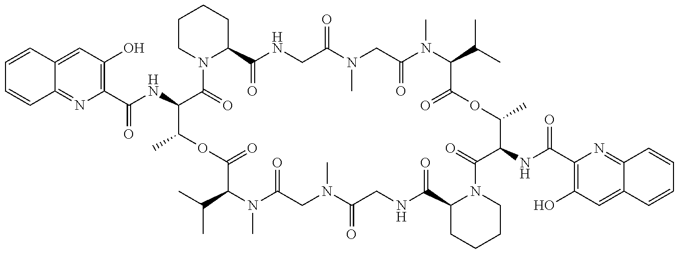 [SMILES: CC(C)[C@H]1C(=O)O[C@H](C)[C@@H](NC(=O)c2nc3ccccc3cc2O)C(=O)N2CCCC[C@H]2C(=O)NCC(=O)N(C)CC(=O)N(C)[C@@H](C(C)C)C(=O)O[C@H](C)[C@@H](NC(=O)c2nc3ccccc3cc2O)C(=O)N2CCCC[C@H]2C(=O)NCC(=O)N(C)CC(=O)N1C] | N,N′-((7R, 8R,11S,19aS,26R,27R,30S,38aS)- 11,30-diisopropyl- 8,12,15,27,31,34-hexamethyl- 6,10,13,16,19,25,29,32,35,38- decaoxodotriacontahydro- 2H,6H,10H,25H- dipyrido[2,1-b₁:2′,1′-l][1,17] dioxa[4,7,10,13,20,23,26,29]octa- azacyclodotriacontine- 7,26-diyl)bis(3- hydroxyquinoline-2-carboxamide), (Compound 22; FD-6) |
| 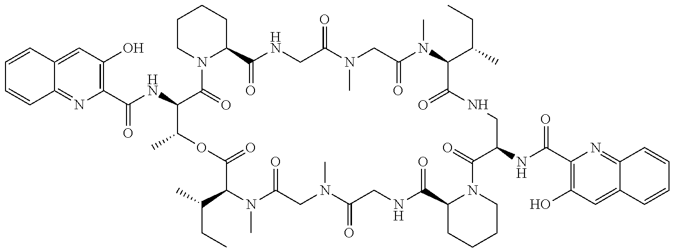 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)CC)C(=O)O[C@H](C)[C@@H](NC(=O)c2nc3ccccc3cc2O)C(=O)N2CCCC[C@H]2C(=O)NCC(=O)N(C)CC(=O)N1C] | N,N′- ((7R,8R,11S,19aS,26R,30S,38aS)- 11,30-di((S)- sec-butyl)-8,12,15,31,34- pentamethyl- 6,10,13,16,19,25,29,32,35,38- decaoxotetratriacontahydro- 2H,6H,10H-dipyrido[2,1-b₁:2′,1′-l] [1]oxa[4,7,10,13,17,20,23,26,29] nonaazacyclodotriacontine- 7,26-diyl)bis(3- hydroxyquinoline-2-carboxamide), (Compound 30; FD-35) |
| 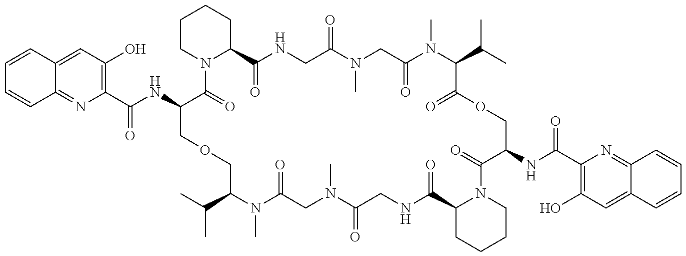 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl-12,15,31,34- tetramethyl- 6,10,13,16,19,25,32,35,38- nonaoxodotriacontahydro- 2H,6H,10H,25H- dipyrido[2,1-b₁:2′,1′-l][1,17]di- oxa[4,7,10,13,20,23,26,29]octa- azacyclodotriacontine- 7,26-diyl)bis(3- hydroxyquinoline-2-carboxamide), (Compound 203; FD-18) |

Bis-lactam compounds

| | |
|---|---|
| 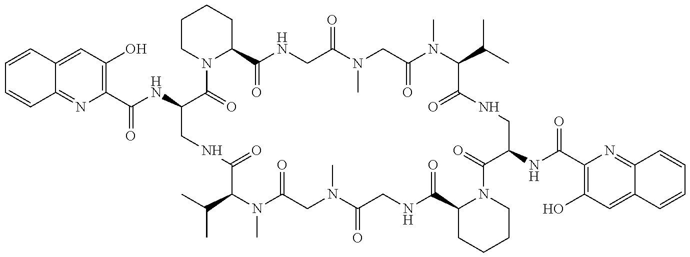 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl-12,15,31,34- tetramethyl- 6,10,13,16,19,25,29,32,35,38- decaoxohexatriacontahydro- 2H,6H-dipyrido[1,2-a:1′,2′-q] [1,4,7,10,13,17,20,23,26,29] decaazacyclodotriacontine- 7,26-diyl)bis(3-hydroxyquinoline- 2-carboxamide), (Compound 35; FD-1) |
| 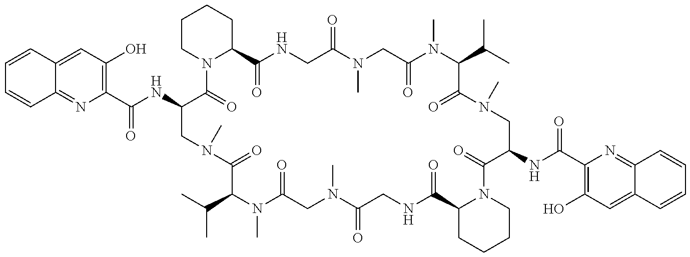 [SMILES: CC(C)[C@H]1C(=O)N(C)C[C@@H](NC(=O)c2nc3ccccc3cc2O)C(=O)N2CCCC[C@H]2C(=O)NCC(=O)N(C)CC(=O)N(C)[C@@H](C(C)C)C(=O)N(C)C[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl- 9,12,15,28,31,34- hexamethyl- 6,10,13,16,19,25,29,32,35,38- decaoxohexatriacontahydro- 2H,6H-dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 42; FD-4) |
| 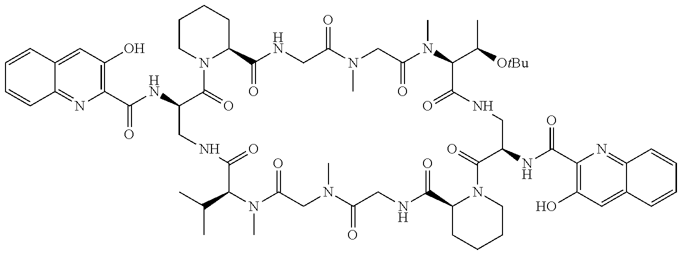 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)OC(C)(C)C)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)-11- ((R)-1-(tert-butoxy)ethyl)-30- isopropyl-12,15,31,34-tetramethyl- 6,10,13,16,19,25,29,32,35,38- decaoxohexatriacontahydro-2H,6H- dipyrido[1,2-a:1′,2′-q] [1,4,7,10,13,17,20,23,26,29] decaaza- cyclodotriacontine-7,26-diyl)bis(3- hydroxyquinoline-2-carboxamide), (Compound 54; FD-7) |
| 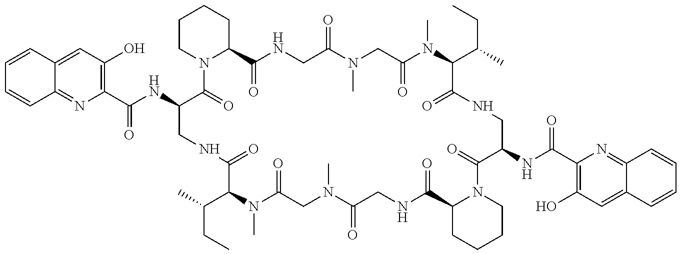 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-di((S)-sec-butyl-12,15,31,34- tetramethyl- 6,10,13,16,19,25,29,32,35,38- decaoxohexatriacontahydro-2H,6H- dipyrido[1,2-a:1′,2′-q] [1,4,7,10,13,17,20,23,26,29] decaaza- cyclodotriacontine-7,26-diyl)bis(3- hydroxyquinoline-2-carboxamide), (Compound 67; FD-9) |
| 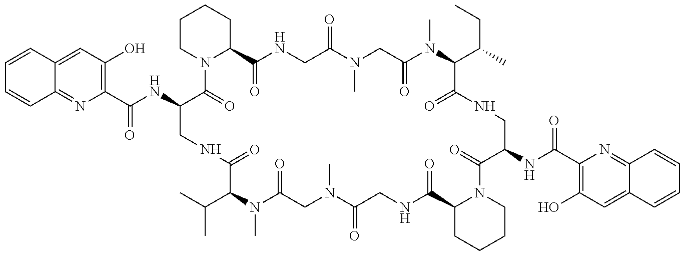 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11-((S)-sec-butyl)-30-isopropyl- 12,15,31,34-tetramethyl- 6,10,13,16,19,25,29,32,35,38- decaoxohexatriacontahydro-2H,6H- dipyrido[1,2-a:1′,2′-q] [1,4,7,10,13,17,20,23,26,29] decaaza- cyclodotriacontine-7,26-diyl)bis(3- hydroxyquinoline-2-carboxamide). (Compound 72;FD-10) |
| 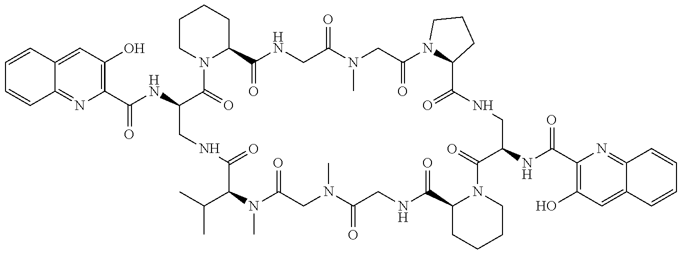 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N2CCC[C@H]2C(=O)NC[C@@H](NC(=O)c2nc3ccccc3cc2O)C(=O)N2CCCC[C@H]2C(=O)NCC(=O)N(C)CC(=O)N1C] | N,N′- ((3aS,7R,13aS,22S,26R,32aS)- 22-isopropyl-18,21,37-trimethyl- 4,8,14,17,20,23,27,33,36,39- decaoxooctatriacontahydro- 1H,10H-dipyrido[1,2-a:1′,2′-q] pyrrolo[1,2-j] [1,4,7,10,13,17,20,23,26,29] decaazacyclodotriacontine- 7,26-diyl)bis(3-hydroxyquinoline- 2-carboxamide), (Compound 77; FD-20) |
| 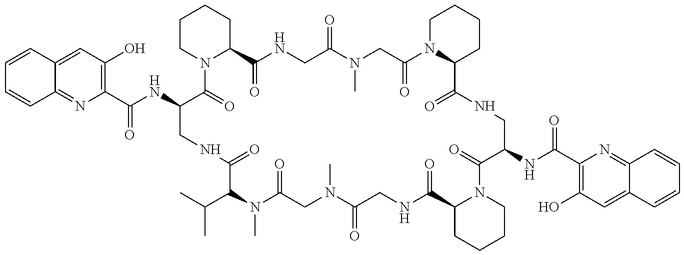 | N,N′- ((4aS,8R,14aS,23S,27R,33aS)- 23-isopropyl-19,22,38-trimethyl- 5,9,15,18,21,24,28,34,37,40- decaoxotetracontahydro-11H- tripyrido[1,2-a:1′,2′-j:1″,2″-q] [1,4,7,10,13,17,20,23,26,29] decaazacyclodotriacontine- 8,27-diyl)bis(3-hydroxy- quinoline-2-carboxamide), (Compound 82; FD-21) |
| 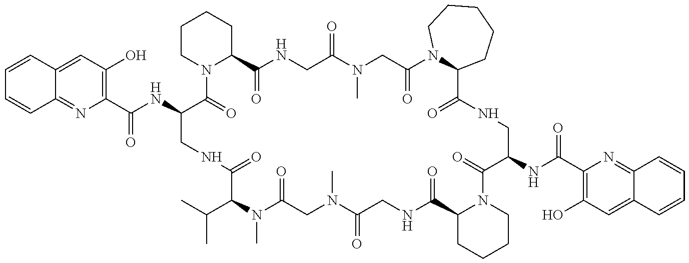 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N2CCCCC[C@H]2C(=O)NC[C@@H](NC(=O)c2nc3ccccc3cc2O)C(=O)N2CCCC[C@H]2C(=O)NCC(=O)N(C)CC(=O)N1C] | N,N′- ((5aS,9R,15aS,24S,28R,34aS)- 24-isopropyl-20,23,39-trimethyl- 6,10,16,19,22,25,29,35,38,41- decaoxotetracontahydro-1H,12H- azepino[1,2-j]dipyrido [1,2-a:1′,2′-q]- [1,4,7,10,13,17,20,23,26,29] decaazacyclodo triacontine-9,28-dihyl)bis(3- hydroxyquinoline-2-carboxamide), (Compound 88; FD-22) |
| 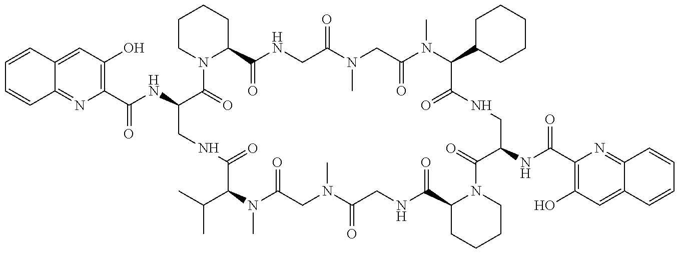 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11-cyclohexyl-30-isopropyl- 12,15,31,34-tetramethyl- 6,10,13,16,19,25,29,32,35,38- decaoxohexatriacontahydro- 2H,6H-dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 97; FD-11) |
| 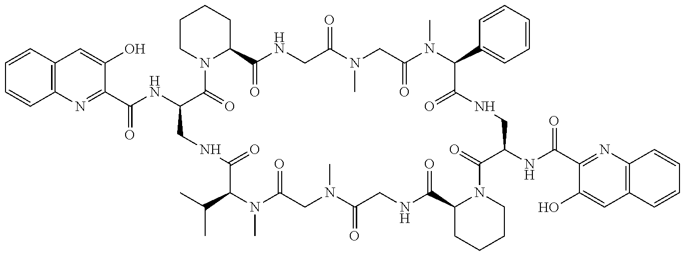 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](c2ccccc2)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11-isopropyl-12,15,31,34- tetramethyl- 6,10,13,16,19,25,29,32,35,38-deca- oxo-30-phenyl]hexatriacontahydro- 2H,6H-dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 103; FD-12) |
| 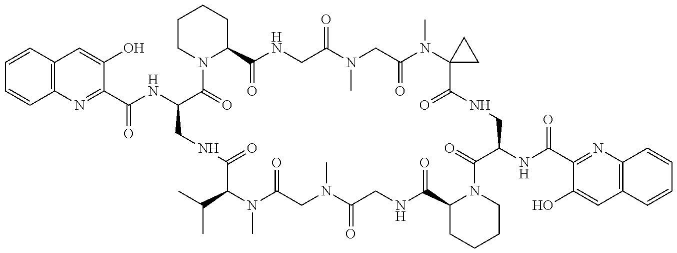 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)C2(CC2)C(=O)NC[C@@H](NC(=O)c2nc3ccccc3cc2O)C(=O)N2CCCC[C@H]2C(=O)NCC(=O)N(C)CC(=O)N1C] | N,N′- ((7′R,19a′S,26′R,30′S,38a′S)-30′- isopropyl-12′,15′,31′,34′- tetramethyl- 6′,10′,13′,16′,19′,25′,29′,32′,35′,38′- decaoxotetratriacontahydro- 2′H,6′H,12′H-spiro[cyclopropane- 1,11′-dipyrido[1,2-a:1′,2′-q] [1,4,7,10,13,17,20,23,26,29] decaazacyclodotriacontine]- 7′,26′-diyl)bis(3- hydroxyquinoline-2-carboxamide), (Compound 109; FD-32) |
| 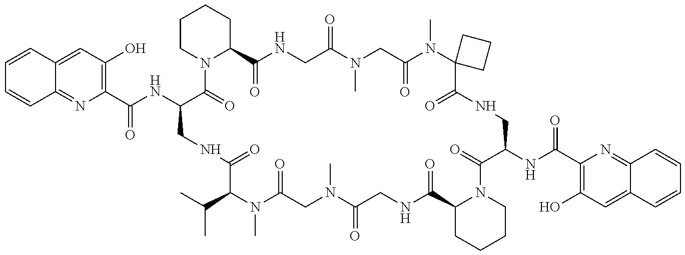 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)C2(CCC2)C(=O)NC[C@@H](NC(=O)c2nc3ccccc3cc2O)C(=O)N2CCCC[C@H]2C(=O)NCC(=O)N(C)CC(=O)N1C] | N,N′- ((7′R,19a′S,26′R,30′S,38a′S)-30′- isopropyl-12′,15′,31′,34′- tetramethyl- 6′,10′,13′,16′,19′,25′,29′,32′,35′,38′- decaoxotetratriacontahydro- 2′H,6′H,12′H-spiro[cyclobutane- 1,11′-dipyrido[1,2-a:1′,2′-q] [1,4,7,10,13,17,20,23,26,29] decaazacyclodotriacontine]- 7′,26′-diyl)bis(3- hydroxyquinoline-2-carboxamide). (Compound 121; FD-34) |
| 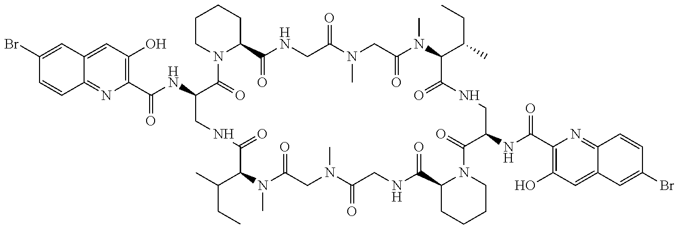 [SMILES: CCC(C)[C@H]1C(=O)NC[C@@H](NC(=O)c2nc3ccc(Br)cc3cc2O)C(=O)N2CCCC[C@H]2C(=O)NCC(=O)N(C)CC(=O)N(C)[C@@H]([C@@H](C)CC)C(=O)NC[C@@H](NC(=O)c2nc3ccc(Br)cc3cc2O)C(=O)N2CCCC[C@H]2C(=O)NCC(=O)N(C)CC(=O)N1C] | N,N′- ((7R,11S,19aS,26R,30S,38aS)- 11,30-di((S)-sec-butyl)- 12,15,31,34-tetramethyl- 6,10,13,16,19,25,29,32,35,38- decaoxohexatriacontahydro-2H,6H- dipyrido[1,2-a:1′,2′-q] [1,4,7,10,13,17,20,23,26,29] decaazacyclodotriacontine- 7,26-diyl)bis(6-bromo- 3-hydroxyquinoline- 2-carboxamide), (Compound 127; FD-36) |
| 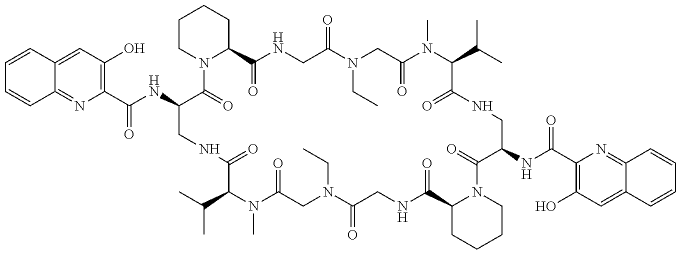 [SMILES: CCN1CC(=O)N(C)[C@@H](C(C)C)C(=O)NC[C@@H](NC(=O)c2nc3ccccc3cc2O)C(=O)N2CCCC[C@H]2C(=O)NCC(=O)N(CC)CC(=O)N(C)[C@@H](C(C)C)C(=O)NC[C@@H](NC(=O)c2nc3ccccc3cc2O)C(=O)N2CCCC[C@H]2C(=O)NCC1=O] | N,N′- ((7R,11S,19aS,26R,30S,38aS)- 15,34-diethyl-11,30-diisopropyl- 12,31-dimethyl- 6,10,13,16,19,25,29,32,35,38- decaoxohexatriacontahydro-2H,6H- dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 132; FD-23) |
| 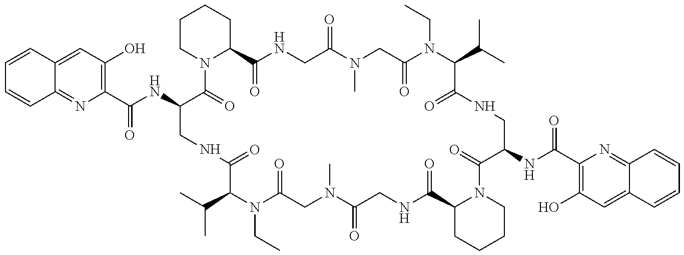 [SMILES: CCN1C(=O)CN(C)C(=O)CNC(=O)[C@@H]2CCCCN2C(=O)[C@H](NC(=O)c2nc3ccccc3cc2O)CNC(=O)[C@H](C(C)C)N(CC)C(=O)CN(C)C(=O)CNC(=O)[C@@H]2CCCCN2C(=O)[C@H](NC(=O)c2nc3ccccc3cc2O)CNC(=O)[C@@H]1C(C)C] | N,N′- ((7R,11S,19aS,26R,30S,38aS)- 12,31-diethyl-11,30-diisopropyl- 15,34-dimethyl- 6,10,13,16,19,25,29,32,35,38- decaoxohexatriacontahydro-2H,6H- dipyrido[1,2-a:1′,2′-q] [1,4,7,10,13,17,20,23,26,29] decaaza- cyclodotriacontine-7,26-diyl)bis(3- hydroxyquinoline-2-carboxamide), (Compound 147; FD-25) |
| 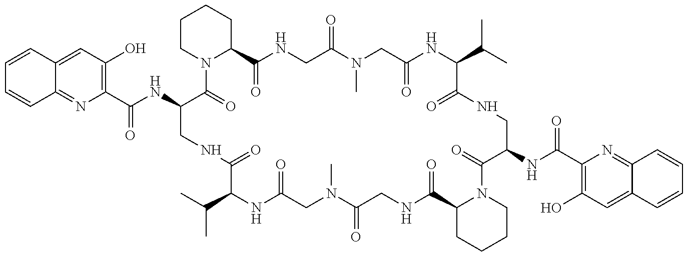 [SMILES: CC(C)[C@@H]1NC(=O)CN(C)C(=O)CNC(=O)[C@@H]2CCCCN2C(=O)[C@H](NC(=O)c2nc3ccccc3cc2O)CNC(=O)[C@H](C(C)C)NC(=O)CN(C)C(=O)CNC(=O)[C@@H]2CCCCN2C(=O)[C@H](NC(=O)c2nc3ccccc3cc2O)CNC1=O] | N,N′- ((7R,11S,19aS,26R,30S,38aS)- 11,30-diisopropyl-15,34-dimethyl- 6,10,13,16,19,25,29,32,35,38- decaoxohexatriacontahydro-2H,6H- dipyrido[1,2-a:1′,2′-q] [1,4,7,10,13,17,20,23,26,29] decaaza- cyclodotriacontine-7,26-diyl)bis(3- hydroxyquinoline-2-carboxamide), (Compound 152; FD-26) |
| 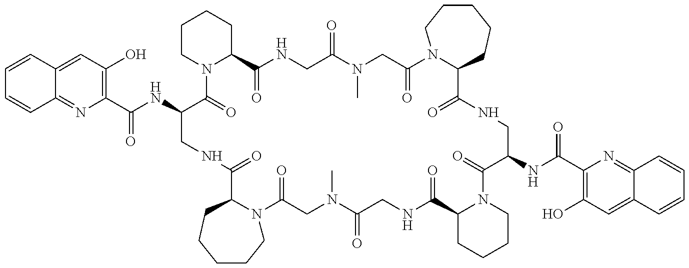 [SMILES: CN1CC(=O)N2CCCCC[C@H]2C(=O)NC[C@@H](NC(=O)c2nc3ccccc3cc2O)C(=O)N2CCCC[C@H]2C(=O)NCC(=O)N(C)CC(=O)N2CCCCC[C@H]2C(=O)NC[C@@H](NC(=O)c2nc3ccccc3cc2O)C(=O)N2CCCC[C@H]2C(=O)NCC1=O] | N,N′- ((7R,10aS,23aS,30R,33aS,46aS)- 19,42-dimethyl- 6,10,17,20,23,29,33,40,43,46- decaoxotetratetracontahydro- 6H,29H- bis(azepino)[1,2-j:1′,2′-z]dipyrido [1,2-a:1′,2′-q] [1,4,7,10,13,17,20,23,26,29] decaazacyclodotriacontine- 7,30-diyl)bis(3- hydroxyquinoline-2-carboxamide), (Compound 157; FD-16) |
| 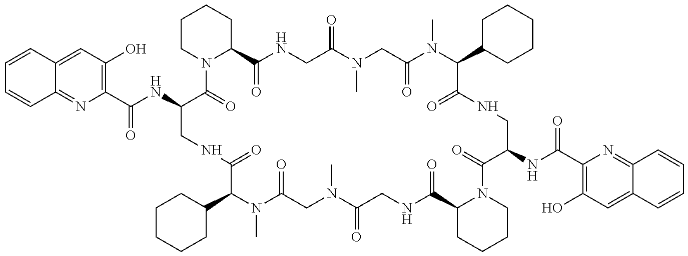 [SMILES: CN1CC(=O)N(C)[C@@H](C2CCCCC2)C(=O)NC[C@@H](NC(=O)c2nc3ccccc3cc2O)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2O)C(=O)N2CCCC[C@H]2C(=O)NCC1=O] | N,N′- ((7R,11S,19aS,26R,30S,38aS)- 11,30-dicyclohexyl-12,15,31,34- tetramethyl- 6,10,13,16,19,25,29,32,35,38- decaoxohexatriacontahydro-2H,6H- dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 162; FD-13) |
| 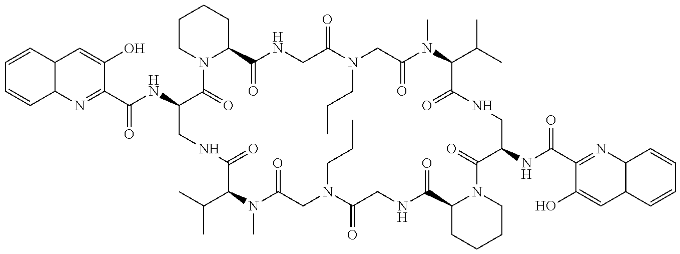 [SMILES: CCCN1CC(=O)N(C)[C@@H](C(C)C)C(=O)NC[C@@H](NC(=O)C2=NC3C=CC=CC3C=C2O)C(=O)N2CCCC[C@H]2C(=O)NCC(=O)N(CCC)CC(=O)N(C)[C@@H](C(C)C)C(=O)NC[C@@H](NC(=O)C2=NC3C=CC=CC3C=C2O)C(=O)N2CCCC[C@H]2C(=O)NCC1=O] | N,N′- ((7R,11S,19aS,26R,30S,38aS)- 11,30-diisopropyl-12,31-dimethyl- 6,10,13,16,19,25,29,32,35,38- decaoxo-15,34- dipropylhexatriacontahydro- 2H,6H-dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 419) |
| 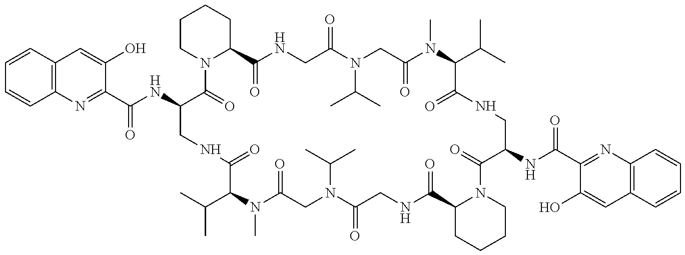 [SMILES: CC(C)[C@H]1C(=O)NC[C@@H](NC(=O)c2nc3ccccc3cc2O)C(=O)N2CCCC[C@H]2C(=O)NCC(=O)N(C(C)C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C(C)C)CC(=O)N1C] | N,N′- ((7R,11S,19aS,26R,30S,38aS)- 11,15,30,34-tetraisopropyl-12,31- dimethyl- 6,10,13,16,19,25,29,32,35,38- decaoxohexatriacontahydro- 2H,6H-dipyrido[1,2-a:1′,2′-q] [1,4,7,10,13,17,20,23,26,29] decaazacyclodotriacontine- 7,26-diyl)bis(3- hydroxyquinoline-2-carboxamide), (Compound 424) |

Ether containing compounds

| | |
|---|---|
| 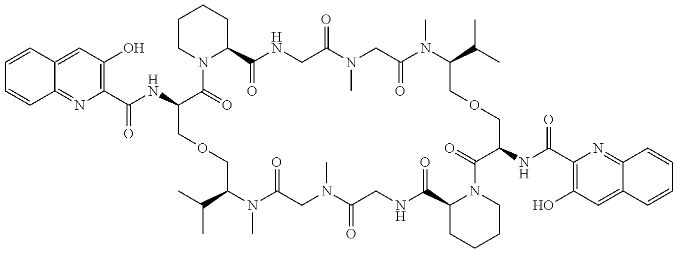 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)COC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl-12,15,31,34- tetramethyl- 6,13,16,19,25,32,35,38- octaoxodotriacontahydro- 2H,6H,10H,25H- dipyrido[2,1-b₁:2′,1′-l][1,17] dioxa[4,7,10,13,20,23,26,29] octaazacyclodotriacontine-7,26- diyl)bis(3-hydroxyquinoline- 2-carboxamide), (Compound 172; FD-17) |
| 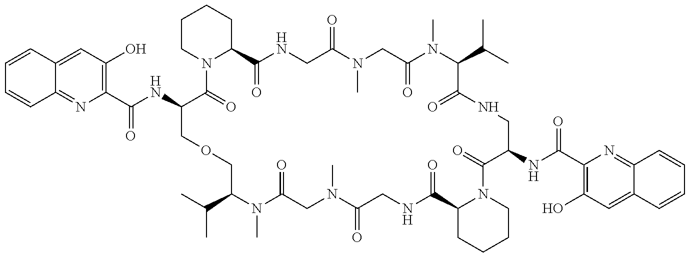 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11,30-diisopropyl-12,15,31,34- tetramethyl- 6,13,16,19,25,29,32,35,3,8- nonaoxotetratriacontahydro- 2H,6H,10H- dipyrido[2,1-b₁:2′,1′-l][1] oxa[4,7,10,13,17,20,23,26,29] nonaazacyclo- dotriacontine-7,26-diyl)bis(3- hydroxyquinoline-2-carboxamide), (Compound 178; FD-3) |
| 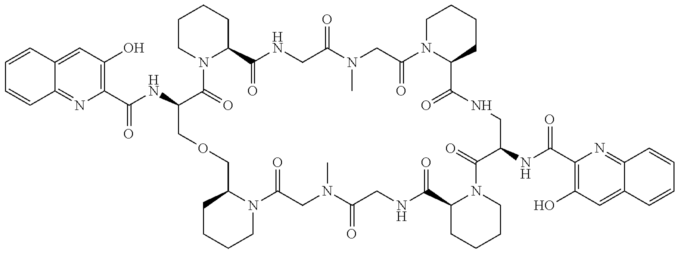 [SMILES: CN1CC(=O)N2CCCC[C@H]2COC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](NC(=O)c2nc3ccccc3cc2O)C(=O)N2CCCC[C@H]2C(=O)NCC1=O] | N,N′- ((7R,10aS,22aS,29R,32aS,44aS)- 18,40-dimethyl- 6,16,19,22,28,32,38,41,44-nona- oxodotetracontahydro-6H-tetra- pyrido[2,1-b₁:2′,1′-c:2″,1″-l:2′′′,1′′′- s][1]oxa[4,7,10,13,17,20,23,26,29] nonaazacyclodotriacontine- 7,29-diyl)bis(3-hydroxyquinoline- 2-carboxamide), (Compound 187; FD-15) |
| 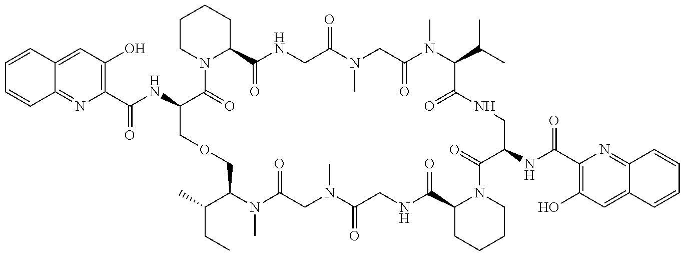 [SMILES: CC[C@H](C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C] | N,N′- ((7R,11S,19aS,26R,30S,38aS)- 11-((S)-sec-butyl)-30-isopropyl- 12,15,31,34-tetramethyl- 6,13,16,19,25,29,32,35,38- nonaoxotetratriacontahydro- 2H,6H,10H- dipyrido[2,1-b₁:2′,1′-l][1]oxa [4,7,10,13,17,20,23,26,29]nonaa- zacyclodotriacontine-7,26-diyl) bis(3-hydroxyquinoline-2- carboxamide), (Compound 197; FD-19) |

TABLE 2
Additional Exemplary Compounds of Formula I

| Chemical Structure | Chemical Name |
|---|---|
| 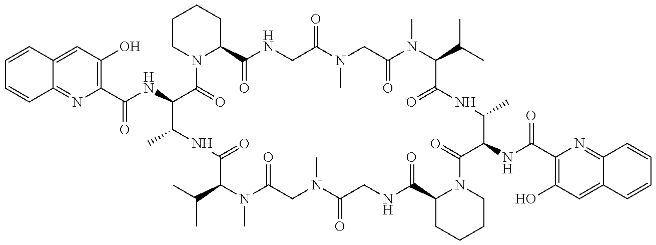 [SMILES: CC(C)[C@H]1C(=O)N[C@H](C)[C@@H](NC(=O)c2nc3ccccc3cc2O)C(=O)N2CCCC[C@H]2C(=O)NCC(=O)N(C)CC(=O)N(C)[C@@H](C(C)C)C(=O)N[C@H](C)[C@@H](NC(=O)c2nc3ccccc3cc2O)C(=O)N2CCCC[C@H]2C(=O)NCC(=O)N(C)CC(=O)N1C] | N,N′- ((7R,8R,11S,19aS,26R,27R,30S,38aS)- 11,30-diisopropyl-8,12,15,27,31,34- hexamethyl- 6,10,13,16,19,25,29,32,35,3,8- decaoxohexatriacontahydro-2H,6H- dipyrido[1,2-a:1′,2′-q] [1,4,7,10,13,17,20,23,26,29]decaazacy- clodotriacontine-7,26-diyl)bis(3- hydroxyquinoline-2-carboxamide), (FD-5) |
| 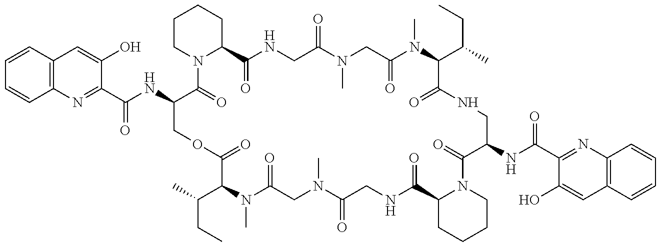 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)CC)C(=O)OC[C@@H](NC(=O)c2nc3ccccc3cc2O)C(=O)N2CCCC[C@H]2C(=O)NCC(=O)N(C)CC(=O)N1C] | N,N′-((7R,11S,19aS,26R,30S,38aS)- 11,30-di((S)-sec-butyl)-12,15,31,34- tetramethyl- 6,10,13,16,19,25,29,32,35,38- decaoxotetratriacontahydro- 2H,6H,10H-dipyrido[2,1-b₁:2′,1′-l] [1]oxa[4,7,10,13,17,20,23,26,29]nonaa- zacyclodotriacontine-7,26-diyl)bis(3- hydroxyquinoline-2-carboxamide), (Compound 425) |
| 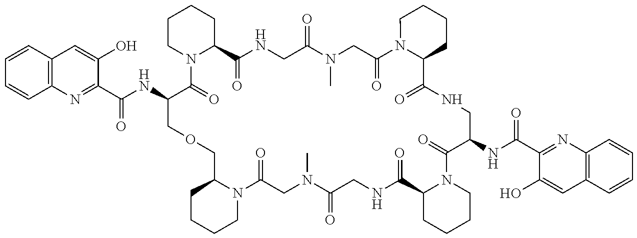 [SMILES: CN1CC(=O)N2CCCC[C@H]2COC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](NC(=O)c2nc3ccccc3cc2O)C(=O)N2CCCC[C@H]2C(=O)NCC1=O] | N,N′-((7R,10aS,22aS,29R,32aS,44aS)- 18,40-dimethyl- 6,10,16,19,22,28,32,38,31,44- decaoxodotetracontahydro-6H-tetra- pyrido[2,1-b₁:2′,1′-c:2″,1″-l:2′′′,1′′′- s][1]oxa[4,7,10,13,17,20,23,26,29] nonaazacyclodotriacontine- 7,29-diyl)bis(3- hydroxyquinoline-2-carboxamide), (Compound 426) |

It is to be understood that reference to compounds of Formula I throughout this disclosure includes, in various embodiments, compounds of Formula II and compounds of Formula III to the same extent as if embodiments reciting each of these formulae individually were specifically recited.

In certain embodiments, a compound of Formula I may possess a sufficiently acidic group, a sufficiently basic group, or both functional groups, and accordingly may react with one of a number of organic and inorganic bases, or organic and inorganic acids, to form a pharmaceutically acceptable salt. The term “pharmaceutically acceptable salt” as used herein, refers to a salt of a compound of Formula I, which is substantially non-toxic to living organisms. Typical pharmaceutically acceptable salts include those salts prepared by reaction of a compound of Formula I with a pharmaceutically acceptable mineral or organic acid or an organic or inorganic base. Such salts are known as acid addition and base addition salts.

Acids commonly employed to form acid addition salts are inorganic acids including, but are not limited to, hydrochloric acid, hydrobromic acid, hydroiodic acid, sulphuric acid, phosphoric acid, and organic acids including, but not limited to, p-toluenesulphonic acid, methanesulphonic acid, oxalic acid, p-bromophenylsulphonic acid, carbonic acid, succinic acid, citric acid, benzoic acid and acetic acid. Examples of pharmaceutically acceptable salts include, but are not limited to, sulphates, pyrosulphates, bisulphates, sulphites, phosphates, monohydrogenphosphates, dihydrogenphosphates, metaphosphates, pyrophosphates, bromides, iodides, acetates, propionates, decanoates, caprylates, acrylates, formates, hydrochlorides, dihydrochlorides, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, hydroxybenzoates, methoxybenzoates, phthalates, xylenesulphonates, phenylacetates, phenylpropionates, phenylbutyrates, citrates, lactates, gamma-hydroxybutyrates, glycolates, tartrates, methanesulphonates, propanesulphonates, naphthalene-1-sulfonates, napththalene-2-sulfonates and mandelates.

Salts of amine groups may also comprise quarternary ammonium salts in which the amino nitrogen carries a suitable organic group such as an alkyl, lower alkenyl, lower alkynyl or aralkyl moiety.

Base addition salts include those derived from inorganic bases, such as ammonium or alkali or alkaline earth metal hydroxides, carbonates, bicarbonates, and the like. Bases useful in preparing pharmaceutically acceptable salts include, but are not limited to, sodium hydroxide, potassium hydroxide, ammonium hydroxide, potassium carbonate, sodium carbonate, sodium bicarbonate, potassium bicarbonate, calcium hydroxide and calcium carbonate.

One skilled in the art will understand that the particular counterion forming a part of a pharmaceutically acceptable salt is usually not of a critical nature, so long as the salt as a whole is pharmacologically acceptable and as long as the counterion does not contribute undesired qualities to the salt as a whole.

Certain embodiments relate to pharmaceutically acceptable solvates of a compound of Formula I. One skilled in the art will appreciate that certain compounds of Formula I may combine with solvents such as water, methanol, ethanol or acetonitrile to form pharmaceutically acceptable solvates such as the corresponding hydrate, methanolate, ethanolate or acetonitrilate. Other examples of solvents that may be used to prepare solvates include isopropanol, dimethyl sulfoxide, ethyl acetate, acetic acid, ethanolamine and acetone, as well as miscible formulations of solvate mixtures as would be known by the skilled artisan.

### Preparation of Bis-Intercalating Peptides

Compounds of Formula I may be considered as having a cyclic peptidic scaffold made up of amino acids and/or amino acid analogues, and two pendant chromophores having the structure

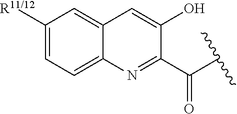

in Formula I. For convenience, the peptidic scaffold of compounds of Formula I and of the intermediates generated in preparing compounds of Formula I is referred to herein as a “decapeptide.” It is to be understood from the foregoing description and the general formulae provided herein that the term “decapeptide” is not limited to amino acids and/or amino acid analogues linked by peptide bonds, but also includes peptidomimetic compounds in which peptide bonds have been replaced with other chemical groups, such as esters, ethers or thioethers. The same applies to the terms “pentapeptide” and “dipeptide” used below.

Compounds of Formula I may be prepared from readily available starting materials. Appropriate starting materials may be obtained from commercial sources (such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd.), or may be synthesized following procedures known in the art (see, for example, March's Advanced Organic Chemistry: Reactions, Mechanisms, and Structure, 7th edition, John Wiley & Sons, Inc., 2013) or as described in the Examples herein.

Compounds of Formula I may be prepared by methods known in the art. For example, solution phase peptide synthesis or solid phase peptide synthesis may be used to prepare the peptidic scaffold of the compound as a linear decapeptide, followed by cyclization via formation of an amide bond between the two termini of the linear decapeptide to yield a cyclic intermediate, then installation of the chromophore moieties (see, for example, Boger, et al., 1996, J. Am. Chem. Soc., 118:1629-1644; U.S. Pat. No. 6,329,497; Ichikawa, et al., 2013, J. Org. Chem., 78:12662-12670).

The linear decapeptide may be synthesized, for example, by first preparing two pentapeptides by solution or solid phase peptide synthesis, coupling the two pentapeptides to provide a linear decapeptide, then cyclization of the linear decapeptide to provide the cyclic intermediate (see Boger, et al., 1996, ibid; Ichikawa, et al., 2013, ibid.).

Alternatively, the linear decapeptide may be synthesized by solid phase synthesis by coupling a first amino acid, amino acid analogue or dipeptide to a solid phase resin, followed by step-wise addition of amino acids, amino acid analogues and/or dipeptides to provide the linear decapeptide, cleavage of the solid phase resin, and then cyclization of the linear decapeptide to provide the cyclic intermediate. Another approach that may be employed to synthesize the linear decapeptide by solid phase synthesis utilizes pre-formed dipeptides as described in the Examples herein (see also, Komatani, et al., 2023, Org. Lett., 25 (3): 543-548).

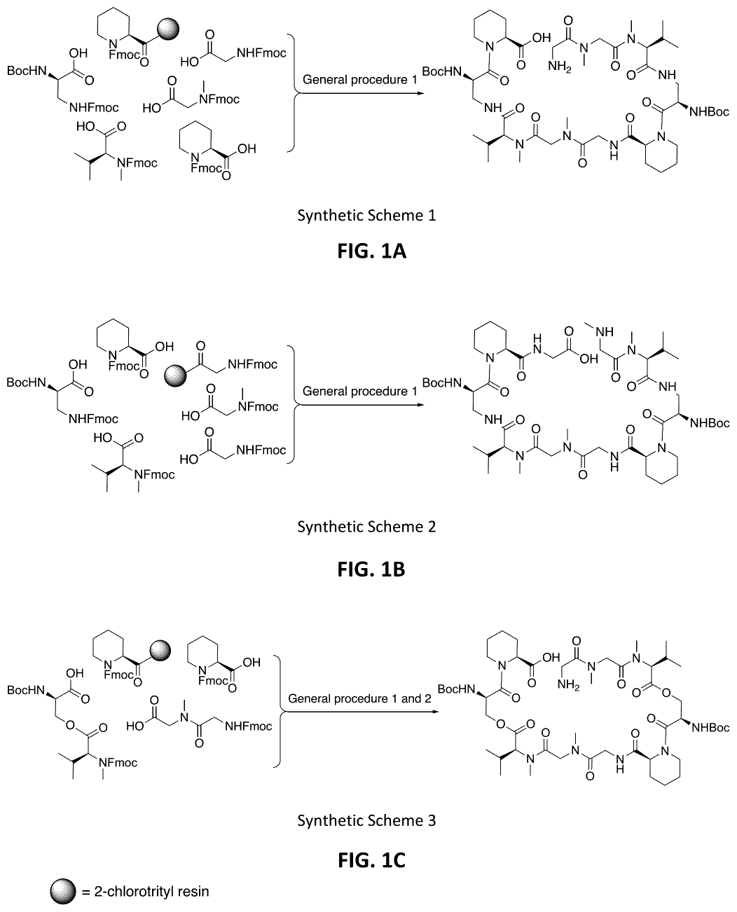

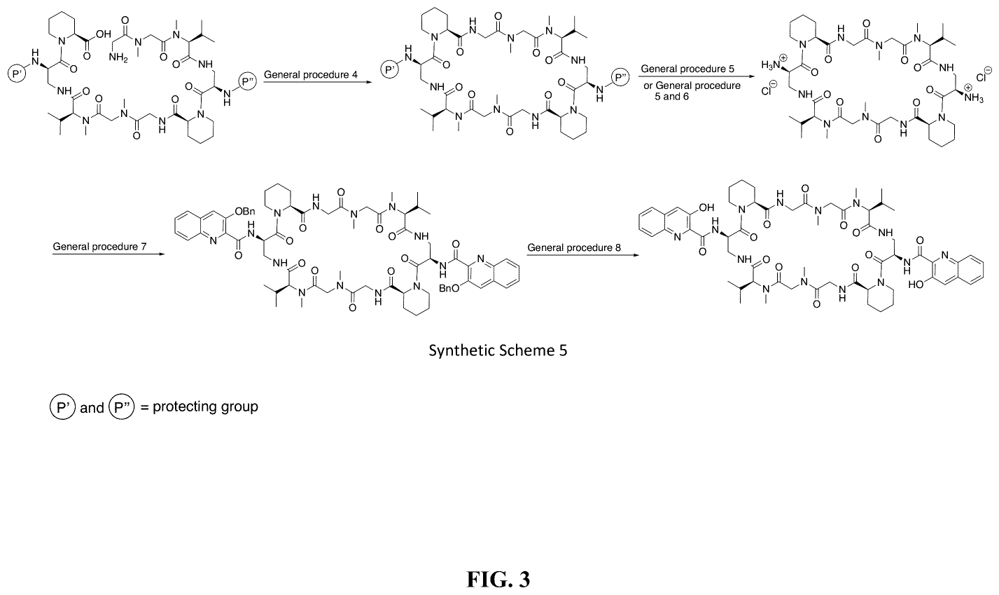

Examples of general synthetic schemes that may be employed in the preparation of compounds of Formula I are shown in FIGS. 1, 2, and 3, and described in the Examples herein.

### Conjugates

Certain embodiments of the present disclosure relate to conjugates comprising compounds of Formula I in which one or more compounds of Formula I are conjugated to a targeting moiety via a linker.

The conjugates of the present disclosure may comprise one or multiple compounds of Formula I conjugated to the targeting moiety. Multiple compounds of Formula I may be conjugated to the targeting moiety, for example, by attaching the compounds at different sites on the targeting moiety and/or by employing a linker that allows for attachment of multiple compounds to a single site on the targeting moiety.

Accordingly, certain embodiments of the present disclosure relate to conjugates of Formula X:

T-[L-(D)ₘ]ₙ (X)
wherein:
T is a targeting moiety;
L is a linker;
D is a compound of Formula I;
m is between 1 and about 10, and
n is between 1 and about 12.

In certain embodiments, m is between 1 and 8, for example, between 1 and 5, or between 1 and 3. In some embodiments, m is between 1 and 2. In some embodiments, m is 1.

In certain embodiments, n is between 1 and 10, for example, between 1 and 8, between 2 and 8, or between 2 and 6. In some embodiments, n is between 2 and 4.

In some embodiments, in conjugates of Formula X, D is a compound of Formula II. In some embodiments, in conjugates of Formula X, D is a compound of Formula III.

As noted above and reflected by parameters m and n in Formula X, a targeting moiety, “T,” can be conjugated to more than one compound of Formula I, “D.” Those skilled in the art will appreciate that, while any particular targeting moiety, T, is conjugated to an integer number of compounds, D, analysis of a preparation of the conjugate to determine the ratio of compound, D, to targeting moiety, T, may give a non-integer result, reflecting a statistical average. This ratio of compound, D, to targeting moiety, T, may generally be referred to as the drug-to-antibody ratio, or “DAR.” Accordingly, conjugate preparations having non-integer DARs are intended to be encompassed by Formula X. One skilled in the art will appreciate that the term “DAR” may be employed to define conjugates comprising targeting moieties other than antibodies.

Certain embodiments of the present disclosure relate to conjugates of Formula X that further comprise one or more hydrophilic moieties. Such hydrophilic moieties may be included in the linker component, L, of the conjugate or may be attached, for example covalently bonded, to the drug component, D, or both. The use of hydrophilic moieties to reduce the hydrophobicity of payload compounds in ADCs is well-known in the art (see, for example, International Patent Publication Nos. WO 2016/001485, WO 2018/234636 and WO 2019/081455; U.S. Pat. No. 9,636,421; and Ekholm, et al., 2019, Separations, 6:1). Examples of hydrophilic moieties are described above and include, but are not limited to, various lengths of PEG or mPEG, polysarcosine, various saccharides (including β-glucuronide), sulfonate groups, sulfonamide groups, carboxylate groups and pyrophosphate diesters. Incorporation of hydrophilic macrocycles such as cyclodextrins and crown ethers into ADCs has also been reported (Evans, et al., 2022, Front. Pharmacol., 13:764540).

In certain embodiments, in conjugates of Formula X, the linker component, L, further comprises a hydrophilic moiety. In some embodiments, in conjugates of Formula X, the linker component, L, further comprises one or more PEG units (for example, as a pendant group), one or more saccharides (such as β-glucuronide), one or more sulfonate groups, or one or more pyrophosphate diesters.

In certain embodiments, in conjugates of Formula X, the drug component, D, further comprises one or more hydrophilic moieties (HM). In some embodiments, in conjugates of Formula X, the drug component, D, further comprises one or more hydrophilic moieties, HM, which can be enzymatically cleaved from the drug molecule. In some embodiments, in conjugates of Formula X, the drug component, D, further comprises one or more hydrophilic moieties, HM, which are enzymatically cleavable and linked to the drug component via one or more self-immolative moieties, L^{A}.

Examples of self-immolative (or self-elimination) moieties are described above and include, but are not limited to, p-aminobenzyl (PAB) groups, p-aminobenzyloxycarbonyl (PABC) groups, methylated ethylene diamine (MED), and aromatic compounds that are electronically similar to a PAB or PABC group (such as heterocyclic derivatives, for example, 2-aminoimidazol-5-methanol derivatives as described in U.S. Pat. No. 7,375,078).

In certain embodiments, in conjugates of Formula X, drug-linker L-(D) m has Formula XI:

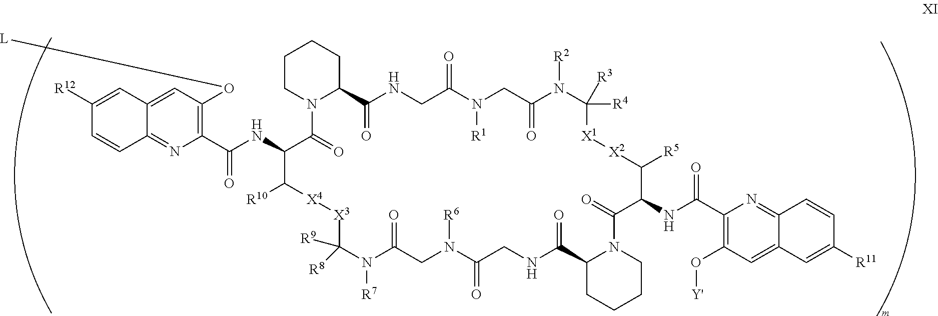

wherein:
L is the linker;
m is between 1 and about 10;
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—;
X² and X⁴ are each independently —NR¹⁴— or —O—, and R¹⁴ is H or C₁-C₂ alkyl, and
Y′ is H or (L^{A})ₚ-HM, where L^{A} is a self-immolative moiety, p is 0, 1 or 2, and HM is a hydrophilic moiety.

In certain embodiments, in drug-linkers of Formula XI, when R² and R³ together with the N and C atoms to which they are bonded form a 6-membered heterocycloalkyl, and R⁷ and R⁸ together with the N and C atoms to which they are bonded form a 6-membered heterocycloalkyl, and X¹ and X³ are each —C(O)—, then one of X² and X⁴ is other than —NH—.

In certain embodiments, in drug-linkers of Formula XI, when R² and R³ together with the N and C atoms to which they are bonded form a 5-membered heterocycloalkyl, and R⁷ and R⁸ together with the N and C atoms to which they are bonded form a 5-membered heterocycloalkyl, and X¹ and X³ are each —C(O)—, then one of X² and X⁴ is other than —NH—.

In certain embodiments, in drug-linkers of Formula XI, R¹¹ and R¹² are each H.

In certain embodiments, in drug-linkers of Formula XI, when X¹ is —CH₂—, X² is —O—, or when X³ is —CH₂—, X⁴ is —O—.

In certain embodiments, in drug-linkers of Formula XI, X¹ is —C(O)— and X² is —NR¹⁴— or —O—, or X³ is —C(O)— and X⁴ is —NR¹⁴— or —O—. In some embodiments, in drug-linkers of Formula XI, X¹ and X³ are each —C(O)—, and X² and X⁴ are each independently —NR¹⁴— or —O—.

In certain embodiments, in drug-linkers of Formula XI, X¹ is —C(O)— and X² is —NR¹⁴—, or X³ is —C(O)— and X⁴ is —NR¹⁴—. In certain embodiments, in drug-linkers of Formula XI, X¹ and X³ are each —C(O)—, and X² and X⁴ are each —NR¹⁴.

Combinations of any of the foregoing embodiments for drug-linkers of Formula XI are also contemplated and each combination forms a separate embodiment for the purposes of the present disclosure.

In certain embodiments, in conjugates of Formula X, drug-linker L-(D) m has Formula XII:

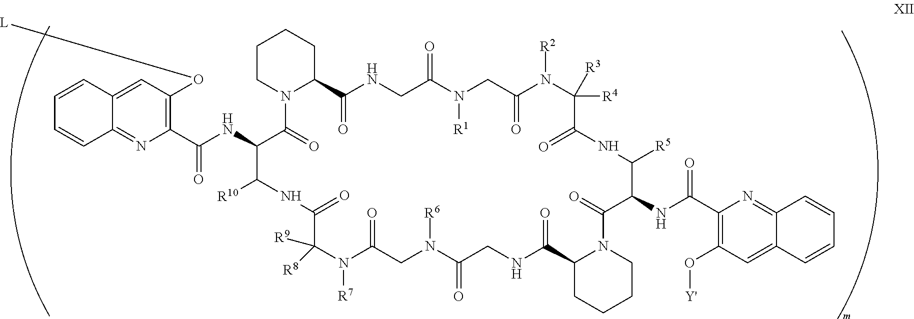

wherein:
L, m, R¹, R², R³, R⁴, R⁵, R⁶, R⁷, R⁸, R⁹, R¹⁰ and Y′ are as defined for Formula XI.

In certain embodiments, in drug-linkers of Formula XI or Formula XII:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 7-membered heterocycloalkyl, and R⁴ is H.

In certain embodiments, in drug-linkers of Formula XI or Formula XII:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl.

In certain embodiments, in drug-linkers of Formula XI or Formula XII:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 7-membered heterocycloalkyl, and R⁹ is H.

In certain embodiments, in drug-linkers of Formula XI or Formula XII:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl.

In certain embodiments, in drug-linkers of Formula XI or Formula XII, R⁵ and R¹⁰ are each H.

In certain embodiments, in drug-linkers of Formula XI or Formula XII, m is between 1 and 3.

In certain embodiments, in drug-linkers of Formula XI or Formula XII, Y′ is H.

In certain embodiments, in drug-linkers of Formula XI or Formula XII, Y′ is (L^{A})ₚ-HM. In some embodiments, in drug-linkers of Formula XI or Formula XII, Y′ is (L^{A})ₚ-HM, HM is an enzymatically cleavable hydrophilic moiety and p is 1. In some embodiments, in drug-linkers of Formula XI or Formula XII, Y′ is (L^{A})ₚ-HM, HM is an enzymatically cleavable hydrophilic moiety and p is 0.

In certain embodiments, in drug-linkers of Formula XI or Formula XII, (L^{A})ₚ-HM has one of the following structures:

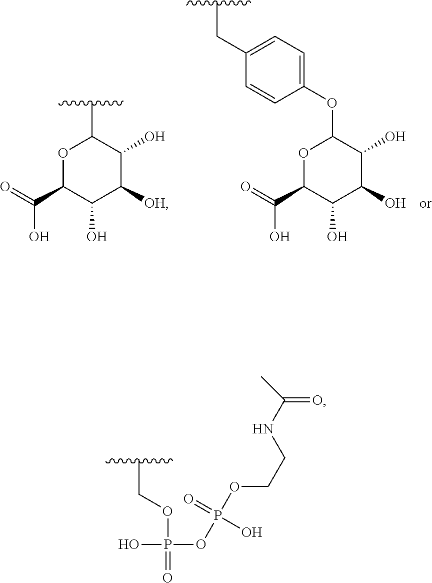 [SMILES: *C1O[C@H](C(=O)O)[C@@H](O)[C@H](O)[C@H]1O ; *Cc1ccc(OC2O[C@H](C(=O)O)[C@@H](O)[C@H](O)[C@H]2O)cc1 ; *COP(=O)(O)OP(=O)(O)OCCNC(C)=O]

where [symbol] indicates the point of attachment to the drug-linker.

Combinations of any of the foregoing embodiments for each of drug-linkers of Formula XI and drug-linkers of Formula XII are also contemplated and each combination forms a separate embodiment for the purposes of the present disclosure.

Certain embodiments of the present disclosure relate to conjugates having Formula XIII:

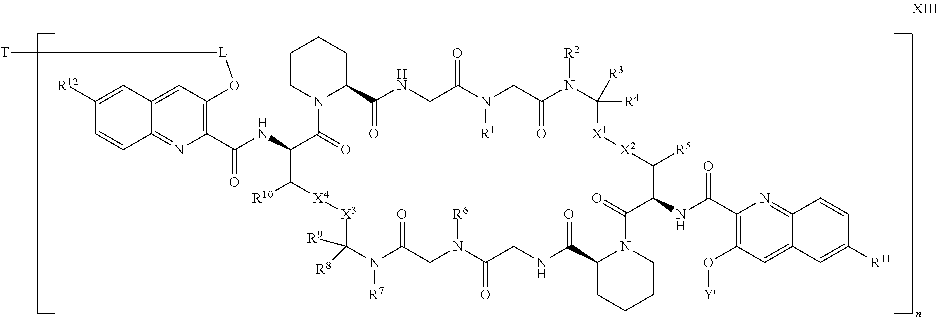

wherein:
T is a targeting moiety;
L is a linker;
n is between 1 and about 12;
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—;
X² and X⁴ are each independently —NR¹⁴— or —O—, and R¹⁴ is H or C₁-C₂ alkyl, and
Y′ is H or (L^{A})ₚ-HM, where L^{A} is a self-immolative moiety, p is 0, 1 or 2, and HM is a hydrophilic moiety.

In certain embodiments, in conjugates of Formula XIII, when R² and R³ together with the N and C atoms to which they are bonded form a 6-membered heterocycloalkyl, and R⁷ and R⁸ together with the N and C atoms to which they are bonded form a 6-membered heterocycloalkyl, and X¹ and X³ are each —C(O)—, then one of X² and X⁴ is other than —NH—.

In certain embodiments, in conjugates of Formula XIII, when R² and R³ together with the N and C atoms to which they are bonded form a 5-membered heterocycloalkyl, and R⁷ and R⁸ together with the N and C atoms to which they are bonded form a 5-membered heterocycloalkyl, and X¹ and X³ are each —C(O)—, then one of X² and X⁴ is other than —NH—.

In certain embodiments, in conjugates of Formula XIII, R¹¹ and R¹² are each H.

In certain embodiments, in conjugates of Formula XIII, when X¹ is —CH₂—, X² is —O—, or when X³ is —CH₂—, X⁴ is —O—.

In certain embodiments, in conjugates of Formula XIII, X¹ is —C(O)— and X² is —NR¹⁴—or —O—, or X³ is —C(O)— and X⁴ is —NR¹⁴— or —O—. In some embodiments, in compounds of Formula I, X¹ and X³ are each —C(O)—, and X² and X⁴ are each independently —NR¹⁴— or —O—.

In certain embodiments, in conjugates of Formula XIII, X¹ is —C(O)— and X² is —NR¹⁴—, or X³ is —C(O)— and X⁴ is —NR¹⁴—. In certain embodiments, in conjugates of Formula XIII, X¹ and X³ are each —C(O)—, and X² and X⁴ are each —NR¹⁴.

Combinations of any of the foregoing embodiments for conjugates of Formula XIII are also contemplated and each combination forms a separate embodiment for the purposes of the present disclosure.

Certain embodiments of the present disclosure relate to conjugates having Formula XIV:

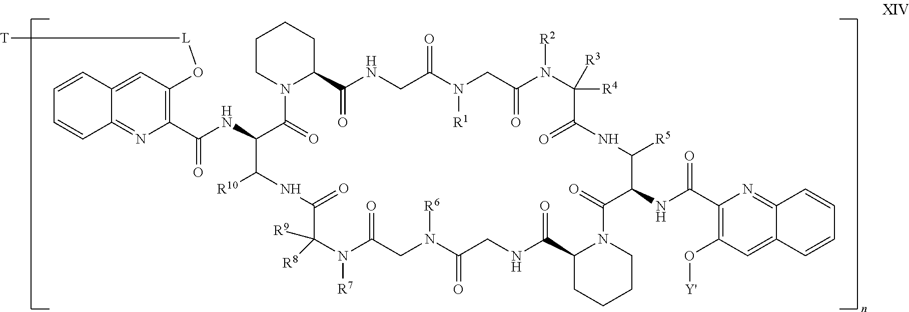

wherein:
T, L, n, R¹, R², R³, R⁴, R⁵, R⁶, R⁷, R⁸, R⁹, R¹⁰ and Y′ are as defined for Formula XIII.

In certain embodiments, in conjugates of Formula XIII or Formula XIV:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 7-membered heterocycloalkyl, and R⁴ is H.

In certain embodiments, in conjugates of Formula XIII or Formula XIV:
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl.

In certain embodiments, in conjugates of Formula XIII or Formula XIV:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 7-membered heterocycloalkyl, and R⁹ is H.

In certain embodiments, in conjugates of Formula XIII or Formula XIV:
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl.

In certain embodiments, in conjugates of Formula XIII or Formula XIV, R⁵ and R¹⁰ are each H.

In certain embodiments, in conjugates of Formula XIII or Formula XIV, n is between 1 and 8. In some embodiments, in conjugates of Formula XIII or Formula XIV, n is between 2 and 8, or between 2 and 4.

In certain embodiments, in conjugates of Formula XIII or Formula XIV, Y′ is H.

In certain embodiments, in conjugates of Formula XIII or Formula XIV, one of Y′ and Y″ is (L^{A})ₚ-HM, and the other of Y′ and Y″ is H. In some embodiments, in conjugates of Formula XIII or Formula XIV, HM is an enzymatically cleavable hydrophilic moiety and p is 1. In some embodiments, in conjugates of Formula XIII or Formula XIV, HM is an enzymatically cleavable hydrophilic moiety and p is 0.

In certain embodiments, in conjugates of Formula XIII or Formula XIV, (L^{A})ₚ-HM has one of the following structures:

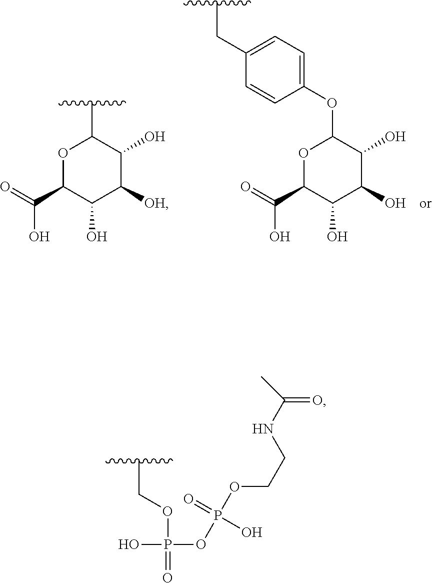 [SMILES: *C1O[C@H](C(=O)O)[C@@H](O)[C@H](O)[C@H]1O ; *Cc1ccc(OC2O[C@H](C(=O)O)[C@@H](O)[C@H](O)[C@H]2O)cc1 ; *COP(=O)(O)OP(=O)(O)OCCNC(C)=O]

where [symbol] indicates the point of attachment to the drug-linker.

Combinations of any of the foregoing embodiments for each of conjugates of Formula XIII and conjugates of Formula XIV are also contemplated and each combination forms a separate embodiment for the purposes of the present disclosure.

It is to be understood that reference to conjugates of Formula X throughout this disclosure includes, in various embodiments, conjugates of Formula XIII and Formula XIV to the same extent as if embodiments reciting each of these formulae individually were specifically recited.

Certain embodiments of the present disclosure relate to conjugates of Formula X in which D is selected from the compounds set forth in Tables 1 and 2. Certain embodiments relate to conjugates of Formula X in which D is selected from the compounds set forth in Tables 1 and 2 that further comprise one or more hydrophilic moieties linked to D optionally via one or more self-immolative moities.

Certain embodiments of the present disclosure relate to conjugates of Formula X comprising a drug-linker selected from the drug-linkers set forth in Table 3.

Certain embodiments of the present disclosure relate to drug-linkers of Formula XI or Formula XII that may be used to prepare conjugates, such as conjugates of Formula X. In certain embodiments, the drug-linker may be selected from the drug-linkers set forth in Table 3.

TABLE 3
Exemplary Drug-Linkers

| Drug- | |
|---|---|
| Linker | Structure |
| DL-1 | 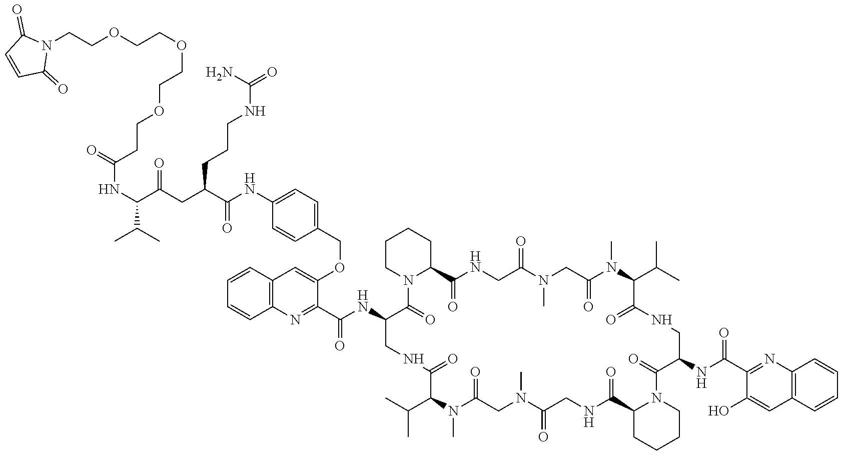 [SMILES: CC(C)[C@H](NC(=O)CCOCCOCCOCCN1C(=O)C=CC1=O)C(=O)C[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-2 | 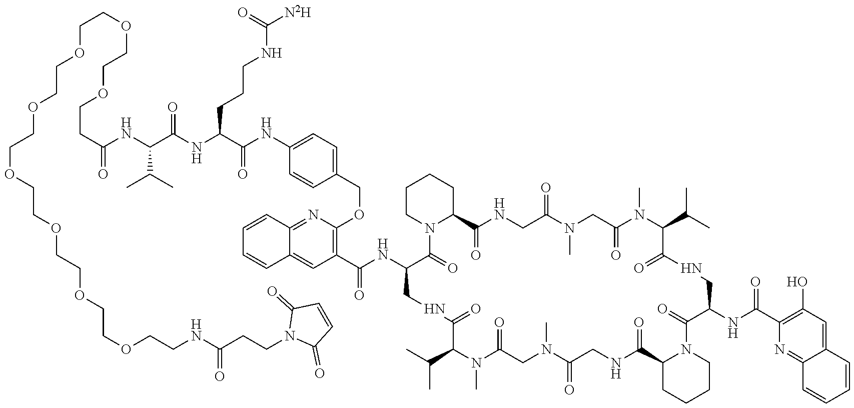 |
| DL-3 | 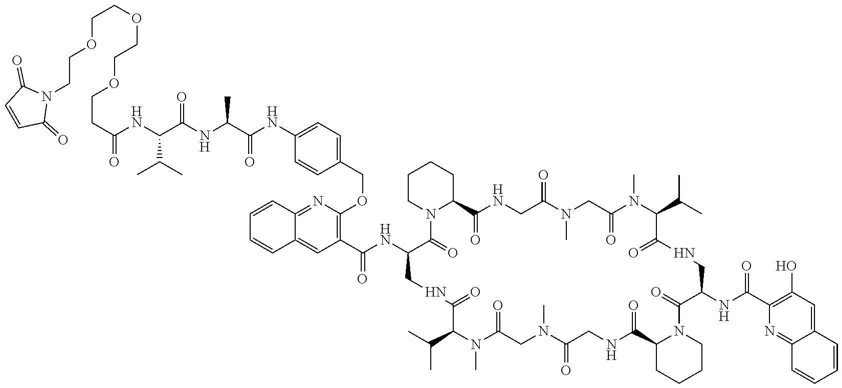 [SMILES: CC(C)[C@H](NC(=O)CCOCCOCCOCCN1C(=O)C=CC1=O)C(=O)N[C@@H](C)C(=O)Nc1ccc(COc2nc3ccccc3cc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-4 | 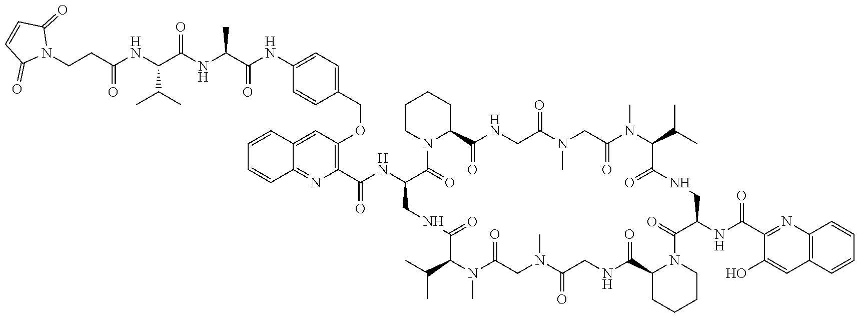 [SMILES: CC(C)[C@H](NC(=O)CCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-5 | 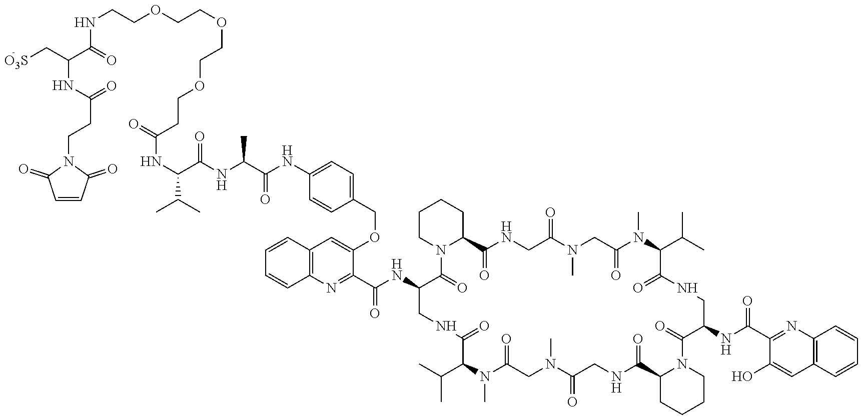 |
| DL-6 | 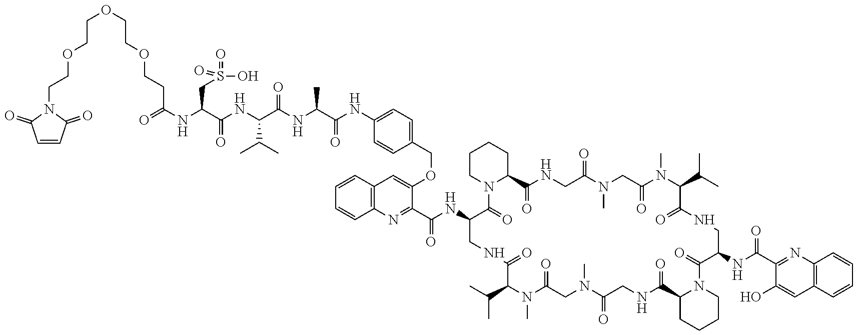 [SMILES: CC(C)[C@H](NC(=O)[C@H](CS(=O)(=O)O)NC(=O)CCOCCOCCOCCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-7 | 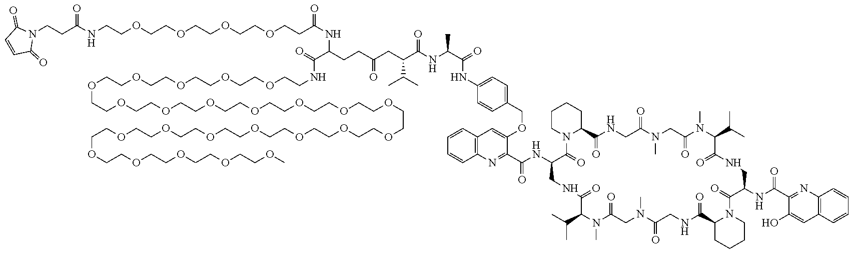 [SMILES: COCCOCCOCCOCCOCCOCCOCCOCCOCCOCCOCCOCCOCCOCCOCCOCCOCCOCCOCCOCCOCCOCCOCCOCCNC(=O)C(CCC(=O)C[C@H](C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1)C(C)C)NC(=O)CCOCCOCCOCCOCCNC(=O)CCN1C(=O)C=CC1=O] |
| DL-8 | 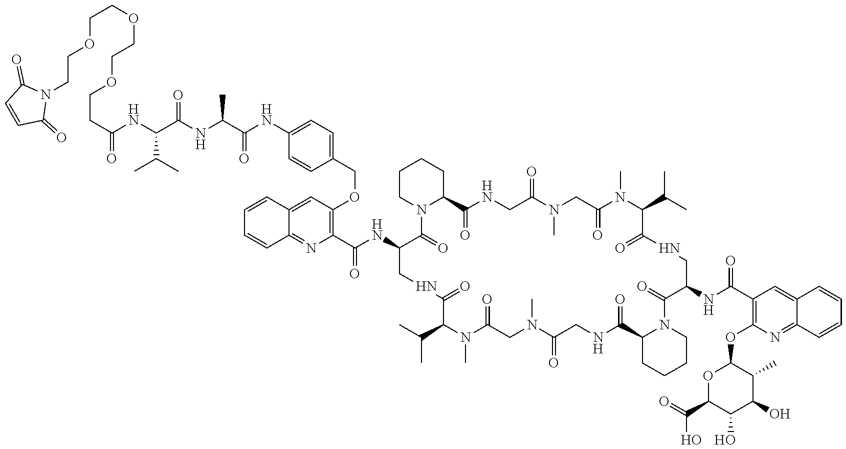 [SMILES: CC(C)[C@H](NC(=O)CCOCCOCCOCCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3cc4ccccc4nc3O[C@@H]3O[C@H](C(=O)O)[C@@H](O)[C@H](O)[C@H]3C)CNC(=O)[C@H](C(C)C)N(C)C(=O)CN(C)C(=O)CNC(=O)[C@@H]3CCCCN3C2=O)cc1] |
| DL-9 | 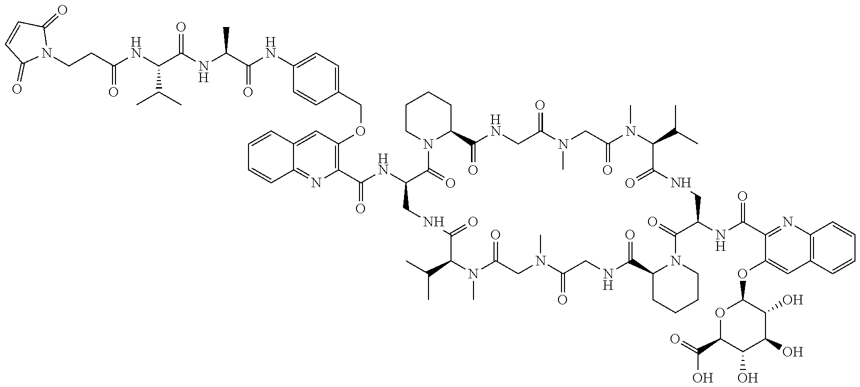 [SMILES: CC(C)[C@H](NC(=O)CCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1] |
| DL-10 | 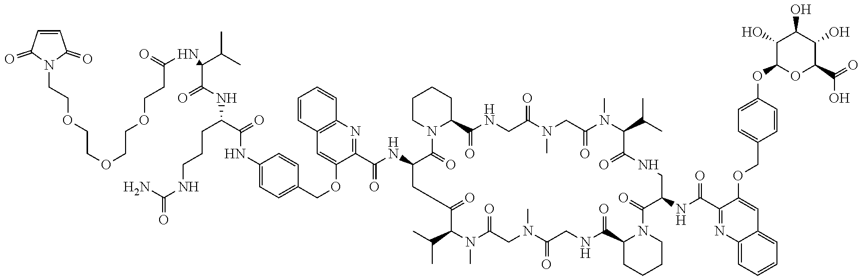 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CCC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(O[C@@H]4O[C@H](C(=O)O)[C@@H](O)[C@H](O)[C@H]4O)cc3)CNC(=O)[C@H](C(C)C)N(C)C(=O)CN(C)C(=O)CNC(=O)[C@@H]3CCCCN3C2=O)cc1] |
| DL-11 | 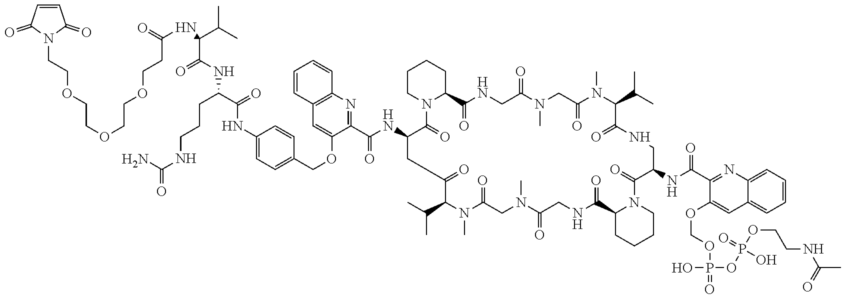 [SMILES: CC(=O)NCCOP(=O)(O)OP(=O)(O)OCOc1cc2ccccc2nc1C(=O)N[C@@H]1CNC(=O)[C@H](C(C)C)N(C)C(=O)CN(C)C(=O)CNC(=O)[C@@H]2CCCCN2C(=O)[C@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)CCC(=O)[C@H](C(C)C)N(C)C(=O)CN(C)C(=O)CNC(=O)[C@@H]2CCCCN2C1=O] |
| DL-12 | 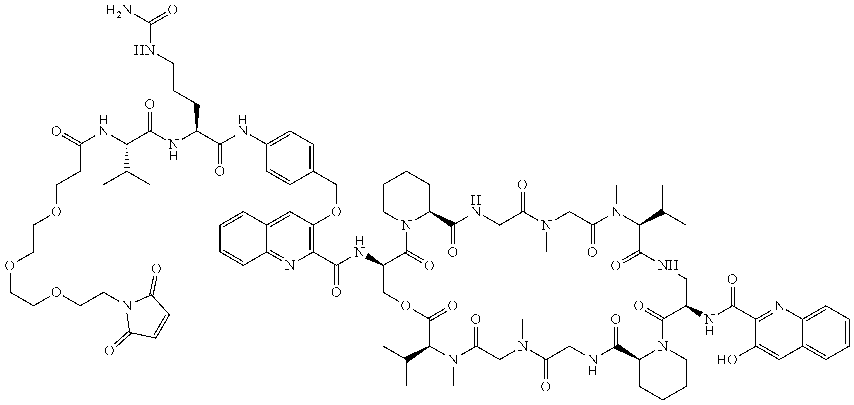 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-13 | 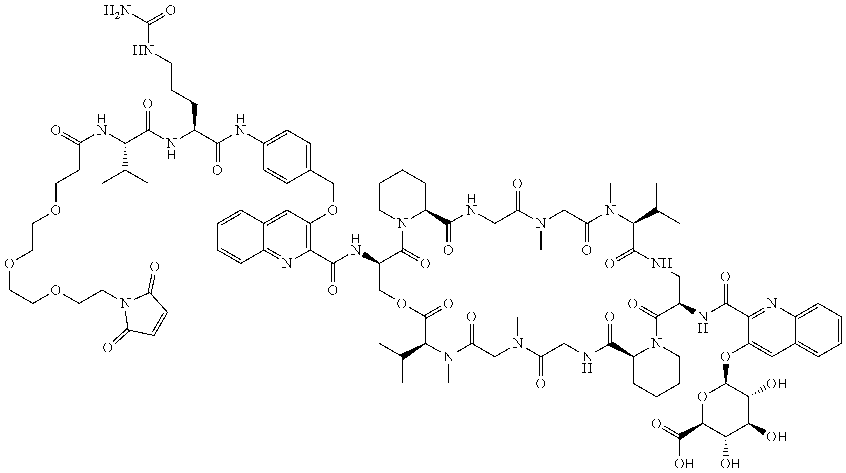 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1] |
| DL-14 | 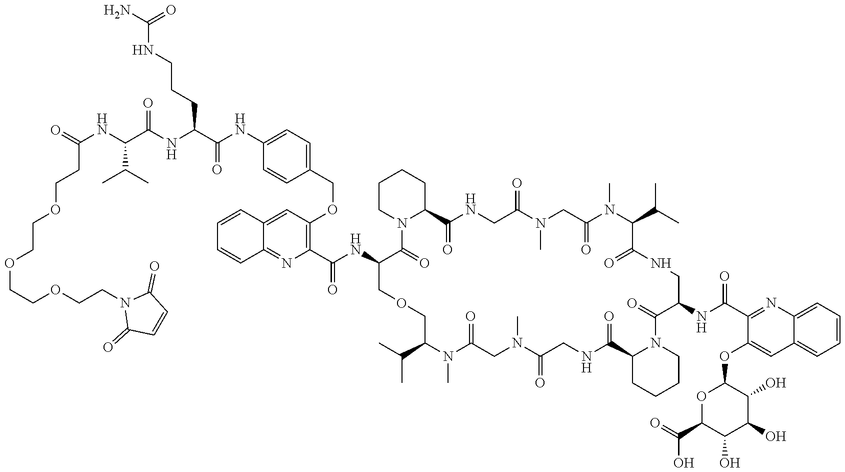 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1] |
| DL-15 | 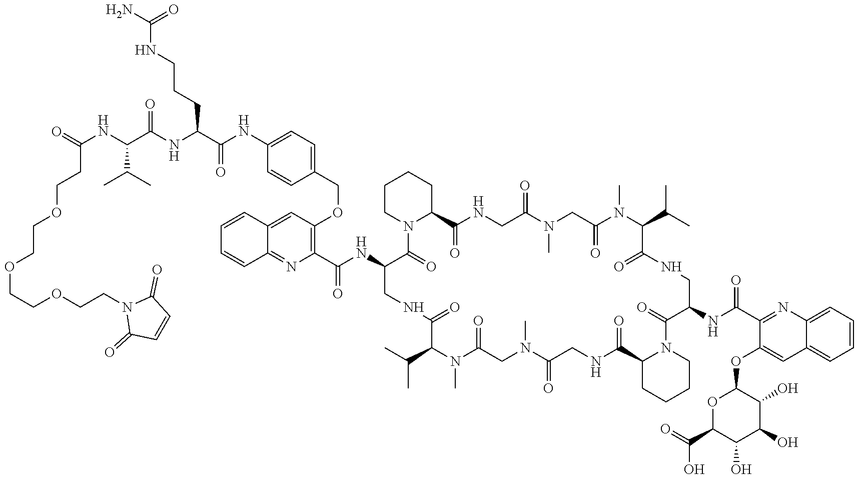 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1] |
| DL-16 | 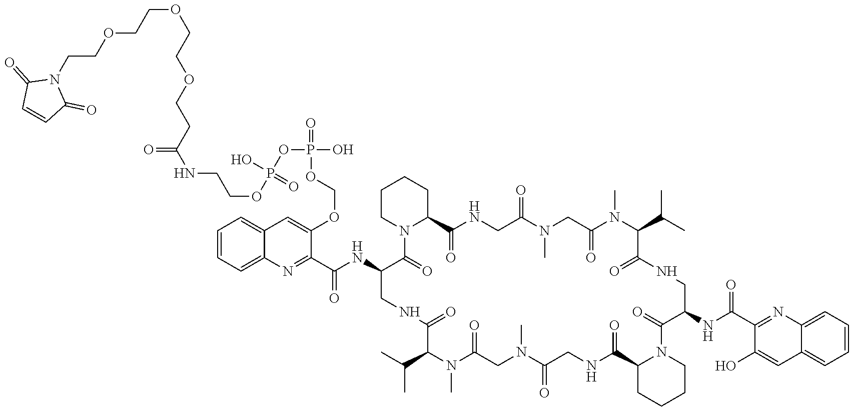 [SMILES: CC(C)[C@H]1C(=O)NC[C@@H](NC(=O)c2nc3ccccc3cc2OCOP(=O)(O)OP(=O)(O)OCCNC(=O)CCOCCOCCOCCN2C(=O)C=CC2=O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C] |
| DL-17 | 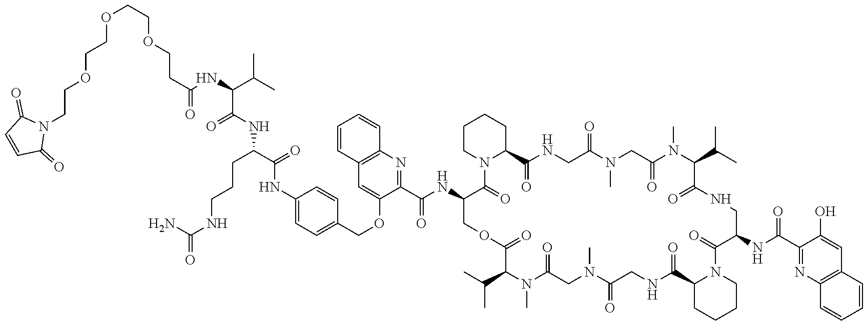 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1] |
| DL-18 | 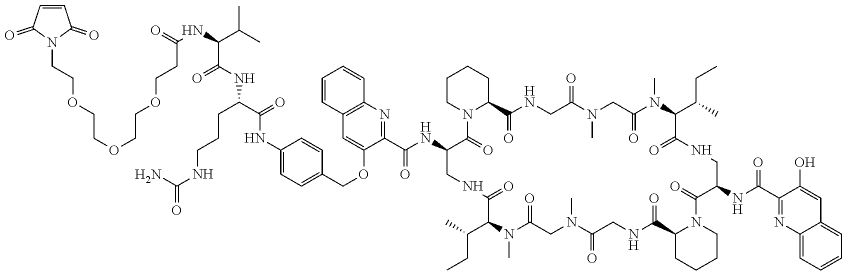 [SMILES: CC[C@H](C)[C@H]1C(=O)NC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2O)C(=O)N2CCCC[C@H]2C(=O)NCC(=O)N(C)CC(=O)N1C] |
| DL-19 | 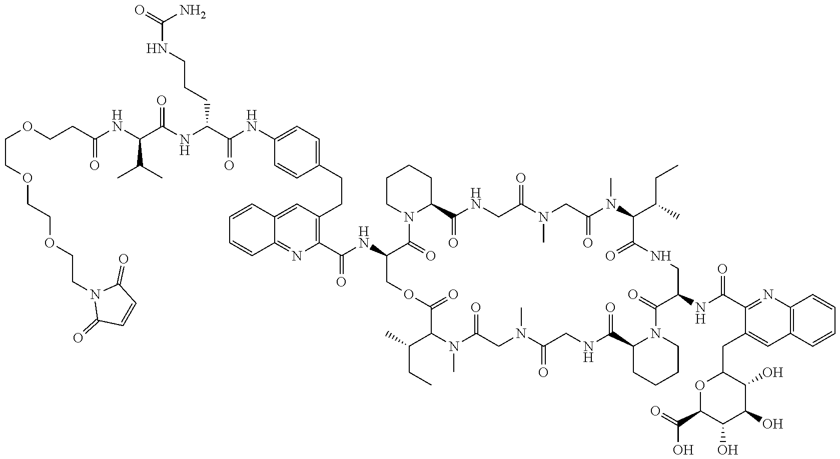 [SMILES: CC[C@H](C)C1C(=O)OC[C@@H](NC(=O)c2nc3ccccc3cc2CCc2ccc(NC(=O)[C@@H](CCCNC(N)=O)NC(=O)[C@H](NC(=O)CCOCCOCCOCCN3C(=O)C=CC3=O)C(C)C)cc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2CC2O[C@H](C(=O)O)[C@@H](O)[C@H](O)[C@H]2O)C(=O)N2CCCC[C@H]2C(=O)NCC(=O)N(C)CC(=O)N1C] |
| DL-20 | 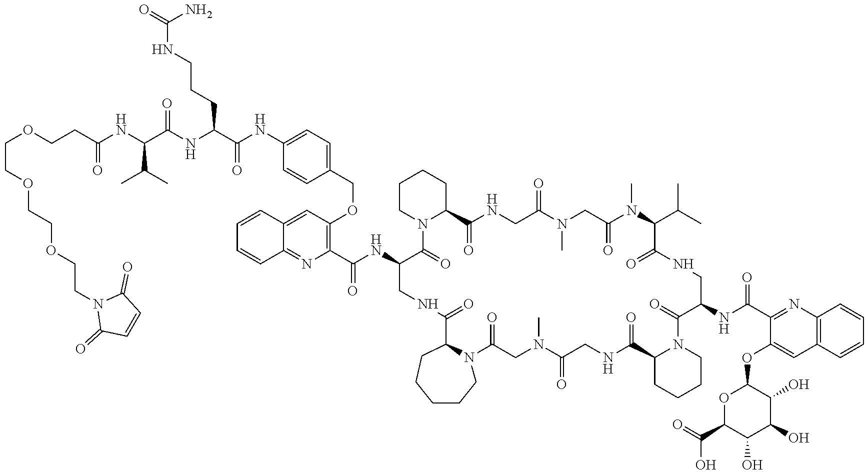 [SMILES: CC(C)[C@H]1C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(C)CC(=O)N2CCCCC[C@H]2C(=O)NC[C@@H](NC(=O)c2nc3ccccc3cc2OCc2ccc(NC(=O)[C@H](CCCNC(N)=O)NC(=O)[C@H](NC(=O)CCOCCOCCOCCN3C(=O)C=CC3=O)C(C)C)cc2)C(=O)N2CCCC[C@H]2C(=O)NCC(=O)N(C)CC(=O)N1C] |
| DL-21 | 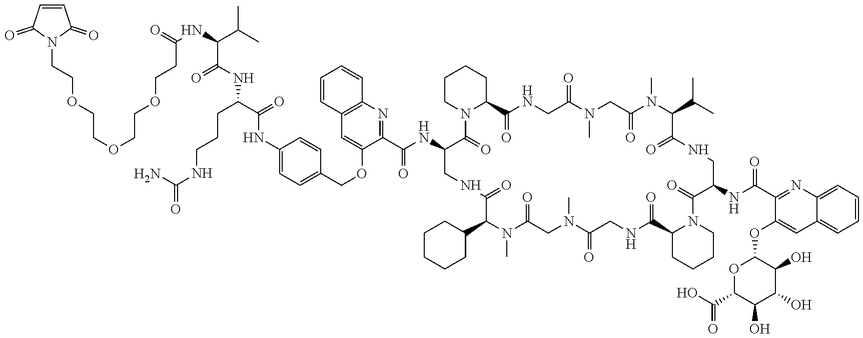 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C3CCCCC3)N(C)C(=O)CN(C)C(=O)CNC(=O)[C@@H]3CCCCN3C(=O)[C@H](NC(=O)c3nc4ccccc4cc3O[C@H]3O[C@@H](C(=O)O)[C@H](O)[C@@H](O)[C@@H]3O)CNC(=O)[C@H](C(C)C)N(C)C(=O)CN(C)C(=O)CNC(=O)[C@@H]3CCCCN3C2=O)cc1] |
| DL-22 | 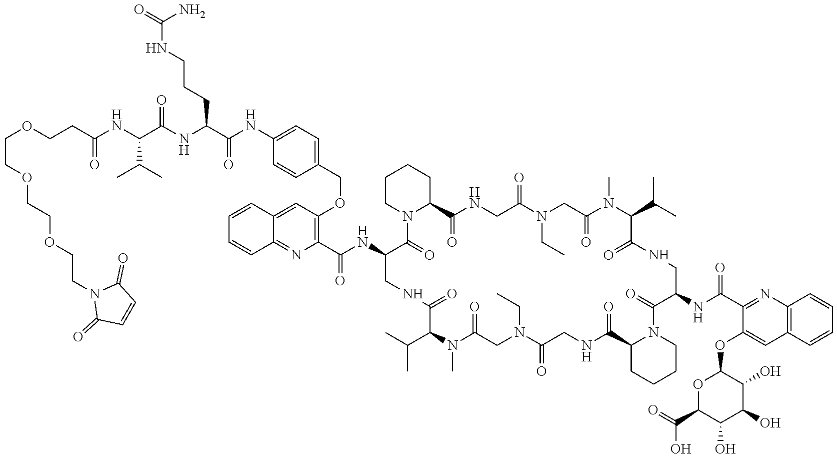 [SMILES: CCN1CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(CC)CC(=O)N(C)[C@@H](C(C)C)C(=O)NC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC1=O] |
| DL-23 | 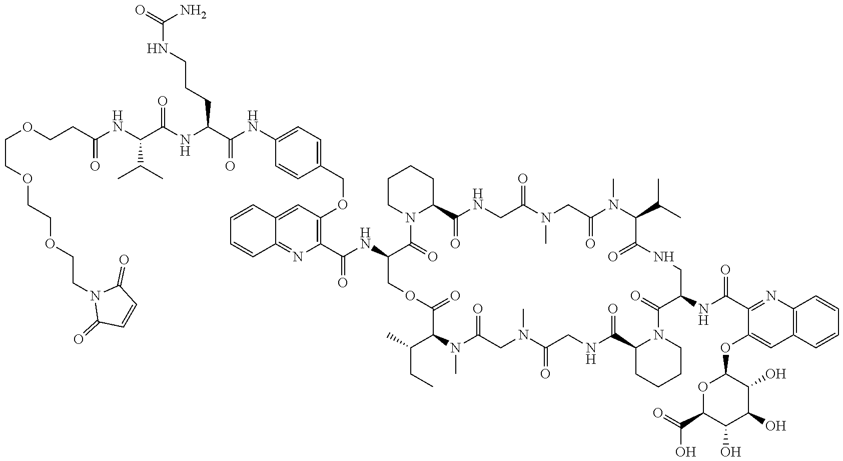 [SMILES: CC[C@H](C)[C@H]1C(=O)OC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(C)CC(=O)N1C] |

### Targeting Moiety, T

The targeting moiety, T, comprised by the conjugates described herein is a molecule that binds, reactively associates or complexes with a receptor, antigen or other receptive moiety associated with a given target cell population. Typically, targeting moiety, T, functions to deliver the compound of Formula I, D, to the target cell population with which targeting moiety, T, reacts. Examples of targeting moieties include, but are not limited to, proteins (such as antibodies, antibody fragments and growth factors), glycoproteins, peptides (such as bombesin and gastrin-releasing peptide), lectins, vitamins (such as folic acid) and nutrient-transport molecules (such as transferrin).

Typically, targeting moiety, T, will be bonded to linker, L, via a heteroatom of the targeting moiety, such as a sulfur (for example, from a sulfhydryl group of targeting moiety, T), oxygen (for example, from a carbonyl, carboxyl or hydroxyl group of targeting moiety, T) or nitrogen (for example, from a primary or secondary amino group of targeting moiety, T). These heteroatoms may be naturally present on targeting moiety, T, introduced through engineering and/or expression, or introduced via chemical modification using techniques known in the art.

In certain embodiments of the present disclosure, targeting moiety, T, is an antibody or antigen-binding antibody fragment. Accordingly, certain embodiments of the present disclosure relate to antibody-drug conjugates (ADCs) comprising compounds of Formula I and having Formula X, Formula XIII or Formula XIV.

When the conjugate according to the present disclosure is an ADC, the targeting moiety may be a full-size polyclonal or monoclonal antibody, a domain antibody (dAb) or an antibody mimetic (such as an affibody, a DARPin, an anticalin, a versabody, a duocalin, a lipocalin or an avimer), or it may be an antigen-binding antibody fragment (such as Fab, scFab, Fab′, F(ab′)₂, Fv or scFv). The antibody or antigen-binding antibody fragment is typically directed to a particular antigen, for example, a tumour-associated antigen.

Methods of producing polyclonal and monoclonal antibodies are known in the art. By way of example, monoclonal antibodies may be produced by methods including, but not limited to, the hybridoma technique originally described by Kohler and Milstein (1975, Nature 256:495-497), the human B cell hybridoma technique (Kozbor et al., 1983, Immunology Today 4:72), the EBV-hybridoma technique (Cole et al., 1985, Monoclonal Antibodies and Cancer Therapy, Alan R. Liss, Inc., pp. 77-96), and the Selected Lymphocyte Antibody Method (SLAM) (Babcook, et al., 1996, Proc Natl Acad Sci USA, 93 (15): 7843-8; McLean et al., 2005, J Immunol., 174 (8): 4768-4778). Antibodies of various immunoglobulin classes including IgG, IgM, IgE, IgA, and IgD and subclasses thereof, may find application in the ADCs in various embodiments. In some embodiments, the antibody is of the IgG class.

In certain embodiments, targeting moiety, T, may be a monoclonal antibody. The monoclonal antibody may be, for example, a non-human monoclonal antibody (such as a mouse antibody), a human monoclonal antibody, a humanized monoclonal antibody or a chimeric antibody (for example, a human-mouse antibody). Human monoclonal antibodies may be made by any of numerous techniques known in the art (see, for example, Teng et al., 1983, Proc. Natl. Acad. Sci. USA 80:7308-7312; Kozbor et al., 1983, Immunology Today 4:72-79; Olsson et al., 1983, Meth. Enzymol. 92:3-16; Huse et al., 1989, Science 246:1275-1281, and U.S. Pat. No. 8,012,714). Chimeric and humanized monoclonal antibodies can be produced by recombinant DNA techniques known in the art, for example using methods described in International Patent Publication Nos. WO 87/02671 and WO 86/01533; European Patent Publication Nos. 0 184 187; 0 171 496 and 0 173 494; U.S. Pat. Nos. 4,816,567 and 5,225,539; Berter et al., 1988, Science 240:1041-1043; Liu et al., 1987, J. Immunol., 139:3521-3526; Sun et al., 1987, Proc. Natl. Acad. Sci. USA, 84:214-218; Wood et al., 1985, Nature, 314:446-449; Shaw et al., 1988, J. Natl. Cancer Inst., 80:1553-1559; Oi et al., 1986, BioTechniques, 4:214; Jones et al., 1986, Nature, 321:552-525, and Beidler et al., 1988, J. Immunol., 141:4053-4060. Antibodies immunospecific for a given target antigen may also obtained commercially.

In certain embodiments, the antibody included in the conjugate may be a bispecific antibody. Methods for making bispecific antibodies are known in the art (see, for example, Milstein et al., 1983, Nature, 305:537-539; Traunecker et al., 1991, EMBO J., 10:3655-3659; Suresh et al., 1986, Meth. Enzymol., 121:210; Rodrigues et al., 1993, J. Immunol., 151:6954-6961; Carter et al., 1992, Bio Technology, 10:163-167; Carter et al., 1995, J. Hematotherapy, 4:463-470; Merchant et al., 1998, Nature Biotechnology, 16:677-681, and International (PCT) Publication Nos. WO 94/04690, WO 2012/032080, WO 2012/058768 and WO 2013/063702).

In certain embodiments, targeting moiety, T, comprised by the conjugate is an antibody or antigen-binding antibody fragment that binds to a tumour-associated antigen (TAA). Examples of TAAs that may be targeted include, but are not limited to, B7H3, B7H4, carbonic anhydrase IX, carcinoembryonic antigen (CEA), CD19, CD20, CD22, CD30, CD44, CD52, CD70, c-Met, colony stimulating factor 1 receptor (CSFIR), CTLA-4, epidermal growth factor receptor (EGFR), EpCAM, folate binding protein, folate receptor-α, glypican-3, gpA33, HER2, HER3, human leukocyte antigen-DR isotype (HLA-DR), insulin-like growth factor 1 receptor (IGF1R), Lewis Y, mesothelin, mucins, nectin-4, platelet-derived growth factor receptor-α (PDGFR-α), PD1, PD-L1, prostate specific membrane antigen (PSMA), RANKL, trophoblast cell-surface antigen 2 (TROP-2), vascular endothelial growth factor (VEGF) and vascular endothelial growth factor receptor (VEGFR).

Examples of antibodies that target TAAs and are useful in the treatment of cancer include, but are not limited to, atezolizumab (Tecentriq™)—a humanized anti-PD-L1 antibody for treatment of urothelial carcinoma and NSCLC; belimumab (Benlysta®)—a human anti-BAFF antibody for treatment of non-Hodgkin's lymphoma; bevacizumab (Avastin®)—a humanized anti-VEGF antibody for treatment of NSCLC, renal cell carcinoma, glioblastoma, cervical, ovarian and colorectal cancers; cetuximab (Erbitux®)—a chimeric anti-EGFR monoclonal antibody for treatment of colorectal and head and neck cancer; denosumab (Xgeva®)—a human anti-RANKL antibody for treatment of bone metastasis from solid tumours; dinutuximab (Unituxin®)—a chimeric anti-GD2 antibody for treatment of pediatric neuroblastoma; ipilimumab (Yervoy®)—a human anti-CTLA-4 antibody for treatment of melanoma; mogamulizumab (Poteligeo®)—a humanized anti-CCR4 antibody for treatment of adult T-cell leukemia/lymphoma; necitumumab (Portrazza™)—a human anti-EGFR monoclonal antibody for treatment of non-small cell lung cancer (NSCLC); nivolumab (Opdivo®)—a human anti-PD1 antibody for treatment of melanoma, NSCLC, colorectal and head and neck cancer; olaratumab (Lartruvo®)—a human anti-PDGFR-α antibody for treatment of soft tissue sarcoma; panitumumab (Vectibix®)—a human anti-EGFR monoclonal antibody for treatment of colorectal cancer; pembrolizumab (Keytruda®)—a humanized anti-PD1 antibody for treatment of melanoma, NSCLC and head and neck cancer; pertuzumab (Perjeta®)—a humanized anti-HER2 monoclonal antibody for treatment of HER2-overexpressing breast cancer; ramucirumab (Ciramza®)—a human anti-VEGFR-2 antibody for treatment of gastric cancer, colorectal cancer and NSCLC; rituximab (Rituxin®)—a chimeric anti-CD20 monoclonal antibody for treatment of non-Hodgkin's lymphoma and chronic lymphocytic leukemia (CLL), and trastuzumab (Herceptin®)—a humanized anti-HER2 monoclonal antibody for treatment of HER2-overexpressing breast and gastric cancer.

### Linker, L

The conjugates of the present disclosure include a linker, L, which is a bifunctional or multifunctional moiety capable of linking one or more compounds of Formula I, D, to targeting moiety, T. In some embodiments, linker L may be bifunctional (or monovalent) such that it links a single compound to a single site on targeting moiety, T. In some embodiments, linker L may be multifunctional (or polyvalent) such that it links more than one compound to a single site on targeting moiety, T. Multifunctional linkers may also be used to link one compound to more than one site on targeting moiety, T, in some embodiments.

Linker, L, includes a functional group capable of reacting with the target group or groups on targeting moiety, T, and one or more functional groups capable of reacting with a target group on the compound of Formula I, D. Suitable functional groups are known in the art and include those described, for example, in Bioconjugate Techniques (G. T. Hermanson, 2013, Academic Press). Groups on targeting moiety, T, and compound, D, that may serve as target groups for linker attachment include, but are not limited to, thiol, hydroxyl, carboxyl, amine, aldehyde and ketone groups.

Non-limiting examples of functional groups for reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups for reacting with amines include activated esters (such as N-hydroxysuccinamide (NHS) esters and sulfo-NHS esters), imido esters (such as Traut's reagent), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidinophosphonium hexafluorophosphate (PyBOP).

Non-limiting examples of functional groups capable of reacting with an electrophilic group (such as an aldehyde or ketone carbonyl group) on targeting moiety, T, or compound, D, include hydrazide, oxime, amino, hydrazine, thiosemicarbazone, hydrazine carboxylate and arylhydrazide.

In certain embodiments in which targeting moiety, T, is an antibody, linker, L, may include a functional group that allows for bridging of two interchain cysteines on the antibody, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl (TCEP)pyridazinedione-based linker (Lee et al., 2016, Chem. Sci., 7:799-802) or a dibromopyridazinedione-based linker (Maruani et al., 2015, Nat. Commun., 6:6645).

Linker, L, may be a cleavable or a non-cleavable linker. A cleavable linker is a linker that is susceptible to cleavage under specific conditions, for example, intracellular conditions (such as in an endosome or lysosome) or within the vicinity of a target cell (such as in the tumour microenvironment). Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

Suitable cleavable linkers include, for example, linkers comprising an amino acid sequence that is a cleavage recognition sequence for a protease. Many such cleavage recognition sequences are known in the art. For conjugates that are not intended to be internalized by a cell, for example, an amino acid sequence that is recognized and cleaved by a protease present in the extracellular matrix in the vicinity of a cancer may be employed in linker, L. Examples of extracellular tumour-associated proteases include, for example, plasmin, matrix metalloproteases (MMPs) and kallikrein-related peptidases.

For conjugates intended to be internalized by a cell, linker, L, may comprise an amino acid sequence that is recognized and cleaved by an endosomal or lysosomal protease. Examples of such proteases include, for example, cathepsins B, C, D, H, L and S, and legumain.

Protease recognition sequences may be, for example, dipeptides, tripeptides or tetrapeptides. Non-limiting examples of suitable dipeptides for inclusion in cleavable linkers include Ala-(D) Asp, Ala-Lys, Ala-Phe, Asn-Lys, Asn-(D) Lys, Asp-Val, His-Val, Ile-Cit, Ile-Pro, Ile-Val, Leu-Cit, Me₃Lys-Pro, Met-Lys, Met-(D) Lys, NorVal-(D) Asp, Phe-Arg, Phe-Cit, Phe-Lys, PhenylGly-(D) Lys, Pro-(D) Lys, Trp-Cit, Val-Ala, Val-(D) Asp, Val-Cit, Val-Gly, Val-Gln and Val-Lys. Examples of suitable tri- and tetrapeptides include, but are not limited to, Ala-Ala-Asn, Ala-Val-Cit, (D) Ala-Phe-Lys, Asp-Val-Ala, Asp-Val-Cit, Gly-Cit-Val, Lys-Val-Ala, Lys-Val-Cit, Met-Cit-Val, (D) Phe-Phe-Lys, Ala-Leu-Ala-Leu, Gly-Gly-Phe-Gly and Gly-Phe-Leu-Gly.

In certain embodiments, linker, L, may comprise an amino acid sequence that is recognized and cleaved by Cathepsin B. Non-limiting examples of cathepsin B cleavable peptide sequences include Val-Ala, Val-Cit, Val-Gly, Val-Gln, Val-Lys, Ala-Val-Cit, Asp-Val-Ala, Asp-Val-Cit, Lys-Val-Ala and Lys-Val-Cit.

Additional suitable cleavable linkers include disulfide-containing linkers. Examples of disulfide-containing linkers include, but are not limited to, N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Other suitable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumour interstitium (see, for example, De Graaf et al., 2002, Curr. Pharm. Des. 8:1391-1403). β-glucuronide may also function to improve the hydrophilicity of linker, L, and thus function as a hydrophilicity moiety in conjugates of Formula X.

Another example of a linker that is cleaved internally within a cell and improves hydrophilicity is a linker comprising a pyrophosphate diester moiety (see, for example, Kern et al., 2016, J Am Chem Soc., 138:2430-1445). Pyrophosphate diesters may also function to improve the hydrophilicity of linker, L, and thus function as a hydrophilicity moiety in conjugates of Formula X.

Cleavable linkers may optionally further comprise one or more additional functionalities such as self-immolative and self-elimination groups, stretchers or hydrophilic moieties.

Self-immolative and self-elimination groups that find use in linkers include those described above, for example, p-aminobenzyl (PAB) and p-aminobenzyloxycarbonyl (PABC) groups, and methylated ethylene diamine (MED). Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the compound, D. Self-immolative/self-elimination groups, alone or in combination are often included in peptide-based linkers, but may also be included in other types of linkers. In some embodiments, the linker may include one or more self-immolative and self-elimination groups, for example, a PAB group, a PABC group, or a combination of a PAB or PABC group and a MED group.

Stretchers that find use in linkers for drug conjugates include, for example, alkylene groups and stretchers based on aliphatic acids, diacids, amines or diamines, such as diglycolate, malonate, caproate and caproamide. Other stretchers include, for example, glycine based stretchers and polyethylene glycol (PEG) or monomethoxy polyethylene glycol (mPEG) stretchers.

PEG and mPEG stretchers also function as hydrophilic moieties within linker, L. PEG or mPEG units may be included in linker, L, either “in-line” or as pendant groups to increase the hydrophilicity of the linker (see, for example, U.S. Patent Application Publication No. US 2016/0310612). Various PEG-containing linkers are commercially available from companies such as Quanta BioDesign, Ltd (Plain City, OH). Other hydrophilic moieties that may be incorporated into linker, L, include, for example, polysarcosine, saccharides (such as β-glucuronide), sulfonate groups, sulfonamide groups, carboxylate groups and pyrophosphate diesters.

As compounds of Formula I may be hydrophobic, certain embodiments of the present disclosure contemplate inclusion of one or more hydrophilic moieties within linker, L, to reduce the potential for ADC aggregation and promote the solubility of the intact conjugate compared to a conjugate having a linker without additional hydrophilic groups.

In certain embodiments, linker, L, is a peptide-based linker and the drug-linker has Formula XV:

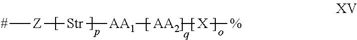

wherein:
Z is a linking group that joins the linker to a target group on targeting moiety, T;
Str is a stretcher, optionally including one or more hydrophilic moieties;
AA₁ and AA₂ are each independently an amino acid, wherein AA₁-[AA₂]ₘ forms a protease cleavage site;
X is a self-immolative group;
p is 0 or 1;
q is 1, 2 or 3;
o is 0, 1 or 2;
# is the point of attachment to the targeting moiety, T, and
% is the point of attachment to the compound of Formula I, D.

In accordance with this embodiment, the final conjugate would have Formula XVI:

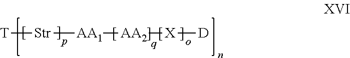

wherein Str, AA₁, AA₂, X, D, p, q and o are as defined for Formula XV;
n is between 1 and about 12, and
T is the targeting moiety.

In some embodiments, in Formula XV and Formula XVI:
Z is

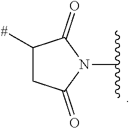

In some embodiments, Str in Formula XV and Formula XVI includes one or more hydrophilic moieties. In some embodiments, Str in Formula XV and Formula XVI includes one or more β-glucuronide, sulfonate groups or pyrophosphate diesters.

In some embodiments, in general Formula XV or Formula XVI:
Str is

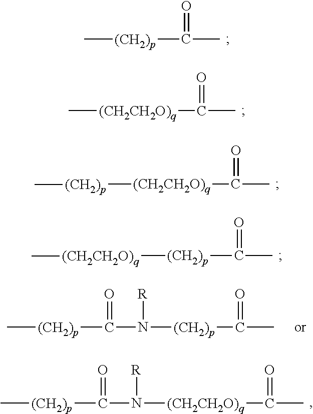

wherein:
R is H or C₁-C₆ alkyl;
p is an integer between 2 and 10, and
q is an integer between 1 and 10.

In some embodiments, linker, L, is a pyrophosphate-containing linker and the drug-linker has Formula XVII:

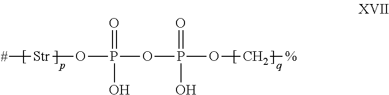

wherein:
Z is a linking group that joins the linker to a target group on targeting moiety, T;
Str is a stretcher, optionally including one or more hydrophilic moieties;
p is 0 or 1;
q is 1, 2 or 3;
# is the point of attachment to the targeting moiety, T, and
% is the point of attachment to the compound of Formula I, D.

In accordance with this embodiment, the final conjugate would have Formula XVIII:

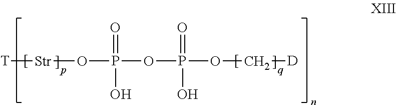

wherein Str, D, p and q are as defined for Formula XVII;
n is between 1 and about 12, and
T is the targeting moiety.

In some embodiments, the linker is a disulfide-containing linker and the conjugate has Formula XIX:

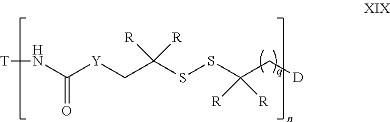

wherein:
T is the targeting moiety, T;
D is a compound of Formula I;
Y is —(CH₂)ₚ— or —(CH₂CH₂O)ᵣ—, wherein p and r are each independently an integer between 1 and 10;
each R is independently H or C₁-C₆ alkyl, and
q is 1, 2 or 3, and
n is between 1 and 12,

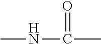

wherein represents an amide bond formed between the linker and the ε-amino group of a surface lysine on targeting moiety, T.

Various non-cleavable linkers are known in the art for linking drugs to targeting moieties and may be useful in the conjugate compositions of the present disclosure in certain embodiments. Examples of non-cleavable linkers include linkers having an N-succinimidyl ester or N-sulfosuccinimidyl ester moiety for reaction with the targeting moiety, T, as well as a maleimido- or haloacetyl-based moiety for reaction with the compound of Formula I, D, or vice versa. An example of such a non-cleavable linker is based on sulfosuccinimidyl-4-[N-maleimidomethyl]cyclohexane-1-carboxylate (sulfo-SMCC). Sulfo-SMCC conjugation typically occurs via a maleimide group which reacts with sulfhydryls (thiols, —SH) on compound, D, while the sulfo-NHS ester is reactive toward primary amines (as found in lysine and at the N-terminus of proteins or peptides) on targeting moiety, T. Other non-limiting examples of such linkers include those based on N-succinimidyl 4-(maleimidomethyl)cyclohexanecarboxylate (SMCC), N-succinimidyl-4-(N-maleimidomethyl)-cyclohexane-1-carboxy-(6-amidocaproate) (“long chain” SMCC or LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N—(α-maleimidoacetoxy)-succinimide ester (AMAS), succinimidyl-6-(β-maleimidopropionamido) hexanoate (SMPH), N-succinimidyl 4-(p-maleimidophenyl)-butyrate (SMPB) and N-(p-maleimidophenyl) isocyanate (PMPI). Other examples include those comprising a haloacetyl-based functional group such as N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido) propionate (SBAP).

Other examples of non-cleavable linkers include maleimidocarboxylic acids, such as maleimidocaproyl (MC).

### Preparation of Conjugates

Conjugates comprising compounds of Formula I may be prepared by standard methods known in the art (see, for example, Bioconjugate Techniques (G. T. Hermanson, 2013, Academic Press)). Various prepared linkers and linker components are commercially available or may be prepared using standard synthetic organic chemistry techniques (see, for example, March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramel Pharma Solutions (Grangemouth, UK).

Typically, preparation of conjugates comprises first preparing drug-linker, D-L, comprising one or more compounds of Formula I, D, and linker, L, and then conjugating drug-linker, D-L, to an appropriate group on targeting moiety, T. Ligation of linker, L, to targeting moiety, T, and subsequent ligation of the targeting moiety-linker, T-L, to one or more compounds of Formula I, D, is however an alternative approach that may be employed in some embodiments.

Examples of suitable groups on compounds of Formula I, D, for attachment of linker, L, in either of the above approaches include amine groups and hydroxyl groups. In some embodiments of the present disclosure, linker, L, is attached to a compound of Formula I via a hydroxyl group on one of the chromophore moieties.

Suitable groups on targeting moiety, T, for attachment of linker, L, in either of the above approaches include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups. In some embodiments of the present disclosure, linker, L, is attached to a targeting moiety, T, via a sulfhydryl group on the side-chain of a cysteine residue.

For example, targeting moiety, T, may comprise one or more naturally occurring sulfhydryl groups allowing the targeting moiety to bond to linker, L, via the sulfur atom of a sulfhydryl group. Alternatively, targeting moiety, T, may comprise one or more lysine residues that can be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio) propionate (“SPDP”) and 2-iminothiolane hydrochloride (Traut's Reagent). Targeting moiety, T, may also comprise one or more carbohydrate groups that can be chemically modified to include one or more sulfhydryl groups.

Carbohydrate groups on targeting moiety, T, may also be oxidized to provide an aldehyde (—CHO) group (see, for example, Laguzza et al., 1989, J. Med. Chem. 32 (3): 548-55), which could subsequently be reacted with linker, L, for example, via a hydrazine or hydroxylamine group on the linker.

Targeting moiety, T, may also be modified to include additional cysteine residues (see, for example, U.S. Pat. Nos. 7,521,541; 8,455,622 and 9,000,130, and International Patent Publication No. WO 2022/198335) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361), to allow for site-specific conjugation.

Other protocols for the modification of proteins for the attachment or association of a linker are known in the art and include those described in Coligan et al., Current Protocols in Protein Science, vol. 2, John Wiley & Sons (2002).

In those embodiments in which targeting moiety, T, is an antibody, several different reactive groups on the antibody may function as a conjugation site, including ¿-amino groups on lysine residues, pendant carbohydrate moieties, side-chain carboxylic acid groups on aspartate or glutamate residues, cysteine-cysteine disulfide groups and cysteine thiol groups. The amino acids used for conjugation may be part of the natural sequence of the antibody, or they may be introduced by site-specific engineering techniques known in the art, as noted above.

Alternatively, antibody-drug conjugates may be prepared using the enzyme transglutaminase, for example, bacterial transglutaminase (BTG) from Streptomyces mobaraensis (see, for example, Jeger et al., 2010, Angew. Chem. Int. Ed., 49:9995-9997). BTG forms an amide bond between the side chain carboxamide of a glutamine (the amine acceptor, typically on the antibody) and an alkyleneamino group (the amine donor, typically on the drug-linker), which can be, for example, the ¿-amino group of a lysine or a 5-amino-n-pentyl group. Antibodies may also be modified to include a glutamine containing peptide, or “tag,” which allows BTG conjugation to be used to conjugate the antibody to a drug-linker (see, for example, U.S. Patent Application Publication No. US 2013/0230543 and International (PCT) Publication No. WO 2016/144608).

A similar conjugation approach utilizes the enzyme sortase A. In this approach, the antibody is typically modified to include the sortase A recognition motif (LPXTG, where X is any natural amino acid) and the drug-linker is designed to include an oligoglycine motif (typically GGG) to allow for sortase A-mediated transpeptidation (see, for example, Beerli, et al., 2015, PLos One, 10:e0131177; Chen et al., 2016, Nature: Scientific Reports, 6:31899).

Once conjugation is complete, the average number of compounds of Formula I conjugated to targeting moiety, T (i.e. the “drug-to-antibody ratio” or DAR) may be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, distribution of drug-linked forms (for example, the fraction of targeting moiety, T, containing zero, one, two, three, etc. compounds of Formula I) may also optionally be analyzed. Various techniques are known in the art to measure such distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

In certain embodiments of the present disclosure in which targeting moiety, T, is an antibody, the conjugate may have a DAR of between about 2 and about 8, for example, between about 2 and about 6, or between about 2 and about 4. In certain embodiments, lower DAR conjugates, such as DAR2 conjugates, may be advantageous when using compounds of Formula I that are particularly hydrophobic, for example, to help minimize aggregation. In certain embodiments in which targeting moiety, T, is an antibody, the conjugate may be prepared using site-specific conjugation methods in order to control the final DAR of the conjugate.

### Pharmaceutical Compositions

Compounds of Formula I and conjugates comprising compounds of Formula I, for example conjugates having Formula X, are typically formulated for therapeutic use. Certain embodiments thus relate to pharmaceutical compositions comprising a compound of Formula I or a conjugate thereof, such as a conjugate of Formula X, and a pharmaceutically acceptable carrier, diluent, or excipient. Such pharmaceutical compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term parenteral as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

Compositions intended for oral use may be prepared in either solid or fluid unit dosage forms. Fluid unit dosage forms may be prepared according to procedures known in the art for the manufacture of pharmaceutical compositions and such compositions may contain one or more agents such as sweetening agents, flavouring agents, colouring agents and preserving agents in order to provide pharmaceutically elegant and palatable preparations. An elixir may be prepared by using a hydroalcoholic (for example, ethanol) carrier with suitable sweeteners such as sugar and/or saccharin, together with an aromatic flavoring agent. Suspensions may be prepared with an aqueous carrier and a suspending agent such as acacia, tragacanth, methylcellulose and the like.

Solid formulations, such as tablets, contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents for example, corn starch, or alginic acid; binding agents, for example starch, gelatin or acacia, and/or lubricating agents, for example magnesium stearate, stearic acid or talc, as well as other conventional ingredients such as dicalcium phosphate, magnesium aluminum silicate, calcium sulfate, starch, lactose, methylcellulose, and functionally similar materials. The tablets may be uncoated or they may be coated by known techniques, for example, in order to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are typically prepared by machine encapsulation of a slurry of the active ingredient with an acceptable vegetable oil, light liquid petrolatum or other inert oil.

Aqueous suspensions contain the active ingredient in admixture with excipients suitable for the manufacture of aqueous suspensions. Such excipients include suspending agents, for example sodium carboxylmethylcellulose, methyl cellulose, hydropropylmethylcellulose, sodium alginate, polyvinylpyrrolidone, gum tragacanth and gum acacia, and dispersing or wetting agents. Dispersing and wetting agents include, for example, naturally-occurring phosphatides (for example, lecithin), condensation products of an alkylene oxide with fatty acids (for example, polyoxyethylene stearate), condensation products of ethylene oxide with long chain aliphatic alcohols (for example, hepta-decaethyleneoxycetanol), condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example, polyethylene sorbitan monooleate). The aqueous suspensions may also contain one or more preservatives (for example ethyl, or n-propyl-p-hydroxybenzoate), one or more colouring agents, one or more flavouring agents and/or one or more sweetening agents (for example, sucrose or saccharin).

Oily suspensions may be formulated by suspending the active ingredient in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. The suspensions may optionally be preserved by the addition of an anti-oxidant such as ascorbic acid.

Dispersible powders and granules suitable for preparation of an aqueous suspension by the addition of water typically provide the active ingredient in admixture with a dispersing or wetting agent, suspending agent and one or more preservatives. Suitable dispersing or wetting agents and suspending agents are exemplified by those already mentioned above. One or more additional excipients, for example sweetening, flavouring and/or colouring agents, may also be present.

Pharmaceutical compositions may also be in the form of oil-in-water emulsions. The oil phase may be a vegetable oil, for example olive oil or peanut oil, or a mineral oil, for example liquid paraffin, or mixtures of such oils. Suitable emulsifying agents may be naturally-occurring gums (for example, gum acacia or gum tragacanth), naturally-occurring phosphatides (for example, soy bean, lecithin), or esters or partial esters derived from fatty acids and hexitol anhydrides (for example, sorbitan monooleate) or condensation products of such partial esters with ethylene oxide (for example polyoxyethylene sorbitan monooleate). The emulsions may also optionally contain sweetening and/or flavoring agents.

The pharmaceutical compositions may be in the form of a sterile injectable aqueous or oleaginous solution or suspension. Such suspensions may be formulated using suitable dispersing or wetting agents and suspending agents such as those described above. The sterile injectable solution or suspension may comprise the active ingredient in a non-toxic parentally acceptable carrier or diluent. Acceptable carriers and diluents that may be employed include, for example, 1,3-butanediol, water, Ringer's solution or isotonic sodium chloride solution. In addition, sterile, fixed oils are conventionally employed as a carrier. For this purpose, various bland fixed oils may be employed including synthetic mono- or diglycerides. In addition, fatty acids such as oleic acid find use in the preparation of injectables. Adjuvants such as local anaesthetics, preservatives and/or buffering agents may also be included in the injectable solution or suspension.

Pharmaceutical compositions may also be formulated as suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Compounds of Formula I are DNA intercalators and thus these compounds and conjugates comprising compounds of Formula I, such as conjugates of Formula X, may be useful as anti-cancer agents, anti-microbial agents (for example, anti-bacterial agents) and/or anti-viral agents. Compounds of Formula I may also find use as DNA labelling compounds.

Some embodiments of the present disclosure thus relate to methods of binding DNA comprising contacting the DNA with a compound of Formula I. Some embodiments relate to methods of binding DNA comprising contacting the DNA with a conjugate comprising a compound of Formula I, such as a conjugate of Formula X. In some embodiments, the DNA is in vitro. In some embodiments, the DNA is in vivo.

Certain embodiments of the present disclosure relate to the use of compounds of Formula I and conjugates comprising compounds of Formula I, such as conjugates of Formula X, as therapeutic agents. Some embodiments relate to the use of compounds of Formula I and conjugates thereof, such as conjugates of Formula X, as labelling agents or diagnostic agents.

Certain embodiments of the present disclosure relate to a method of inhibiting the proliferation of tumour cells, microbial cells or viral cells comprising contacting the cells with a compound of Formula I. Certain embodiments of the present disclosure relate to a method of inhibiting the proliferation of tumour cells, microbial cells or viral cells comprising contacting the cells with a conjugate comprising a compound of Formula I, such as a conjugate of Formula X. Some embodiments relate to a method of killing tumour cells, microbial cells or viral cells comprising contacting the cells with a compound of Formula I. Some embodiments relate to a method of killing tumour cells, microbial cells or viral cells comprising contacting the cells with a conjugate comprising a compound of Formula I, such as a conjugate of Formula X. In some embodiments, the cells are contacted in vitro. In some embodiments, the cells are contacted in vivo. In some embodiments, the microbial cells are bacterial cells.

In certain embodiments, compounds of Formula I may be used in methods of treating a microbial infection in a subject. In certain embodimennts, conjugates comprising a compound of Formula I, such as conjugates of Formula X, may be used in methods of treating a microbial infection in a subject. In some embodiments, compounds of general Formula I may be used in methods of treating a bacterial infection in a subject. In some embodiments, conjugates comprising a compound of Formula I, such as conjugates of Formula X, may be used in methods of treating a bacterial infection in a subject. In some embodiments, compounds of general Formula I may be used in methods of treating a viral infection in a subject. In some embodiments, conjugates comprising a compound of Formula I, such as conjugates of Formula X, may be used in methods of treating a viral infection in a subject.

In certain embodiments, compounds of general Formula I may be used in methods of treating cancer. In certain embodiments, conjugates comprising a compound of Formula I, such as conjugates of Formula X, may be used in methods of treating cancer. Some embodiments of the present disclosure thus relate to the use of compounds of Formula I and conjugates comprising compounds of Formula I, such as conjugates of Formula X, as anti-cancer agents.

Some embodiments relate to methods of treating a subject having a cancer by administering to the subject a compound of Formula I. Some embodiments relate to methods of treating a subject having a cancer by administering to the subject a conjugate comprising a compound of Formula I, such as a conjugate of Formula X. In this context, the compounds of Formula I and conjugates comprising compounds of Formula I, such as conjugates of Formula X, may exert either a cytotoxic or cytostatic effect and treatment may result in one or more of a reduction in the size of a tumour, the slowing or prevention of an increase in the size of a tumour, an increase in the disease-free survival time between the disappearance or removal of a tumour and its reappearance, prevention of a subsequent occurrence of a tumour (for example, metastasis), an increase in the time to progression, reduction of one or more adverse symptom associated with a tumour, and/or an increase in the overall survival time of a subject having cancer.

Certain embodiments relate to the use of a compound of Formula I in a method of inhibiting tumour growth in a subject. Certain embodiments relate to the use of a conjugate comprising a compound of Formula I, such as a conjugate of Formula X, in a method of inhibiting tumour growth in a subject. Some embodiments relate to the use of a compound of Formula I in a method of inhibiting proliferation of and/or killing cancer cells in vitro. Some embodiments relate to the use of a conjugate comprising a compound of Formula I, such as a conjugate of Formula X, in a method of inhibiting proliferation of and/or killing cancer cells in vitro. Some embodiments relate to the use of a compound of Formula I in a method of inhibiting proliferation of and/or killing cancer cells in vivo in a subject having a cancer. Some embodiments relate to the use of a conjugate comprising a compound of Formula I, such as a conjugate of Formula X, in a method of inhibiting proliferation of and/or killing cancer cells in vivo in a subject having a cancer.

Examples of cancers which may be treated in certain embodiments include haematologic neoplasms, such as leukaemias, myelomas and lymphomas; carcinomas, including adenocarcinomas and squamous cell carcinomas; melanomas and sarcomas. Carcinomas and sarcomas are also frequently referred to as “solid tumours.” Examples of commonly occurring solid tumours include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumour and, therefore, may also be considered to be solid tumours in certain situations.

When used in the treatment of cancer or a microbial infection, the dosage of the compound of Formula I or conjugate comprising a compound of Formula I, such as a conjugate of Formula X, to be administered to the subject will be a therapeutically effective amount. A therapeutically effective amount is an amount that, when administered to a subject, is sufficient to exert a therapeutic effect. The compounds and conjugates may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

### Pharmaceutical Kits

In certain embodiments, a pharmaceutical composition comprising a compound of Formula I or a conjugate thereof, such as a conjugate of Formula X, may be provided as part of a pharmaceutical kit or pack. Individual components of the kit would typically be packaged in separate containers. Suitable containers include, for example, bottles, blister packs, intravenous solution bags, vials and the like, depending on the formulation of the pharmaceutical composition. In certain embodiments, the container may be in a form allowing for administration to a subject, for example, an inhaler, syringe, pipette, eye dropper, pre-soaked gauze or pad, or other such like apparatus, from which the contents may be administered to the subject.

The kit may further comprise a label or package insert on or associated with the container(s). The term “package insert” is used to refer to instructions customarily included in commercial packages of therapeutic products that contain information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. The label or package insert typically indicates that the compound or conjugate is for use to treat the defined condition, for example, cancer.

If appropriate, one or more components of the kit may be lyophilized or provided in a dry form, such as a powder or granules, and the kit can optionally contain a suitable solvent for reconstitution of the lyophilized or dried component(s).

The following Examples are provided for illustrative purposes and are not intended to limit the scope of the invention in any way.

### EXAMPLES

The following Examples illustrate various methods of making compounds of general Formula (I). It is understood that one skilled in the art may be able to make these compounds by similar methods or by combining other methods known in the art. It is also understood that one skilled in the art would be able to make, using the methods described below or similar methods, other compounds of general Formula (I) not specifically illustrated below by using the appropriate starting components and modifying the parameters of the synthesis as needed. In general, starting components may be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

The following abbreviations are used throughout the Examples. CDI=carbonyl diimidazole; DBU=1,8-diazabicyclo[5.4.0]undec-7-ene; DCM=dichloromethane; DMAP=dimethylaminopyridine; DMF=dimethylformamide; DPPA=diphenylphosphoryl azide; EDC=1-ethyl-3-(3-dimethylaminopropyl) carbodiimide; HATU=hexafluorophosphate azabenzotriazole tetramethyl uronium; HFIP=hexafluoroisopropanol; LC/MS=Liquid Chromatography/Mass Spectrometry; LC/MSD=Liquid Chromatography/Mass Selective Detector; MT=maleimido triethylene glycolate; NEt(i-Pr)₂=N,N-di-isopropylethylamine; NMM=N-methylmorpholine; PAB=p-aminobenzyl; rb=round bottom; rt=room temperature; TBAI=tetrabutylammonium iodide; TFA=trifluoroacetic acid; THF=tetrahydrofuran; VA=valine-alanine; VC=valine-citrulline.

### Example 1: General Synthetic Procedures

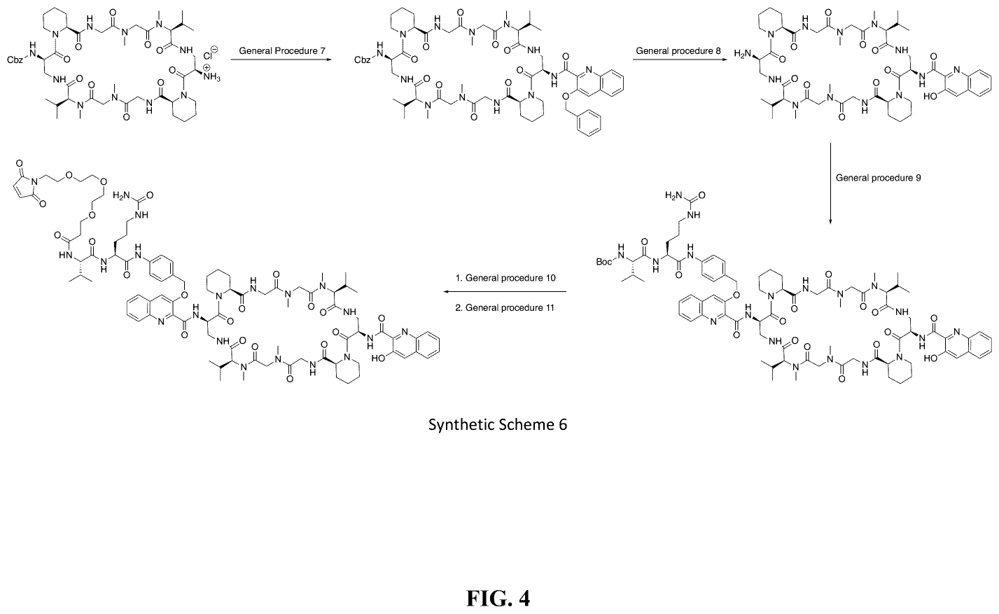

The following general synthetic procedures were employed in the preparation of compounds of general Formula (I). Examples of synthetic schemes employing these procedures are provided in FIGS. 1 to 4.

### 1.1 General Procedure 1: Fmoc Solid-Phase Peptide Synthesis

Resin activation: 500 mg of 2-chlorotrityl resin (Chempep, 0.81 mmol/g) in a 15 mL fritted cartridge was swollen in DCM (5 mL) and DMF (1 mL) for 10 min, drained, and rinsed with DCM. The resin was then incubated in 2% SOCl₂/DCM for 2 h, drained, washed with DCM, incubated in 2% SOCl₂/DCM overnight, then drained and washed with DCM.

Resin loading: The resin containing cartridge was charged with a solution of the first amino acid (0.81 umol, 2 equiv.), and NEt (i-Pr)₂ (2 mmol, 5 equiv.) in DCM (6 mL) and rotated for 3 h, then drained and washed with DCM. Unreacted sites were capped with a solution of MeOH (2 mL), NEt(i-Pr)₂ (1 mL), in DCM (7 mL). The capping solution was drained, and the resin washed with DMF.

Deprotection: Fmoc removal was accomplished by treatment of the resin with a 20% piperidine solution in DMF (3×(4 mL for 3 min)). After the final piperidine solution was drained, the resin was washed thoroughly with DMF.

N-Nosyl protecting groups were removed on resin with a solution of a thiophenol (2 mL), DBU (2 mL) in DMF (6 mL) (2×30 min) followed by rinsing with DMF.

Amino acid coupling: To the resin was added a solution of pre-activated amino acid (1.2 mmol, 3 equiv.), HATU (1.1 mmol, 3 equiv.) and NEt(i-Pr)₂ (2 mmol, 5 equiv.) in DMF (4 mL) and the cartridge was rotated for 1 h. The coupling solution was drained and the resin washed with DMF. Complete coupling was confirmed through cleavage of a small aliquot of resin (100 uL TFA) for 5 min, dilution in acetonitrile, filtration of cleaved resin, and analysis by LC/MS.

Resin Cleavage and peptide precipitation: Protected peptides were cleaved from resin with a solution of 20% HFIP in DCM (3×(10 mL for 15 min)). The combined filtrates were evaporated to a yellow oil that was precipitated in cold diethyl ether (40 mL) in a 50 ml conical Falcon® tube. The resulting white precipitate was separated by centrifugation (2000 rpm for 10 min) and decanting of the ether. The pellet was resuspended in ether, centrifuged, and the ether decanted once more. The final pellet was allowed to dry, open to air, for >3 h affording the crude peptide as a white powder.

### 1.2 General Procedure 2: Solid Phase Synthesis of Ester-Containing Compounds

Ester-containing compounds were generating by incorporating the ester linkage as a pre-formed depsi-dipeptide (see FIG. 1C, Synthetic Scheme 3). The esters were found to be highly susceptible to cleavage through diketopiperazine formation during deprotection of the subsequent amino acid (e.g. sarcosine). Therefore, successful solid-phase synthesis of the ester-containing analogues required that the next 2 amino acids were incorporated as a pre-formed dipeptide (e.g. Fmoc-Gly-Sar-OH).

Depsi-dipeptides (1.2 mmol, 3 equiv.) were pre-activated with HATU (1.1 mmol, 3 equiv.) and NEt(i-Pr)₂ (2 mmol, 5 equiv.) in DMF (4 mL), and this solution was added to resin and the cartridge rotated for 1 h.

### 1.3 General Procedure 3: Solid Phase Synthesis of Ether-Containing Compounds

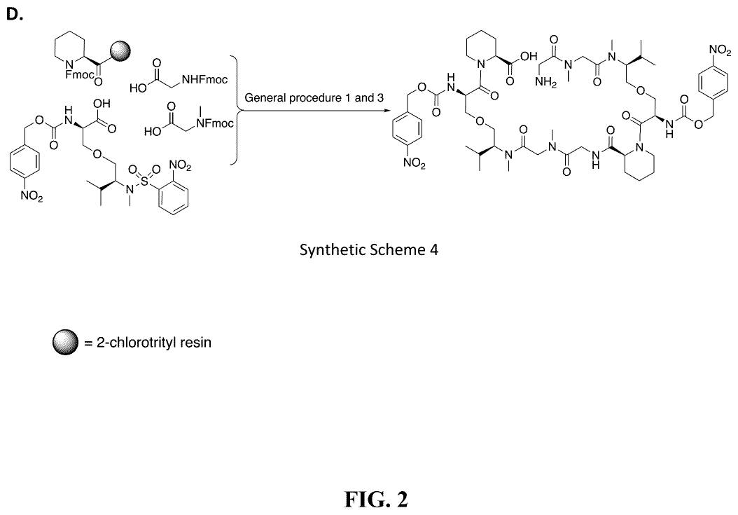

N-Nosyl protected pseudo-dipeptides (1.2 mmol, 3 equiv.) were pre-activated with HATU (1.1 mmol, 3 equiv.) and NEt(i-Pr)₂ (2 mmol, 5 equiv.) in DMF (4 mL), added to the resin cartridge and rotated for 1 h (see FIG. 2, Synthetic Scheme 4). N-Nosyl protecting groups were removed on resin with a solution of a thiophenol (2 mL), DBU (2 mL) in DMF (6 mL) (2× 30 min) followed by rinsing with DMF.

### 1.4 General Procedure 4: Cyclization of Linear Decapeptides

A stirring solution of linear decapeptide in DMF (0.003M) was cooled to 0° C. followed by the addition of NaHCO₃ (10 equiv) and diphenyl phosphoryl azide (DPPA) (4 equiv). This solution was kept in a 4° C. fridge for 16 to 72 h. Once cyclization was complete, DMF was removed by evaporation and the residue taken up in EtOAc, washed with an aqueous 0.1 M HCl solution, H₂O, a saturated aqueous solution of NaHCO₃, and finally with a saturated aqueous solution of NaCl. The organic layer was dried over Na₂SO₄, concentrated and flash purified. Solvent removal by rotovap or lyophilization afforded the cyclized peptide (typically as a white powder).

### 1.5 General Procedure 5: Deprotection of Boc Protecting Groups

The Boc-protected amine, in a rb flask, was cooled to 0° C. followed by the addition of a 4M HCl/dioxane solution. The ice bath was removed, and the solution was stirred at rt for 1 h. Once cleavage was complete, excess HCl/dioxane was removed by rotovap and the residue precipitated by the addition of EtOAc. The EtOAc was then removed by rotovap affording the free amine as the HCl salt, which was typically used without further purification.

1.6 General Procedure 6: Hydrogenation of Cbz, Benzyl, and pNO₂Cbz Protected Amines

To a solution of protected amine, in a rb flask, in MeOH (0.1 M to 0.01 M) was added Pd/C (10 wt. %, 100 mg/mmol). The flask was purged with H₂ and the solution stirred vigorously under an H₂ atmosphere for >1 h. Following complete removal of the protecting group, Pd/C was removed by filtration through a bed of Celite® and washing with copious amounts of EtOAc and MeOH. The filtrate was concentrated by rotovap to afford the free amine.

### 1.7 General Procedure 7: Coupling of the Benzyl-Chromophore

To the solid cyclic decapeptide (containing either 1 or 2 free amines) was added a pre-mixed solution of benzyl-chromophore (1.1 equiv./amine), HATU (1.05 equiv./amine) and NEt(i-Pr)₂ (2 equiv./amine) in DMF (0.05M). This solution was stirred at rt for 1 hour then concentrated in vacuo and purified to afford the decapeptide with either one or two benzyl-chromophores.

### 1.8 General Procedure 8: Deprotection of the Benzyl-Chromophore

To a solution of cyclic decapeptide with either one or two benzyl-chromophores (Compound 4) in a round bottom flask in MeOH (0.1 M to 0.005 M) was added Pd/C (10 wt. %, 100 mg/mmol). The flask was purged with H₂ and the solution stirred vigorously under an H₂ atmosphere for >1 h. Following complete removal of the protecting group, Pd/C was removed by filtration through a bed of Celite® and washing with copious amounts of EtOAc and/or MeOH. The filtrate was concentrated by rotovap and the residue purified by flash chromatography to afford the cyclic decapeptide with deprotected chromophore(s) typically as a pale-yellow to bright-yellow powder.

### 1.9 General Procedure 9: Coupling of Boc-VC-PAB-Chromophore

To the solid cyclic decapeptide was added a pre-mixed solution of Boc-VC-PAB-chromophore (1.2 equiv.), HATU (1.1 equiv.) and NEt(i-Pr)₂ (3 equiv.) in DMF (0.05M). This solution was stirred for 1 h at rt. Upon completion (typically within 30 min), the reaction mixture was purified by reverse-phase HPLC.

1.10 General Procedure 10: Deprotection of Boc Protecting Groups with TFA

A stirring solution of Boc-protected amine in DCM (0.01 to 0.001 M) was cooled to 0° C. and TFA (4:1 DCM: TFA v/v) was added dropwise. The ice bath was removed and the reaction mixture stirred at rt for 1 h. Once cleavage was complete, excess TFA was removed by rotovap. The residue was co-evaporated (2×5 mL acetonitrile and/or 2×5 mL toluene) to afford the free amine as the TFA salt, which was typically used without further purification.

### 1.11 General Procedure 11: Anchor Installation

To the amine salt (0.05 M) was added a solution of NEt(i-Pr)₂ (3 eq) and either Compound 212 (1.1 eq.) or N-succinimidyl 3-maleimidopropionate (1.1 eq.) in DMF (0.01 M). This solution was stirred at rt for 1 h, concentrated, and purified by reverse-phase HPLC to afford the desired compound-linker construct after lyophilization.

### 1.12 General Procedure 12: Compound Purification

Flash Chromatography: Crude reaction products were purified with Biotage Snap Ultra columns (10, 25, 50, or 100 g), and elution with linear gradients of EtOAc/hexanes or MeOH/DCM on a Biotage® Isolera™ automated flash system. Alternatively, reverse-phase flash purification was conducting using Biotage® Snap Ultra C18 columns (12, 30, 60, or 120 g) and elution with linear gradients of 0.1% TFA in acetonitrile/0.1% TFA in water. Purified compounds were isolated either by removal of organic solvents by rotavap or by lyophilization of acetonitrile/water mixtures.

Preparative HPLC: Reverse-phase HPLC of crude compounds was performed using a Phenomenex Kinetex® 5-μm EVO C18 100 Å (250×21.2 mm) column on an Agilent 1260 Infinity II preparative LC/MSD system, and elution with linear gradients of 0.1% TFA in acetonitrile/0.1% TFA in water. Purified compounds were isolated by lyophilization of acetonitrile/water mixtures.

### 1.13 General Procedure 13: Compound Analysis

LC/MS: Purified compounds were analyzed using a Phenomenex Kinetex® 2.6-μm C18 100 Å (30×3 mm) column on an Agilent 1290 HPLC/6120 single quad LC/MS system. Elution with a linear gradient of 10-100 0.1% TFA in acetonitrile/0.1% TFA in water.

NMR: ¹H NMR spectra were collected with a Bruker Avance™ III 300 Spectrometer (300 MHz). Chemical shifts are in parts per million (ppm).

### Example 2: Synthesis of Compounds of General Formula (I)

### 2.1 3-(2-nitrobenzoyl)pentane-2,4-dione (Compound 1)

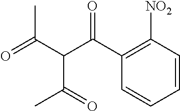 [SMILES: CC(=O)C(C(C)=O)C(=O)c1ccccc1[N+](=O)[O-]]

To a stirred solution of magnesium chloride (5.70 g, 59.8 mmol) in CH₂Cl₂ (60 mL) was added acetylacetone (6.1 mL, 59.8 mmol). The reaction mixture was cooled to 0° C. and pyridine (9.64 mL, 120 mmol) was added, followed by 2-nitrobenzoyl chloride (11.1 g, 59.8 mmol). The solution was allowed to warm to room temperature and stir for 1 h, then cooled to 0° C. once again and quenched with aqueous 6M HCl (60 mL). Additional H₂O (50 mL) was added and this biphasic solution was extracted EtOAc (2×100 mL). The combined organic extracts were washed with brine, dried over Na₂SO₄, then concentrated in vacuo. Purification according to General Procedure 12, using a 100 g column and eluting with 0 to 40% EtOAc/hexanes provided the title compound (10.2 g, 40.9 mmol, 68% yield) as a white solid.

LC/MS: calc'd m/z=249.1 for C₁₂H₁NO₅, found [M+H]⁻=248.2. ¹H NMR (300 MHZ, acetone-d6) δ 8.12-8.04 (m, 1H), 7.89-7.80 (m, 3H), 2.17 (s, 6H).

### 2.2 1-(3-hydroxyquinolin-2-yl)ethan-1-one (Compound 2)

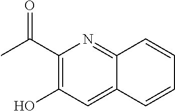

A stirred solution of Compound 1 (4.40 g, 17.6 mmol) in KOH (10.8 g in 54 mL H₂O) was heated to 100° C. for 1 h. The solution was cooled to rt, then the pH was adjusted to ˜5 with aqueous 6M HCl. The precipitate was collected by filtration, washed with H₂O (2×50 mL), then dried under vacuum to afford the title compound (2.80 g, 15.0 mmol, 85% yield) as a yellow solid.

LC/MS: Calc'd m/z=187.1 for C₁₁H₉NO₂, found [M+H]⁺=188.0. ¹H NMR (300 MHZ, chloroform-d) δ 11.21 (s, 1H), 8.09-8.03 (m, 1H), 7.73-7.69 (m, 1H), 7.65 (s, 1H), 7.60-7.53 (m, 2H), 2.95 (s, 3H).

### 2.3 1-(3-(benzyloxy)quinolin-2-yl)ethan-1-one (Compound 3)

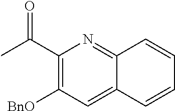 [SMILES: CC(=O)c1nc2ccccc2cc1OCc1ccccc1]

To a stirred solution of Compound 2 (2.40 g, 12.8 mmol) in CH₃CN (50 mL) was added C₅₂CO₃ (4.60 g, 14 mmol) then benzyl bromide (1.80 mL, 15.4 mmol). After 1 h, the reaction mixture was concentrated and purified as described in General Procedure 12, using a 50 g column and eluting with 0 to 30% EtOAc/hexanes to provide 2.51 g (9.06 mmol, 71%) of the title compound as an off-white solid.

LC/MS: Calc'd m/z=277.1 for C₁₈H₁₅NO₂, found [M+H]⁺=278.2. ¹H NMR (300 MHZ, chloroform-d) δ 8.08 (d, J=8.2 Hz, 1H), 7.71 (dd, J=8.2, 1.8 Hz, 1H), 7.62-7.54 (m, 3H), 7.51 (d, J=7.5 Hz, 2H), 7.41 (t, J=7.2 Hz, 2H), 7.37-7.31 (m, 1H), 5.27 (s, 2H), 2.78 (s, 3H).

### 2.4 3-(benzyloxy)quinoline-2-carboxylic acid (Compound 4)

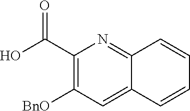 [SMILES: O=C(O)c1nc2ccccc2cc1OCc1ccccc1]

To a stirred solution of Compound 3 (2.40 g, 8.66 mmol) in 1,4-dioxane (60 mL) was added undiluted household bleach (80 mL). The solution was stirred for 1 h then quenched with aqueous 1M Na₂S₂O₃ (40 mL). The pH of the solution was adjusted to ˜2 with aqueous 1M HCl then extracted EtOAc (3×150 mL). The combined organic extracts were dried over Na₂SO₄ then concentrated in vacuo to afford the title compound (2.39 g, 8.57 mmol, 99%) as a brown solid.

LC/MS: Calc'd m/z=279.1 for C₁₇H₁₃NO₃, found [M−H]⁻=278.2. ¹H NMR (300 MHZ, acetone-d6) δ 8.05-7.99 (m, 2H), 7.94-7.89 (m, 1H), 7.70-7.56 (m, 3H), 7.45-7.38 (m, 2H), 7.37-7.30 (m, 1H), 5.40 (s, 2H).

### 2.5 N,N′-((7R,11S,19aS,26R,30S,38aS)-11,30-diisopropyl-12,15,31,34-tetramethyl-6,10,13,16,19,25,29,32,35,38-decaoxodotriacontahydro-2H,6H,10H,25H-dipyrido[2,1-b1:2′,1′-l][1,17]dioxa[4,7,10,13,20,23,26,29]octaazacyclodotriacontine-7,26-diyl)bis(3-hydroxyquinoline-2-carboxamide) (Sandramycin; Compound 5)

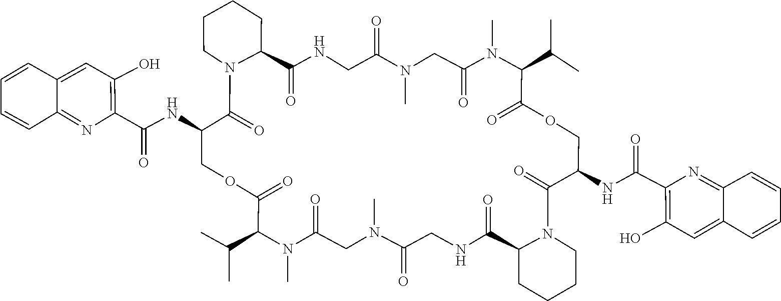 [SMILES: CC(C)[C@H]1C(=O)OC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)c2nc3ccccc3cc2O)C(=O)N2CCCC[C@H]2C(=O)NCC(=O)N(C)CC(=O)N1C]

The title compound was prepared according to Boger et al., 1996, J. American Chem Soc, 118:1629.

LC/MS: Calc'd m/z=1220.6 for C₆₀H₇₆N₁₂O₁₆ found [M+2H]²⁺=611.6.

### 2.6 Benzyl (tert-butoxycarbonyl)-D-serinate (Boc-D-Ser-OH; Compound 6)

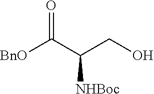 [SMILES: CC(C)(C)OC(=O)N[C@H](CO)C(=O)OCc1ccccc1]

To a stirring solution of D-Ser-OBn*HCl (1.0 g, 5.1 mmol) in DCM (5 mL) at rt was added triethylamine (1.4 mL, 10 mmol, 2 equiv) followed by Boc anhydride (1.3 mL, 5.6 mmol, 1.1 equiv.). This solution was stirred for 30 min, then solvent and excess triethylamine were removed in vacuo to afford the title compound as a thick oil that was used without purification.

LC/MS: Calc'd m/z=295.1 for C₁₅H₂₁NO₅, found [M−Boc+H]⁺=196.2.

### 2.7 (R)-3-(benzyloxy)-2-((tert-butoxycarbonyl)amino)-3-oxopropyl N-(((9H-fluoren-9-yl)methoxy)carbonyl)-N-methyl-L-valinate (Fmoc-Val-D-Ser(Boc)-OBn; Compound 7)

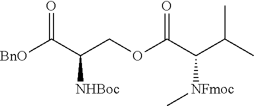

To a solution of Compound 6 (1.5 g, 5.1 mmol, 1 equiv.) and Fmoc-N-methyl-Valine-OH (1.8 g, 5.1 mmol, 1 equiv.) in DCM (50 mL, 0.1 M) at 0° C. was added EDC*HCl (1.2 g, 6.1 mmol, 1.2 equiv.) and DMAP (0.62 g, 5.1 mmol, 1 equiv.). This solution was kept at 4° C. overnight then partitioned between EtOAc (200 mL) and brine (100 mL). The organic layer was washed with brine (2×50 mL), dried over Na₂SO₄, and concentrated to a thick oil. Purification according to General Procedure 12, using a 25 g column and eluting with 30 to 70% EtOAc/hexanes afforded the title compound (1.85 g, 2.9 mmol, 58% yield, 2 steps) as a white solid.

LC/MS: Calc'd m/z=630.3 for C₃₆H₄₂N₂O₈, found [M−Boc+H]⁺=531.4. ¹H NMR (300 MHz, Chloroform-d, 50° C.): δ 7.84-7.74 (m, 2H), 7.61 (td, J=5.6, 2.9 Hz, 2H), 7.48-7.28 (m, 10H), 5.44 (d, J=8.6 Hz, 1H), 5.15 (d, J=17.5 Hz, 2H), 4.64 (d, J=4.2 Hz, 1H), 4.62-4.42 (m, 4H), 4.42-4.30 (m, 1H), 4.30-4.21 (m, 1H), 2.87 (d, J=18.3 Hz, 3H), 2.07 (s, 2H), 1.44 (d, J=7.2 Hz, 9H), 0.89 (td, J=17.3, 16.1, 6.6 Hz, 5H), 0.71 (d, J=6.7 Hz, 1H).

### 2.8 O—(N-(((9H-fluoren-9-yl)methoxy)carbonyl)-N-methyl-L-valyl)-N-(tert-butoxycarbonyl)-D-serine (Fmoc-Val-D-Ser(Boc)-OH; Compound 8)

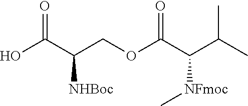

To a solution of Compound 7 (1.85 g, 2.9 mmol, 1 equiv.) in EtOAc (50 mL) was added 5% Pd/C (290 mg, 100 mg/mmol). The flask was purged with H₂ then allowed to stir vigorously under an atmosphere of H₂ for 2 h. This solution was filtered through a plug of celite, washed with EtOAc, and evaporated to dryness. The crude oil was purified as described in General Procedure 12, using a 25 g column and eluting with 0 to 10% MeOH/DCM, affording the title compound (1.47 g, 2.72 mmol, 93% yield) as a fluffy white powder.

LC/MS: Calc'd m/z=540.3 for C₂₉H₃₆N₂O₈, found [M−Boc, +H]⁺=441.2. ¹H NMR (300 MHz, Chloroform-d): δ 8.02 (s, 1H), 7.78 (d, J=7.5 Hz, 2H), 7.61 (d, J=7.3 Hz, 2H), 7.50-7.24 (m, 5H), 5.59 (d, J=8.2 Hz, 1H), 4.73-4.33 (m, 6H), 4.26 (q, J=6.9 Hz, 1H), 2.87 (d, J=22.9 Hz, 3H), 2.27-1.93 (m, 1H), 1.44 (d, J=11.1 Hz, 9H), 0.97 (d, J=6.6 Hz, 2H), 0.85 (dd, J=11.3, 6.5 Hz, 3H), 0.67 (d, J=6.7 Hz, 1H).

### 2.9 N-((((9H-fluoren-9-yl)methoxy)carbonyl)glycyl)-N-methylglycine (Fmoc-Gly-Sar-OH; Compound 9)

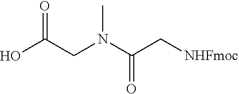

To a stirring solution of H-Gly-Sar-OH (2.5 g, 17.1 mmol, 1 equiv.) in a mixture of dioxane (50 mL) and H₂O (30 mL), at rt, was added K₂CO₃ (2.6 g, 18.8 mmol, 1.1 equiv.) and FmocCl (4.2 g, 17.1 mmol, 1 equiv.). After 1 h, this solution was acidified by the addition of aqueous 1 M HCl until a pH<3 was achieved. The dioxane was removed in vacuo and the cloudy aqueous solution was extracted with EtOAc (3×100 mL). The organic layers were combined, and solvent removed. Purification according to General Procedure 12, using a 120 g C18 column and eluting with 0 to 90% CH₃CN/H₂O, afforded the title compound (6.2 g, 16.8 mmol, 98% yield) as a white powder.

LC/MS: Calc'd m/z=368.1 for C₂₀H₂₀N₂O₅, found [M+H]⁺=369.2.

### 2.10 (S)-1-(O)—(N—(N—(((S)-1-((R)-3-((S)-2-(2-(2-amino-N-methylacetamido)-N-methylacetamido)-3-methylbutanamido)-2-((tert-butoxycarbonyl)amino)propanoyl)piperidine-2-carbonyl)glycyl)-N-methylglycyl)-N-methyl-L-valyl)-N-(tert-butoxycarbonyl)-D-seryl) piperidine-2-carboxylic acid (Compound 10)

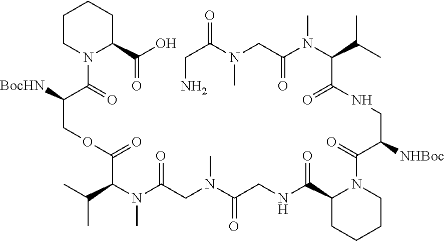 [SMILES: CC(C)[C@@H](C(=O)NC[C@@H](NC(=O)OC(C)(C)C)C(=O)N1CCCC[C@H]1C(=O)NCC(=O)N(C)CC(=O)N(C)[C@H](C(=O)OC[C@@H](NC(=O)OC(C)(C)C)C(=O)N1CCCC[C@H]1C(=O)O)C(C)C)N(C)C(=O)CN(C)C(=O)CN]

The title compound was synthesized according to general procedures 1 and 2 utilizing Compound 8, Compound 9 and Fmoc-Pip-OH, Boc-D-Dap (Fmoc)-OH, Fmoc-N-methyl-Val-OH, Fmoc-Sar-OH and Fmoc-Gly-OH, on a 0.2 mmol scale. The crude peptide (150 mg, 0.137 mmol, 68% yield) was isolated, post ether precipitation, as a white powder and was used without additional purification.

LC/MS: Calc'd m/z=1095.6 for C₅₀H₈₅N₁₁O₁₆, found [M−2Boc+2H]²⁺=449.0, [M−Boc+2H]²⁺=499.0.

### 2.11 Di-tert-butyl ((7R,11S,19aS,26R,30S,38aS)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)dicarbamate (Compound 11)

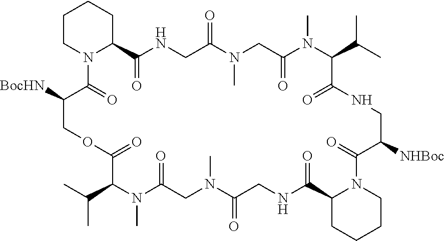 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 10 (130 mg, 0.12 mmol). Purification according to General Procedure 12, using a 10 g column and eluting with 0 to 20% MeOH/DCM, afforded the title compound (109 mg, 0.101 mmol, 86% yield) as a white solid.

LC/MS: Calc'd m/z=1077.61 for C₅₀H₈₃N₁₁O₁₅, found [M−2Boc+2H]²⁺=440.0, [M−Boc,+H]⁺=978.8.

### 2.12 (7R,11S,19aS,26R,30S,38aS)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diaminium chloride (Compound 12)

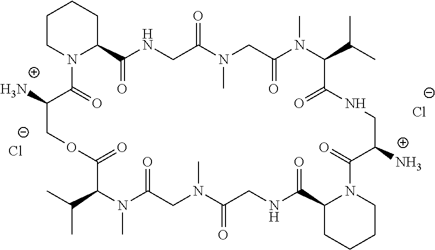 [SMILES: [Cl-] ; CC(C)[C@H]1C(=O)NC[C@@H]([NH3+])C(=O)N2CCCC[C@H]2C(=O)NCC(=O)N(C)CC(=O)N(C)[C@@H](C(C)C)C(=O)OC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 11 (18 mg, 0.017 mmol) and 2 mL of 4 M HCl/dioxane.

LC/MS: Calc'd m/z=877.5 for C₄₀H₆₇N₁₁O₁₁, found [M+2H]²⁺=440.0, [M+H]⁺=878.8.

### 2.13 N,N′-((7R,11S,19aS,26R,30S,38aS)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-(benzyloxy)quinoline-2-carboxamide) (Compound 13)

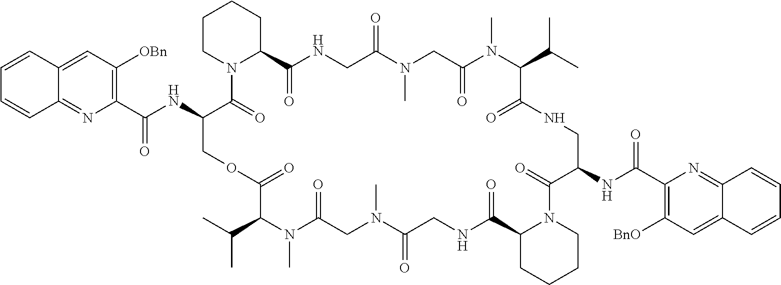 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)OC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 12 (0.017 mmol) and NaHCO₃ as the base instead of NEt(i-Pr)₂. Purification according to General Procedure 12, using a 10 g column and eluting 0 to 20% MeOH/DCM, afforded the title compound (11.1 mg, 0.008 mmol, 47% yield) as a white solid.

LC/MS: Calc'd m/z=1399.6 for C₇₄H₈₉N₁₃O₁₅, found [M+2H]²⁺=701.4.

### 2.14 N,N′-((7R,11S,19aS,26R,30S,38aS)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-hydroxyquinoline-2-carboxamide) (Compound 14; FD-2)

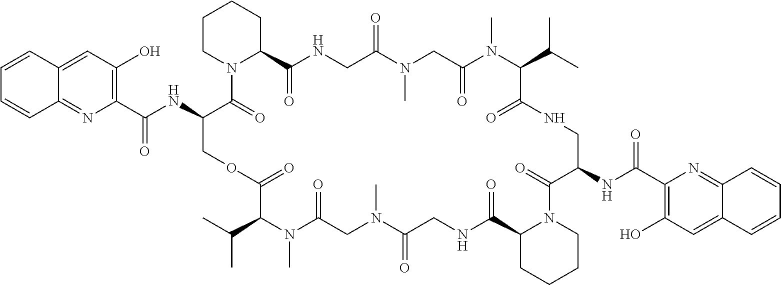 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)c2nc3ccccc3cc2O)C(=O)N2CCCC[C@H]2C(=O)NCC(=O)N(C)CC(=O)N1C]

Compound 14 was synthesized as described in General Procedure 6 starting from Compound 13 (5.5 mg, 0.004 mmol), 10% Pd/C (2.5 mg) and MeOH (2 mL). Following filtration, the MeOH was diluted with EtOAc (20 mL) and washed with a saturated aqueous solution of NaCl. The organic layer was dried over Na₂SO₄ and concentrated under reduced pressure to afford the title compound (2.8 mg, 0.0023 mmol, 57% yield) as a vibrant yellow solid.

LC/MS: Calc'd m/z=1219.6 for C₆₀H₇₇N₁₃O₁₅, found [M+2H]²⁺=611.2.

### 2.15 Benzyl (tert-butoxycarbonyl)-D-allothreoninate (Boc-D-AlloThr-OBn; Compound 15)

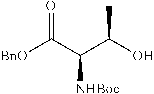 [SMILES: C[C@@H](O)[C@@H](NC(=O)OC(C)(C)C)C(=O)OCc1ccccc1]

To a stirring solution of Boc-D-allothreonine (2.4 g, 11 mmol) in DMF (50 mL), at rt, was added NaHCO₃ (2.8 g, 33 mmol, 3 equiv.) followed by benzyl bromide (3.9 mL, 33 mmol, 3 equiv.). This solution was stirred for 3 days, then DMF was removed by rotavap. The crude residue was purified as described in General Procedure 12, using a 100 g column and eluting with 10 to 50% EtOAc/hexanes to afford the title compound (2.61 g, 8.4 mmol, 50% yield) as a clear oil that solidified upon standing at 4° C. overnight.

LC/MS: Calc'd m/z=309.2 for C₁₆H₂₃NO₅, found [M−Boc+H]⁺=210.2, found [M+Na]⁺=332.2. ¹H NMR (300 MHz, chloroform-d): δ 7.44-7.32 (m, 5H), 5.50-5.42 (m, 1H), 5.23 (d, J=2.5 Hz, 2H), 4.45 (s, 1H), 4.17 (s, 1H), 2.95 (s, 1H), 1.47 (s, 9H), 1.16 (d, J=6.4 Hz, 3H).

### 2.16 Benzyl O—(N-(((9H-fluoren-9-yl)methoxy)carbonyl)-N-methyl-L-valyl)-N-(tert-butoxycarbonyl)-D-allothreoninate (Fmoc-N-methyl-Val-D-AlloThr(Boc)-OBn; Compound 16)

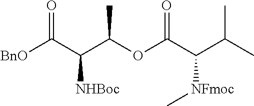

To a stirring solution of Compound 15 (1.3 g, 4.2 mmol, 1 equiv.) and Fmoc-N-methyl-Val-OH (1.5 g, 4.2 mmol, 1 equiv.) in DCM (42 mL, 0.1 M), at 0° C., was added EDC*HCl (960 mg, 5.0 mmol, 1.2 equiv.) and DMAP (510 mg, 4.2 mmol, 1 equiv.). This solution was kept at 4° C. overnight, then diluted with DCM (100 mL) and washed with aqueous 0.1 M HCl (50 mL) and saturated aqueous NaCl (50 mL). The organic layer was dried over Na₂SO₄ and the solvent removed by rotavap. Purification according to General Procedure 12, using a 50 g column and eluting with 10 to 70% EtOAc/hexanes, afforded the title compound (2.2 g, 3.4 mmol, 81% yield) as a white solid.

LC/MS: Calc'd m/z=644.3 for C₃₇H₄₄N₂O₈, found [M−Boc+H]⁺=545.4, found [M+Na]⁺=668.5. ¹H NMR (300 MHz, chloroform-d): δ 7.79 (d, J=7.5 Hz, 2H), 7.62 (d, J=7.4 Hz, 2H), 7.47-7.27 (m, 8H), 5.48 (d, J=8.5 Hz, 1H), 5.29-5.19 (m, 2H), 5.14 (d, J, 12.3 Hz, 2H), 4.60-4.36 (m, 3H), 4.35-4.24 (q, J=9 Hz, 1H), 2.89 (d, J=19.3 Hz, 3H), 2.30-2.10 (m, 1H). 1.69 (s, 1H), 1.42 (d, J=5.0 Hz, 9H), 1.36-1.25 (m, 3H), 1.00 (d, J=6.6 Hz, 2H), 0.93 (d, J=6.6 Hz, 1H), 0.89 (d, J=6.6 Hz, 2H), 0.79 (d, J=6.6 Hz, 1H).

### 2.17 O—(N-(((9H-fluoren-9-yl)methoxy)carbonyl)-N-methyl-L-valyl)-N-(tert-butoxycarbonyl)-D-allothreonine (Fmoc-N-methyl-Val-D-AlloThr(Boc)-OH; Compound 17)

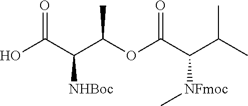

The title compound was synthesized as described in General Procedure 6 starting from Compound 16 (2.2 g, 3.4 mmol), with 5% Pd/C (340 mg) and EtOAc (34 mL, 0.1 M). Following removal of Pd/C, the title compound (1.86 g, 3.35 mmol, 99% yield) was isolated as white solid and used without additional purification.

LC/MS: Calc'd m/z=554.3 for C₃₀H₃₆N₂O₈, found [M−Boc+H]⁺=455.4, found [M+Na]⁺=577.4. ¹H NMR (300 MHz, chloroform-d): δ 7.78 (dd, J=7.2, 1.4 Hz, 2H), 7.68-7.55 (m, 2H), 7.47-7.16 (m, 4H), 5.51 (d, J=8.4 Hz, 1H), 5.30-5.21 (m, 1H), 4.59-4.43 (m, 1H), 4.46 (s, 6H), 4.29 (dd, J=12.5, 5.8 Hz, 2H), 2.93 (s, 2H), 2.79 (s, 1H), 2.23 (m, 1H), 1.4 (m, 9H), 1.01 (d, J=6.5 Hz, 2H), 0.89 (d, J=6.7 Hz, 3H), 0.68 (d, J=6.7 Hz, 1H).

### 2.18 (S)-1-(N-(tert-butoxycarbonyl)-O—(N—(N—(((S)-1-(N-(tert-butoxycarbonyl)-O—(N—(N—glycyl-N-methylglycyl)-N-methyl-L-valyl)-D-allothreonyl) piperidine-2-carbonyl)glycyl)-N-methylglycyl)-N-methyl-L-valyl)-D-allothreonyl) piperidine-2-carboxylic acid (Compound 18)

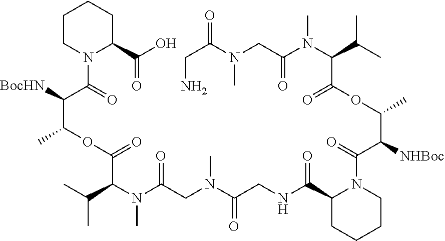 [SMILES: CC(C)[C@@H](C(=O)O[C@H](C)[C@@H](NC(=O)OC(C)(C)C)C(=O)N1CCCC[C@H]1C(=O)O)N(C)C(=O)CN(C)C(=O)CNC(=O)[C@@H]1CCCCN1C(=O)[C@H](NC(=O)OC(C)(C)C)[C@@H](C)OC(=O)[C@H](C(C)C)N(C)C(=O)CN(C)C(=O)CN]

The title compound was synthesized according to General Procedures 1 and 2 utilizing Fmoc-Pip-OH, Compound 9, and Compound 17, on a 0.1 mmol scale. The crude peptide (23 mg, 0.02 mmol, 20% yield) was isolated, post ether precipitation, as white powder and was used without additional purification.

LC/MS: Calc'd m/z=1124.6 for C₅₂H₈₈N₁₀O₁₇, found [M−2Boc, +2H]²⁺=463.4, [M−Boc+2H]²⁺=513.4.

### 2.19 Di-tert-butyl ((7R,8R,11S,19aS,26R,27R,30S,38aS)-11,30-diisopropyl-8,12,15,27,31,34-hexamethyl-6,10,13,16,19,25,29,32,35,38-decaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)dicarbamate (Compound 19)

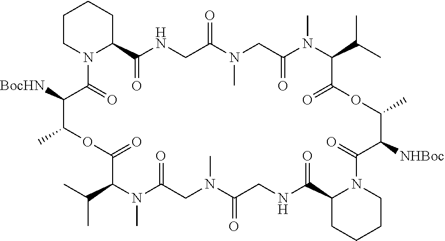 [SMILES: CC(C)[C@H]1C(=O)O[C@H](C)[C@@H](NC(=O)OC(C)(C)C)C(=O)N2CCCC[C@H]2C(=O)NCC(=O)N(C)CC(=O)N(C)[C@@H](C(C)C)C(=O)O[C@H](C)[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 18 (23 mg, 0.02 mmol). Purification according to General Procedure 12, using a 10 g column and eluting with 0 to 15% MeOH/DCM, afforded the title compound (11 mg, 0.01 mmol, 50% yield) as a white solid.

LC/MS: Calc'd m/z=1106.6 for C₅₂H₈₆N₁₀O₁₆, found [M−2Boc+2H]²⁺=454.4.

### 2.20 (7R,8R,11S,19aS,26R,27R,30S,38aS)-11,30-diisopropyl-8,12,15,27,31,34-hexamethyl-6,10,13,16,19,25,29,32,35,38-decaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diaminium chloride (Compound 20)

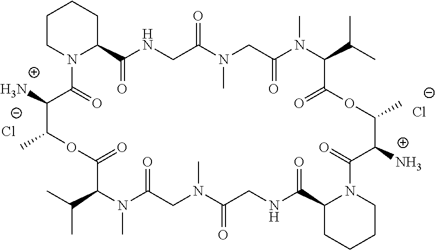 [SMILES: [Cl-] ; CC(C)[C@H]1C(=O)O[C@H](C)[C@@H]([NH3+])C(=O)N2CCCC[C@H]2C(=O)NCC(=O)N(C)CC(=O)N(C)[C@@H](C(C)C)C(=O)O[C@H](C)[C@@H]([NH3+])C(=O)N2CCCC[C@H]2C(=O)NCC(=O)N(C)CC(=O)N1C ; [Cl-]]

The title compound was prepared according to General Procedure 5 starting from Compound 19 (11 mg, 0.01 mmol) and 2 mL of 4 M HCl/dioxane.

LC/MS: Calc'd m/z=906.5 for C₄₂H₇₀N₁₀O₁₂, found [M+2H]²⁺=454.4, [M+H]⁺=908.6.

### 2.21 N,N′-((7R,8R,11S,19aS,26R,27R,30S,38aS)-11,30-diisopropyl-8,12,15,27,31,34-hexamethyl-6,10,13,16,19,25,29,32,35,38-decaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)bis(3-(benzyloxy)quinoline-2-carboxamide) (Compound 21)

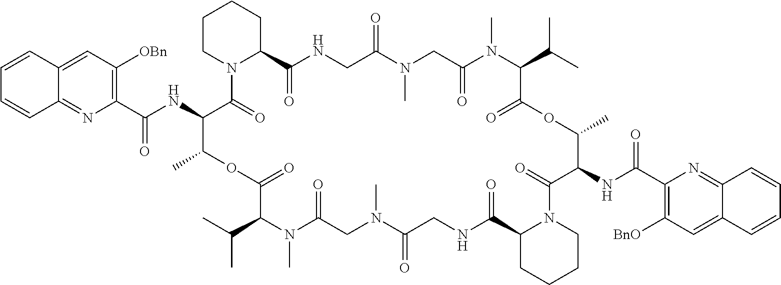 [SMILES: CC(C)[C@H]1C(=O)O[C@H](C)[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)O[C@H](C)[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 20 (0.01 mmol) and NMM as a base rather than NEt(i-Pr)₂. Purification according to General Procedure 12, using a 10 g column and eluting with 0 to 20% MeOH/DCM, afforded the title compound (14 mg, 0.01 mmol, 100% yield) as a white solid.

LC/MS: Calc'd m/z=1428.7 for C₇₆H₉₂N₁₂O₁₆, found [M+2H]²⁺=715.8.

### 2.22 N,N′-((7R,8R,11S,19aS,26R,27R,30S,38aS)-11,30-diisopropyl-8,12,15,27,31,34-hexamethyl-6,10,13,16,19,25,29,32,35,38-decaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)bis(3-hydroxyquinoline-2-carboxamide) (Compound 22; FD-6)

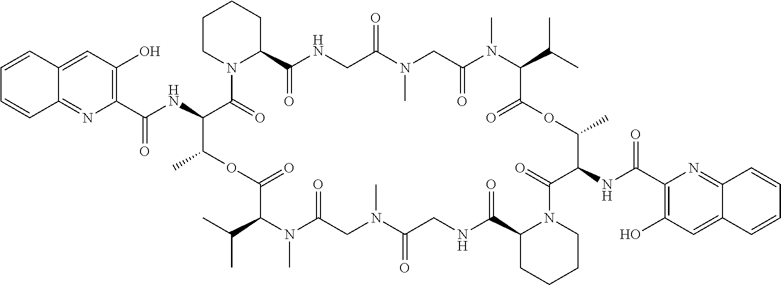 [SMILES: CC(C)[C@H]1C(=O)O[C@H](C)[C@@H](NC(=O)c2nc3ccccc3cc2O)C(=O)N2CCCC[C@H]2C(=O)NCC(=O)N(C)CC(=O)N(C)[C@@H](C(C)C)C(=O)O[C@H](C)[C@@H](NC(=O)c2nc3ccccc3cc2O)C(=O)N2CCCC[C@H]2C(=O)NCC(=O)N(C)CC(=O)N1C]

Compound 22 was synthesized as described in General Procedure 6 starting from Compound 21 (14 mg, 0.01 mmol), 10% Pd/C (2 mg) and MeOH (2 mL). Following removal of Pd/C, the title compound (9.1 mg, 0.07 mmol, 70% yield) was isolated as a vibrant yellow solid.

LC/MS: Calc'd m/z=1248.6 for C₆₂H₈₀N₁₂O₁₆, found [M+2H]²⁺=625.6, [M+H]⁺=1249.5.

### 2.23 (2R,3R)-4-(benzyloxy)-3-((tert-butoxycarbonyl)amino)-4-oxobutan-2-yl N-(((9H-fluoren-9-yl)methoxy)carbonyl)-N-methyl-L-isoleucinate (Fmoc-N-methyl-Ile-D)-AlloThr(Boc)-OBn; Compound 23)

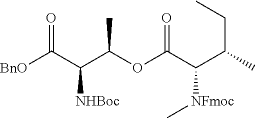

To a stirring solution of Compound 15 (1.3 g, 4.2 mmol, 1 equiv.) and Fmoc-N-methyl-Ile-OH (1.54 g, 4.2 mmol, 1 equiv.) in DCM (42 mL, 0.1 M), at 0° C., was added EDC*HCl (960 mg, 5.0 mmol, 1.2 equiv.) and DMAP (510 mg, 4.2 mmol, 1 equiv.). This solution was kept at 4° C. overnight then diluted with DCM (100 mL) and washed with an aqueous 0.1M HCl (50 mL) and saturated aqueous NaCl (50 mL). The organic layer was dried over Na₂SO₄, and the solvent removed by rotavap. Purification according to General Procedure 12, using a 100 g column and eluting with a linear gradient of 10 to 70% EtOAc/hexanes, afforded the title compound (2.27 g, 3.5 mmol, 83% yield) as a white solid.

LC/MS: Calc'd m/z=658.3 for C₃₈H₄₆N₂O₈, found [M−Boc+H]⁺=559.4, found [M+Na]⁺=681.6.

### 2.24 O—(N-(((9H-fluoren-9-yl)methoxy)carbonyl)-N-methyl-L-isoleucyl)-N-(tert-butoxycarbonyl)-D-allothreonine (Compound 24)

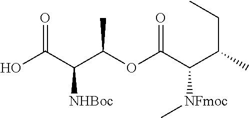

The title compound was synthesized as described in General Procedure 6 starting from Compound 23 (2.27 g, 3.5 mmol), 5% Pd/C (350 mg), and EtOAc (35 mL, 0.1 M). Following removal of Pd/C, the title compound (1.81 g, 3.2 mmol, 91% yield) was isolated as white solid and used without additional purification.

LC/MS: Calc'd m/z=568.3 for C₃₁H₄₀N₂O₈, found [M−Boc+H]⁺=469.4, found [M+Na]⁺=591.4.

### 2.25 (S)-1-(O—(N—(N—(((S)-1-((R)-3-((2S,3S)-2-(2-(2-amino-N-methylacetamido)-N-methylacetamido)-3-methylpentanamido)-2-(((benzyloxy)carbonyl)amino)propanoyl)piperidine-2-carbonyl)glycyl)-N-methylglycyl)-N-methyl-L-isoleucyl)-N-(tert-butoxycarbonyl)-D-allothreonyl) piperidine-2-carboxylic acid (Compound 25)

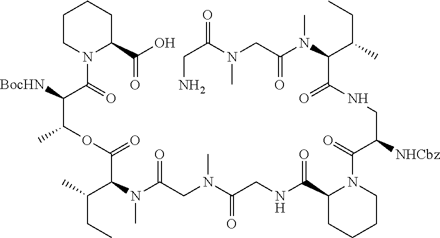 [SMILES: CC[C@H](C)[C@@H](C(=O)NC[C@@H](NC(=O)OCc1ccccc1)C(=O)N1CCCC[C@H]1C(=O)NCC(=O)N(C)CC(=O)N(C)[C@H](C(=O)O[C@H](C)[C@@H](NC(=O)OC(C)(C)C)C(=O)N1CCCC[C@H]1C(=O)O)[C@@H](C)CC)N(C)C(=O)CN(C)C(=O)CN]

The title compound was synthesized according to General Procedures 1 and 2 utilizing Compound 9, Compound 24, Fmoc-Pip-OH, Z-Dap(Fmoc)OH, Fmoc-N-methyl-Ile-OH, Fmoc-Sar-OH and Fmoc-Gly-OH, on a 0.2 mmol scale. The crude peptide (150 mg, 0.128 mmol, 64% yield) was isolated, post ether precipitation, as a white powder and was used without additional purification.

LC/MS: Calc'd m/z=1171.6 for C₅₆H₈₉N₁₁O₁₆, found [M−Boc+2H]²⁺=537.0, [M+H]⁺=1172.8.

### 2.26 Benzyl tert-butyl ((7R,8R,11S,19aS,26R,30S,38aS)-11,30-di((S)-sec-butyl)-8,12,15,31,34-pentamethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)dicarbamate (Compound 26)

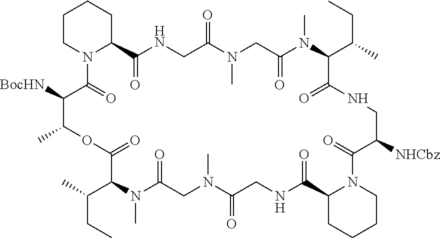 [SMILES: CC[C@H](C)[C@H]1C(=O)NC[C@@H](NC(=O)OCc2ccccc2)C(=O)N2CCCC[C@H]2C(=O)NCC(=O)N(C)CC(=O)N(C)[C@@H]([C@@H](C)CC)C(=O)O[C@H](C)[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 25 (150 mg, 0.128 mmol). Purification according to General Procedure 12, using a 10 g column and eluting with 0 to 15% MeOH/DCM, afforded the title compound (105 mg, 0.091 mmol, 71% yield) as a white solid.

LC/MS: Calc'd m/z=1153.6 for C₅₆H₈₇N₁₁O₁₅, found [M−Boc+2H]²⁺=528.0, [M−Boc, +H]⁺=1054.8, [M+H]⁺=1154.8.

### 2.27 Tert-butyl ((7R,8R,11S,19aS,26R,30S,38aS)-26-amino-11,30-di((S)-sec-butyl)-8,12,15,31,34-pentamethyl-6,10,13,16,19,25,29,32,35,38-decaoxotetratriacontahydro-2H,6H,10H-dipyrido[2,1-b₁:2′,1′-l][1]oxa[4,7,10,13,17,20,23,26,29]nonaazacyclodotriacontin-7-yl)carbamate (Compound 27)

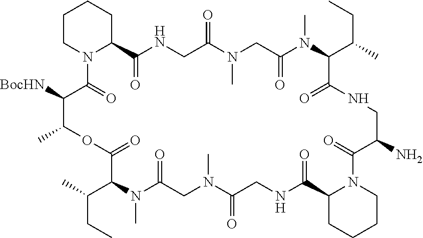 [SMILES: CC[C@H](C)[C@H]1C(=O)NC[C@@H](N)C(=O)N2CCCC[C@H]2C(=O)NCC(=O)N(C)CC(=O)N(C)[C@@H]([C@@H](C)CC)C(=O)O[C@H](C)[C@@H](NC(=O)OC(C)(C)C)C(=O)N2CCCC[C@H]2C(=O)NCC(=O)N(C)CC(=O)N1C]

The title compound was synthesized as described in General Procedure 6 starting from Compound 26 (18 mg, 0.015 mmol), 10% Pd/C (2 mg) and MeOH (2 mL). Following removal of Pd/C, the title compound was isolated as a white solid.

LC/MS: Calc'd m/z=1019.6 for C₄₆H₈₁N₁₁O₁₃, found [M−Boc+2H]²⁺=461.0, [M+H]⁺=1021.0.

### 2.28 (7R,8R,11S,19aS,26R,30S,38aS)-11,30-di((S)-sec-butyl)-8,12,15,31,34-pentamethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diaminium chloride (Compound 28)

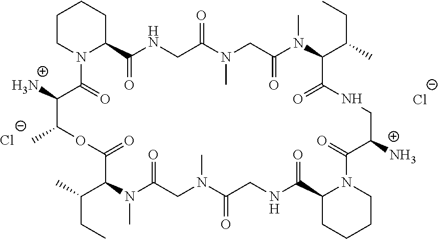 [SMILES: [Cl-] ; CC[C@H](C)[C@H]1C(=O)NC[C@@H]([NH3+])C(=O)N2CCCC[C@H]2C(=O)NCC(=O)N(C)CC(=O)N(C)[C@@H]([C@@H](C)CC)C(=O)O[C@H](C)[C@@H]([NH3+])C(=O)N2CCCC[C@H]2C(=O)NCC(=O)N(C)CC(=O)N1C ; [Cl-]]

The title compound was prepared according to General Procedure 5 starting from Compound 27 (0.015 mmol) and 2 mL of 4 M HCl/dioxane.

LC/MS: Calc'd m/z=919.5 for C₄₃H₇₃N₁₁O₁₁, found [M+2H]²⁺=460.0, [M+H]⁺=921.0.

### 2.29 N,N′-((7R,8R,11S,19aS,26R,30S,38aS)-11,30-di((S)-sec-butyl)-8,12,15,31,34-pentamethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-(benzyloxy)quinoline-2-carboxamide) (Compound 29)

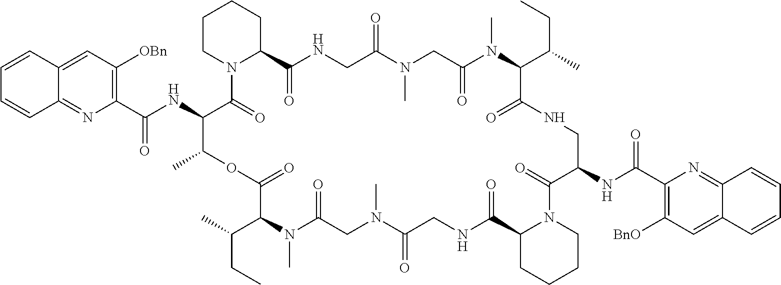 [SMILES: CC[C@H](C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H]([C@@H](C)CC)C(=O)O[C@H](C)[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 28 (0.015 mmol). Preparative HPLC purification according to General Procedure 12, eluting with 20 to 90% CH₃CN/H₂O over 20 min, afforded the title compound (5 mg, 0.003 mmol, 23% yield) as a white solid.

LC/MS: Calc'd m/z=1441.7 for C₇₇H₉₃N₁₃O₁₅, found [M+2H]²⁺=722.4.

### 2.30 N,N′-((7R,8R,11S,19aS,26R,30S,38aS)-11,30-di((S)-sec-butyl)-8,12,15,31,34-pentamethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-hydroxyquinoline-2-carboxamide) (Compound 30; FD-30)

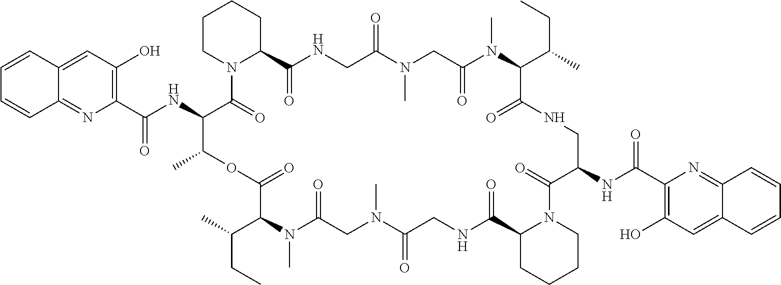 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)CC)C(=O)O[C@H](C)[C@@H](NC(=O)c2nc3ccccc3cc2O)C(=O)N2CCCC[C@H]2C(=O)NCC(=O)N(C)CC(=O)N1C]

Compound 30 was synthesized as described in General Procedure 6 starting from Compound 29 (5 mg, 0.003 mmol), 10% Pd/C (2 mg), NaHCO₃ (0.5 mg, 0.006 mmol, 2 equiv.) and EtOAc (2 mL). Following removal of Pd/C and solvents, the title compound (2 mg, 0.002 mmol, 66% yield) was isolated as a vibrant yellow solid.

LC/MS: Calc'd m/z=1261.6 for C₆₃H₈₃N₁₃O₁₅, found [M+2H]²⁺=632.2, [M+H]⁺=1262.8.

### 2.31 (S)-1-((9S,13R)-1-((S)-1-((R)-3-((S)-2-(2-(2-amino-N-methylacetamido)-N-methylacetamido)-3-methylbutanamido)-2-((tert-butoxycarbonyl)amino)propanoyl) piperidin-2-yl)-13-((tert-butoxycarbonyl)amino)-9-isopropyl-5,8-dimethyl-1,4,7,10-tetraoxo-2,5,8,11-tetraazatetradecan-14-oyl) piperidine-2-carboxylic acid (Compound 31)

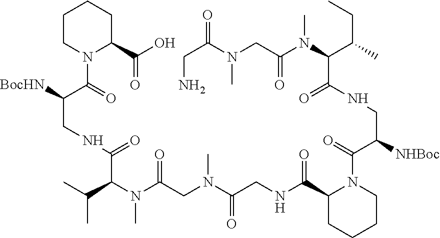 [SMILES: CC[C@H](C)[C@@H](C(=O)NC[C@@H](NC(=O)OC(C)(C)C)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)O)C(C)C)N(C)C(=O)CN(C)C(=O)CN]

The title compound was synthesized according to General Procedure 1 utilizing Fmoc-Pip-OH, Boc-D-Dap(Fmoc)-OH, Fmoc-N-methyl-Val-OH, Fmoc-Sar-OH and Fmoc-Gly-OH, on a 0.05 mmol scale. The crude peptide was purified as described in General Procedure 12, eluting with 10 to 70% CH₃CN/H₂O, afforded the title compound (16 mg, 0.015 mmol, 29% yield) as a white solid.

LC/MS: Calc'd m/z=1094.6 for C₅₀H₈₆N₁₂O₁₅, found [M−Boc+2H]²⁺=498.6, [M−2Boc+2H]²⁺=448.4.

### 2.32 di-tert-butyl ((7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 32)

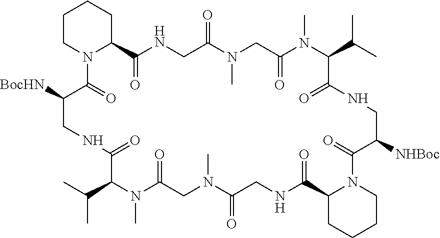 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)[C@@H](C(C)C)C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 31 (24 mg, 0.0.022 mmol). Purification according to General Procedure 12, using a 12 g C18 column and eluting with 20 to 70% CH₃CN/H₂O, afforded the title compound (8 mg, 0.007 mmol, 34% yield) as a white solid.

LC/MS: Calc'd m/z=1076.6 for C₅₀H₈₄N₁₂O₁₄, found [M−Boc, +H]⁺=977.8, [M−Boc+2H]²⁺=439.4.

### 2.33 (7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium chloride (Compound 33)

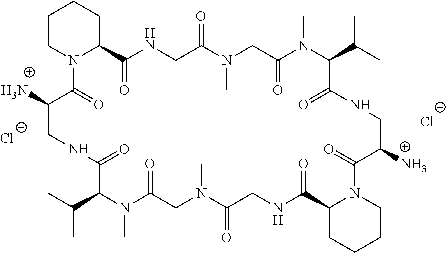 [SMILES: [Cl-] ; CC(C)[C@H]1C(=O)NC[C@@H]([NH3+])C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 32 (8 mg, 0.007 mmol), and 2 mL of 4 M HCl/dioxane.

LC/MS: Calc'd m/z=948.5 for C₄₀H₇₀C₁₂N₁₂O₁₀, found [M+2H]²⁺=439.4, [M+H]⁺=877.8.

### 2.34 N,N′-((7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 34)

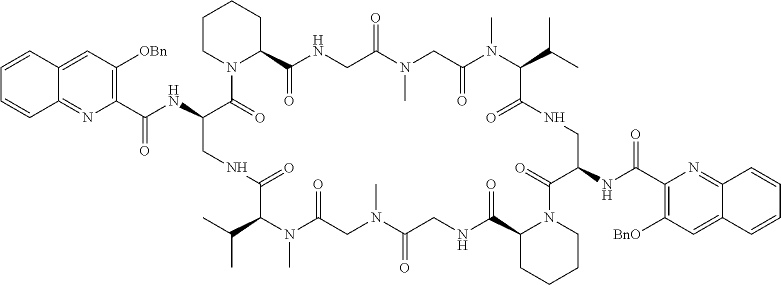 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 33 (0.007 mmol). Preparative HPLC purification according to General Procedure 12, eluting with 20 to 50% CH₃CN/H₂O over 20 min, afforded the title compound (8 mg, 0.006 mmol, 82% yield, 2 steps) as a white solid.

LC/MS: Calc'd m/z=1398.7 for C₇₄H₉₀N₁₄O₁₄, found [M+2H]²⁺=700.8.

### 2.35 N,N′-((7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 35; FD-1)

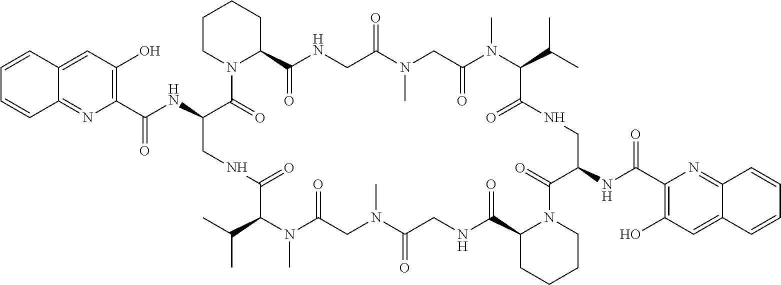 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

Compound 35 was synthesized as described in General Procedure 6 starting from Compound 34 (8 mg, 0.003 mmol), 10% Pd/C (2 mg) and EtOAc (2 mL). Following removal of Pd/C, the title compound (6 mg, 0.005 mmol, 86% yield) was isolated as a pale-yellow solid.

LC/MS: Calc'd m/z=1218.6 for C₆₀H₇₈N₁₄O₁₄, found [M+2H]²⁺=610.8, [M+H]⁺=1219.8.

### 2.36 (R)-3-(benzyl(methyl)amino)-2-((tert-butoxycarbonyl)amino) propanoic acid (Compound 36)

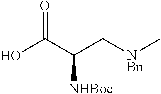 [SMILES: CN(Cc1ccccc1)C[C@@H](NC(=O)OC(C)(C)C)C(=O)O]

The title compound was prepared as described in Ryszard, A., 1988, Polish Journal of Chemistry, 62:257.

LC/MS: Calc'd m/z=308.38 for C₁₅H₂₄N₂O₄, found [M+H]⁺=309.2. ¹H NMR: (300 MHz, methanol-d₄) δ 7.64-7.42 (m, 5H), 4.53-4.25 (m, 1H), 3.55 (d, J=6.1 Hz, 1H), 3.41 (dd, J=13.1, 8.7 Hz, 1H), 2.91 (s, 3H), 1.49 (s, 9H).

### 2.37 (R)-3-((((9H-fluoren-9-yl)methoxy)carbonyl)(methyl)amino)-2-((tert-butoxycarbonyl)amino) propanoic acid (Compound 37)

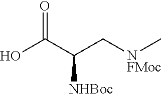

The title compound was prepared in two steps from Compound 36 (740 mg, 1.75 mmol). First, the benzyl group was removed as described in General Procedure 6 using 10% Pd/C (350 mg) and MeOH (24 mL). Following removal of Pd/C and methanol, the free amine was dissolved in dioxane (50 mL) and H₂O (40 mL). To this solution was added K₂CO₃ (480 mg, 3.5 mmol, 2 equiv.) followed by FmocCl (500 mg, 1.9 mmol, 1.1 equiv.). This solution was stirred for 1 h, concentrated to half-volume and acidified (pH<3) with aqueous 1 M HCl. The resulting cloudy solution was extracted with EtOAc (200 mL). The organic layer was then washed with brine (2×50 mL), dried over Na₂SO₄ and concentrated to dryness. Purification according to General Procedure 12, using a 60 g C18 column and eluting with 30 to 100% CH₃CN/H₂O, afforded the title compound (660 mg, 1.5 mmol, 86% yield) as a white powder.

LC/MS: Calc'd m/z=440.50 for C₂₄H₂₈N₂O₆, found [M−Boc+H]⁺=342.2, [M+Na]⁺=463. ¹H NMR: (300 MHZ, Acetone-d₆) δ 7.88 (d, J=7.3 Hz, 2H), 7.71 (d, J=7.1 Hz, 2H), 7.40 (dt, J=22.1, 7.3 Hz, 4H), 4.39 (tt, J=15.2, 7.9 Hz, 4H), 3.79 (dd, J=14.3, 5.2 Hz, 1H), 3.69-3.47 (m, 1H), 3.00 (d, J=23.2 Hz, 3H), 1.41 (s, 9H).

### 2.38 (S)-1-((9S,13R)-1-((S)-1-((R)-3-((S)-2-(2-(2-amino-N-methylacetamido)-N-methylacetamido)-N,3-dimethylbutanamido)-2-((tert-butoxycarbonyl)amino)propanoyl) piperidin-2-yl)-13-((tert-butoxycarbonyl)amino)-9-isopropyl-5,8,11-trimethyl-1,4,7,10-tetraoxo-2,5,8,11-tetraazatetradecan-14-oyl) piperidine-2-carboxylic acid (Compound 38)

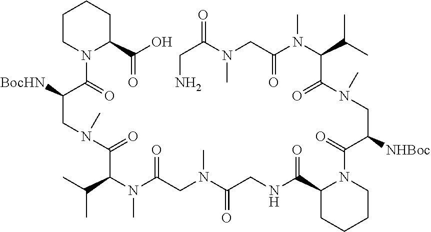 [SMILES: CC(C)[C@@H](C(=O)N(C)C[C@@H](NC(=O)OC(C)(C)C)C(=O)N1CCCC[C@H]1C(=O)O)N(C)C(=O)CN(C)C(=O)CNC(=O)[C@@H]1CCCCN1C(=O)[C@@H](CN(C)C(=O)[C@H](C(C)C)N(C)C(=O)CN(C)C(=O)CN)NC(=O)OC(C)(C)C]

The title compound was synthesized according to General Procedure 1 utilizing Compound 37, Fmoc-Pip-OH, Fmoc-N-methyl-Val-OH, Fmoc-Sar-OH and Fmoc-Gly-OH, on a 0.2 mmol scale. The crude peptide (80 mg, 0.07 mmol, 36% yield) was isolated, post ether precipitation, as white powder and was used without additional purification.

LC/MS: Calc'd m/z=1123.36 for C₅₂H₉₀N₁₂O₁₅, found [M+H]⁺=1123.8, [M−Boc+2H]²⁺=512.4.

### 2.39 Di-tert-butyl ((7R,11S,19aS,26R,30S,38aS)-11,30-diisopropyl-9,12,15,28,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 39)

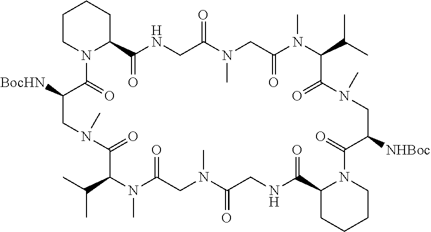 [SMILES: CC(C)[C@H]1C(=O)N(C)C[C@@H](NC(=O)OC(C)(C)C)C(=O)N2CCCC[C@H]2C(=O)NCC(=O)N(C)CC(=O)N(C)[C@@H](C(C)C)C(=O)N(C)C[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 38 (80 mg, 0.0.07 mmol). Purification according to General Procedure 12, using a 10 g column and eluting with 0 to 20% MeOH/CH₂Cl₂, afforded the title compound (51 mg, 0.046 mmol, 65% yield) as a white solid.

LC/MS: Calc'd m/z=1105.35 for C₅₂H₈₈N₁₂O₁₄, found [M+H]⁺=1105.8, [M−Boc+H]⁺=1005.8, [M−Boc+2H]²⁺=503.6.

### 2.40 (7R,11S,19aS,26R,30S,38aS)-11,30-diisopropyl-9,12,15,28,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium (Compound 40)

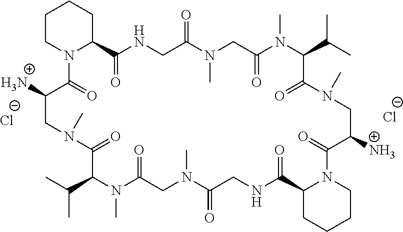 [SMILES: [Cl-] ; CC(C)[C@H]1C(=O)N(C)C[C@@H]([NH3+])C(=O)N2CCCC[C@H]2C(=O)NCC(=O)N(C)CC(=O)N(C)[C@@H](C(C)C)C(=O)N(C)C[C@@H]([NH3+])C(=O)N2CCCC[C@H]2C(=O)NCC(=O)N(C)CC(=O)N1C ; [Cl-]]

The title compound was prepared according to General Procedure 5 starting from Compound 39 (40 mg, 0.036 mmol), and 2 mL of 4 M HCl/dioxane.

LC/MS: Calc'd m/z=904.5 for C₄₂H₇₂N₁₂O₁₀, found [M+H]⁺=905.8, [M+2H]²⁺=453.4.

### 2.41 N,N′-((7R,11S,19aS,26R,30S,38aS)-11,30-diisopropyl-9,12,15,28,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 41)

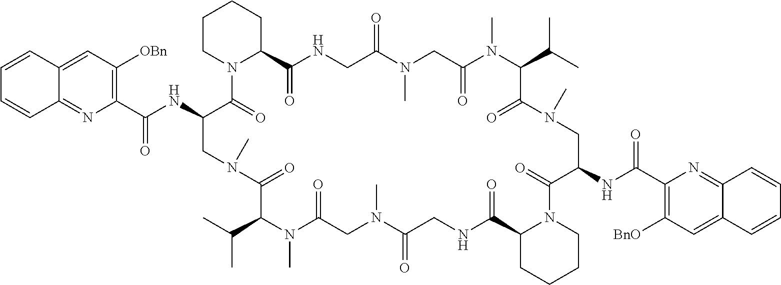 [SMILES: CC(C)[C@H]1C(=O)N(C)C[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)N(C)C[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 40 (0.028 mmol). Purification according to General Procedure 12, using a 10 g column and eluting with 0 to 20% MeOH/CH₂Cl₂, afforded the title compound (40 mg, 0.028 mmol, 100% yield) as a white solid.

LC/MS: Calc'd m/z=1427.7 for C₇₆H₉₄N₁₄O₁₄, found [M+H]⁺=1428.8, [M+2H]²⁺=714.8.

### 2.42 N,N′-((7R,11S,19aS,26R,30S,38aS)-11,30-diisopropyl-9,12,15,28,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 42; FD-4)

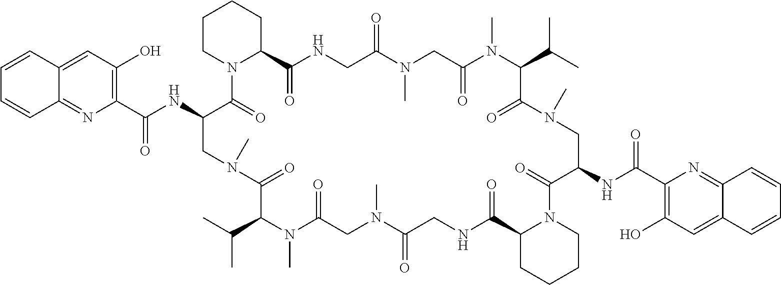 [SMILES: CC(C)[C@H]1C(=O)N(C)C[C@@H](NC(=O)c2nc3ccccc3cc2O)C(=O)N2CCCC[C@H]2C(=O)NCC(=O)N(C)CC(=O)N(C)[C@@H](C(C)C)C(=O)N(C)C[C@@H](NC(=O)c2nc3ccccc3cc2O)C(=O)N2CCCC[C@H]2C(=O)NCC(=O)N(C)CC(=O)N1C]

Compound 42 was synthesized as described in General Procedure 6 starting from Compound 41 (20 mg, 0.014 mmol), 10% Pd/C (10 mg) and EtOAc (2 mL). Following removal of Pd/C, purification according to General Procedure 12, using a 10 g column and eluting with 0 to 20% MeOH/CH₂Cl₂, afforded the title compound (11.6 mg, 0.009 mmol, 69% yield) as a pale-yellow solid.

LC/MS: Calc'd m/z=1247.4 for C₆₂H₈₂N₁₄O₁₄, found [M+H]⁺=1247.8, [M+2H]²⁺=624.8.

### 2.43 (S)-1-((R)-3-((S)-1-(N—(((S)-1-((R)-2-(((benzyloxy)carbonyl)amino)-3-((S)-1-(N-glycyl-N-methylglycyl) piperidine-2-carboxamido) propanoyl)piperidine-2-carbonyl)glycyl)-N-methylglycyl) piperidine-2-carboxamido)-2-((tert-butoxycarbonyl)amino)propanoyl)piperidine-2-carboxylic acid (Compound 44)

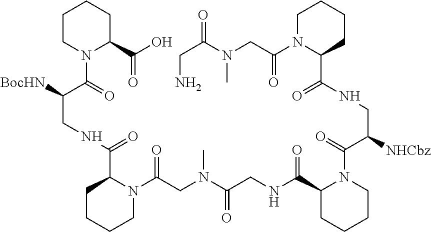 [SMILES: CN(CC(=O)N1CCCC[C@H]1C(=O)NC[C@@H](NC(=O)OCc1ccccc1)C(=O)N1CCCC[C@H]1C(=O)NCC(=O)N(C)CC(=O)N1CCCC[C@H]1C(=O)NC[C@@H](NC(=O)OC(C)(C)C)C(=O)N1CCCC[C@H]1C(=O)O)C(=O)CN]

The title compound was synthesized according to General Procedure 1 utilizing Fmoc-Pip-OH, Boc-D-Dap(Fmoc)-OH, Z-D-Dap(Fmoc)-OH, Fmoc-Sar-OH and Fmoc-Gly-OH, on a 0.2 mmol scale. The crude linear peptide (51 mg, 23%), post ether precipitation, was used without additional purification.

LC/MS: Calc'd m/z=1124.6 for C₅₃H₈₀N₁₂O₁₅ found [M+H]⁺=1125.8.

### 2.44 Benzyl N-[(3R,7S,20S,27R,31S,44S)-27-[(tert-butoxycarbonyl)amino]-15,39-dimethyl-2,6,13,16,19,26,30,37,40,43-decaoxo-1,5,12,15,18,25,29,36,39,42-decaazapentacyclo[42.4.0.0^{7,12}.0^{20,25}.0^{31,36}]octatetracontan-3-yl]carbamate (Compound 45)

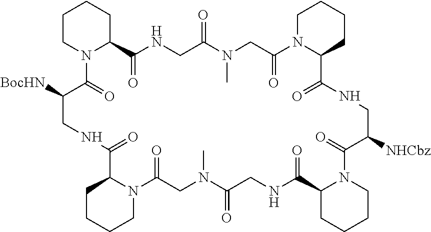 [SMILES: CN1CC(=O)N2CCCC[C@H]2C(=O)NC[C@@H](NC(=O)OC(C)(C)C)C(=O)N2CCCC[C@H]2C(=O)NCC(=O)N(C)CC(=O)N2CCCC[C@H]2C(=O)NC[C@@H](NC(=O)OCc2ccccc2)C(=O)N2CCCC[C@H]2C(=O)NCC1=O]

The title compound was synthesized according to General Procedure 4, starting from Compound 44 (51 mg, 45 umol). Preparative HPLC purification according to General Procedure 12 afforded the title compound (20 mg, 40%) as a white powder.

LC/MS: Calc'd m/z=1106.6 for C₅₃H₇₈N₁₂O₁₄ found [M+2H]²⁺=554.6, [M−Boc+2H]²⁺=504.4.

### 2.45 (7R,10aS,22aS,29R,32aS,44aS)-29-(((benzyloxy)carbonyl)amino)-18,40-dimethyl-6,10,16,19,22,28,32,38,41,44-decaoxotetratetracontahydrotetrapyrido[1,2-a:1′,2′-j:1″,2″-q:1″,2′″-z][1,4,7,10,13,17,20,23,26,29]decaazacyclodotriacontin-7-aminium chloride (Compound 46)

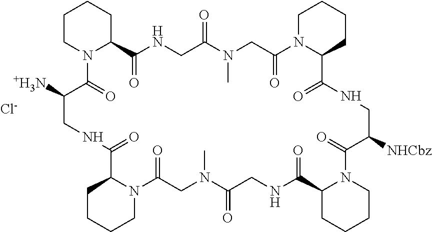 [SMILES: [Cl-] ; CN1CC(=O)N2CCCC[C@H]2C(=O)NC[C@@H](NC(=O)OCc2ccccc2)C(=O)N2CCCC[C@H]2C(=O)NCC(=O)N(C)CC(=O)N2CCCC[C@H]2C(=O)NC[C@@H]([NH3+])C(=O)N2CCCC[C@H]2C(=O)NCC1=O]

The title compound was synthesized from Compound 45 (20 mg, 18 μmol) according to General Procedure 5, and isolated as a white solid that was used without purification.

LC/MS: Calc'd m/z=1006.5 for C₄₈H₇₀N₁₂O₁₂ found [M+H]⁺=1007.6, [M+2H]²⁺=504.6.

### 2.46 (7R,10aS,22aS,29R,32aS,44aS)-29-amino-18,40-dimethyl-6,10,16,19,22,28,32,38,41,44-decaoxotetratetracontahydrotetrapyrido[1,2-a:1′,2′-j:1″,2″-q:1″,2″-z][1,4,7,10,13,17,20,23,26,29]decaazacyclodotriacontin-7-aminium chloride (Compound 47)

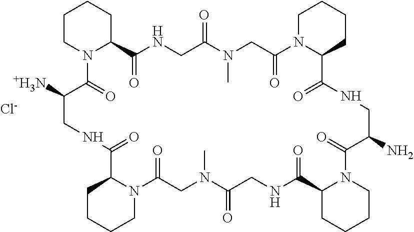 [SMILES: [Cl-] ; CN1CC(=O)N2CCCC[C@H]2C(=O)NC[C@@H]([NH3+])C(=O)N2CCCC[C@H]2C(=O)NCC(=O)N(C)CC(=O)N2CCCC[C@H]2C(=O)NC[C@@H](N)C(=O)N2CCCC[C@H]2C(=O)NCC1=O]

The title compound was synthesized from Compound 46 (7 mg, 6.7 umol) according to General Procedure 6, and isolated as a white solid that was used without purification.

LC/MS: Calc'd m/z=872.5 for C₄₀H₆₄N₁₂O₁₀ found [M+H]⁺=873.6, [M+2H]²⁺=437.4.

### 2.47 N,N′-((7R,10aS,22aS,29R,32aS,44aS)-18,40-dimethyl-6,10,16,19,22,28,32,38,41,44-decaoxotetratetracontahydrotetrapyrido[1,2-a:1′,2′-j:1″,2″-q:1′″,2″-z][1,4,7,10,13,17,20,23,26,29]decaazacyclodotriacontine-7,29-diyl)bis(3-(benzyloxy)quinoline-2-carboxamide) (Compound 48)

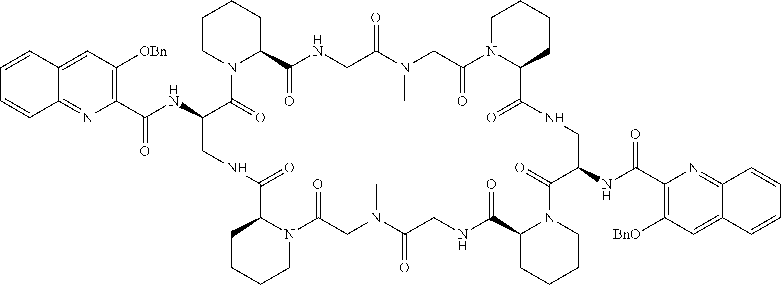 [SMILES: CN1CC(=O)N2CCCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N2CCCC[C@H]2C(=O)NC[C@@H](NC(=O)c2nc3ccccc3cc2OCc2ccccc2)C(=O)N2CCCC[C@H]2C(=O)NCC1=O]

The title compound was synthesized from Compound 47 (5.8 mg, 6.7 umol) according to General Procedure 7. Purification according to General Procedure 12, using a 10 g column and eluting with 0 to 10% MeOH/CH₂Cl₂, afforded the title compound (11 mg) as a pale-yellow solid.

LC/MS: Calc'd m/z=1394.6 for C₇₄H₈₆N₁₄O₁₄ found [M+H]⁺=1395.8, (M+2H)²⁺=698.8.

### 2.48 N,N′-((7R,10aS,22aS,29R,32aS,44aS)-18,40-dimethyl-6,10,16,19,22,28,32,38,41,44-decaoxotetratetracontahydrotetrapyrido[1,2-a:1′,2′-j:1″,2″-q:1″,2′″-z][1,4,7,10,13,17,20,23,26,29]decaazacyclodotriacontine-7,29-diyl)bis(3-hydroxyquinoline-2-carboxamide) (Compound 49; FD-14)

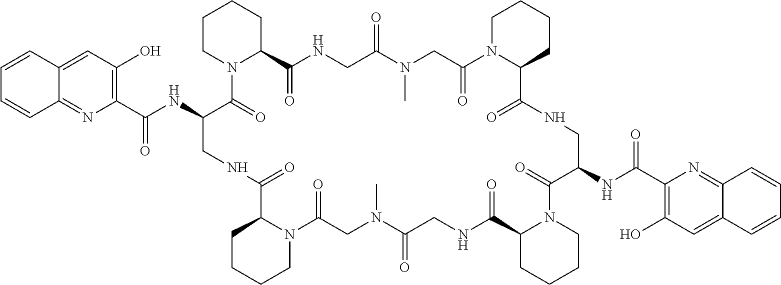 [SMILES: CN1CC(=O)N2CCCC[C@H]2C(=O)NC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](NC(=O)c2nc3ccccc3cc2O)C(=O)N2CCCC[C@H]2C(=O)NCC1=O]

The title compound was synthesized from Compound 48 (11 mg) according to General Procedure 8 and purified according to General Procedure 12, using a 10 g column and eluting with 0 to 10% MeOH/CH₂Cl₂, to afford the title compound (5.8 mg, 71%) as a yellow solid.

LC/MS: Calc'd m/z=1214.6 for C₆₀H₇₄N₁₄O₁₄ found [M+H]⁺=1215.8, [M+2H]²⁺=608.6.

### 2.49 (S)-1-((9S,13R)-1-((S)-1-((R)-3-((2S,3R)-2-(2-(2-amino-N-methylacetamido)-N-methylacetamido)-3-(tert-butoxy) butanamido)-2-(((benzyloxy)carbonyl)amino)propanoyl) piperidin-2-yl)-13-(((benzyloxy)carbonyl)amino)-9-isopropyl-5,8-dimethyl-1,4,7,10-tetraoxo-2,5,8,11-tetraazatetradecan-14-oyl) piperidine-2-carboxylic acid (Compound 50)

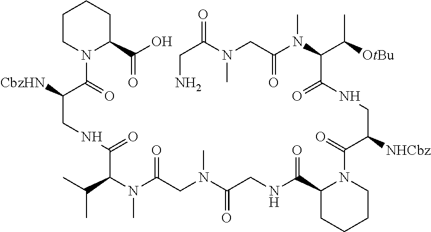 [SMILES: CC(C)[C@@H](C(=O)NC[C@@H](NC(=O)OCc1ccccc1)C(=O)N1CCCC[C@H]1C(=O)O)N(C)C(=O)CN(C)C(=O)CNC(=O)[C@@H]1CCCCN1C(=O)[C@@H](CNC(=O)[C@H]([C@@H](C)OC(C)(C)C)N(C)C(=O)CN(C)C(=O)CN)NC(=O)OCc1ccccc1]

The title compound was synthesized according to General Procedure 1 utilizing Fmoc-Pip-OH, Cbz-D-Dap(Fmoc)-OH, Fmoc-N-methyl-Val-OH, Fmoc-N-methyl-Thr(OtBu)-OH, Fmoc-Sar-OH and Fmoc-Gly-OH. The crude peptide (350 mg, 0.287 mmol, 72% yield) was isolated, post ether precipitation, as white powder and was used without additional purification.

LC/MS: Calc'd m/z=1220.6 for C₅₉H₈₈N₁₂O₁₆, found [M+H]⁺=1221.8, [M−Bu+2H]²⁺=583.6.

### 2.50 Dibenzyl ((7R,11S,19aS,26R,30S,38aS)-11-((R)-1-(tert-butoxy)ethyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 51)

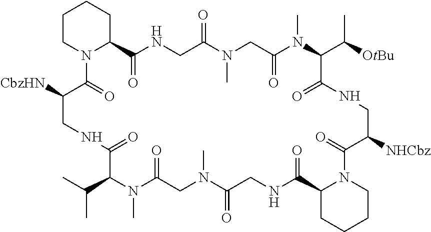

The title compound was synthesized as described in General Procedure 4 starting from Compound 50 (150 mg, 0.123 mmol). Purification according to General Procedure 12, using a 10 g column and eluting with 0 to 20% MeOH/CH₂Cl₂, afforded the title compound (135 mg, 0.112 mmol, 91% yield) as a white solid.

LC/MS: Calc'd m/z=1202.6 for C₅₉H₈₆N₁₂O₁₅, found [M+H]⁺=1204.3, [M−(Bu+2H]²⁺=574.6

### 2.51 (7R,11S,19aS,26R,30S,38aS)-7,26-diamino-11-((R)-1-(tert-butoxy)ethyl)-30-isopropyl-12,15,31,34-tetramethyltetracosahydro-2H,6H-dipyrido[1,2-a:1′,2′-q][1,4,7,10,13,17,20,23,26,29]decaazacyclodotriacontine-6,10,13,16,19,25,29,32,35,38 (7H,19aH,26H)-decaone (Compound 52)

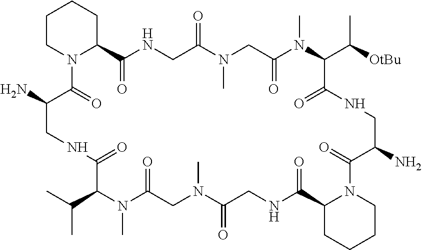

The title compound was prepared according to General Procedure 6 starting from Compound 51 (135 mg, 0.112 mmol) and 10% Pd/C (12 mg) in MeOH (5 mL).

LC/MS: Calc'd m/z=934.6 for C₄₃H₇₄N₁₂O₁₁, found [M+H]⁺=936.8, [M+2H]²⁺=468.6.

### 2.52 N,N′-((7R,11S,19aS,26R,30S,38aS)-11-((R)-1-(tert-butoxy)ethyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 53)

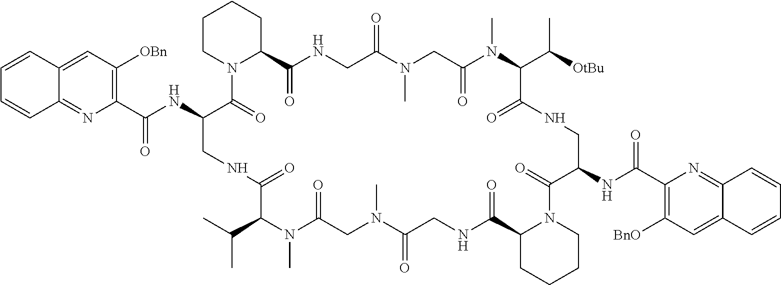 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H]([C@@H](C)OC(C)(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 52 (0.112 mmol). Purification according to General Procedure 12, using a 10 g column and eluting with 0 to 20% MeOH/CH₂Cl₂, afforded the title compound (114 mg, 0.078 mmol, 70% yield over 2 steps) as a white solid.

LC/MS: Calc'd m/z=1456.7 for C₇₇H₉₆N₁₄O₁₅, found [M+H]⁺=1457.6, [M+2H]²⁺=729.8.

### 2.53 N,N′-((7R,11S,19aS,26R,30S,38aS)-11-((R)-1-(tert-butoxy)ethyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 54; FD-7)

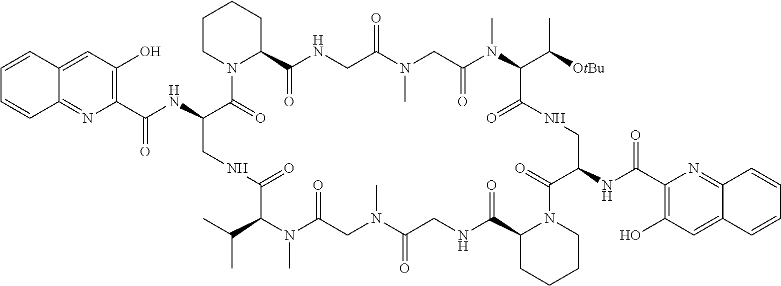 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)OC(C)(C)C)C(=O)NC[C@@H](NC(=O)c2nc3ccccc3cc2O)C(=O)N2CCCC[C@H]2C(=O)NCC(=O)N(C)CC(=O)N1C]

Compound 54 was synthesized as described in General Procedure 6 starting from Compound 53 (22 mg, 0.015 mmol), 10% Pd/C (10 mg) and MeOH (2 mL). Following removal of Pd/C, purification according to General Procedure 12, using a 10 g column and eluting with 0 to 20% MeOH/CH₂Cl₂, afforded the title compound (17 mg, 0.013 mmol, 89% yield) as a pale-yellow solid.

LC/MS: Calc: 1276.6 for C₆₃H₈₄N₁₄O₁₅, found [M−tBu+H]⁺=1222.2, [M−(Bu+2H]²⁺=611.6.

### 2.54 N,N′-((7R,11S,19aS,26R,30S,38aS)-11-((R)-1-hydroxyethyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 55; FD-8)

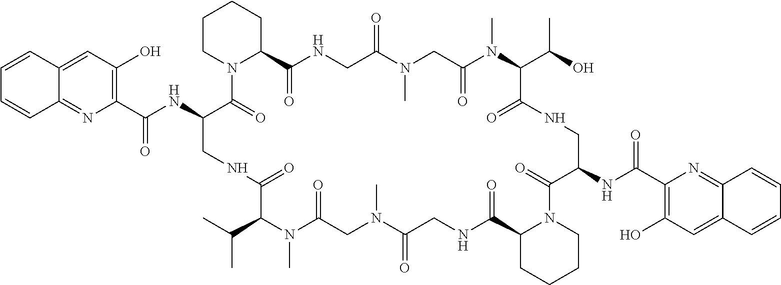 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)O)C(=O)NC[C@@H](NC(=O)c2nc3ccccc3cc2O)C(=O)N2CCCC[C@H]2C(=O)NCC(=O)N(C)CC(=O)N1C]

The titled compound was prepared according to General Procedure 5 starting from Compound 54 (7 mg, 0.006 mmol) and 2 mL of 4 M HCl/dioxane. Compound 55 (5 mg, 0.004 mmol, 68% yield) was isolated as vibrant yellow solid.

LC/MS: Calc'd m/z=1220.6 for C₅₉H₇₆N₁₄O₁₅, found [M+H]⁺=1222.2, [M+2H]²⁺=611.6

### 2.55 (S)-1-((9S,13R)-1-((S)-1-((R)-3-((S)-2-(2-(2-amino-N-methylacetamido)-N-methylacetamido)-3-methylbutanamido)-2-(((benzyloxy)carbonyl)amino)propanoyl) piperidin-2-yl)-13-((tert-butoxycarbonyl)amino)-9-isopropyl-5,8-dimethyl-1,4,7,10-tetraoxo-2,5,8,11-tetraazatetradecan-14-oyl) piperidine-2-carboxylic acid (Compound 56)

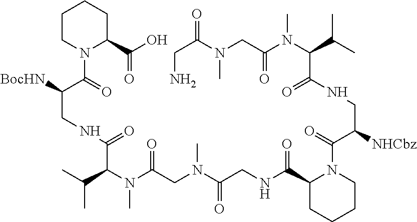 [SMILES: CC(C)[C@@H](C(=O)NC[C@@H](NC(=O)OCc1ccccc1)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)O)C(C)C)N(C)C(=O)CN(C)C(=O)CN]

The title compound was synthesized according to General Procedure 1 utilizing Fmoc-Pip-OH, Boc-D-Dap(Fmoc)-OH, Z-D-Dap(Fmoc)-OH, Fmoc-N-Methyl-Val-OH, Fmoc-Sar-OH and Fmoc Gly-OH, on a 1.5 mm scale. The crude linear peptide (1.36 g, 72%), post ether precipitation, was used without additional purification.

LC/MS: Calc'd m/z=1128.6 for C₅₃H₈₄N₁₂O₁₅ found [M+H]⁺=1129.8.

### 2.56 Benzyl tert-butyl ((7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 57)

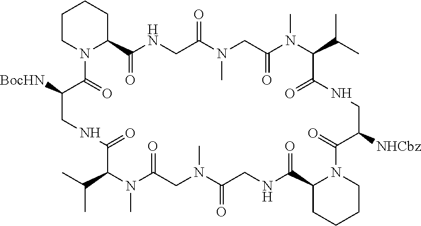 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)[C@@H](C(C)C)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 56 (950 mg, 841 umol). Purification according to General Procedure 12, using a 10 g column and eluting with 0 to 20% MeOH/CH₂Cl₂, afforded the title compound (625 mg, 69%) as a white solid.

LC/MS: Calc'd m/z=1110.6 for C₅₃H₈₂N₁₂O₁₄ found [M+H]⁺=1111.8, [M−Boc+H]⁺=1011.8, [M−Boc+2H]²⁺=505.6.

### 2.57 (7R,11S,19aS,26R,30S,38aS)-26-(((benzyloxy)carbonyl)amino)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-aminium chloride (Compound 58)

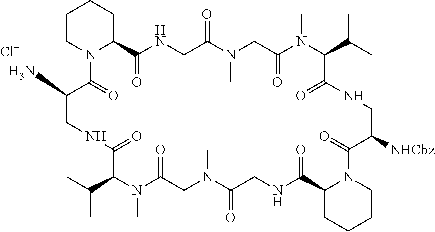 [SMILES: [Cl-] ; CC(C)[C@H]1C(=O)NC[C@@H](NC(=O)OCc2ccccc2)C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C]

The title compound was synthesized from Compound 57 (310 mg, 279 umol) according to General Procedure 5, and isolated as a white solid that was used without purification.

LC/MS: Calc'd m/z=1010.6 for C₄₈H₇₄N₁₂O₁₂, found [M+H]⁺=1011.8, [M+2H]²⁺=506.6.

### 2.58 ((S)-1-((R)-2-(((benzyloxy)carbonyl)amino)-3-((2S,3S)-2-(2-(2-((S)-1-((R)-2-((tert-butoxycarbonyl)amino)-3-((2S,3S)-3-methyl-2-(N-methyl-2-(methylamino) acetamido) pentanamido) propanoyl)piperidine-2-carboxamido)-N-methylacetamido)-N-methylacetamido)-3-methylpentanamido) propanoyl)piperidine-2-carbonyl)glycine (Compound 63)

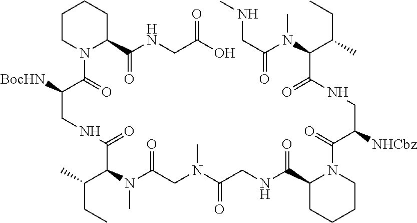 [SMILES: CC[C@H](C)[C@@H](C(=O)NC[C@@H](NC(=O)OCc1ccccc1)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)NCC(=O)O)[C@@H](C)CC)N(C)C(=O)CNC]

The title compound was synthesized according to General Procedure 1 utilizing Fmoc-Pip-OH, Boc-D-Dap(Fmoc)-OH, Z-D-Dap(Fmoc)-OH, Fmoc-N-methyl-Ile-OH, Fmoc-Gly-OH and Fmoc-Sar-OH, on a 0.5 mm scale. The crude linear peptide (447 mg, 77%), post ether precipitation, was used without additional purification.

LC/MS: Calc'd m/z=1156.6 for C₅₅H₈₈N₁₂O₁₅, found [M+H]⁺=1157.8, [M−Boc+2H]²⁺=529.6.

### 2.59 Benzyl tert-butyl ((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 64)

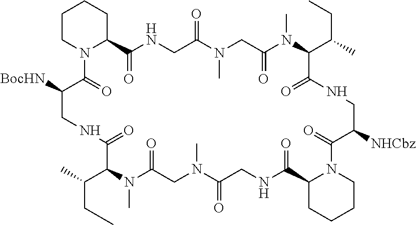 [SMILES: CC[C@H](C)[C@H]1C(=O)NC[C@@H](NC(=O)OC(C)(C)C)C(=O)N2CCCC[C@H]2C(=O)NCC(=O)N(C)CC(=O)N(C)[C@@H]([C@@H](C)CC)C(=O)NC[C@@H](NC(=O)OCc2ccccc2)C(=O)N2CCCC[C@H]2C(=O)NCC(=O)N(C)CC(=O)N1C]

The title compound was synthesized as described in General Procedure 4, starting from Compound 63 (447 mg, 0.386 mmol). Purification according to General Procedure 12, eluting with 0 to 20% MeOH/CH₂Cl₂ gradient, afforded the title compound (397 mg, 0.348 mmol, 90% yield) as a white solid film.

LC/MS: Calc'd m/z=1138.6 for C₅₅H₈₆N₁₂O₁₄, found [M+H]⁺=1139.8, [M−Boc+H]⁺=1039.8, [M−Boc+2H]²⁺=520.6.

### 2.60 (7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium chloride (Compound 65)

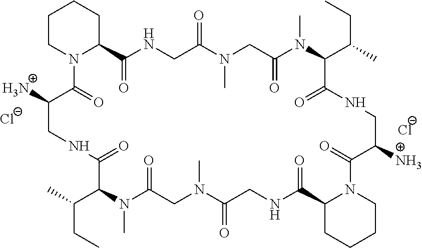 [SMILES: [Cl-] ; CC[C@H](C)[C@H]1C(=O)NC[C@@H]([NH3+])C(=O)N2CCCC[C@H]2C(=O)NCC(=O)N(C)CC(=O)N(C)[C@@H]([C@@H](C)CC)C(=O)NC[C@@H]([NH3+])C(=O)N2CCCC[C@H]2C(=O)NCC(=O)N(C)CC(=O)N1C ; [Cl-]]

The title compound was prepared from Compound 64 (40 mg, 0.035 mmol) according to General Procedure 5 followed by General Procedure 6.

LC/MS: Calc'd m/z=904.6 for C₄₂H₇₂N₁₂O₁₀, found [M+H]⁺=905.8, [M+2H]²⁺=453.4.

### 2.61 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 66)

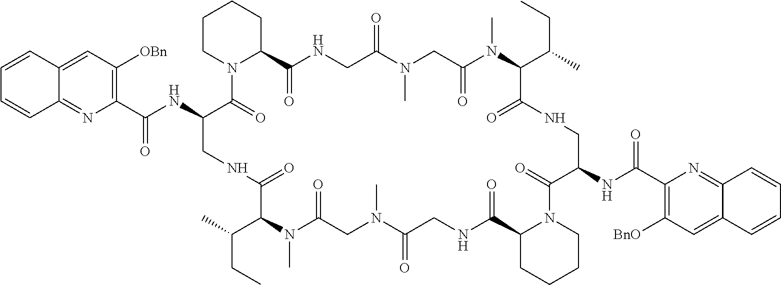 [SMILES: CC[C@H](C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared from crude Compound 65 (0.035 mmol) according to General Procedure 7. Purification according to General Procedure 12, eluting with 0 to 20% MeOH/CH₂Cl₂, afforded the title compound (47 mg, 0.033 mmol, 96% yield) as a white solid.

LC/MS: Calc'd m/z=1426.7 for C₇₆H₉₄N₁₄O₁₄, found [M+2H]²⁺=714.8.

### 2.62 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 67; FD-9)

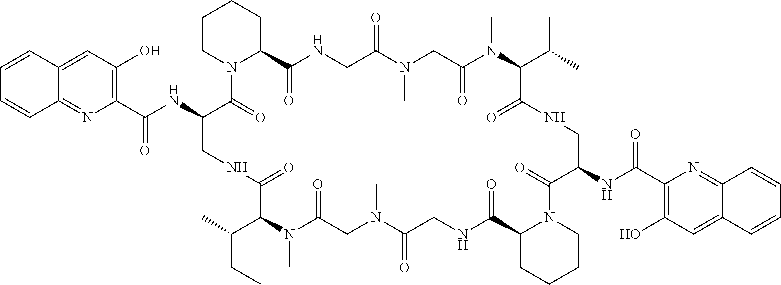

Compound 67 was synthesized from Compound 66 (34 mg, 0.024 mmol) as described in General Procedure 6. Purification according to General Procedure 12, eluting with 30 to 70% CH₃CN/H₂O, afforded the title compound (4.7 mg, 0.004 mmol, 16% yield) as a light-yellow solid.

LC/MS: Calc'd m/z=1246.6, for C₆₂H₈₂N₁₄O₁₄, found [M+H]⁺=1247.8, [M+2H]²⁺=624.6.

### 2.63 (S)-1-((9S,13R)-1-((S)-1-((R)-3-((2S,3S)-2-(2-(2-amino-N-methylacetamido)-N-methylacetamido)-3-methylpentanamido)-2-((tert-butoxycarbonyl)amino)propanoyl) piperidin-2-yl)-13-(((benzyloxy)carbonyl)amino)-9-isopropyl-5,8-dimethyl-1,4,7,10-tetraoxo-2,5,8,11-tetraazatetradecan-14-oyl) piperidine-2-carboxylic acid (Compound 68)

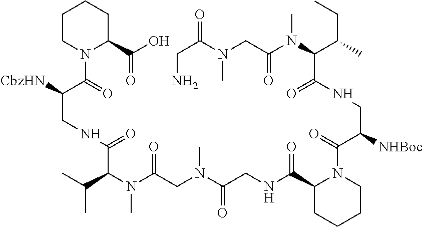 [SMILES: CC[C@H](C)[C@@H](C(=O)NC[C@@H](NC(=O)OC(C)(C)C)C(=O)N1CCCC[C@H]1C(=O)NCC(=O)N(C)CC(=O)N(C)[C@H](C(=O)NC[C@@H](NC(=O)OCc1ccccc1)C(=O)N1CCCC[C@H]1C(=O)O)C(C)C)N(C)C(=O)CN(C)C(=O)CN]

The title compound was synthesized according to General Procedure 1 utilizing Fmoc-Pip-OH, Boc-D-Dap(Fmoc)-OH, Z-D-Dap(Fmoc)-OH, Fmoc-N-methyl-Val-OH and Fmoc-N-methyl-Ile-OH, and Compound 9, on a 0.15 mm scale. The crude linear peptide (142 mg, 0.12 mmol, 80% yield), post ether precipitation, was used without further purification.

LC/MS: Calc'd m/z=1142.6 for C₅₄H₈₆N₁₂O₁₅, found [M+H]⁺=1143.8, [M−Boc, +2H]²⁺=522.4.

### 2.64 Benzyl tert-butyl ((7R,11S,19aS,26R,30S,38aS)-11-((S)-sec-butyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 69)

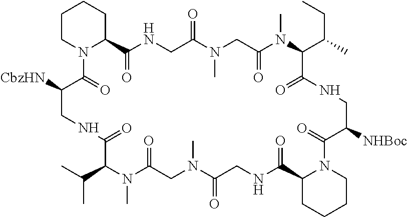 [SMILES: CC[C@H](C)[C@H]1C(=O)NC[C@@H](NC(=O)OC(C)(C)C)C(=O)N2CCCC[C@H]2C(=O)NCC(=O)N(C)CC(=O)N(C)[C@@H](C(C)C)C(=O)NC[C@@H](NC(=O)OCc2ccccc2)C(=O)N2CCCC[C@H]2C(=O)NCC(=O)N(C)CC(=O)N1C]

The title compound was synthesized as described in General Procedure 4, starting from Compound 68 (142 mg, 0.124 mmol). Purification according to General Procedure 12, eluting with 0 to 20% MeOH/CH₂Cl₂, afforded the title compound (104 mg, 0.0925 mmol, 75% yield) as a white film.

LC/MS: Calc'd m/z=1124.6 for C₅₄H₈₄N₁₂O₁₄, found [M+H]⁺=1125.8, [M−Boc+H]⁺=1025.8, [M−Boc+2H]²⁺=513.4.

### 2.65 (7R,11S,19aS,26R,30S,38aS)-11-((S)-sec-butyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium dichloride (Compound 70)

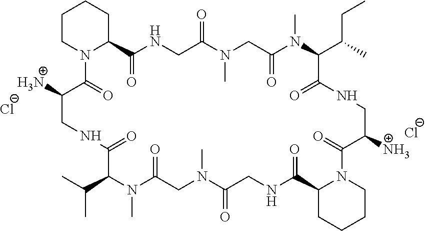 [SMILES: [Cl-] ; CC[C@H](C)[C@H]1C(=O)NC[C@@H]([NH3+])C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C ; [Cl-]]

The title compound was prepared from Compound 69 (20 mg, 0.0178 mmol) according to General Procedure 6 followed by General Procedure 5.

LC/MS: Calc'd m/z=890.5 for C₄₁H₇₀N₁₂O₁₀, found [M+2H]²⁺=448.4.

### 2.66 N,N′-((7R,11S,19aS,26R,30S,38aS)-11-((S)-sec-butyl)-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 71)

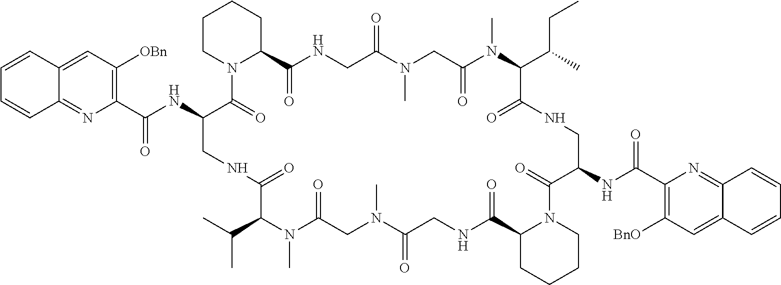 [SMILES: CC[C@H](C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared from crude Compound 70 (0.0178 mmol) according to General Procedure 7. Purification according to General Procedure 12, eluting with 0 to 20% MeOH/CH₂Cl₂, afforded the title compound (30 mg) as a white solid.

LC/MS: Calc'd m/z=1412.7 for C₇₅H₉₂N₁₄O₁₄, found [M+2H]²⁺=707.8.

### 2.67 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 72; FD-10)

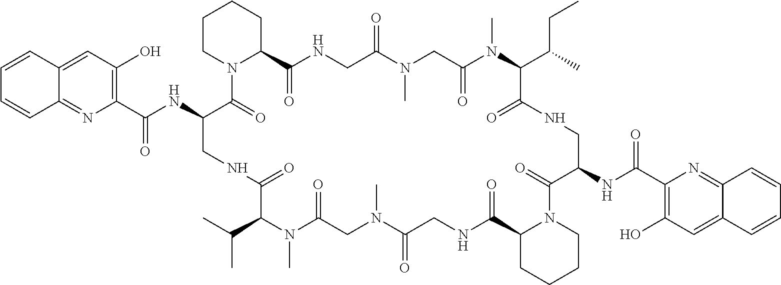 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

Compound 72 was synthesized from impure Compound 71 (30 mg, 0.02 mmol) as described in General Procedure 6. Purification according to General Procedure 12, eluting with 30 to 60% CH₃CN/H₂O, afforded the title compound (7.9 mg, 0.006 mmol, 36% yield over 2 steps) as a white solid.

LC/MS: Calc'd m/z=1232.6 for C₆₁H₈₀N₁₄O₁₄, found [M+H]⁺=1233.8, [M+2H]²⁺=617.6.

### 2.68 (S)-1-((R)-2-(((benzyloxy)carbonyl)amino)-3-((S)-2-(2-(2-((S)-1-((R)-2-((tert-butoxycarbonyl)amino)-3-((S)-1-(N-glycyl-N-methylglycyl) pyrrolidine-2-carboxamido) propanoyl)piperidine-2-carboxamido)-N-methylacetamido)-N-methylacetamido)-3-methylbutanamido) propanoyl)piperidine-2-carboxylic acid (Compound 73)

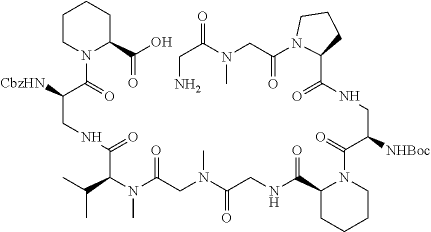 [SMILES: CC(C)[C@@H](C(=O)NC[C@@H](NC(=O)OCc1ccccc1)C(=O)N1CCCC[C@H]1C(=O)O)N(C)C(=O)CN(C)C(=O)CNC(=O)[C@@H]1CCCCN1C(=O)[C@@H](CNC(=O)[C@@H]1CCCN1C(=O)CN(C)C(=O)CN)NC(=O)OC(C)(C)C]

The title compound was synthesized according to General Procedure 1 utilizing Fmoc-Pip-OH, Boc-D-Dap(Fmoc)-OH, Z-D-Dap(Fmoc)-OH, Fmoc-N-methyl-Val-OH and Fmoc-Pro-OH, and Compound 9, on a 0.15 mmol scale. The crude linear peptide (147 mg, 0.13 mmol, 87% yield), post ether precipitation, was used without further purification.

LC/MS: Calc'd m/z=1112.6 for C₅₂H₈₀N₁₂O₁₅, found [M+H]⁺=1113.8, [M+2H]²⁺=507.4.

### 2.69 Benzyl tert-butyl ((3aS,7R,13aS,22S,26R,32aS)-22-isopropyl-18,21,37-trimethyl-4,8,14,17,20,23,27,33,36,39-decaoxooctatriacontahydro-1H,10H-dipyrido[1,2-a:1′,2′-q]pyrrolo[1,2-j][1,4,7,10,13,17,20,23,26,29]decaazacyclodotriacontine-7,26-diyl)dicarbamate (Compound 74)

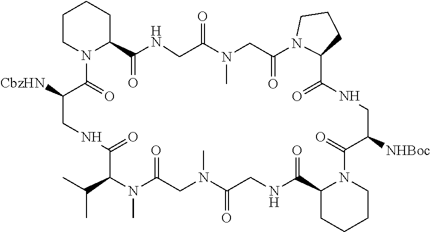 [SMILES: CC(C)[C@H]1C(=O)NC[C@@H](NC(=O)OCc2ccccc2)C(=O)N2CCCC[C@H]2C(=O)NCC(=O)N(C)CC(=O)N2CCC[C@H]2C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 4, starting from Compound 73 (147 mg, 0.132 mmol). Purification according to General Procedure 12, eluting with 0 to 20% MeOH/CH₂Cl₂, afforded the title compound (90 mg, 0.082 mmol, 62% yield) as a white film.

LC/MS: Calc'd m/z=1094.6 for C₅₂H₇₈N₁₂O₁₄, found [M+H]⁺=1095.8, [M−Boc+H]⁺=995.8, [M−Boc+2H]²⁺=498.6.

### 2.70 (3aS,7R,13aS,22S,26R,32aS)-22-isopropyl-18,21,37-trimethyl-4,8,14,17,20,23,27,33,36,39-decaoxooctatriacontahydro-1H,10H-dipyrido[1,2-a:1′,2′-q]pyrrolo[1,2-j][1,4,7,10,13,17,20,23,26,29]decaazacyclodotriacontine-7,26-diaminium dichloride (Compound 75)

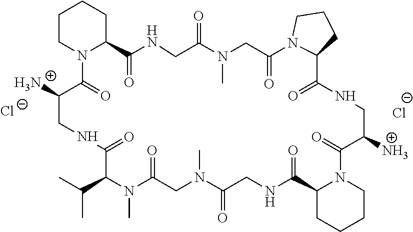 [SMILES: [Cl-] ; CC(C)[C@H]1C(=O)NC[C@@H]([NH3+])C(=O)N2CCCC[C@H]2C(=O)NCC(=O)N(C)CC(=O)N2CCC[C@H]2C(=O)NC[C@@H]([NH3+])C(=O)N2CCCC[C@H]2C(=O)NCC(=O)N(C)CC(=O)N1C ; [Cl-]]

The title compound was prepared from Compound 74 (20 mg, 0.018 mmol) according to General Procedure 6 followed by General Procedure 5.

LC/MS: Calc'd m/z=860.5 for C₃₉H₆₄N₁₂O₁₀, found [M+H]⁺=861.8, [M+2H]²⁺=431.4.

### 2.71 N,N′-((3aS,7R,13aS,22S,26R,32aS)-22-isopropyl-18,21,37-trimethyl-4,8,14,17,20,23,27,33,36,39-decaoxooctatriacontahydro-1H,10H-dipyrido[1,2-a:1′,2′-q]pyrrolo[1,2-j][1,4,7,10,13,17,20,23,26,29]decaazacyclodotriacontine-7,26-diyl)bis(3-(benzyloxy)quinoline-2-carboxamide) (Compound 76)

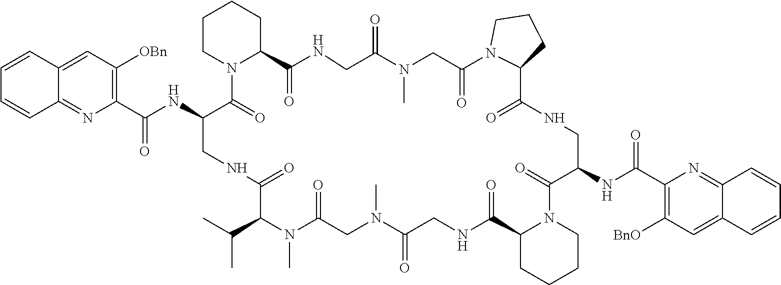 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N2CCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared from crude Compound 75 (0.0183 mmol) according to General Procedure 7. Purification according to General Procedure 12, eluting with 0 to 20% MeOH/CH₂Cl₂, afforded the title compound (22 mg, 0.016 mmol, 87% yield) as a white solid.

LC/MS: Calc'd m/z=1382.6 for C₇₃H₈₆N₁₄O₁₄, found [M+2H]²⁺=692.6.

### 2.72 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 77; FD-20)

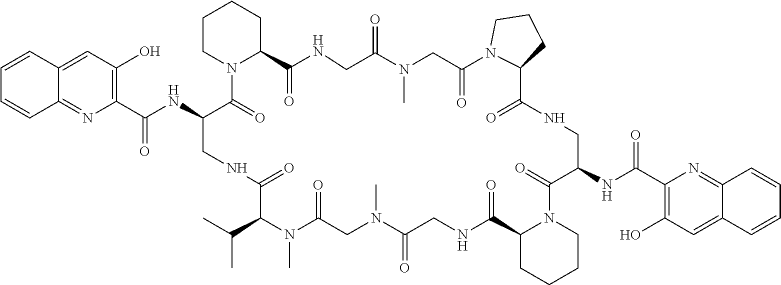 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N2CCC[C@H]2C(=O)NC[C@@H](NC(=O)c2nc3ccccc3cc2O)C(=O)N2CCCC[C@H]2C(=O)NCC(=O)N(C)CC(=O)N1C]

Compound 77 was synthesized from Compound 76 (22 mg, 0.016 mmol) as described in General Procedure 6. Purification according to General Procedure 12, eluting with 30 to 60% CH₃CN/H₂O, afforded the title compound (1.8 mg, 0.0015 mmol, 9% yield) as a white solid.

LC/MS: Calc'd m/z=1202.6 for C₅₉H₇₄N₁₄O₁₄, found [M+H]⁺=1203.8, [M+2H]²⁺=602.6.

### 2.73 (S)-1-((R)-2-(((benzyloxy)carbonyl)amino)-3-((S)-2-(2-(2-((S)-1-((R)-2-((tert-butoxycarbonyl)amino)-3-((S)-1-(N-glycyl-N-methylglycyl) piperidine-2-carboxamido) propanoyl)piperidine-2-carboxamido)-N-methylacetamido)-N-methylacetamido)-3-methylbutanamido) propanoyl)piperidine-2-carboxylic acid (Compound 78)

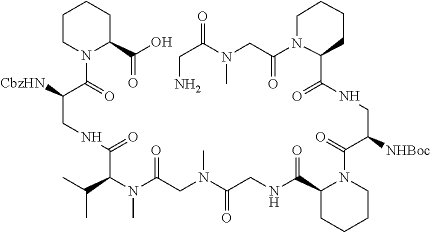 [SMILES: CC(C)[C@@H](C(=O)NC[C@@H](NC(=O)OCc1ccccc1)C(=O)N1CCCC[C@H]1C(=O)O)N(C)C(=O)CN(C)C(=O)CNC(=O)[C@@H]1CCCCN1C(=O)[C@@H](CNC(=O)[C@@H]1CCCCN1C(=O)CN(C)C(=O)CN)NC(=O)OC(C)(C)C]

The title compound was synthesized according to General Procedure 1 utilizing Fmoc-Pip-OH, Boc-D-Dap(Fmoc)-OH, Z-D-Dap(Fmoc)-OH and Fmoc-N-methyl-Val-OH, and Compound 9, on a 0.15 mmol scale. The crude linear peptide (150 mg, 0.13 mmol, 87% yield), post ether precipitation, was used without further purification.

LC/MS: calc'd m/z=1126.6 for C₅₃H₈₂N₁₂O₁₅, found [M+H]⁺=1127.8, [M−Boc+2H]²⁺=514.8.

### 2.74 Benzyl tert-butyl ((4aS,8R,14aS,23S,27R,33aS)-23-isopropyl-19,22,38-trimethyl-5,9,15,18,21,24,28,34,37,40-decaoxotetracontahydro-11H-tripyrido[1,2-a:1′,2′-j:1″,2″-q][1,4,7,10,13,17,20,23,26,29]decaazacyclodotriacontine-8,27-diyl)dicarbamate (Compound 79)

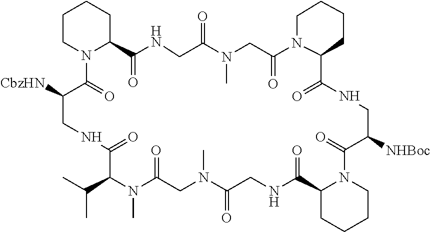 [SMILES: CC(C)[C@H]1C(=O)NC[C@@H](NC(=O)OCc2ccccc2)C(=O)N2CCCC[C@H]2C(=O)NCC(=O)N(C)CC(=O)N2CCCC[C@H]2C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 4, starting from Compound 78 (150 mg, 0.133 mmol). Purification according to General Procedure 12, eluting with 0 to 20% MeOH/CH₂Cl₂, afforded the title compound (95 mg, 0.0822 64% yield) as a white film.

LC/MS: Calc. 1108.6 for C₅₃H₈₀N₁₂O₁₄, found [M+H]⁺=1109.8, [M−Boc+H]⁺=1009.8, [M−Boc+2H]²⁺=505.4.

### 2.75 (4aS,8R,14aS,23S,27R,33aS)-23-isopropyl-19,22,38-trimethyl-5,9,15,18,21,24,28,34,37,40-decaoxotetracontahydro-11H-tripyrido[1,2-a:1′,2′-j:1″,2″-q][1,4,7,10,13,17,20,23,26,29]decaazacyclodotriacontine-8,27-diaminium dichloride (Compound 80)

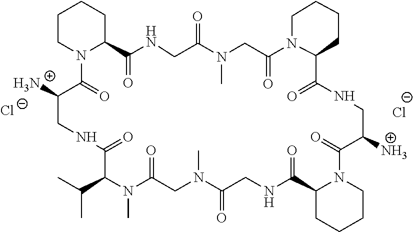 [SMILES: [Cl-] ; CC(C)[C@H]1C(=O)NC[C@@H]([NH3+])C(=O)N2CCCC[C@H]2C(=O)NCC(=O)N(C)CC(=O)N2CCCC[C@H]2C(=O)NC[C@@H]([NH3+])C(=O)N2CCCC[C@H]2C(=O)NCC(=O)N(C)CC(=O)N1C ; [Cl-]]

The title compound was prepared from Compound 79 (20 mg, 0.018 mmol) according to General Procedure 6 followed General Procedure 5.

LC/MS: Calc'd m/z=874.5 for C₄₀H₆₆N₁₂O₁₀, found [M+H]⁺=875.6, [M+2H]²⁺=438.4.

### 2.76 N,N′-((4aS,8R,14aS,23S,27R,33aS)-23-isopropyl-19,22,38-trimethyl-5,9,15,18,21,24,28,34,37,40-decaoxotetracontahydro-11H-tripyrido[1,2-a:1′,2′-j:1″,2″-q][1,4,7,10,13,17,20,23,26,29]decaazacyclodotriacontine-8,27-diyl)bis(3-(benzyloxy)quinoline-2-carboxamide) (Compound 81)

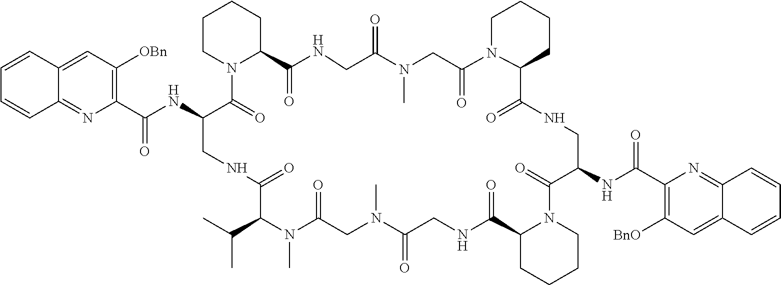 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N2CCCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared from crude Compound 80 (0.018 mmol) according to General Procedure 7. Purification according to General Procedure 12, eluting with 0 to 20% MeOH/CH₂Cl₂, afforded the title compound (16 mg, 0.012 mmol, 64% yield) as a white solid.

LC/MS: Calc'd m/z=1396.7 for C₇₄H₈₈N₁₄O₁₄, found [M+2H]²⁺=699.6.

### 2.77 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 82; FD-21)

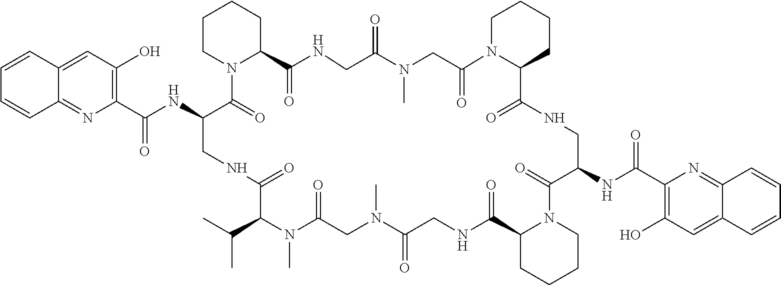 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](NC(=O)c2nc3ccccc3cc2O)C(=O)N2CCCC[C@H]2C(=O)NCC(=O)N(C)CC(=O)N1C]

Compound 82 was synthesized from Compound 81 (16 mg, 0.012 mmol) as described in General Procedure 6. Purification according to General Procedure 12, eluting with 30 to 60% CH₃CN/H₂O, afforded the title compound (1.9 mg, 0.002 mmol, 14% yield) as a white solid.

LC/MS: Calc'd m/z=1216.6 for C₆₀H₇₆N₁₄O₁₄, found [M+H]⁺=1217.8, [M+2H]²⁺=609.6.

### 2.78 (S)-1-((R)-2-(((benzyloxy)carbonyl)amino)-3-((S)-2-(2-(2-((S)-1-((R)-2-((tert-butoxycarbonyl)amino)-3-((S)-1-(N-glycyl-N-methylglycyl) azepane-2-carboxamido) propanoyl)piperidine-2-carboxamido)-N-methylacetamido)-N-methylacetamido)-3-methylbutanamido) propanoyl)piperidine-2-carboxylic acid (Compound 83)

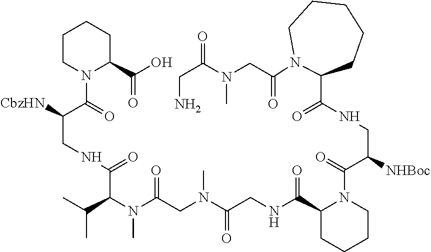 [SMILES: CC(C)[C@@H](C(=O)NC[C@@H](NC(=O)OCc1ccccc1)C(=O)N1CCCC[C@H]1C(=O)O)N(C)C(=O)CN(C)C(=O)CNC(=O)[C@@H]1CCCCN1C(=O)[C@@H](CNC(=O)[C@@H]1CCCCCN1C(=O)CN(C)C(=O)CN)NC(=O)OC(C)(C)C]

The title compound was synthesized according to General Procedure 1 utilizing Fmoc-Pip-OH, Fmoc-azepane-OH Cbz-D-Dap(Fmoc)-OH, Boc-D-Dap(Fmoc)-OH, Fmoc-N-methyl-Val-OH, Fmoc-Sar-OH and Fmoc-Gly-OH, and on a 0.15 mmol scale. The crude peptide (160 mg, 0.14 mmol, 94% yield) was isolated, post ether precipitation, as white powder and was used without further purification.

LC/MS: Calc'd m/z=1140.6 for C₅₄H₈₄N₁₂O₁₅, found [M+H]⁺=1141.8, [M−Boc+2H]²⁺=521.0.

### 2.79 Benzyl tert-butyl ((5aS,9R,15aS,24S,28R,34aS)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontine-9,28-diyl)dicarbamate (Compound 84)

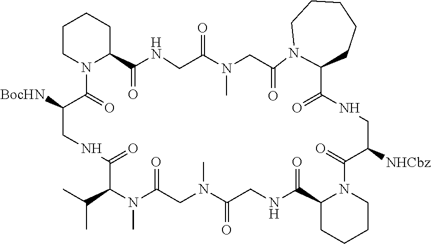 [SMILES: CC(C)[C@H]1C(=O)NC[C@@H](NC(=O)OC(C)(C)C)C(=O)N2CCCC[C@H]2C(=O)NCC(=O)N(C)CC(=O)N2CCCCC[C@H]2C(=O)NC[C@@H](NC(=O)OCc2ccccc2)C(=O)N2CCCC[C@H]2C(=O)NCC(=O)N(C)CC(=O)N1C]

The title compound was synthesized as described in General Procedure 4 starting from Compound 83 (160 mg, 0.14 mmol). Purification according to General Procedure 12, using a 10 g column and eluting with 0 to 15% MeOH/CH₂Cl₂, afforded the title compound (34 mg, 0.03 mmol, 22% yield) as a white solid.

LC/MS: Calc'd m/z=1122.6 for C₅₄H₈₂N₁₂O₁₄, found [M+H]⁺=1123.8, [M−Boc+H]⁺=1024.8, [M−Boc+2H]²⁺=512.6.

### 2.80 Tert-butyl ((5aS,9R,15aS,24S,28R,34aS)-28-amino-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontin-9-yl)carbamate (Compound 85)

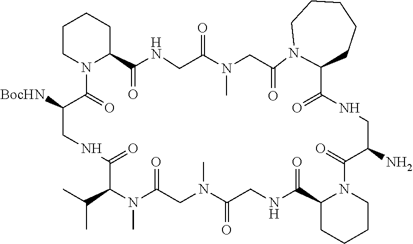 [SMILES: CC(C)[C@H]1C(=O)NC[C@@H](NC(=O)OC(C)(C)C)C(=O)N2CCCC[C@H]2C(=O)NCC(=O)N(C)CC(=O)N2CCCCC[C@H]2C(=O)NC[C@@H](N)C(=O)N2CCCC[C@H]2C(=O)NCC(=O)N(C)CC(=O)N1C]

The title compound was synthesized as described in General Procedure 6 starting from Compound 84 (34 mg, 0.03 mmol), 10% Pd/C (10 mg) and MeOH (2 mL). Following removal of Pd/C, the title compound was isolated as a white solid.

LC/MS: Calc'd m/z=988.6 for C₄₆H₇₆N₁₂O₁₂, found [M−Boc+H]⁺=889.8, [M−Boc+2H]²⁺=445.2.

### 2.81 (5aS,9R,15aS,24S,28R,34aS)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontine-9,28-diaminium chloride (Compound 86)

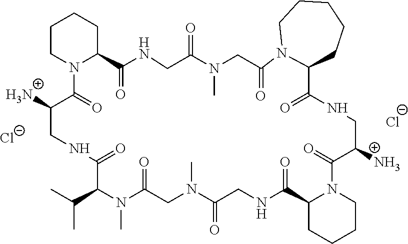 [SMILES: [Cl-] ; CC(C)[C@H]1C(=O)NC[C@@H]([NH3+])C(=O)N2CCCC[C@H]2C(=O)NCC(=O)N(C)CC(=O)N2CCCCC[C@H]2C(=O)NC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 85 (0.03 mmol), and 2 mL of 4 M HCl/dioxane.

LC/MS: Calc'd m/z=888.5 for C₄₁H₆₈N₁₂O₁₀, found [M+H]⁺=889.8, [M+2H]²⁺=445.2.

### 2.82 N,N′-((5aS,9R,15aS,24S,28R,34aS)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontine-9,28-diyl)bis(3-(benzyloxy)quinoline-2-carboxamide) (Compound 87)

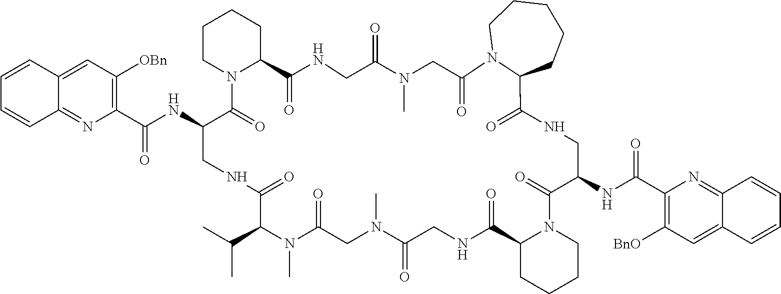 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N2CCCCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 86 (0.03 mmol). Purification according to General Procedure 12, using a 10 g column and eluting with 0 to 20% MeOH/CH₂Cl₂, afforded the title compound (38 mg, 0.027 mmol, 90% yield over 3 steps) as a white solid.

LC/MS: Calc'd m/z=1410.7 for C₇₅H₉₀N₁₄O₁₄, found [M+H]⁺=1412.8, [M+2H]²⁺=706.8.

### 2.83 N,N′-((5aS,9R,15aS,24S,28R,34aS)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontine-9,28-diyl)bis(3-hydroxyquinoline-2-carboxamide) (Compound 88; FD-22)

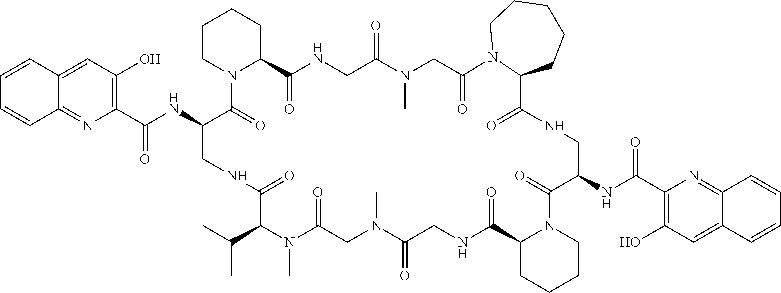 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N2CCCCC[C@H]2C(=O)NC[C@@H](NC(=O)c2nc3ccccc3cc2O)C(=O)N2CCCC[C@H]2C(=O)NCC(=O)N(C)CC(=O)N1C]

Compound 88 was synthesized as described in General Procedure 6 starting from Compound 87 (30 mg, 0.021 mmol), 10% Pd/C (10 mg) and MeOH (2 mL). Following removal of Pd/C, purification according to General Procedure 12, using a 12 g C18 column and eluting with a linear gradient of 10 to 100% CH₃CN/H₂O, afforded the title compound (23 mg, 0.019 mmol, 89% yield) as a pale-yellow solid.

LC/MS: Calc'd m/z=1230.6 for C₆₁H₇₆N₁₄O₁₄, found [M+H]⁺=1233.8, [M+2H]²⁺=616.6.

### 2.84 (S)-cyclohexyl(2-nitrobenzenesulfonamido)acetic acid (Compound 89)

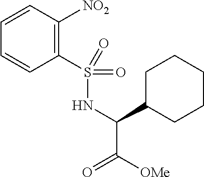 [SMILES: COC(=O)[C@@H](NS(=O)(=O)c1ccccc1[N+](=O)[O-])C1CCCCC1]

To a solution of(S)-amino (cyclohexyl) acetic acid (1.75 g, 11.13 mmol, 1 equiv.) in dioxane (22 mL) and water (18 mL) was added potassium carbonate (4.61 g, 33.39 mmol, 3 equiv.) and 2-nitrobenzenesulfonyl chloride (2.47 g, 11.13 mmol, 1 equiv.). The reaction mixture was then stirred, at rt, until completion, then evaporated to half volume. This solution was diluted with EtOAc, neutralized with NaHSO₄ and extracted with EtOAc (3×15 mL). The combined organic phase was washed with brine, dried over MgSO₄, and evaporated to dryness. Purification according to General Procedure 12, using a 100 g C18 column and eluting with 5 to 60% CH₃CN/H₂O, afforded the title compound (1.29 g, 3.77 mmol, Yield 34%) as a white crystalline solid.

LC/MS: Calc'd m/z=342.1 for C₁₄H₁₈N₂O₆S found [M−H]⁻=341.2. ¹H NMR (300 MHZ, chloroform-d) δ 8.07 (dd, J=5.9, 3.4 Hz, 1H), 7.93-7.84 (m, 1H), 7.71 (dd, J=5.9, 3.3 Hz, 2H), 6.08 (d, J=9.0 Hz, 1H), 4.00 (s, 1H), 1.79 (d, J=26.7 Hz, 6H), 1.38-0.91 (m, 5H).

### 2.85 Methyl(S)-2-cyclohexyl-2-((N-methyl-2-nitrophenyl)sulfonamido)acetate (Compound 90)

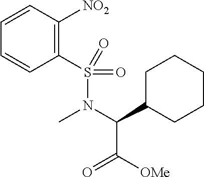 [SMILES: COC(=O)[C@H](C1CCCCC1)N(C)S(=O)(=O)c1ccccc1[N+](=O)[O-]]

To a solution of Compound 89 (1.29 g, 3.77 mmol, 1 equiv.) in dry DMF (25 mL) was added potassium carbonate (1.82 g, 13.19 mmol, 3.5 equiv.) and methyl iodide (1.60 g, 0.704 mL, 11.3 mmol, 3 equiv.). This solution was stirred for 8 h then poured into water (25 mL). The resulting solution was extracted with ether (3×25 mL). The combined organic layers were washed with water, dried over Na₂SO₄, and concentrated. Purification according to General Procedure 12, using a 50 g column and eluting with 20% EtOAc/hexanes, afforded the title compound (1.13 g, 3.05 mmol, Yield 81%) as a white solid.

LC/MS: Calc'd m/z=370.1 for C₁₆H₂₂N₂O₆S found [M+H]⁺=371.2. ¹H NMR (300 MHz, chloroform-d) δ 8.06-7.95 (m, 1H), 7.74-7.57 (m, 3H), 4.20 (d, J=10.3 Hz, 1H), 3.54 (s, 3H), 3.04 (s, 3H), 1.95-1.51 (m, 6H), 1.34-0.86 (m, 5H).

### 2.86 (S)-2-cyclohexyl-2-((N-methyl-2-nitrophenyl)sulfonamido)acetic acid (Compound 91)

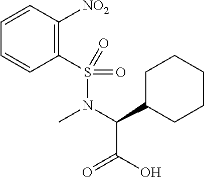 [SMILES: CN([C@H](C(=O)O)C1CCCCC1)S(=O)(=O)c1ccccc1[N+](=O)[O-]]

To a stirring solution of Compound 90 (1.13 g, 3.05 mmol, 1 equiv.) in EtOAc (15 mL), in a 50 mL rb flask wrapped in aluminum foil, was added lithium iodide (2.04 g, 15.25 mmol, 5 equiv.). This solution was heated at reflux until full conversion, then water (15 mL) was added, and the mixture was acidified with aqueous 0.1 M HCl until pH<3. The layers were separated, and the aqueous layer was extracted with EtOAc (2×15 mL). The organic phase was then washed with water (20 mL), aqueous 1M Na₂S₂O₃ (2×20 mL) and brine (20 mL), then the product was extracted with aqueous 0.1 M NaOH (3×50 mL). The combined aqueous phase was washed with CH₂Cl₂ (3×50 mL), acidified with aqueous 1 M HCl and the product was extracted with EtOAc (3×50 mL). The combined organic phase was dried and evaporated to dryness. Purification of the obtained residue by preparative HPLC according to General Procedure 12 afforded the title compound (0.96 g, 2.69 mmol, yield 88%) as a pale yellowish oily residue.

LC/MS: Calc'd m/z=356.1 for C₁₅H₂₀N₂O₆S found [M+H]⁺=357.2. ¹H NMR (300 MHZ, chloroform-d) δ 8.69 (brs, 1H), 8.11-7.96 (m, 1H), 7.78-7.66 (m, 2H), 7.66-7.58 (m, 1H), 4.23 (d, J=9.9 Hz, 1H), 3.04 (s, 3H), 1.92-1.58 (m, 6H), 1.37-1.02 (m, 4H), 1.02-0.80 (m, 1H).

### 2.87 (S)-1-((9S,13R)-1-((S)-1-((R)-3-((S)-2-(2-(2-amino-N-methylacetamido)-N-methylacetamido)-2-cyclohexylacetamido)-2-((tert-butoxycarbonyl)amino)propanoyl) piperidin-2-yl)-13-(((benzyloxy)carbonyl)amino)-9-isopropyl-5,8-dimethyl-1,4,7,10-tetraoxo-2,5,8,11-tetraazatetradecan-14-oyl) piperidine-2-carboxylic acid (Compound 92)

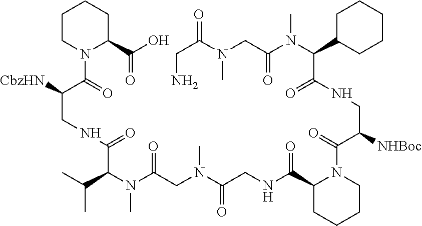 [SMILES: CC(C)[C@@H](C(=O)NC[C@@H](NC(=O)OCc1ccccc1)C(=O)N1CCCC[C@H]1C(=O)O)N(C)C(=O)CN(C)C(=O)CNC(=O)[C@@H]1CCCCN1C(=O)[C@@H](CNC(=O)[C@H](C1CCCCC1)N(C)C(=O)CN(C)C(=O)CN)NC(=O)OC(C)(C)C]

The title compound was synthesized according to General Procedure 1 utilizing Compound 91, Fmoc-Pip-OH, Cbz-D-Dap(Fmoc)-OH, Boc-D-Dap(Fmoc)-OH, Fmoc-N-methyl-Val-OH, Fmoc-Sar-OH and Fmoc-Gly-OH, on a 0.15 mmol scale. The crude peptide (130 mg, 0.11 mmol, 74% yield) was isolated, post ether precipitation, as white powder and was used without further purification.

LC/MS: Calc'd m/z=1168.6 for C₅₆H₈₈N₁₂O₁₅, found [M+H]⁺=1169.8, [M−Boc+2H]²⁺=535.6.

### 2.88 Benzyl tert-butyl ((7R,11S,19aS,26R,30S,38aS)-11-cyclohexyl-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 93)

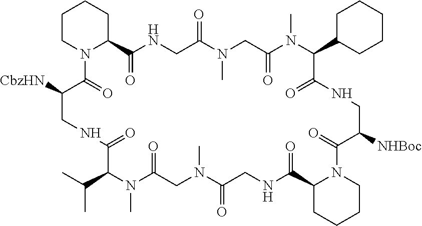 [SMILES: CC(C)[C@H]1C(=O)NC[C@@H](NC(=O)OCc2ccccc2)C(=O)N2CCCC[C@H]2C(=O)NCC(=O)N(C)CC(=O)N(C)[C@@H](C2CCCCC2)C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 92 (130 mg, 0.11 mmol). Purification according to General Procedure 12, using a 10 g column and eluting with 0 to 15% MeOH/CH₂Cl₂, afforded the title compound (73 mg, 0.063 mmol, 58% yield) as a white solid.

LC/MS: Calc'd m/z=1150.6 for C₅₆H₈₆N₁₂O₁₄, found [M+H]⁺=1151.8, [M−Boc+2H]²⁺=526.6.

### 2.89 Tert-butyl ((7R,11S,19aS,26R,30S,38aS)-26-amino-11-cyclohexyl-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 94)

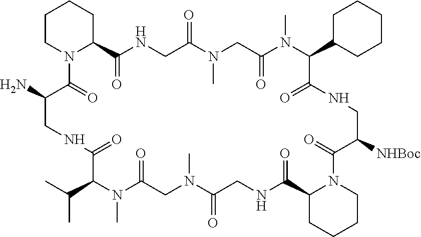 [SMILES: CC(C)[C@H]1C(=O)NC[C@@H](N)C(=O)N2CCCC[C@H]2C(=O)NCC(=O)N(C)CC(=O)N(C)[C@@H](C2CCCCC2)C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 6 starting from Compound 93 (24 mg, 0.021 mmol), 10% Pd/C (10 mg) and MeOH (2 mL). Following removal of Pd/C, the title compound was isolated as a white solid.

LC/MS: Calc'd m/z=1016.6 for C₄₈H₈₀N₁₂O₁₂, found [M+H]⁺=1017.8, [M−Boc+2H]²⁺=459.4.

### 2.90 (7R,11S,19aS,26R,30S,38aS)-11-cyclohexyl-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium chloride (Compound 95)

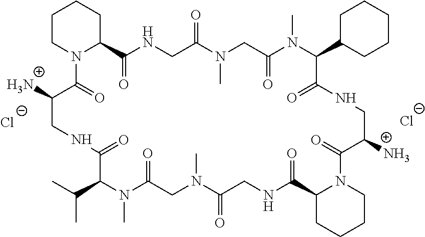 [SMILES: [Cl-] ; CC(C)[C@H]1C(=O)NC[C@@H]([NH3+])C(=O)N2CCCC[C@H]2C(=O)NCC(=O)N(C)CC(=O)N(C)[C@@H](C2CCCCC2)C(=O)NC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 94 (0.03 mmol), and 2 mL of 4 M HCl/dioxane.

LC/MS: Calc'd m/z=916.6 for C₄₃H₇₂N₁₂O₁₀, found [M+H]⁺=917.8, [M+2H]²⁺=459.3.

### 2.91 N,N′-((7R,11S,19aS,26R,30S,38aS)-11-cyclohexyl-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 96)

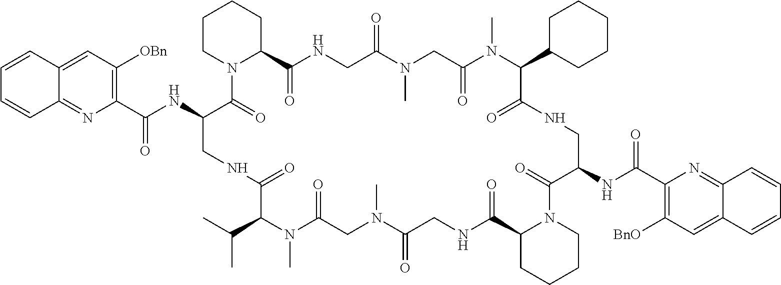 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 95 (0.021 mmol). Purification according to General Procedure 12, using a 10 g column and eluting 0 to 20% MeOH/CH₂Cl₂, afforded the title compound (28 mg, 0.019 mmol, 93% yield over 3 steps) as a white solid.

LC/MS: Calc'd m/z=1438.7 for C₇₇H₉₄N₁₄O₁₄, found [M+H]⁺=1440.4, [M+2H]²⁺=720.8.

### 2.92 N,N′-((7R,11S,19aS,26R,30S,38aS)-11-cyclohexyl-30-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 97; FD-11)

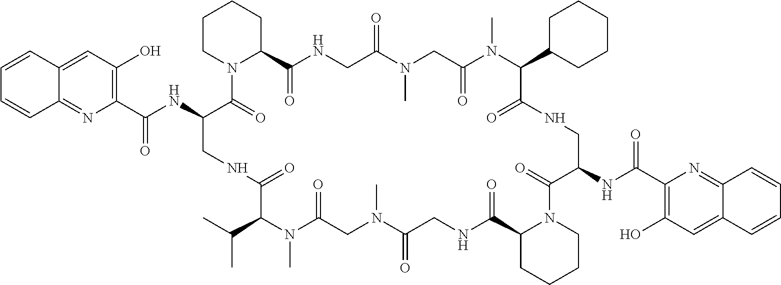 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2O)C(=O)N2CCCC[C@H]2C(=O)NCC(=O)N(C)CC(=O)N1C]

Compound 97 was synthesized as described in General Procedure 6 starting from Compound 96 (28 mg, 0.019 mmol), 10% Pd/C (10 mg) and MeOH (2 mL). Following removal of Pd/C, purification according to General Procedure 12, using a 12 g C18 column and eluting with 10 to 100% MeCN/H₂O, afforded the title compound (22 mg, 0.017 mmol, 90% yield) as a pale-yellow solid.

LC/MS: Calc'd m/z=1258.6 for C₆₃H₈₂N₁₄O₁₄, found [M+H]⁺=1259.8, [M+2H]²⁺=630.6.

### 2.93 (S)-1-((R)-3-((S)-2-(2-(2-((S)-1-((R)-2-(((benzyloxy)carbonyl)amino)-3-((S)-2-(N-methyl-2-(methylamino) acetamido)-2-phenylacetamido) propanoyl)piperidine-2-carboxamido)-N-methylacetamido)-N-methylacetamido)-3-methylbutanamido)-2-((tert-butoxycarbonyl)amino)propanoyl)piperidine-2-carbonyl)glycine (Compound 98)

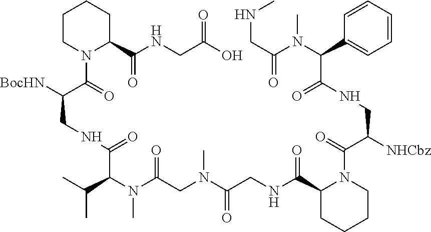 [SMILES: CNCC(=O)N(C)[C@H](C(=O)NC[C@@H](NC(=O)OCc1ccccc1)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)NCC(=O)O)C(C)C)c1ccccc1]

The title compound was synthesized according to General Procedure 1 utilizing Fmoc-Gly-OH, Fmoc-Pip-OH, Boc-D-Dap(Fmoc)-OH, Z-D-Dap(Fmoc)-OH, Fmoc-N-Methyl-Val-OH, Fmoc-Sar-OH and Fmoc-N-methyl-Phenyl-glycine-OH on a 0.13 mmol scale. The crude linear peptide (79 mg, 54%), post ether precipitation, was used without further purification.

LC/MS: Calc'd m/z=1162.6 for C₅₆H₈₂N₁₂O₁₅ found [M+2H-Boc]²⁺=532.2.

### 2.94 Benzyl tert-butyl ((7R,11S,19aS,26R,30S,38aS)-11-isopropyl-12,15,31,34-tetramethyl-6,10,13,16,19,25,29,32,35,38-decaoxo-30-phenylhexatriacontahydro-2H,6H-dipyrido[1,2-a:1′,2′-q][1,4,7,10,13,17,20,23,26,29]decaazacyclodotriacontine-7,26-diyl)dicarbamate (Compound 99)

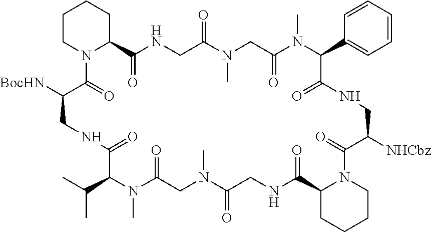 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)[C@@H](c2ccccc2)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 98 (79 mg, 68 umol). Purification according to General Procedure 12, using a 10 g column and eluting with 0 to 10% MeOH/CH₂Cl₂, afforded the title compound as a white solid (20 mg, 17 umol, 26% yield).

LC/MS: Calc'd m/z=1144.6 for C₅₆H₈₀N₁₂O₁₄, found [M+H]⁺=1145.8, [M+2H−Boc]²⁺=1045.8, [M+2H-Boc]²⁺=523.4.

### 2.95 (7R,11S,19aS,26R,30S,38aS)-26-(((benzyloxy)carbonyl)amino)-11-isopropyl-12,15,31,34-tetramethyl-6,10,13,16,19,25,29,32,35,38-decaoxo-30-phenylhexatriacontahydro-2H,6H-dipyrido[1,2-a:1′,2′-q][1,4,7,10,13,17,20,23,26,29]decaazacyclodotriacontin-7-aminium chloride (Compound 100)

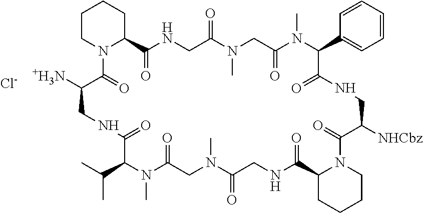 [SMILES: [Cl-] ; CC(C)[C@H]1C(=O)NC[C@@H]([NH3+])C(=O)N2CCCC[C@H]2C(=O)NCC(=O)N(C)CC(=O)N(C)[C@@H](c2ccccc2)C(=O)NC[C@@H](NC(=O)OCc2ccccc2)C(=O)N2CCCC[C@H]2C(=O)NCC(=O)N(C)CC(=O)N1C]

The title compound was synthesized from Compound 99 (7 mg, 6.1 umol) according to General Procedure 5, and isolated as a white solid.

LC/MS: Calc'd m/z=1044.5 for C₅₁H₇₂N₁₂O₁₂, found [M+H]⁺=1045.8, [M+2H]²⁺=523.4.

### 2.96 (7R,11S,19aS,26R,30S,38aS)-7,26-diamino-11-isopropyl-12,15,31,34-tetramethyl-30-phenyltetracosahydro-2H,6H-dipyrido[1,2-a:1′,2′-q][1,4,7,10,13,17,20,23,26,29]decaazacyclodotriacontine-6,10,13,16,19,25,29,32,35,38 (7H,19aH,26H)-decaone monohydrochloride (Compound 101)

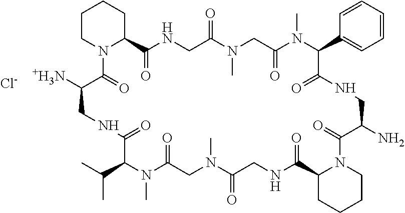 [SMILES: [Cl-] ; CC(C)[C@H]1C(=O)NC[C@@H]([NH3+])C(=O)N2CCCC[C@H]2C(=O)NCC(=O)N(C)CC(=O)N(C)[C@@H](c2ccccc2)C(=O)NC[C@@H](N)C(=O)N2CCCC[C@H]2C(=O)NCC(=O)N(C)CC(=O)N1C]

The title compound was synthesized from Compound 100 (6.1 umol) according to General Procedure 6, and isolated as a white solid (3.1 mg, 53% over 2 steps).

LC/MS: Calc'd mz/=910.5 for C₄₃H₆₆N₁₂O₁₀ found [M+H]⁺=911.6, [M+2H]²⁺=456.4.

### 2.97 N,N′-((7R,11S,19aS,26R,30S,38aS)-11-isopropyl-12,15,31,34-tetramethyl-6,10,13,16,19,25,29,32,35,38-decaoxo-30-phenylhexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 102)

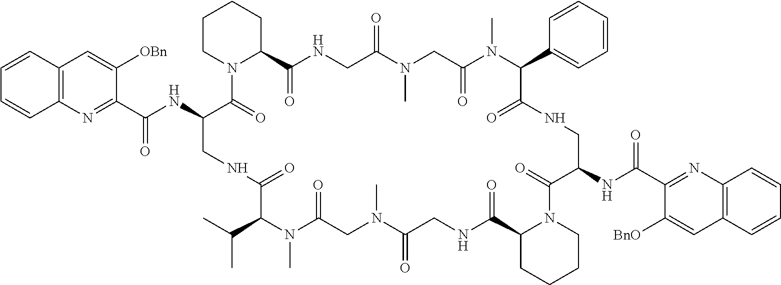 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](c2ccccc2)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was synthesized from Compound 101 (3.1 mg, 3.3 umol) according to General Procedure 7. Purification according to General Procedure 12, using a 10 g column and eluting with 0 to 10% MeOH/CH₂Cl₂, afforded the title compound as a white solid (4.8 mg).

LC/MS: Calc'd m/z=1432.7 for C₇₇H₈₈N₁₄O₁₄ found [M+2H]²⁺=717.8.

### 2.98 N,N′-((7R,11S,19aS,26R,30S,38aS)-11-isopropyl-12,15,31,34-tetramethyl-6,10,13,16,19,25,29,32,35,38-decaoxo-30-phenylhexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 103; FD-12)

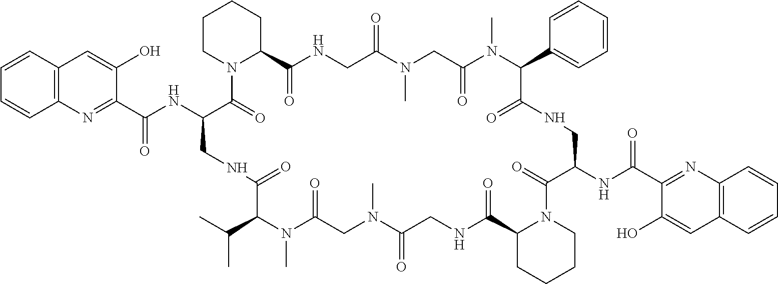 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](c2ccccc2)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Compound 102 (4.8 mg, 3.3 umol) according to General Procedure 8. Preparative HPLC purification according to General Procedure 12 afforded the title compound as a yellow solid (2.0 mg, 48%).

LC/MS: Calc'd m/z=1252.6 for C₆₃H₇₆N₁₄O₁₄ found [M+2H]²⁺=627.8.

### 2.99 ((S)-1-((R)-3-((S)-2-(2-(2-((S)-1-((R)-2-(((benzyloxy)carbonyl)amino)-3-(1-(N-methyl-2-(methylamino) acetamido)cyclopropane-1-carboxamido) propanoyl)piperidine-2-carboxamido)-N-methylacetamido)-N-methylacetamido)-3-methylbutanamido)-2-((tert-butoxycarbonyl)amino)propanoyl)piperidine-2-carbonyl)glycine (Compound 104)

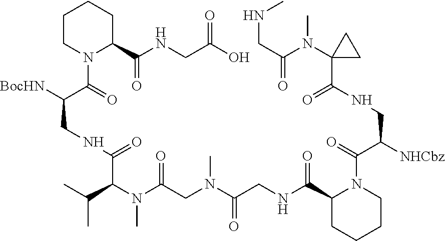 [SMILES: CNCC(=O)N(C)C1(C(=O)NC[C@@H](NC(=O)OCc2ccccc2)C(=O)N2CCCC[C@H]2C(=O)NCC(=O)N(C)CC(=O)N(C)[C@H](C(=O)NC[C@@H](NC(=O)OC(C)(C)C)C(=O)N2CCCC[C@H]2C(=O)NCC(=O)O)C(C)C)CC1]

The title compound was synthesized according to General Procedure 1 utilizing Fmoc-Gly-OH, Fmoc-Pip-OH, Boc-D-Dap(Fmoc)-OH, Z-D-Dap(Fmoc)-OH, Fmoc-N-Methyl-Val-OH, Fmoc-Sar-OH and Fmoc-1-aminocyclopropane-1-carboxylic acid, on a 0.18 mmol scale. The crude linear peptide (227 mg), post ether precipitation, was used without further purification.

LC/MS: Calc'd m/z=1112.6 for C₅₂H₈₀N₁₂O₁₅ found [M+2H-Boc]²⁺=507.3.

### 2.100 Benzyl tert-butyl ((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclopropane-1,11′-dipyrido[1,2-a:1′,2′-q][1,4,7,10,13,17,20,23,26,29]decaazacyclodotriacontine]-7′,26′-diyl)dicarbamate (Compound 105)

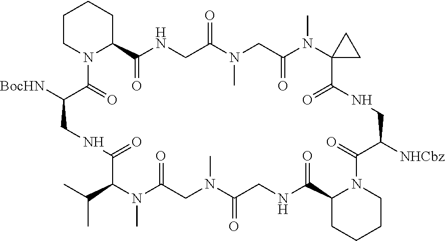 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)C2(CC2)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 104 (227 mg, 204 umol). Purification according to General Procedure 12, using a 10 g column and eluting with 0 to 10% MeOH/CH₂Cl₂, afforded the title compound as a white solid (108 mg, 98 μmol, 48% yield).

LC/MS: Calc'd m/z=1094.6 for C₅₂H₇₈N₁₂O₁₄ found [M+H]⁺=1095.8, [M+H−Boc]⁺=995.8, [M+2H-Boc]²⁺=498.4.

### 2.101 (7′R,19a'S,26′R,30'S,38a'S)-7′-(((benzyloxy)carbonyl)amino)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclopropane-1,11′-dipyrido[1,2-a:1′,2′-q][1,4,7,10,13,17,20,23,26,29]decaazacyclodotriacontin]-26′-aminium trifluoroacetate (Compound 106)

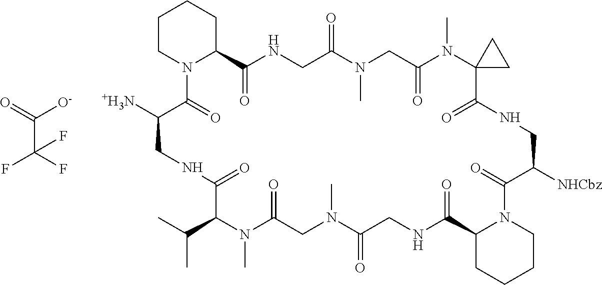 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C2(CC2)C(=O)NC[C@@H](NC(=O)OCc2ccccc2)C(=O)N2CCCC[C@H]2C(=O)NCC(=O)N(C)CC(=O)N1C]

The title compound was synthesized from Compound 105 (15 mg, 13.7 μmol) according to General Procedure 10. Purification according to General Procedure 12, using a 12 g C18 column and eluting with 0 to 100% CH₃CN/H₂O, afforded the title compound (6.8 mg, 6.1 μmol, 45% yield) as a white solid.

LC/MS: Calc'd m/z=994.5 for C₄₇H₇₀N₁₂O₁₂ found [M+H]⁺=995.8, [M+2H]²⁺=498.4.

### 2.102 (7′R,19a'S,26′R,30'S,38a'S)-7′,26′-diamino-30′-isopropyl-12′,15′,31′,34′-tetramethyldocosahydro-2′H,6′H,12′H-spiro[cyclopropane-1,11′-dipyrido[1,2-a:1′,2′-q][1,4,7,10,13,17,20,23,26,29]decaazacyclodotriacontine]-6′,10′,13′,16′,19′,25′,29′,32′,35′,38′ (7′H,19a′H,26′H)-decaone monotrifluoroacetate salt (Compound 107)

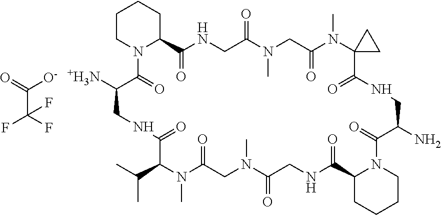 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C2(CC2)C(=O)NC[C@@H](N)C(=O)N2CCCC[C@H]2C(=O)NCC(=O)N(C)CC(=O)N1C]

The title compound was synthesized from Compound 106 (6.8 mg, 6.1 μmol) according to General Procedure 6, and isolated as a white solid (5.9 mg, 6.1 μmol, 99% yield).

LC/MS: Calc'd m/z=860.5 for C₃₉H₆₄N₁₂O₁₀ found [M+H]⁺=861.8, [M+2H]²⁺=431.4.

### 2.103 N,N′-((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclopropane-1,11′-dipyrido[1,2-a:1′,2′-q][1,4,7,10,13,17,20,23,26,29]decaazacyclodotriacontine]-7′,26′-diyl)bis(3-(benzyloxy)quinoline-2-carboxamide) (Compound 108)

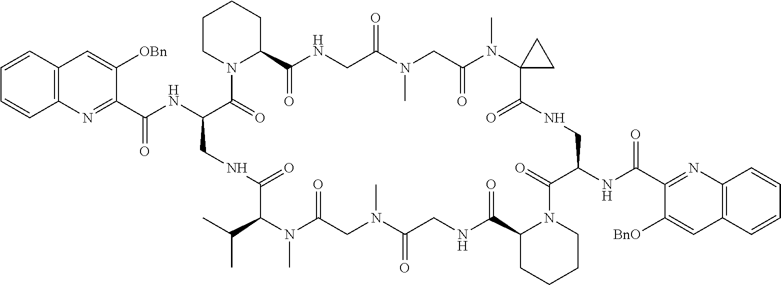 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)C2(CC2)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was synthesized from Compound 107 (5.9 mg, 6.1 μmol) according to General Procedure 7. Purification according to General Procedure 12, using a 10 g column and eluting with 0 to 10% MeOH/CH₂Cl₂, afforded the title compound as a white solid (8.3 mg, 6.0 μmol, 98% yield).

LC/MS: Calc'd m/z=1382.6 for C₇₃H₈₆N₁₄O₁₄ found [M+2H]²⁺=692.8.

### 2.104 N,N′-((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclopropane-1,11′-dipyrido[1,2-a:1′,2′-q][1,4,7,10,13,17,20,23,26,29]decaazacyclodotriacontine]-7′,26′-diyl)bis(3-hydroxyquinoline-2-carboxamide) (Compound 109; FD-32)

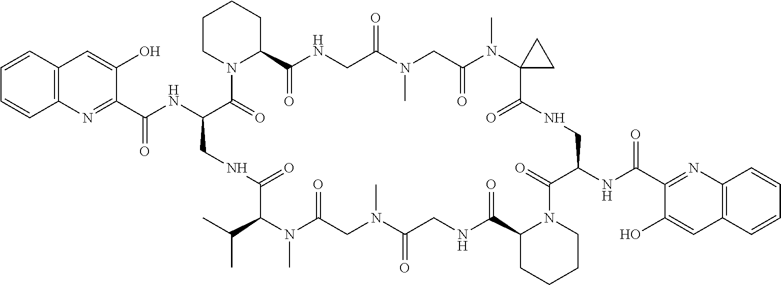 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)C2(CC2)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Compound 108 (8.3 mg, 6.0 μmol) according to General Procedure 8. Purification according to General Procedure 12, using a 12 g C18 column and eluting with 20 to 70% CH₃CN/H₂O, afforded the title compound (2.6 mg, 2.0 μmol, 36% yield) as a yellow solid.

LC/MS: Calc'd m/z=1202.6 for C₅₉H₇₄N₁₄O₁₄ found [M+H]⁺=1203.8, [M+2H]²⁺=602.6.

### 2.105 ((S)-1-((R)-3-((S)-2-(2-(2-((S)-1-((R)-2-(((benzyloxy)carbonyl)amino)-3-(2-methyl-2-(N-methyl-2-(methylamino) acetamido) propanamido) propanoyl)piperidine-2-carboxamido)-N-methylacetamido)-N-methylacetamido)-3-methylbutanamido)-2-((tert-butoxycarbonyl)amino)propanoyl)piperidine-2-carbonyl)glycine (Compound 110)

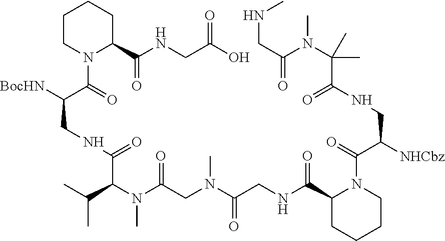 [SMILES: CNCC(=O)N(C)C(C)(C)C(=O)NC[C@@H](NC(=O)OCc1ccccc1)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)NCC(=O)O)C(C)C]

The title compound was synthesized according to General Procedure 1 utilizing Fmoc-Gly-OH, Fmoc-Pip-OH, Boc-D-Dap(Fmoc)-OH, Z-D-Dap(Fmoc)-OH, Fmoc-N-Methyl-Val-OH, Fmoc-Sar-OH and Fmoc-N-Me-Aib-OH, on a 0.18 mmol scale. The crude linear peptide (206 mg), post ether precipitation, was used without additional purification.

LC/MS: Calc'd m/z=1114.6 for C₅₂H₈₂N₁₂O₁₅ found [M+2H-Boc]²⁺=508.3.

### 2.106 Benzyl tert-butyl ((7R,19aS,26R,30S,38aS)-30-isopropyl-11,11,12,15,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 111)

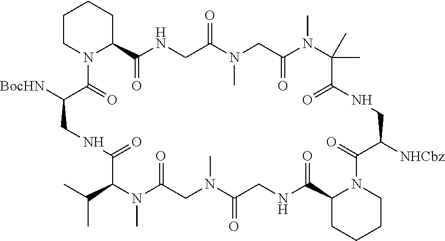 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)C(C)(C)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 110 (206 mg, 185 μmol), and isolated as a white solid (74 mg, 67 μmol, 37% yield).

LC/MS: Calc'd m/z=1096.6 for C₅₂H₇₈N₁₂O₁₄ found [M+H]⁺=1097.8, [M+H−Boc]⁺=997.8, [M+2H-Boc]²⁺=499.6.

### 2.107 (7R,11S,19aS,26R,38aS)-26-(((benzyloxy)carbonyl)amino)-11-isopropyl-12,15,30,30,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-aminium trifluoroacetate (Compound 112)

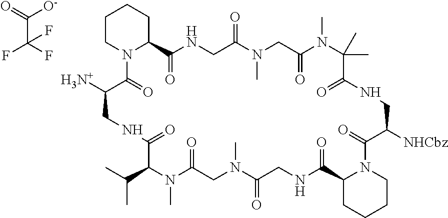 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C(C)(C)C(=O)NC[C@@H](NC(=O)OCc2ccccc2)C(=O)N2CCCC[C@H]2C(=O)NCC(=O)N(C)CC(=O)N1C]

Compound 111 (74 mg, 67 μmol) was deprotected according to General Procedure 10. Under these conditions the macrocycle was also hydrolyzed to the give the linear decapeptide, which could be recyclized according to General Procedure 4. Preparative reverse phase purification according to General Procedure 12 afforded the title compound (33 mg, 30 μmol, 45% yield) as a white solid.

LC/MS: Calc. 996.5 for C₄₇H₇₂N₁₂O₁₂ found 997.8 (M+H)⁺, 499.6 (M+2H)²⁺

### 2.108 (7R,19aS,26R,30S,38aS)-7,26-diamino-30-isopropyl-11,11,12,15,31,34-hexamethyltetracosahydro-2H,6H-dipyrido[1,2-a:1′,2′-q][1,4,7,10,13,17,20,23,26,29]decaazacyclodotriacontine-6,10,13,16,19,25,29,32,35,38 (7H,19aH,26H)-decaone monotrifluoroacetate salt (Compound 113)

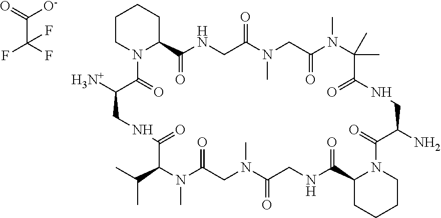 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C(C)(C)C(=O)NC[C@@H](N)C(=O)N2CCCC[C@H]2C(=O)NCC(=O)N(C)CC(=O)N1C]

The title compound was synthesized from Compound 112 (16.5 mg, 14.8 μmol) according to General Procedure 6, and isolated as a white solid (12.4 mg, 12.6 μmol, 85% yield).

LC/MS: Calc'd m/z=862.5 for C₃₉H₆₆N₁₂O₁₀ found [M+H]⁺=863.8, [M+2H]²⁺=432.4.

### 2.109 N,N′-((7R,19aS,26R,30S,38aS)-30-isopropyl-11,11,12,15,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 114)

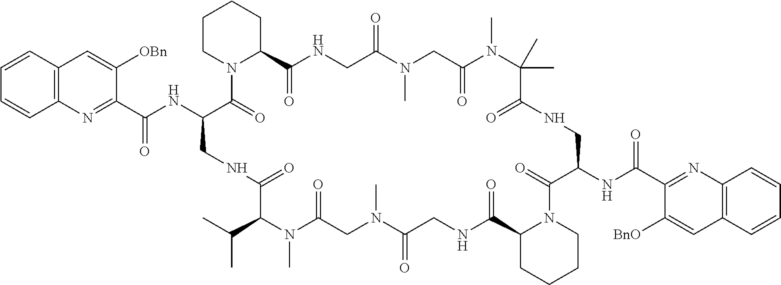 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)C(C)(C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound in mixture with the corresponding cyclic nonapeptide was synthesized from Compound 113 (12.4 mg, 12.7 μmol) according to General Procedure 7. Purification according to General Procedure 12, using a 10 g column and eluting 0 to 10% MeOH/CH₂Cl₂, afforded the title compound as a white solid (20.4 mg).

LC/MS: Calc'd m/z=1384.7 for C₇₃H₈₈N₁₄O₁₄ found [M+2H]²⁺=693.8.

### 2.110 N,N′-((7R,19aS,26R,30S,38aS)-30-isopropyl-11,11,12,15,31,34-hexamethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 115; FD-33)

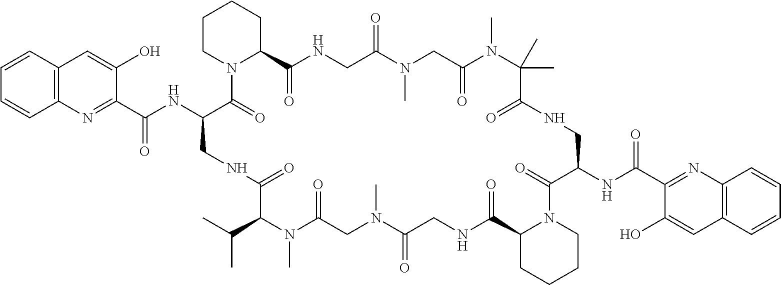 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)C(C)(C)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Compound 114 (17.5 mg, 12.6 μmol) according to General Procedure 8. Purification according to General Procedure 12, using a 12 g C18 column and eluting with 20 to 70% CH₃CN/H₂O, afforded the title compound (6.1 mg, 5.0 μmol, 40% yield) as a yellow solid.

LC/MS: Calc'd m/z=1204.6 for C₅₉H₇₆N₁₄O₁₄ found [M+H]⁺=1205.8, [M+2H]²⁺=603.6.

### 2.111 ((S)-1-((R)-3-((S)-2-(2-(2-((S)-1-((R)-2-(((benzyloxy)carbonyl)amino)-3-(1-(N-methyl-2-(methylamino) acetamido)cyclobutane-1-carboxamido) propanoyl)piperidine-2-carboxamido)-N-methylacetamido)-N-methylacetamido)-3-methylbutanamido)-2-((tert-butoxycarbonyl)amino)propanoyl)piperidine-2-carbonyl)glycine (Compound 116)

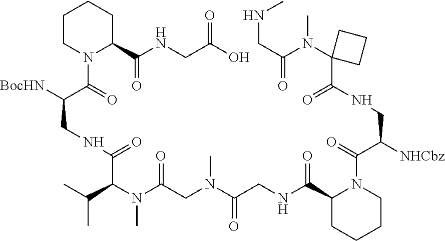 [SMILES: CNCC(=O)N(C)C1(C(=O)NC[C@@H](NC(=O)OCc2ccccc2)C(=O)N2CCCC[C@H]2C(=O)NCC(=O)N(C)CC(=O)N(C)[C@H](C(=O)NC[C@@H](NC(=O)OC(C)(C)C)C(=O)N2CCCC[C@H]2C(=O)NCC(=O)O)C(C)C)CCC1]

The title compound was synthesized according to General Procedure 1 utilizing Fmoc-Gly-OH, Fmoc-Pip-OH, Boc-D-Dap(Fmoc)-OH, Z-D-Dap(Fmoc)-OH, Fmoc-N-Methyl-Val-OH, Fmoc-Sar-OH and Fmoc-1-aminocyclobutane-1-carboxylic acid, on a 0.13 mmol scale. The crude linear peptide (33 mg, 21% yield), post ether precipitation, was used without further purification.

LC/MS: Calc'd m/z=1126.6 for C₅₃H₈₂N₁₂O₁₅ found [M+2H-Boc]²⁺=514.3.

### 2.112 Benzyl tert-butyl ((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclobutane-1,11′-dipyrido[1,2-a:1′,2′-q][1,4,7,10,13,17,20,23,26,29]decaazacyclodotriacontine]-7′,26′-diyl)dicarbamate (Compound 117)

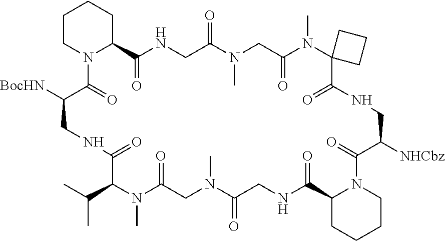 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)C2(CCC2)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 116 (33 mg, 29 μmol). Purification according to General Procedure 12, using a 10 g column and eluting 0 to 10% MeOH/CH₂Cl₂, afforded the title compound (14.0 mg, 12.6 μmol 43%) as a white solid.

LC/MS: Calc'd m/z=1108.6 for C₅₃H₈₀N₁₂O₁₄ found [M+H]⁺=1109.8, [M+H−Boc]⁺=1009.8, [M+2H-Boc]²⁺=505.4.

### 2.113 (7′R,19a'S,26′R,30'S,38a'S)-7′-(((benzyloxy)carbonyl)amino)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclobutane-1,11′-dipyrido[1,2-a:1′,2′-q][1,4,7,10,13,17,20,23,26,29]decaazacyclodotriacontin]-26′-aminium trifluoroacetate (Compound 118)

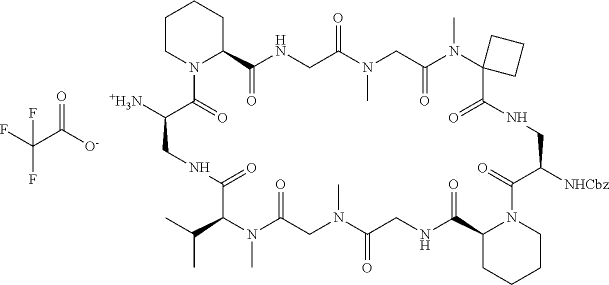 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C2(CCC2)C(=O)NC[C@@H](NC(=O)OCc2ccccc2)C(=O)N2CCCC[C@H]2C(=O)NCC(=O)N(C)CC(=O)N1C]

The title compound was synthesized from Compound 117 (14 mg, 12.6 μmol) according to General Procedure 10, and isolated as a white solid (18 mg).

LC/MS: Calc'd m/z=1008.5 for C₄₈H₇₂N₁₂O₁₂ found [M+H]⁺=1009.8, [M+2H]²⁺=505.4.

### 2.114 (7′R,19a'S,26′R,30'S,38a'S)-7′,26′-diamino-30′-isopropyl-12′,15′,31′,34′-tetramethyldocosahydro-2′H,6′H,12′H-spiro[cyclobutane-1,11′-dipyrido[1,2-a:1′,2′-q][1,4,7,10,13,17,20,23,26,29]decaazacyclodotriacontine]-6′,10′,13′,16′,19′,25′,29′,32′,35′,38′ (7′H,19a′H,26′H)-decaone mono trifluoroacetate salt (Compound 119)

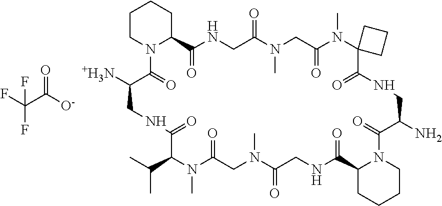 [SMILES: O=C([O-])C(F)(F)F ; CC(C)[C@H]1C(=O)NC[C@@H]([NH3+])C(=O)N2CCCC[C@H]2C(=O)NCC(=O)N(C)CC(=O)N(C)C2(CCC2)C(=O)NC[C@@H](N)C(=O)N2CCCC[C@H]2C(=O)NCC(=O)N(C)CC(=O)N1C]

The title compound was synthesized from Compound 118 (7.5 mg, 5.3 μmol) according to General Procedure 6, and isolated as a white solid (5.3 mg, 5.3 μmol, 99% yield). LC/MS: Calc'd m/z=874.5 for C₄₀H₆₆N₁₂O₁₀ found [M+2H]²⁺=438.4.

### 2.115 N,N′-((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclobutane-1,11′-dipyrido[1,2-a:1′,2′-q][1,4,7,10,13,17,20,23,26,29]decaazacyclodotriacontine]-7′,26′-diyl)bis(3-(benzyloxy)quinoline-2-carboxamide) (Compound 120)

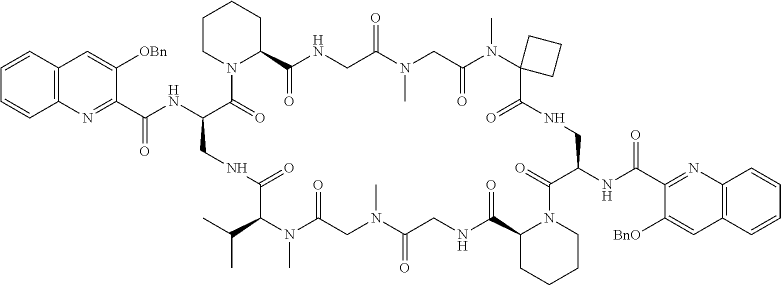 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)C2(CCC2)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was synthesized from Compound 119 (5.3 mg, 5.4 μmol) according to General Procedure 7. Purification according to General Procedure 12, using a 10 g column and eluting 0 to 10% MeOH/CH₂Cl₂, afforded the title compound as a white solid (9.3 mg).

LC/MS: Calc'd m/z=1396.7 for C₇₄H₈₈N₁₄O₁₄ found [M+2H]²⁺=699.8.

### 2.116 N,N′-((7′R,19a'S,26′R,30'S,38a'S)-30′-isopropyl-12′,15′,31′,34′-tetramethyl-6′,10′,13′,16′,19′,25′,29′,32′,35′,38′-decaoxotetratriacontahydro-2′H,6′H,12′H-spiro[cyclobutane-1,11′-dipyrido[1,2-a:1′,2′-q][1,4,7,10,13,17,20,23,26,29]decaazacyclodotriacontine]-7′,26′-diyl)bis(3-hydroxyquinoline-2-carboxamide) (Compound 121; FD-34)

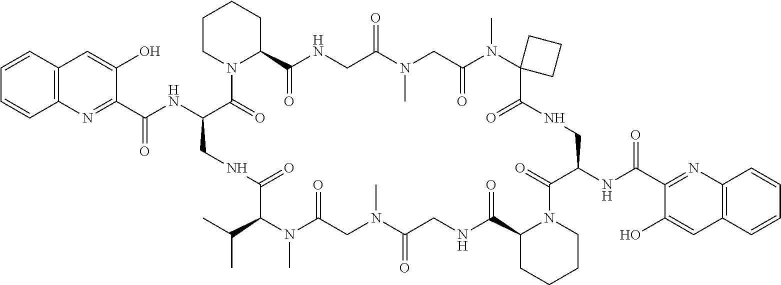 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)C2(CCC2)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Example 120 (8.2 mg, 5.3 μmol) according to General Procedure 8. Preparative HPLC purification according to General Procedure 12 afforded the title compound (2.0 mg, 1.6 μmol, 31% yield) as a yellow solid.

LC/MS: Calc'd m/z=1216.6 for C₆₀H₇₆N₁₄O₁₄, found [M+H]⁺=1217.8, [M+2H]²⁺=609.6.

### 2.117 3-(5-bromo-2-nitrobenzoyl) pentane-2,4-dione (Compound 122)

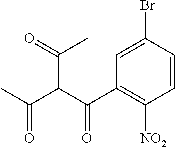 [SMILES: CC(=O)C(C(C)=O)C(=O)c1cc(Br)ccc1[N+](=O)[O-]]

The title compound was prepared from 5-bromo-2-nitrobenzoyl chloride using the same procedure as for Compound 1.

LC/MS: Calc'd m/z=327.0 for C₁₂H₁₀BrNO₅, found [M+H]⁺=328.1. ¹H NMR (300 MHZ, chloroform-d) δ 8.05 (d, J=8.6 Hz, 1H), 8.03 (d, J=2.2 Hz, 1H), 7.98 (dd, J=8.6, 2.1 Hz, 1H), 2.19 (s, 6H).

### 2.118 1-(6-bromo-3-hydroxyquinolin-2-yl)ethan-1-one (Compound 123)

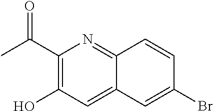

A stirred solution of Compound 122 (3.1 g, 9.45 mmol) in KOH (5.8 g in 29 mL H₂O) was heated at 100° C. for 1 hr. The solution was cooled to rt, then the pH was adjusted to ˜5 with aqueous 6 M HCl. The precipitate was filtered and washed H₂O (2×50 mL), then dried under vacuum to yield 2.41 g (9.02 mmol, 95% yield) of the title compound as an orange solid.

LC/MS: Calc'd m/z=265.0 for C₁₁H₈BrNO₂, found [M+H]⁺=266.0.

### 2.119 1-(6-bromo-3-((4-methoxybenzyl)oxy)quinolin-2-yl)ethan-1-one (Compound 124)

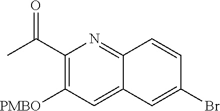

To a stirred solution of crude Compound 123 (670 mg, 2.53 mmol) in acetonitrile (20 mL) was added p-methoxybenzyl chloride (0.41 mL, 3.0 mmol) followed by cesium carbonate (908 mg, 2.79 mmol). The reaction mixture was stirred for 18 h then partitioned between EtOAc (100 mL) and water (50 mL). The organic layer was dried over Na₂SO₄ then concentrated. Purification according to General Procedure 12 eluting with 0 to 50% EtOAc/hexanes afforded the title compound (290 mg, 0.75 mmol, 30% yield) as an orange solid.

LC/MS: Calc'd m/z=385.0 for C₁₉H₁₆BrNO₃, found [M+H]⁺=386.0. ¹H (300 MHZ, acetone-d6) δ 8.11 (d, J=2.2 Hz, 1H), 7.93-7.90 (m, 2H), 7.73 (dd, J=8.8, 2.2 Hz, 1H), 7.49 (d, J=8.8 Hz, 2H), 6.97 (d, J=8.8 Hz, 2H), 5.26 (s, 3H), 2.63 (s, 3H).

### 2.120 6-bromo-3-((4-methoxybenzyl)oxy)quinoline-2-carboxylic acid (Compound 125)

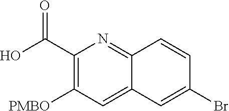

To a stirred solution of Compound 124 (290 mg, 0.75 mmol) in dioxane (10 mL) was added bleach (20 mL). After 1 hr, the reaction mixture was quenched with aqueous 1 M Na₂S₂O₃ (20 mL). The pH of the solution was adjusted to ˜2 with aqueous 1 M HCl then extracted EtOAc (2×50 mL). The combined organic layer was dried over Na₂SO₄ then concentrated in vacuo to afford the title compound (210 mg, 0.541 mmol, 72%) as an orange solid.

LC/MS: Calc'd m/z=387.0 for C₁₈H₁₄BrNO₄, found [M−H]⁻=386.0.

### 2.121 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(6-bromo-3-((4-methoxybenzyl)oxy)quinoline-2-carboxamide)) (Compound 126)

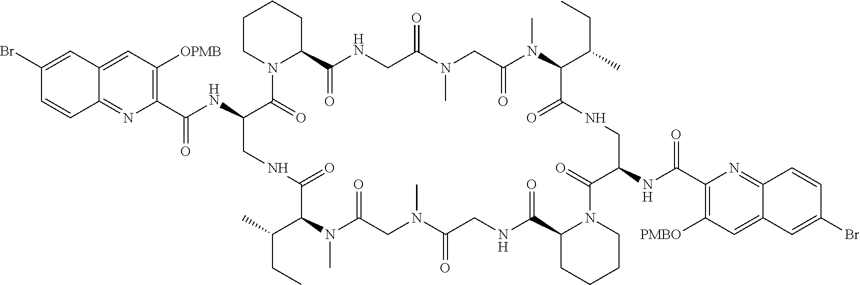

The title compound was prepared from crude Compound 65 (4.0 mg, 0.0044 mmol) according to General Procedure 7 and utilizing Compound 125. Purification according to General Procedure 12, eluting with 0 to 20% MeOH/CH₂Cl₂, afforded the title compound (5.1 mg, 0.0031 mmol, 70% yield) as a white solid.

LC/MS: Calc's m/z=1642.6 for C₇₈H₉₆Br₂N₁₄O₁₆, found [M+2H]²⁺=823.8.

### 2.122 N,N′-((7R,11S,19aS,26R,30S,38aS)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(6-bromo-3-hydroxyquinoline-2-carboxamide) (Compound 127; FD-31)

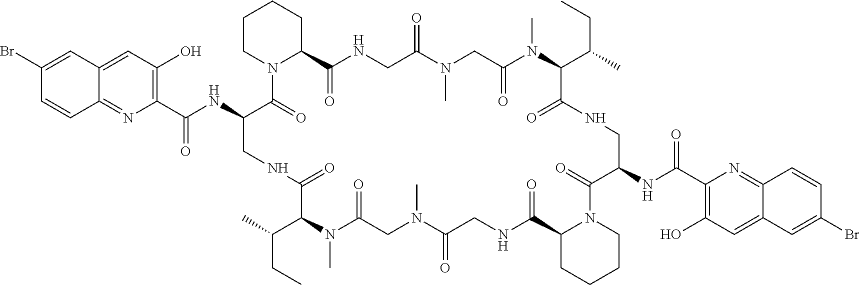 [SMILES: CC[C@H](C)[C@H]1C(=O)NC[C@@H](NC(=O)c2nc3ccc(Br)cc3cc2O)C(=O)N2CCCC[C@H]2C(=O)NCC(=O)N(C)CC(=O)N(C)[C@@H]([C@@H](C)CC)C(=O)NC[C@@H](NC(=O)c2nc3ccc(Br)cc3cc2O)C(=O)N2CCCC[C@H]2C(=O)NCC(=O)N(C)CC(=O)N1C]

A stirred solution of Compound 126 (5 mg, 0.0030 mmol) in glacial acetic acid (1 mL) was heated at 90° C. for 1 h. Reverse phase HPLC purification according to General Procedure 12, eluting with 35 to 75% CH₃CN/H₂O, afforded the title compound (2.3 mg, 0.0016 mmol, 54%) as a faint yellow powder.

LC/MS: Calc'd m/z=1402.4 for C₆₂H₈₀Br₂N₁₄O₁₄, found [M+H]⁺=1405.6, [M+2H]²⁺=703.6.

### 2.123 (S)-1-((9S,13R)-1-((S)-1-((R)-3-((S)-2-(2-(2-amino-N-ethylacetamido)-N-methylacetamido)-3-methylbutanamido)-2-((tert-butoxycarbonyl)amino)propanoyl) piperidin-2-yl)-13-(((benzyloxy)carbonyl)amino)-5-ethyl-9-isopropyl-8-methyl-1,4,7,10-tetraoxo-2,5,8,11-tetraazatetradecan-14-oyl) piperidine-2-carboxylic acid (Compound 128)

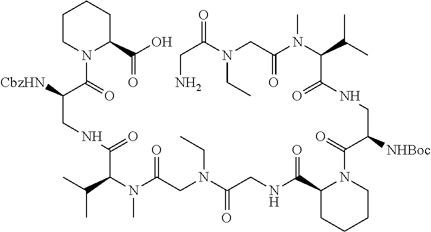 [SMILES: CCN(CC(=O)N(C)[C@H](C(=O)NC[C@@H](NC(=O)OC(C)(C)C)C(=O)N1CCCC[C@H]1C(=O)NCC(=O)N(CC)CC(=O)N(C)[C@H](C(=O)NC[C@@H](NC(=O)OCc1ccccc1)C(=O)N1CCCC[C@H]1C(=O)O)C(C)C)C(C)C)C(=O)CN]

The title compound was synthesized according to General Procedure 1 utilizing Fmoc-Pip-OH, Boc-D-Dap(Fmoc)-OH, Z-D-Dap(Fmoc)-OH, Fmoc-N-methyl-Val-OH, Fmoc-N-methyl-Ile-OH, Fmoc-Gly-OH and Fmoc-N-ethylglycine-OH, on a 0.25 mmol scale. The crude linear peptide (254 mg, 0.22 mmol, 88% yield), post ether precipitation, was used without further purification.

LC/MS: calc. 1156.6 for C₅₅H₈₈N₁₂O₁₅, found [M+H]⁺=1157.8, [M−Boc, +2H]²⁺=529.6.

### 2.124 Benzyl tert-butyl ((7R,11S,19aS,26R,30S,38aS)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 129)

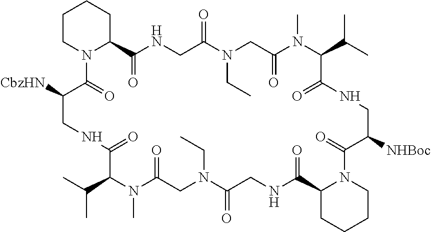 [SMILES: CCN1CC(=O)N(C)[C@@H](C(C)C)C(=O)NC[C@@H](NC(=O)OC(C)(C)C)C(=O)N2CCCC[C@H]2C(=O)NCC(=O)N(CC)CC(=O)N(C)[C@@H](C(C)C)C(=O)NC[C@@H](NC(=O)OCc2ccccc2)C(=O)N2CCCC[C@H]2C(=O)NCC1=O]

The title compound was synthesized as described in General Procedure 4, starting from Compound 128 (254 mg, 0.219 mmol). Purification according to General Procedure 12, eluting with 0 to 20% MeOH/CH₂Cl₂, afforded the title compound (168 mg, 0.148 mmol, 68% yield) as a white film.

LC/MS: Calc'd m/z=1138.6 for C₅₅H₈₆N₁₂O₁₄, found [M+H]⁺=1139.8, [M−Boc+H]⁺=1039.8, [M−Boc+2H]²⁺=520.4.

### 2.125 (7R,11S,19aS,26R,30S,38aS)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium dichloride (Compound 130)

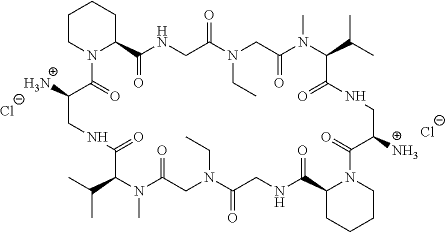 [SMILES: [Cl-] ; CCN1CC(=O)N(C)[C@@H](C(C)C)C(=O)NC[C@@H]([NH3+])C(=O)N2CCCC[C@H]2C(=O)NCC(=O)N(CC)CC(=O)N(C)[C@@H](C(C)C)C(=O)NC[C@@H]([NH3+])C(=O)N2CCCC[C@H]2C(=O)NCC1=O ; [Cl-]]

The title compound was prepared from Compound 129 (30 mg, 0.0264 mmol) according to General Procedure 6 followed by General Procedure 5.

LC/MS: Calc'd m/z=904.5 for C₄₂H₇₂N₁₂O₁₀, found [M+2H]²⁺=453.4.

### 2.126 N,N′-((7R,11S,19aS,26R,30S,38aS)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 131)

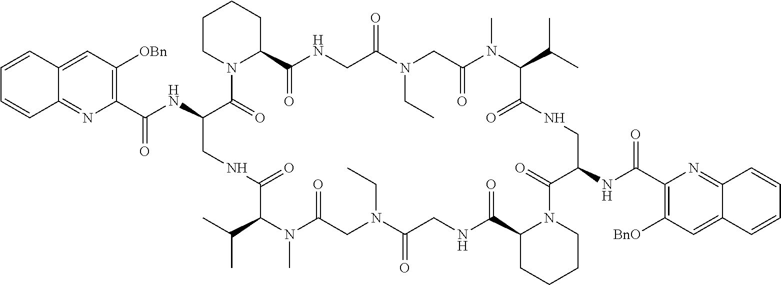 [SMILES: CCN1CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(CC)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC1=O]

The title compound was prepared from crude Compound 130 (0.0264 mmol) according to General Procedure 7. Purification according to General Procedure 12, eluting with 0 to 20% MeOH/CH₂Cl₂, afforded the title compound (26 mg, 0.018 mmol, 69% yield) as a white solid.

LC/MS: Calc'd m/z=1426.7 for C₇₆H₉₄N₁₄O₁₄, found [M+2H]²⁺=714.8.

### 2.127 N,N′-((7R,11S,19aS,26R,30S,38aS)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 132; FD-23)

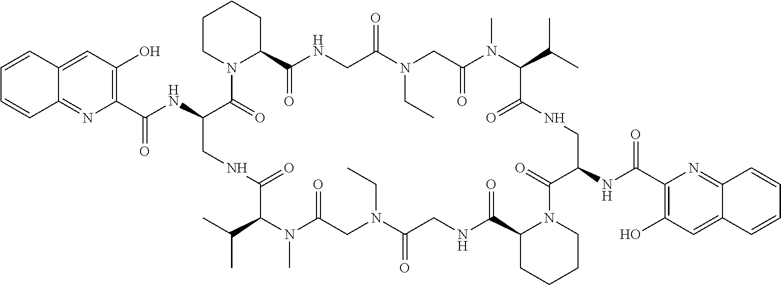 [SMILES: CCN1CC(=O)N(C)[C@@H](C(C)C)C(=O)NC[C@@H](NC(=O)c2nc3ccccc3cc2O)C(=O)N2CCCC[C@H]2C(=O)NCC(=O)N(CC)CC(=O)N(C)[C@@H](C(C)C)C(=O)NC[C@@H](NC(=O)c2nc3ccccc3cc2O)C(=O)N2CCCC[C@H]2C(=O)NCC1=O]

Compound 132 was synthesized from Compound 131 (26 mg, 0.018 mmol) as described in General Procedure 6. Purification according to General Procedure 12, eluting with 30 to 70% CH₃CN/H₂O, afforded the title compound (7.5 mg, 0.006 mmol, 33% yield) as a white solid.

LC/MS: Calc'd m/z=1246.6 for C₆₂H₈₂N₁₄O₁₄, found [M+H]⁺=1247.8, [M+2H]²⁺=624.6.

### 2.128 (S)-1-((9S,13R)-1-((S)-1-((R)-3-((S)-2-(2-(2-aminoacetamido)-N-methylacetamido)-3-methylbutanamido)-2-((tert-butoxycarbonyl)amino)propanoyl) piperidin-2-yl)-13-(((benzyloxy)carbonyl)amino)-9-isopropyl-8-methyl-1,4,7,10-tetraoxo-2,5,8,11-tetraazatetradecan-14-oyl) piperidine-2-carboxylic acid (Compound 133)

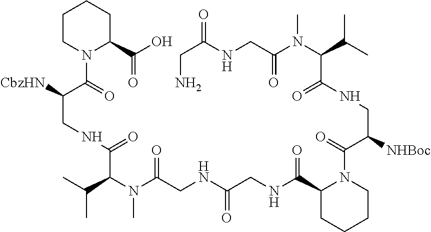 [SMILES: CC(C)[C@@H](C(=O)NC[C@@H](NC(=O)OCc1ccccc1)C(=O)N1CCCC[C@H]1C(=O)O)N(C)C(=O)CNC(=O)CNC(=O)[C@@H]1CCCCN1C(=O)[C@@H](CNC(=O)[C@H](C(C)C)N(C)C(=O)CNC(=O)CN)NC(=O)OC(C)(C)C]

The title compound was synthesized according to General Procedure 1 utilizing Fmoc-Pip-OH, Boc-D-Dap(Fmoc)-OH, Z-D-Dap(Fmoc)-OH, Fmoc-N-methyl-Val-OH, and Fmoc-Gly-Gly-OH, on a 0.15 mmol scale. The crude linear peptide (117 mg, 0.106 mmol, 71% yield), post ether precipitation, was used without further purification.

LC/MS: Calc'd m/z=1100.6 for C₅₁H₈₀N₁₂O₁₅, found [M+H]⁺=1101.8, [M−Boc+2H]²⁺=501.4.

### 2.129 Benzyl tert-butyl ((7R,11S,19aS,26R,30S,38aS)-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 134)

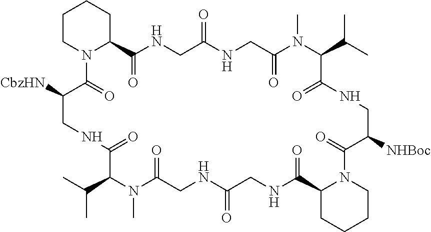 [SMILES: CC(C)[C@H]1C(=O)NC[C@@H](NC(=O)OCc2ccccc2)C(=O)N2CCCC[C@H]2C(=O)NCC(=O)NCC(=O)N(C)[C@@H](C(C)C)C(=O)NC[C@@H](NC(=O)OC(C)(C)C)C(=O)N2CCCC[C@H]2C(=O)NCC(=O)NCC(=O)N1C]

The title compound was synthesized as described in General Procedure 4, starting from Compound 133 (117 mg, 0.106 mmol). Purification according to General Procedure 12, eluting with 0 to 20% MeOH/CH₂Cl₂, afforded the title compound (61 mg, 0.056 mmol, 53% yield) as a white film.

LC/MS: Calc'd m/z=1082.6 for C₅₁H₇₈N₁₂O₁₄, found [M+H]⁺=1083.8, [M−Boc+H]⁺=983.8, [M−Boc+2H]²⁺=492.4.

2.130 (7R,11S,19aS,26R,30S,38aS)-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium dichloride (Compound 135)

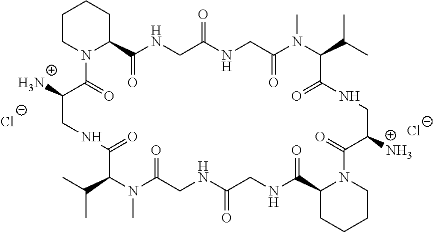 [SMILES: [Cl-] ; CC(C)[C@H]1C(=O)NC[C@@H]([NH3+])C(=O)N2CCCC[C@H]2C(=O)NCC(=O)NCC(=O)N(C)[C@@H](C(C)C)C(=O)NC[C@@H]([NH3+])C(=O)N2CCCC[C@H]2C(=O)NCC(=O)NCC(=O)N1C ; [Cl-]]

The title compound was prepared from Compound 134 (30 mg, 0.278 mmol) according to General Procedure 6 followed by General Procedure 5.

LC/MS: Calc'd m/z=848.5 for C₃₈H₆₄N₁₂O₁₀, found [M+H]⁺=849.6, [M+2H]²⁺=425.4.

### 2.131 N,N′-((7R,11S,19aS,26R,30S,38aS)-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 136)

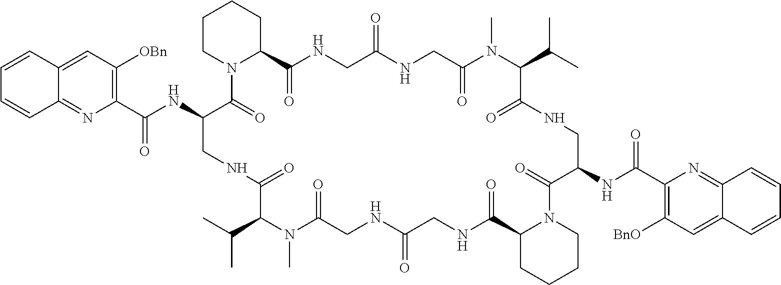 [SMILES: CC(C)[C@H]1C(=O)NC[C@@H](NC(=O)c2nc3ccccc3cc2OCc2ccccc2)C(=O)N2CCCC[C@H]2C(=O)NCC(=O)NCC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)NCC(=O)N1C]

The title compound was prepared from crude Compound 135 (0.028 mmol) according to General Procedure 7. Purification according to General Procedure 12, eluting with 0 to 20% MeOH/CH₂Cl₂, afforded the title compound (28 mg, 0.020 mmol, 73% yield) as a white solid.

LC/MS: Calc'd m/z=1370.6 for C₇₂H₈₆N₁₄O₁₄, found [M+2H]²⁺=686.6.

### 2.132 N,N′-((7R,11S,19aS,26R,30S,38aS)-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 137; FD-24)

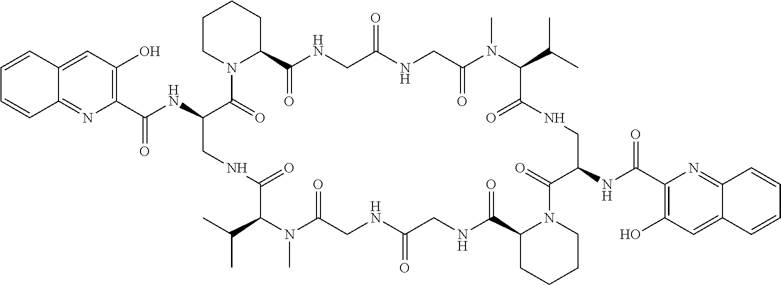 [SMILES: CC(C)[C@H]1C(=O)NC[C@@H](NC(=O)c2nc3ccccc3cc2O)C(=O)N2CCCC[C@H]2C(=O)NCC(=O)NCC(=O)N(C)[C@@H](C(C)C)C(=O)NC[C@@H](NC(=O)c2nc3ccccc3cc2O)C(=O)N2CCCC[C@H]2C(=O)NCC(=O)NCC(=O)N1C]

The title compound was synthesized from Compound 136 (28 mg, 0.0204 mmol) as described in General Procedure 6. Purification according to General Procedure 12, eluting with 30 to 65% CH₃CN/H₂O, afforded the title compound (6.4 mg, 0.0054 mmol, 26% yield) as a white solid.

LC/MS: Calc'd m/z=1190.6 for C₅₈H₇₄N₁₄O₁₄, found [M+H]⁺=1191.8, [M+2H]²⁺=596.6.

### 2.133 (S)-1-((9S,13R)-1-((S)-1-((R)-3-((S)-2-(2-(2-amino-N-methylacetamido)-N-ethylacetamido)-3-methylbutanamido)-2-((tert-butoxycarbonyl)amino)propanoyl) piperidin-2-yl)-13-(((benzyloxy)carbonyl)amino)-8-ethyl-9-isopropyl-5-methyl-1,4,7,10-tetraoxo-2,5,8,11-tetraazatetradecan-14-oyl) piperidine-2-carboxylic acid (Compound 143)

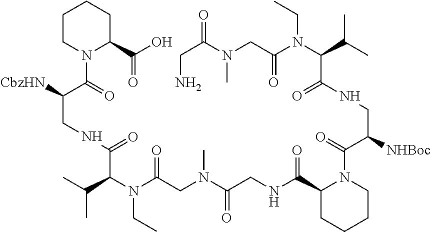 [SMILES: CCN(C(=O)CN(C)C(=O)CN)[C@H](C(=O)NC[C@@H](NC(=O)OC(C)(C)C)C(=O)N1CCCC[C@H]1C(=O)NCC(=O)N(C)CC(=O)N(CC)[C@H](C(=O)NC[C@@H](NC(=O)OCc1ccccc1)C(=O)N1CCCC[C@H]1C(=O)O)C(C)C)C(C)C]

The title compound was synthesized according to General Procedure 1 utilizing Fmoc-Pip-OH, Boc-D-Dap(Fmoc)-OH and Z-D-Dap(Fmoc)-OH, Compound 205 and Compound 9, on a 0.15 mmol scale. The crude linear peptide (112 mg, 0.097 mmol, 65% yield), post ether precipitation, was used without further purification.

LC/MS: Calc'd m/z=1156.6 for C₅₅H₈₈N₁₂O₁₅, found [M+H]⁺=1157.8, [M−Boc+2H]²⁺=529.6.

### 2.134 Benzyl tert-butyl ((7R,11S,19aS,26R,30S,38aS)-12,31-diethyl-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 144)

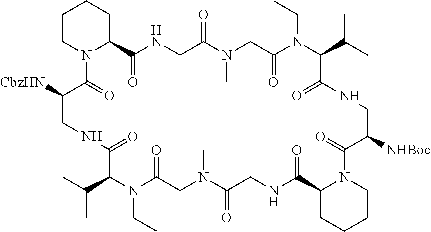 [SMILES: CCN1C(=O)CN(C)C(=O)CNC(=O)[C@@H]2CCCCN2C(=O)[C@H](NC(=O)OCc2ccccc2)CNC(=O)[C@H](C(C)C)N(CC)C(=O)CN(C)C(=O)CNC(=O)[C@@H]2CCCCN2C(=O)[C@H](NC(=O)OC(C)(C)C)CNC(=O)[C@@H]1C(C)C]

The title compound was synthesized as described in General Procedure 4, starting from Compound 143 (112 mg, 0.0969 mmol). Purification was according to General Procedure 12, eluting with 0 to 20% MeOH/CH₂Cl₂ gradient. The peptide was then purified by reverse-phase chromatography eluting with a 20 to 60% CH₃CN/H₂O to afford the title compound (44 mg, 0.0387 mmol, 40% yield) as a white film.

LC/MS: Calc'd m/z=1138.6 for C₅₅H₈₆N₁₂O₁₄, found [M−Boc+H]⁺=1039.8, [M−Boc+2H]²⁺=520.4

### 2.135 (7R,11S,19aS,26R,30S,38aS)-12,31-diethyl-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium dichloride (Compound 145)

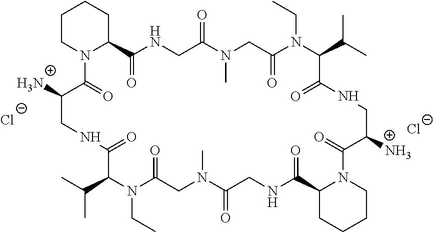 [SMILES: [Cl-] ; CCN1C(=O)CN(C)C(=O)CNC(=O)[C@@H]2CCCCN2C(=O)[C@H]([NH3+])CNC(=O)[C@H](C(C)C)N(CC)C(=O)CN(C)C(=O)CNC(=O)[C@@H]2CCCCN2C(=O)[C@H]([NH3+])CNC(=O)[C@@H]1C(C)C ; [Cl-]]

The title compound was prepared from Compound 144 (26 mg, 0.023 mmol) according to General Procedure 6 followed by General Procedure 5.

LC/MS: Calc'd m/z=904.6 for C₄₂H₇₂N₁₂O₁₀, found [M+H]⁺=905.8, [M+2H]²⁺=453.4.

### 2.136 N,N′-((7R,11S,19aS,26R,30S,38aS)-12,31-diethyl-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 146)

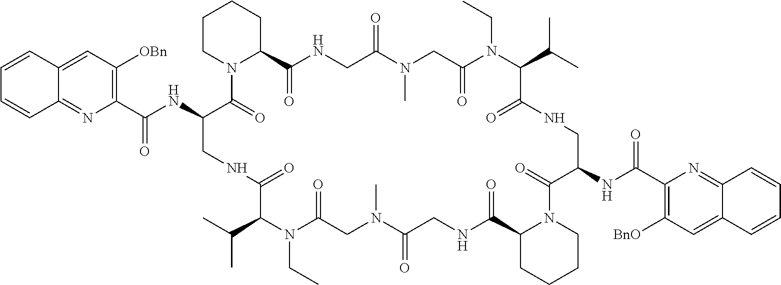 [SMILES: CCN1C(=O)CN(C)C(=O)CNC(=O)[C@@H]2CCCCN2C(=O)[C@H](NC(=O)c2nc3ccccc3cc2OCc2ccccc2)CNC(=O)[C@H](C(C)C)N(CC)C(=O)CN(C)C(=O)CNC(=O)[C@@H]2CCCCN2C(=O)[C@H](NC(=O)c2nc3ccccc3cc2OCc2ccccc2)CNC(=O)[C@@H]1C(C)C]

The title compound was prepared from crude Compound 145 (0.023 mmol) according to General Procedure 7. Purification according to General Procedure 12, eluting with 0 to 10% MeOH/CH₂Cl₂, afforded the title compound (31 mg, 0.022 mmol, 95% yield) as a white solid.

LC/MS: Calc'd m/z=1426.7 for C₇₆H₉₄N₁₄O₁₄, found [M+2H]²⁺=714.8.

### 2.137 N,N′-((7R,11S,19aS,26R,30S,38aS)-12,31-diethyl-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 147; FD-25)

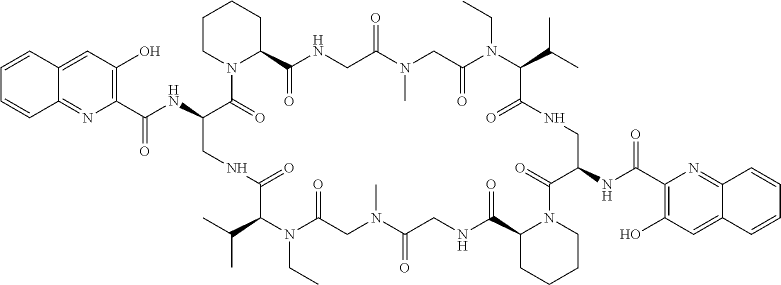 [SMILES: CCN1C(=O)CN(C)C(=O)CNC(=O)[C@@H]2CCCCN2C(=O)[C@H](NC(=O)c2nc3ccccc3cc2O)CNC(=O)[C@H](C(C)C)N(CC)C(=O)CN(C)C(=O)CNC(=O)[C@@H]2CCCCN2C(=O)[C@H](NC(=O)c2nc3ccccc3cc2O)CNC(=O)[C@@H]1C(C)C]

The title compound was synthesized from Compound 146 (31 mg, 0.22 mmol) as described in General Procedure 6. Purification according to General Procedure 12, eluting with 10 to 60% CH₃CN/H₂O, afforded the title compound (8.2 mg, 0.00658 mmol, 30% yield) as a light-yellow solid.

LC/MS: Calc'd m/z=1246.6 for C₆₂H₈₂N₁₄O₁₄, found [M+H]⁺=1247.8, [M+2H]²⁺=624.6.

### 2.138 (S)-1-((9S,13R)-1-((S)-1-((R)-3-((S)-2-(2-(2-amino-N-methylacetamido) acetamido)-3-methylbutanamido)-2-((tert-butoxycarbonyl)amino)propanoyl) piperidin-2-yl)-13-(((benzyloxy)carbonyl)amino)-9-isopropyl-5-methyl-1,4,7,10-tetraoxo-2,5,8,11-tetraazatetradecan-14-oyl) piperidine-2-carboxylic acid (Compound 148)

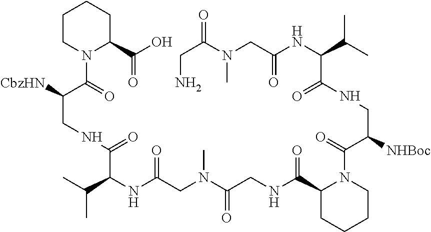 [SMILES: CC(C)[C@H](NC(=O)CN(C)C(=O)CN)C(=O)NC[C@@H](NC(=O)OC(C)(C)C)C(=O)N1CCCC[C@H]1C(=O)NCC(=O)N(C)CC(=O)N[C@H](C(=O)NC[C@@H](NC(=O)OCc1ccccc1)C(=O)N1CCCC[C@H]1C(=O)O)C(C)C]

The title compound was synthesized according to General Procedure 1 utilizing Fmoc-Pip-OH, Boc-D-Dap(Fmoc)-OH, Z-D-Dap(Fmoc)-OH, N-Fmoc-L-valine and N-hydroxysuccinimide ester, and Compound 9, on a 0.15 mmol scale. The crude linear peptide (120 mg, 0.11 mmol, 73% yield), post ether precipitation, was used without further purification.

LC/MS: Calc'd m/z=1100.6 for C₅₁H₈₀N₁₂O₁₅, found [M+H]⁺=1101.8, [M+2H]²⁺=551.0.

### 2.139 Benzyl tert-butyl ((7R,11S,19aS,26R,30S,38aS)-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 149)

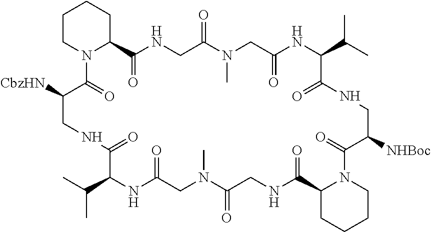 [SMILES: CC(C)[C@@H]1NC(=O)CN(C)C(=O)CNC(=O)[C@@H]2CCCCN2C(=O)[C@H](NC(=O)OCc2ccccc2)CNC(=O)[C@H](C(C)C)NC(=O)CN(C)C(=O)CNC(=O)[C@@H]2CCCCN2C(=O)[C@H](NC(=O)OC(C)(C)C)CNC1=O]

The title compound was synthesized as described in General Procedure 4, starting from Compound 148 (120 mg, 0.109 mmol). Purification according to General Procedure 12, eluting with 0 to 20% MeOH/CH₂Cl₂ followed by preparative HPLC purification as described in General Procedure 12, eluting with a 30 to 50% CH₃CN/H₂O, afforded the title compound (24 mg, 0.022 mmol, 20% yield) as a white film.

LC/MS: Calc'd m/z=1082.6 for C₅₁H₇₈N₁₂O₁₄, found [M+H]⁺=1083.8, [M−Boc+2H]²⁺=542.

### 2.140 (7R,11S,19aS,26R,30S,38aS)-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium (Compound 150)

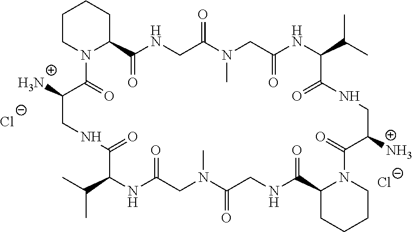 [SMILES: [Cl-] ; CC(C)[C@@H]1NC(=O)CN(C)C(=O)CNC(=O)[C@@H]2CCCCN2C(=O)[C@H]([NH3+])CNC(=O)[C@H](C(C)C)NC(=O)CN(C)C(=O)CNC(=O)[C@@H]2CCCCN2C(=O)[C@H]([NH3+])CNC1=O ; [Cl-]]

The title compound was prepared from Compound 149 (24 mg, 0.022 mmol) according to General Procedure 6 followed by General Procedure 5.

LC/MS: Calc'd m/z=848.5 for C₃₈H₆₄N₁₂O₁₀, found [M+2H]²⁺=424.4.

### 2.141 N,N′-((7R,11S,19aS,26R,30S,38aS)-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 151)

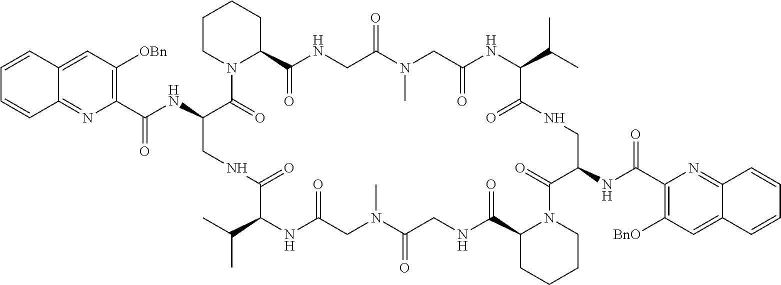 [SMILES: CC(C)[C@@H]1NC(=O)CN(C)C(=O)CNC(=O)[C@@H]2CCCCN2C(=O)[C@H](NC(=O)c2nc3ccccc3cc2OCc2ccccc2)CNC(=O)[C@H](C(C)C)NC(=O)CN(C)C(=O)CNC(=O)[C@@H]2CCCCN2C(=O)[C@H](NC(=O)c2nc3ccccc3cc2OCc2ccccc2)CNC1=O]

The title compound was prepared from crude Compound 150 (0.022 mmol) according to General Procedure 7. Purification according to General Procedure 12, eluting with 0 to 20% MeOH/CH₂Cl₂ gradient, afforded the title compound (19 mg, 0.014 mmol, 63% yield) as a white solid.

LC/MS: Calc'd m/z=1370.6 for C₇₂H₈₆N₁₄O₁₄, found [M+2H]²⁺=686.8.

### 2.142 N,N′-((7R,11S,19aS,26R,30S,38aS)-12,31-diethyl-11,30-diisopropyl-15,34-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 152; FD-26)

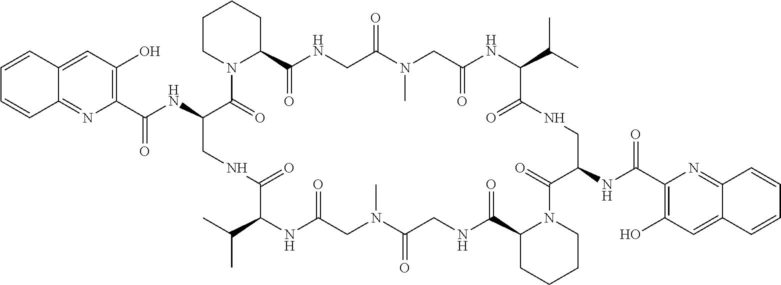 [SMILES: CC(C)[C@@H]1NC(=O)CN(C)C(=O)CNC(=O)[C@@H]2CCCCN2C(=O)[C@H](NC(=O)c2nc3ccccc3cc2O)CNC(=O)[C@H](C(C)C)NC(=O)CN(C)C(=O)CNC(=O)[C@@H]2CCCCN2C(=O)[C@H](NC(=O)c2nc3ccccc3cc2O)CNC1=O]

The title compound was synthesized from Compound 151 (19 mg, 0.014 mmol) as described in General Procedure 6. Purification according to General Procedure 12, eluting with 20 to 60% CH₃CN/H₂O, afforded the title compound (1.4 mg, 0.0066 mmol, 30% yield) as a white solid.

LC/MS: Calc'd m/z=1190.6 for C₅₈H₇₄N₁₄O₁₄, found [M+H]⁺=1191.8, [M+2H]²⁺=596.6.

### 2.143 (S)-1-((R)-2-(((benzyloxy)carbonyl)amino)-3-((S)-1-(N—(((S)-1-((R)-2-((tert-butoxycarbonyl)amino)-3-((S)-1-(N-glycyl-N-methylglycyl) azepane-2-carboxamido) propanoyl)piperidine-2-carbonyl)glycyl)-N-methylglycyl) azepane-2-carboxamido) propanoyl)piperidine-2-carboxylic acid (Compound 153)

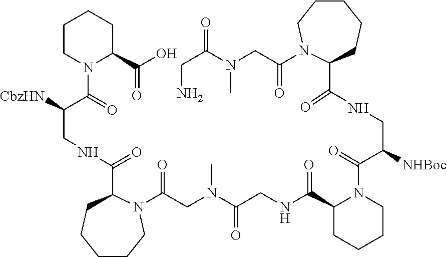 [SMILES: CN(CC(=O)N1CCCCC[C@H]1C(=O)NC[C@@H](NC(=O)OC(C)(C)C)C(=O)N1CCCC[C@H]1C(=O)NCC(=O)N(C)CC(=O)N1CCCCC[C@H]1C(=O)NC[C@@H](NC(=O)OCc1ccccc1)C(=O)N1CCCC[C@H]1C(=O)O)C(=O)CN]

The title compound was synthesized according to General Procedure 1 utilizing Fmoc-Pip-OH, Boc-D-Dap(Fmoc)-OH, Z-D-Dap(Fmoc)-OH, Fmoc and(S)-1-(((9H-fluoren-9-yl)methoxy)carbonyl) azepane-2-carboxylic acid, and Compound 9. The crude linear peptide (357 mg, 0.31 mmol, 77% yield), post ether precipitation, was used without further purification.

LC/MS: Calc'd m/z=1152.6 for C₅₅H₈₄N₁₂O₁₅, found [M+H]⁺=1153.8, [M−Boc+2H]²⁺=527.6.

### 2.144 Benzyl tert-butyl ((7R,10aS,23aS,30R,33aS,46aS)-19,42-dimethyl-6,10,17,20,23,29,33,40,43,46-decaoxotetratetracontahydro-6H,29H-bis(azepino)[1,2-j:1′,2′-z]dipyrido[1,2-a:1′,2′-q][1,4,7,10,13,17,20,23,26,29]decaazacyclodotriacontine-7,30-diyl)dicarbamate (Compound 154)

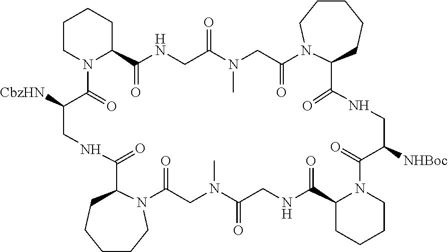 [SMILES: CN1CC(=O)N2CCCCC[C@H]2C(=O)NC[C@@H](NC(=O)OC(C)(C)C)C(=O)N2CCCC[C@H]2C(=O)NCC(=O)N(C)CC(=O)N2CCCCC[C@H]2C(=O)NC[C@@H](NC(=O)OCc2ccccc2)C(=O)N2CCCC[C@H]2C(=O)NCC1=O]

The title compound was synthesized as described in General Procedure 4, starting from Compound 153 (357 mg, 0.310 mmol). Purification according to General Procedure 12, eluting with 0 to 20% MeOH/CH₂Cl₂, afforded the title compound (262 mg, 0.231 mmol, 74% yield) as a white solid.

LC/MS: Calc'd m/z=1134.61 for C₅₅H₈₂N₁₂O₁₄, found [M+H]⁺=1135.8, [M−Boc+H]⁺=1025.8, [M−Boc+2H]²⁺=518.4.

### 2.145 (7R,10aS,23aS,30R,33aS,46aS)-19,42-dimethyl-6,10,17,20,23,29,33,40,43,46-decaoxotetratetracontahydro-6H,29H-bis(azepino) [1,2-j:1′,2′-z]dipyrido[1,2-a:1′,2′-q][1,4,7,10,13,17,20,23,26,29]decaazacyclodotriacontine-7,30-diaminium (Compound 155)

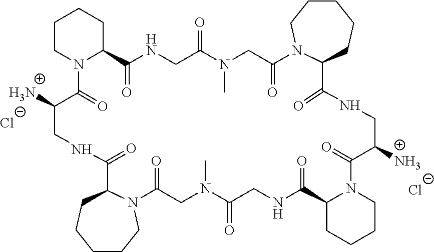 [SMILES: [Cl-] ; CN1CC(=O)N2CCCCC[C@H]2C(=O)NC[C@@H]([NH3+])C(=O)N2CCCC[C@H]2C(=O)NCC(=O)N(C)CC(=O)N2CCCCC[C@H]2C(=O)NC[C@@H]([NH3+])C(=O)N2CCCC[C@H]2C(=O)NCC1=O ; [Cl-]]

The title compound was prepared from Compound 154 (29 mg, 0.026 mmol) according to General Procedure 6 followed by General Procedure 5.

LC/MS: Calc'd m/z=900.5 for C₄₂H₆₈N₁₂O₁₀, found [M+H]⁺=901.8, [M+2H]²⁺=451.4.

### 2.146 N,N′-((7R,10aS,23aS,30R,33aS,46aS)-19,42-dimethyl-6,10,17,20,23,29,33,40,43,46-decaoxotetratetracontahydro-6H,29H-bis(azepino)[1,2-j:1′,2′-z]dipyrido[1,2-a:1′,2′-q][1,4,7,10,13,17,20,23,26,29]decaazacyclodotriacontine-7,30-diyl)bis(3-(benzyloxy)quinoline-2-carboxamide) (Compound 156)

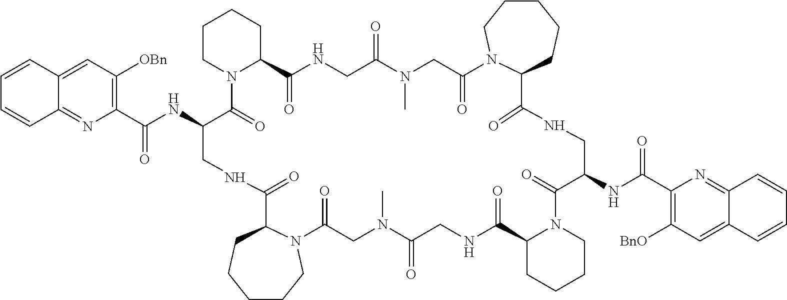 [SMILES: CN1CC(=O)N2CCCCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N2CCCCC[C@H]2C(=O)NC[C@@H](NC(=O)c2nc3ccccc3cc2OCc2ccccc2)C(=O)N2CCCC[C@H]2C(=O)NCC1=O]

The title compound was prepared from crude Compound 155 (23 mg 0.0256 mmol) according to General Procedure 7. Purification according to General Procedure 12, eluting with 0 to 20% MeOH/CH₂C₁₂, afforded the title compound (20 mg, 0.014 mmol, 55% yield) as a white solid.

LC/MS: Calc'd m/z=1422.7 for C₇₆H₉₀N₁₄O₁₄, found [M+2H]²⁺=712.8.

### 2.147 N,N′-((7R,10aS,23aS,30R,33aS,46aS)-19,42-dimethyl-6,10,17,20,23,29,33,40,43,46-decaoxotetratetracontahydro-6H,29H-bis(azepino) [1,2-j:1′,2′-z]dipyrido[1,2-a:1′,2′-q][1,4,7,10,13,17,20,23,26,29]decaazacyclodotriacontine-7,30-diyl)bis(3-hydroxyquinoline-2-carboxamide) (Compound 157; FD-16)

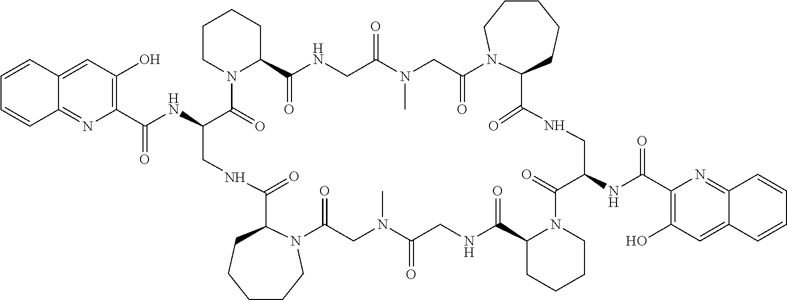 [SMILES: CN1CC(=O)N2CCCCC[C@H]2C(=O)NC[C@@H](NC(=O)c2nc3ccccc3cc2O)C(=O)N2CCCC[C@H]2C(=O)NCC(=O)N(C)CC(=O)N2CCCCC[C@H]2C(=O)NC[C@@H](NC(=O)c2nc3ccccc3cc2O)C(=O)N2CCCC[C@H]2C(=O)NCC1=O]

The title compound was synthesized from Compound 156 (20 mg, 0.014 mmol) as described in General Procedure 8. Purification according to General Procedure 12, eluting with 30 to 60% CH₃CN/H₂O, afforded the title compound (3.3 mg, 0.0027 mmol, 19% yield) as a light-yellow solid.

LC/MS: Calc'd m/z=1242.6 for C₆₂H₇₈N₁₄O₁₄, found [M+H]⁺=1243.8, [M+2H]²⁺=622.8.

### 2.148 (S)-1-((9S,13R)-1-((S)-1-((R)-3-((S)-2-(2-(2-amino-N-methylacetamido)-N-methylacetamido)-2-cyclohexylacetamido)-2-((tert-butoxycarbonyl)amino)propanoyl) piperidin-2-yl)-13-(((benzyloxy)carbonyl)amino)-9-cyclohexyl-5,8-dimethyl-1,4,7,10-tetraoxo-2,5,8,11-tetraazatetradecan-14-oyl) piperidine-2-carboxylic acid (Compound 158)

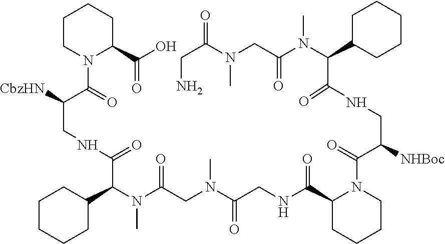 [SMILES: CN(CC(=O)N(C)[C@H](C(=O)NC[C@@H](NC(=O)OC(C)(C)C)C(=O)N1CCCC[C@H]1C(=O)NCC(=O)N(C)CC(=O)N(C)[C@H](C(=O)NC[C@@H](NC(=O)OCc1ccccc1)C(=O)N1CCCC[C@H]1C(=O)O)C1CCCCC1)C1CCCCC1)C(=O)CN]

The title compound was synthesized according to General Procedure 1 utilizing Fmoc-Pip-OH, Boc-D-Dap(Fmoc)-OH and Z-D-Dap(Fmoc)-OH, and Fmoc, Compound 91, and Compound 9. The crude linear peptide (332 mg, 0.27 mmol, 69% yield), post ether precipitation, was used without further purification.

LC/MS: Calc'd m/z=1208.7 for C₅₉H₉₂N₁₂O₁₅, found [M+H]⁺=1209.8, [M−Boc+2H]²⁺=555.6.

### 2.149 Benzyl tert-butyl ((7R,11S,19aS,26R,30S,38aS)-11,30-dicyclohexyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 159)

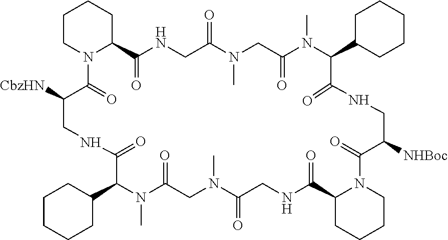 [SMILES: CN1CC(=O)N(C)[C@@H](C2CCCCC2)C(=O)NC[C@@H](NC(=O)OC(C)(C)C)C(=O)N2CCCC[C@H]2C(=O)NCC(=O)N(C)CC(=O)N(C)[C@@H](C2CCCCC2)C(=O)NC[C@@H](NC(=O)OCc2ccccc2)C(=O)N2CCCC[C@H]2C(=O)NCC1=O]

The title compound was synthesized as described in General Procedure 4, starting from Compound 158 (332 mg, 0.275 mmol). Purification according to General Procedure 12, eluting with 0 to 10% MeOH/CH₂Cl₂, afforded the title compound (237 mg, 0.199 mmol, 72% yield) as a white solid.

LC/MS: Calc'd m/z=1190.7 for C₅₉H₉₀N₁₂O₁₄, found [M−Boc+H]⁺=1091.8, [M−Boc+2H]²⁺=546.6.

### 2.150 (7R,11S,19aS,26R,30S,38aS)-11,30-dicyclohexyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diaminium (Compound 160)

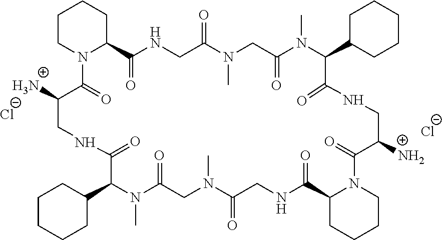 [SMILES: [Cl-] ; CN1CC(=O)N(C)[C@@H](C2CCCCC2)C(=O)NC[C@@H]([NH3+])C(=O)N2CCCC[C@H]2C(=O)NCC(=O)N(C)CC(=O)N(C)[C@@H](C2CCCCC2)C(=O)NC[C@@H]([NH3+])C(=O)N2CCCC[C@H]2C(=O)NCC1=O ; [Cl-]]

The title compound was prepared from Compound 159 (26 mg, 0.022 mmol) according to General Procedure 6 followed by General Procedure 5.

LC/MS: Calc'd m/z=956.6 for C₄₆H₇₆N₁₂O₁₀, found [M+2H]²⁺=479.6.

### 2.151 N,N′-((7R,11S,19aS,26R,30S,38aS)-11,30-dicyclohexyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 161)

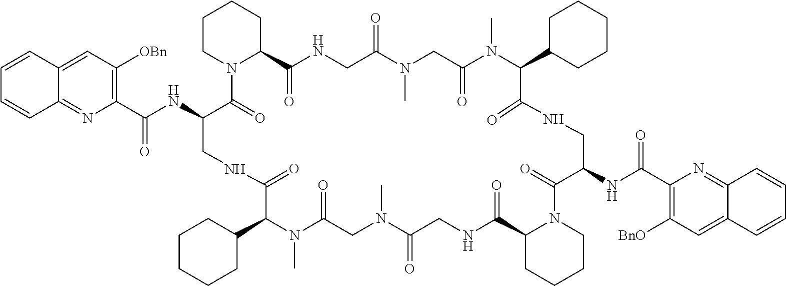 [SMILES: CN1CC(=O)N(C)[C@@H](C2CCCCC2)C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2OCc2ccccc2)C(=O)N2CCCC[C@H]2C(=O)NCC1=O]

The title compound was prepared from crude Compound 160 (21 mg, 0.022 mmol) according to General Procedure 7. Purification according to General Procedure 12, eluting with 0 to 10% MeOH/CH₂Cl₂, afforded the title compound (26 mg, 0.018 mmol, 80% yield) as a white solid.

LC/MS: Calc'd m/z=1478.7 for C₈₀H₉₈N₁₄O₁₄, found [M+2H]²⁺=740.8.

### 2.152 N,N′-((7R,11S,19aS,26R,30S,38aS)-11,30-dicyclohexyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 162; FD-13)

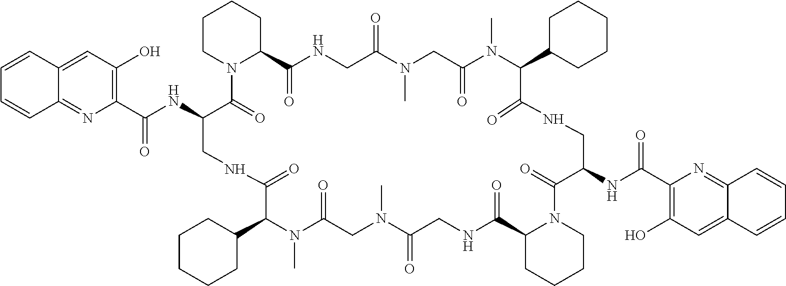 [SMILES: CN1CC(=O)N(C)[C@@H](C2CCCCC2)C(=O)NC[C@@H](NC(=O)c2nc3ccccc3cc2O)C(=O)N2CCCC[C@H]2C(=O)NCC(=O)N(C)CC(=O)N(C)[C@@H](C2CCCCC2)C(=O)NC[C@@H](NC(=O)c2nc3ccccc3cc2O)C(=O)N2CCCC[C@H]2C(=O)NCC1=O]

The title compound was synthesized from Compound 161 (26 mg, 0.018 mmol) as described in General Procedure 8. Purification according to General Procedure 12, eluting with 20 to 70% CH₃CN/H₂O, afforded the title compound (3.5 mg, 0.0027 mmol, 15% yield) as a light-yellow solid.

LC/MS: Calc'd m/z=1298.6 for C₆₆H₈₆N₁₄O₁₄, found [M+2H]²⁺=650.8.

### 2.153 (S)-1-((9S,13R)-1-((S)-1-((R)-3-((S)-2-(2-(2-aminoethoxy)-N-methylacetamido)-3-methylbutanamido)-2-(((benzyloxy)carbonyl)amino)propanoyl) piperidin-2-yl)-13-((tert-butoxycarbonyl)amino)-9-isopropyl-8-methyl-1,7,10-trioxo-5-oxa-2,8,11-triazatetradecan-14-oyl) piperidine-2-carboxylic acid (Compound 400)

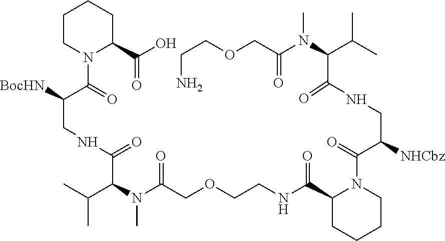 [SMILES: CC(C)[C@@H](C(=O)NC[C@@H](NC(=O)OC(C)(C)C)C(=O)N1CCCC[C@H]1C(=O)O)N(C)C(=O)COCCNC(=O)[C@@H]1CCCCN1C(=O)[C@@H](CNC(=O)[C@H](C(C)C)N(C)C(=O)COCCN)NC(=O)OCc1ccccc1]

The title compound was synthesized on 200 μM scale according to General Procedure 1 utilizing Fmoc-Pip-OH, Boc-D-Dap(Fmoc)-OH and Z-D-Dap(Fmoc)-OH, Fmoc-N-methyl-Val-OH, and Fmoc-PEG1-OH. The crude linear peptide (126 mg, 59% yield), post ether precipitation, was used without further purification.

LC/MS: Calc'd m/z=1074.6 for C₅₁H₈₂N₁₀O₁₅, found [M−Boc+2H]²⁺=488.6.

### 2.154 Benzyl tert-butyl ((7R,11S,19aS,26R,30S,38aS)-11,30-diisopropyl-12,31-dimethyl-6,10,13,19,25,29,32,38-octaoxodotriacontahydro-2H,6H,16H,25H-dipyrido[1,2-a₁:1′,2′-k][1,17]dioxa[4,7,11,14,20,23,27,30]octaazacyclodotriacontine-7,26-diyl)dicarbamate (Compound 401)

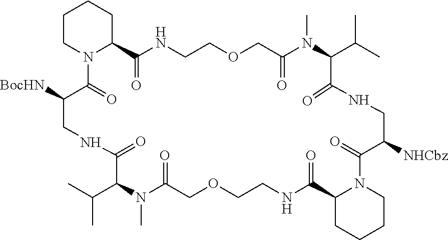 [SMILES: CC(C)[C@H]1C(=O)NC[C@@H](NC(=O)OCc2ccccc2)C(=O)N2CCCC[C@H]2C(=O)NCCOCC(=O)N(C)[C@@H](C(C)C)C(=O)NC[C@@H](NC(=O)OC(C)(C)C)C(=O)N2CCCC[C@H]2C(=O)NCCOCC(=O)N1C]

The title compound was synthesized as described in General Procedure 4, starting from Compound 400 (126 mg, 0.117 mmol). Purification according to General Procedure 12, eluting with 0 to 10% MeOH/CH₂Cl₂, afforded the title compound (72 mg, 0.068 mmol, 58% yield) as a white solid.

LC/MS: Calc'd m/z=1056.6 for C₅₁H₈₀N₁₀O₁₄, found [M+H]⁺=1057.8, [M−Boc+H]⁺=956.8, [M−Boc+2H]²⁺=479.6.

### 2.155 Benzyl ((7R,11S,19aS,26R,30S,38aS)-26-amino-11,30-diisopropyl-12,31-dimethyl-6,10,13,19,25,29,32,38-octaoxodotriacontahydro-2H,6H,16H,25H-dipyrido[1,2-a₁: 1′,2′-k][1,17]dioxa[4,7,11,14,20,23,27,30]octaazacyclodotriacontin-7-yl)carbamate (Compound 402)

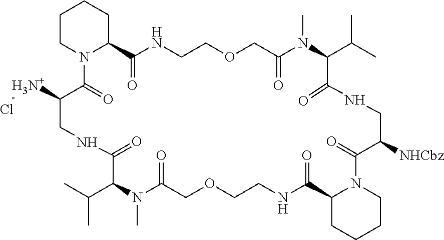 [SMILES: Cl ; CC(C)[C@H]1C(=O)NC[C@@H]([NH3+])C(=O)N2CCCC[C@H]2C(=O)NCCOCC(=O)N(C)[C@@H](C(C)C)C(=O)NC[C@@H](NC(=O)OCc2ccccc2)C(=O)N2CCCC[C@H]2C(=O)NCCOCC(=O)N1C]

The title compound was prepared from Compound 401 (72 mg, 0.068 mmol) as decribed in General Procedure 5 and yielded the title compound (70 mg) as a white solid.

LC/MS: Calc'd m/z=956.5 for C₄₆H₇₂N₁₀O₁₂, found [M+H]⁺=957.8, [M+2H]²⁺=479.6.

### 2.156 N,N′-((7R,11S,19aS,26R,30S,38aS)-11,30-diisopropyl-12,31-dimethyl-6,10,13,19,25,29,32,38-octaoxodotriacontahydro-2H,6H,16H,25H-dipyrido[1,2-a₁: 1′,2′-k][1,17]dioxa[4,7,11,14,20,23,27,30]octaazacyclodotriacontine-7,26-diyl)bis(3-(benzyloxy)quinoline-2-carboxamide) (Compound 403)

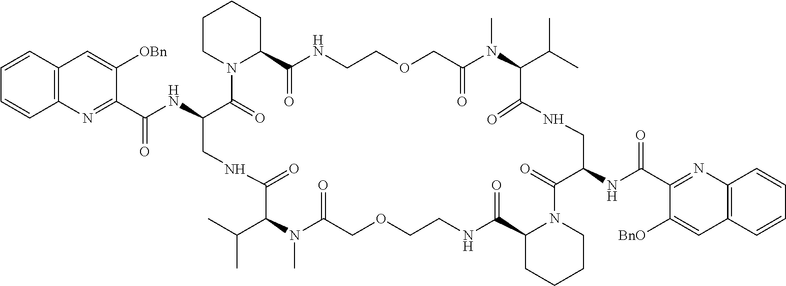 [SMILES: CC(C)[C@H]1C(=O)NC[C@@H](NC(=O)c2nc3ccccc3cc2OCc2ccccc2)C(=O)N2CCCC[C@H]2C(=O)NCCOCC(=O)N(C)[C@@H](C(C)C)C(=O)NC[C@@H](NC(=O)c2nc3ccccc3cc2OCc2ccccc2)C(=O)N2CCCC[C@H]2C(=O)NCCOCC(=O)N1C]

Starting from Compound 402 (7 mg, 0.007 mmol) the Cbz protecting group was removed as described in General Procedure 6. To the resulting free amine was coupled Compound 4 (5.4 mg, 0.02 mmol) according to General Procedure 7. Purification was accomplished as described in General Procedure 12 using a 10 g silica column and eluting with 0 to 10% MeOH/DCM to give the title compound (5.2 mg, 0.004 mmol, 60% yield) as a faint yellow solid.

LC/MS: Calc'd m/z=1344.7 for C₇₂H₈₈N₁₂O₁₄, found [M+2H]²⁺=673.8.

### 2.157 N,N′-((7R,11S,19aS,26R,30S,38aS)-11,30-diisopropyl-12,31-dimethyl-6,10,13,19,25,29,32,38-octaoxodotriacontahydro-2H,6H,16H,25H-dipyrido[1,2-a₁: 1′,2′-k][1,17]dioxa[4,7,11,14,20,23,27,30]octaazacyclodotriacontine-7,26-diyl)bis(3-hydroxyquinoline-2-carboxamide) (Compound 404; FD-27)

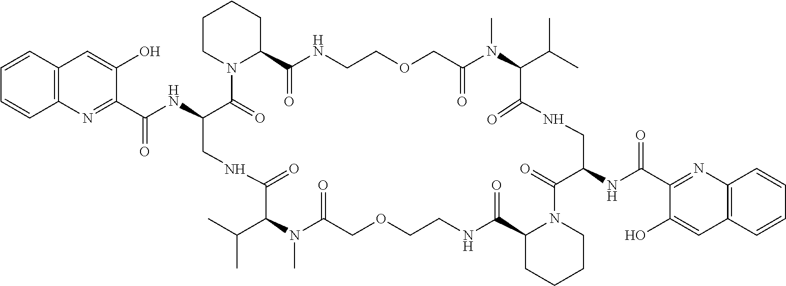 [SMILES: CC(C)[C@H]1C(=O)NC[C@@H](NC(=O)c2nc3ccccc3cc2O)C(=O)N2CCCC[C@H]2C(=O)NCCOCC(=O)N(C)[C@@H](C(C)C)C(=O)NC[C@@H](NC(=O)c2nc3ccccc3cc2O)C(=O)N2CCCC[C@H]2C(=O)NCCOCC(=O)N1C]

The title compound was synthesized from Compound 403 (5.2 mg, 0.004 mmol) as described in General Procedure 8. Purification according to General Procedure 12 using a 10 g silica column and eluting with 0 to 10% MeOH/DCM, afforded the title compound (3.4 mg, 0.0029 mmol, 75% yield) as a yellow solid.

LC/MS: Calc'd m/z=1165.6 for C₅₈H₇₆N₁₂O₁₄, found [M+2H]²⁺=583.6.

### 2.158 (S)-1-((R)-3-((S)-1-(2-(2-((S)-1-((R)-3-((S)-1-(2-(2-aminoethoxy) acetyl) piperidine-2-carboxamido)-2-(((benzyloxy)carbonyl)amino)propanoyl)piperidine-2-carboxamido) ethoxy) acetyl) piperidine-2-carboxamido)-2-((tert-butoxycarbonyl)amino)propanoyl)piperidine-2-carboxylic acid (Compound 405)

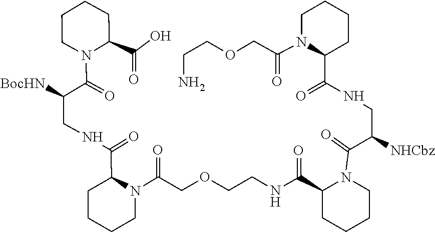 [SMILES: CC(C)(C)OC(=O)N[C@H](CNC(=O)[C@@H]1CCCCN1C(=O)COCCNC(=O)[C@@H]1CCCCN1C(=O)[C@@H](CNC(=O)[C@@H]1CCCCN1C(=O)COCCN)NC(=O)OCc1ccccc1)C(=O)N1CCCC[C@H]1C(=O)O]

The title compound was synthesized on 200 μM scale according to General Procedure 1 utilizing Fmoc-Pip-OH, Boc-D-Dap(Fmoc)-OH and Z-D-Dap(Fmoc)-OH, and Fmoc-PEG1-OH. The crude linear peptide (138 mg), post ether precipitation, was used without further purification.

LC/MS: Calc'd m/z=1070.6 for C₅₁H₇₈N₁₀O₁₅, found [M−Boc+2H]²⁺=486.4.

### 2.159 Benzyl tert-butyl ((7R,10aS,22aS,29R,32aS,44aS)-6,10,16,22,28,32,38,44-octaoxotetracontahydro-2H,19H-tetrapyrido[1,2-a₁: 1′,2′-d: 1″,2″-k: 1′″,2′″-t][1,17]dioxa[4,7,11,14,20,23,27,30]octaazacyclodotriacontine-7,29-diyl)dicarbamate (Compound 406)

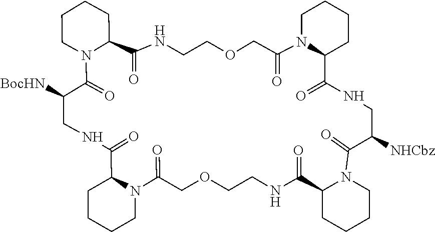 [SMILES: CC(C)(C)OC(=O)N[C@@H]1CNC(=O)[C@@H]2CCCCN2C(=O)COCCNC(=O)[C@@H]2CCCCN2C(=O)[C@H](NC(=O)OCc2ccccc2)CNC(=O)[C@@H]2CCCCN2C(=O)COCCNC(=O)[C@@H]2CCCCN2C1=O]

The title compound was synthesized as described in General Procedure 4, starting from Compound 405 (138 mg, 0.129 mmol). Purification according to General Procedure 12, eluting with 0 to 10% MeOH/CH₂Cl₂, afforded the title compound (60 mg, 0.057 mmol, 44% yield) as a white solid.

LC/MS: Calc'd m/z=1052.6 for C₅₁H₇₆N₁₀O₁₄, found [M−Boc+H]⁺=953.8, [M−Boc+2H]²⁺=477.4.

### 2.160 Benzyl ((7R,10aS,22aS,29R,32aS,44aS)-29-amino-6,10,16,22,28,32,38,44-octaoxotetracontahydro-2H,19H-tetrapyrido[1,2-a₁: 1′,2′-d: 1″,2″-k: 1′″,2′″-t][1,17]dioxa[4,7,11,14,20,23,27,30]octaazacyclodotriacontin-7-yl)carbamate (Compound 407)

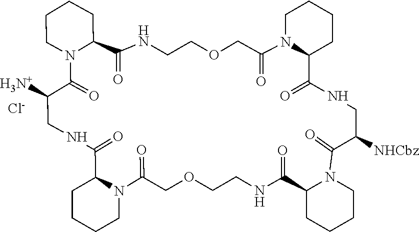 [SMILES: [Cl-] ; [NH3+][C@@H]1CNC(=O)[C@@H]2CCCCN2C(=O)COCCNC(=O)[C@@H]2CCCCN2C(=O)[C@H](NC(=O)OCc2ccccc2)CNC(=O)[C@@H]2CCCCN2C(=O)COCCNC(=O)[C@@H]2CCCCN2C1=O]

The title compound was prepared from Compound 406 (60 mg, 0.057 mmol) as described in General Procedure 5 and yielded the title compound (60 mg) as a white solid.

LC/MS: Calc'd m/z=952.5 for C₄₆H₆₈N₁₀O₁₂, found [M+H]⁺=953.8, [M+2H]²⁺=477.6.

### 2.161 N,N′-((7R,10aS,22aS,29R,32aS,44aS)-6,10,16,22,28,32,38,44-octaoxotetracontahydro-2H,19H-tetrapyrido[1,2-a₁: 1′,2′-d: 1″,2″-k: 1′″,2′″-t][1,17]dioxa[4,7,11,14,20,23,27,30]octaazacyclodotriacontine-7,29-diyl)bis(3-(benzyloxy)quinoline-2-carboxamide) (Compound 408)

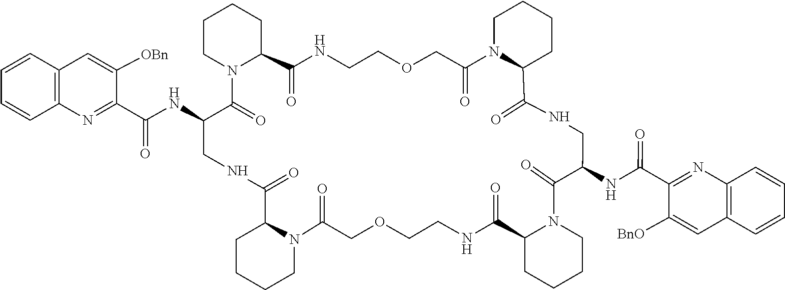 [SMILES: O=C(N[C@@H]1CNC(=O)[C@@H]2CCCCN2C(=O)COCCNC(=O)[C@@H]2CCCCN2C(=O)[C@H](NC(=O)c2nc3ccccc3cc2OCc2ccccc2)CNC(=O)[C@@H]2CCCCN2C(=O)COCCNC(=O)[C@@H]2CCCCN2C1=O)c1nc2ccccc2cc1OCc1ccccc1]

Starting from Compound 407 (7 mg, 0.007 mmol) the Cbz protecting group was removed as described in General Procedure 6. To the resulting free amine was coupled Compound 4 (4.9 mg, 0.017 mmol) according to General Procedure 7. Purification was accomplished as described in General Procedure 12 using a 10 g silica column and eluting with 0 to 10% MeOH/DCM to give the title compound (7.8 mg, 0.006 mmol, 86% yield) as a faint yellow solid.

LC/MS: Calc'd m/z=1340.6 for C₇₂H₈₄N₁₂O₁₄, found [M+2H]²⁺=671.8.

### 2.162 N,N′-((7R,10aS,22aS,29R,32aS,44aS)-6,10,16,22,28,32,38,44-octaoxotetracontahydro-2H,19H-tetrapyrido[1,2-a₁: 1′,2′-d: 1″,2″-k: 1′″,2′″-t][1,17]dioxa[4,7,11,14,20,23,27,30]octaazacyclodotriacontine-7,29-diyl)bis(3-hydroxyquinoline-2-carboxamide) (Compound 409; FD-28)

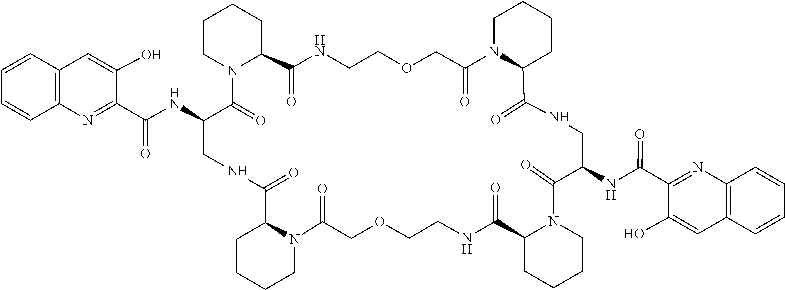 [SMILES: O=C(N[C@@H]1CNC(=O)[C@@H]2CCCCN2C(=O)COCCNC(=O)[C@@H]2CCCCN2C(=O)[C@H](NC(=O)c2nc3ccccc3cc2O)CNC(=O)[C@@H]2CCCCN2C(=O)COCCNC(=O)[C@@H]2CCCCN2C1=O)c1nc2ccccc2cc1O]

The title compound was synthesized from Compound 408 (7.8 mg, 0.006 mmol) as described in General Procedure 8. Purification according to General Procedure 12 using a 10 g silica column and eluting with 0 to 10% MeOH/DCM afforded the title compound (4.2 mg, 0.004 mmol, 62% yield) as a yellow solid.

LC/MS: Calc'd m/z=1160.5 for C₅₈H₇₂N₁₂O₁₄, found [M+H]⁺=1163.8, [M+2H]²⁺=581.6.

### 2.163 N—((R)-2-(((benzyloxy)carbonyl)amino)-3-((S)-1-(N—N—((R)-2-((tert-butoxycarbonyl)amino)-3-((S)-1-(N-glycyl-N-methylglycyl) piperidine-2-carboxamido) propanoyl)-N-methyl-L-valylglycyl-N-methylglycyl) piperidine-2-carboxamido) propanoyl)-N-methyl-L-valine (Compound 410)

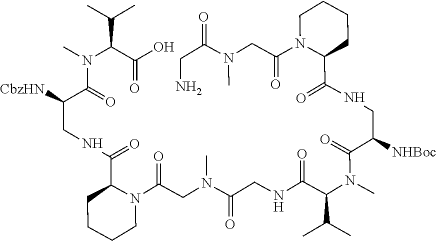 [SMILES: CC(C)[C@@H](C(=O)O)N(C)C(=O)[C@@H](CNC(=O)[C@@H]1CCCCN1C(=O)CN(C)C(=O)CNC(=O)[C@H](C(C)C)N(C)C(=O)[C@@H](CNC(=O)[C@@H]1CCCCN1C(=O)CN(C)C(=O)CN)NC(=O)OC(C)(C)C)NC(=O)OCc1ccccc1]

The title compound was synthesized on a 200 μM scale according to General Procedure 1 utilizing Fmoc-Pip-OH, Fmoc-N-methyl-Val-OH, Boc-D-Dap(Fmoc)-OH and Z-D-Dap (Fmoc)—OH, and Compound 9. The crude linear peptide (125 mg, 0.111 mmol), post ether precipitation, was used without further purification.

LC/MS: Calc'd m/z=1128.6 for C₅₃H₈₄N₁₂O₁₅, found [M−Boc+2H]²⁺=515.6.

### 2.164 Benzyl tert-butyl ((13S,16R,19aS,32S,35R,38aS)-13,32-diisopropyl-8,14,27,33-tetramethyl-6,9,12,15,19,25,28,31,34,38-decaoxohexatriacontahydro-2H,6H-dipyrido[2,1-c: 2′,1′-s][1,4,7,10,13,17,20,23,26,29]decaazacyclodotriacontine-16,35-diyl)dicarbamate (Compound 411)

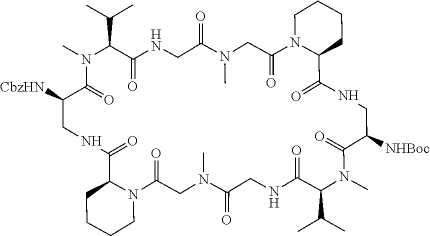 [SMILES: CC(C)[C@H]1C(=O)NCC(=O)N(C)CC(=O)N2CCCC[C@H]2C(=O)NC[C@@H](NC(=O)OC(C)(C)C)C(=O)N(C)[C@@H](C(C)C)C(=O)NCC(=O)N(C)CC(=O)N2CCCC[C@H]2C(=O)NC[C@@H](NC(=O)OCc2ccccc2)C(=O)N1C]

The title compound was synthesized as described in General Procedure 4, starting from Compound 410 (125 mg, 0.111 mmol). Purification according to General Procedure 12, eluting with 0 to 15% MeOH/CH₂Cl₂, afforded the title compound (70 mg, 0.063 mmol, 57% yield) as a white solid.

LC/MS: Calc'd m/z=1110.6 for C₅₃H₈₂N₁₂O₁₄, found [M−Boc+2H]²⁺=506.5.

### 2.165 (13S,16R,19aS,32S,35R,38aS)-16,35-diamino-13,32-diisopropyl-8,14,27,33-tetramethyltetracosahydro-2H,6H-dipyrido[2,1-c: 2′,1′-s][1,4,7,10,13,17,20,23,26,29]decaazacyclodotriacontine-6,9,12,15,19,25,28,31,34,38 (16H,19aH,35H)-decaone (Compound 412)

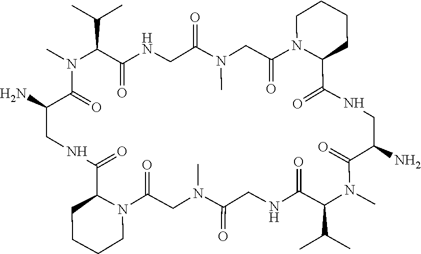 [SMILES: CC(C)[C@H]1C(=O)NCC(=O)N(C)CC(=O)N2CCCC[C@H]2C(=O)NC[C@@H](N)C(=O)N(C)[C@@H](C(C)C)C(=O)NCC(=O)N(C)CC(=O)N2CCCC[C@H]2C(=O)NC[C@@H](N)C(=O)N1C]

The title compound was prepared from Compound 411 (15 mg, 0.014 mmol) according to General Procedure 6 followed by General Procedure 5 and yielded the title compound (13 mg), which was used crude.

LC/MS: Calc'd m/z=876.5 for C₄₀H₆₈N₁₂O₁₀, found [M+2H]²⁺=439.4.

### 2.166 N,N′-((13S,16R,19aS,32S,35R,38aS)-13,32-diisopropyl-8,14,27,33-tetramethyl-6,9,12,15,19,25,28,31,34,38-decaoxohexatriacontahydro-2H,6H-dipyrido[2,1-c: 2′,1′-s][1,4,7,10,13,17,20,23,26,29]decaazacyclodotriacontine-16,35-diyl)bis(3-(benzyloxy)quinoline-2-carboxamide) (Compound 413)

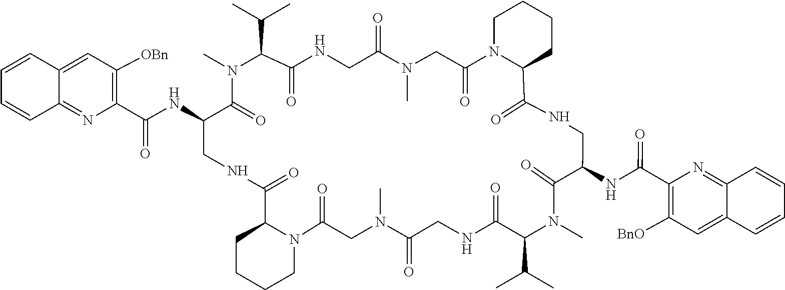 [SMILES: CC(C)[C@H]1C(=O)NCC(=O)N(C)CC(=O)N2CCCC[C@H]2C(=O)NC[C@@H](NC(=O)c2nc3ccccc3cc2OCc2ccccc2)C(=O)N(C)[C@@H](C(C)C)C(=O)NCC(=O)N(C)CC(=O)N2CCCC[C@H]2C(=O)NC[C@@H](NC(=O)c2nc3ccccc3cc2OCc2ccccc2)C(=O)N1C]

The Title compound was prepared from Compound 412 (13 mg, 0.014 mmol) and Compound 4 (9 mg, 0.03 mmol) according to General Procedure 7. Purification as described in General Procedure 12 using a 10 g silica column and eluting with 0 to 10% MeOH/DCM afforded the title compound (19 mg, 0.013 mmol, 93%) as a faint yellow solid.

LC/MS: Calc'd m/z=1398.6 for C₇₄H₉₀N₁₄O₁₄, found [M+2H]²⁺=700.8.

### 2.167 N,N′-((13S,16R,19aS,32S,35R,38aS)-13,32-diisopropyl-8,14,27,33-tetramethyl-6,9,12,15,19,25,28,31,34,38-decaoxohexatriacontahydro-2H,6H-dipyrido[2,1-c: 2′,1′-s][1,4,7,10,13,17,20,23,26,29]decaazacyclodotriacontine-16,35-diyl)bis(3-hydroxyquinoline-2-carboxamide) (Compound 414; FD-29)

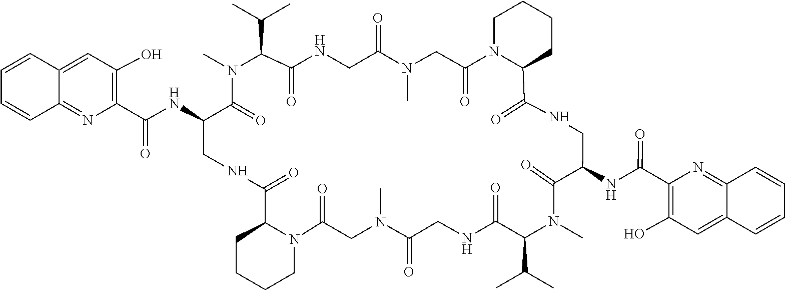 [SMILES: CC(C)[C@H]1C(=O)NCC(=O)N(C)CC(=O)N2CCCC[C@H]2C(=O)NC[C@@H](NC(=O)c2nc3ccccc3cc2O)C(=O)N(C)[C@@H](C(C)C)C(=O)NCC(=O)N(C)CC(=O)N2CCCC[C@H]2C(=O)NC[C@@H](NC(=O)c2nc3ccccc3cc2O)C(=O)N1C]

The title compound was synthesized from Compound 413 (19 mg, 0.013 mmol) as described in General Procedure 8. Preparative HPLC as decribed in General Procedure 12 eluting with a 10-70% gradient (CH₃CN/H₂O+0.1% TFA) afforded the title compound (1.2 mg, 8% yield) as a white solid post lyophilization.

LC/MS: Calc'd m/z=1218.6 for C₆₀H₇₈N₁₄O₁₄, found [M+2H]²⁺=610.6.

### 2.168 (S)-1-((9S,13R)-1-((S)-1-((R)-3-((S)-2-(2-(2-amino-N-propylacetamido)-N-methylacetamido)-3-methylbutanamido)-2-(((benzyloxy)carbonyl)amino)propanoyl) piperidin-2-yl)-13-((tert-butoxycarbonyl)amino)-9-isopropyl-8-methyl-1,4,7,10-tetraoxo-5-propyl-2,5,8,11-tetraazatetradecan-14-oyl) piperidine-2-carboxylic acid (Compound 415)

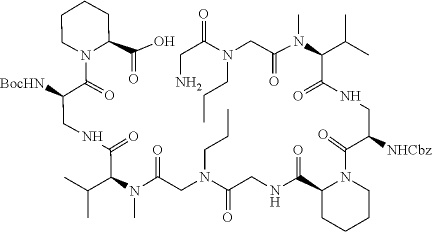 [SMILES: CCCN(CC(=O)N(C)[C@H](C(=O)NC[C@@H](NC(=O)OCc1ccccc1)C(=O)N1CCCC[C@H]1C(=O)NCC(=O)N(CCC)CC(=O)N(C)[C@H](C(=O)NC[C@@H](NC(=O)OC(C)(C)C)C(=O)N1CCCC[C@H]1C(=O)O)C(C)C)C(C)C)C(=O)CN]

The title compound was synthesized on a 400 μM scale according to General Procedure 1 utilizing Fmoc-Pip-OH, Fmoc-N-methyl-Val-OH, Boc-D-Dap(Fmoc)-OH and Z-D-Dap (Fmoc)-OH, and Fmoc-Gly-OH, and Fmoc-N-propyl-Val-OH. The crude linear peptide (454 mg, 0.383 mmol), post ether precipitation, was used without further purification.

LC/MS: Calc'd m/z=1184.7 for C₅₇H₉₂N₁₂O₁₅, found [M−Boc+2H]²⁺=593.6.

### 2.169 Benzyl tert-butyl ((7R,11S,19aS,26R,30S,38aS)-11,30-diisopropyl-12,31-dimethyl-6,10,13,16,19,25,29,32,35,38-decaoxo-15,34-dipropylhexatriacontahydro-2H,6H-dipyrido[1,2-a:1′,2′-q][1,4,7,10,13,17,20,23,26,29]decaazacyclodotriacontine-7,26-diyl)dicarbamate (Compound 416)

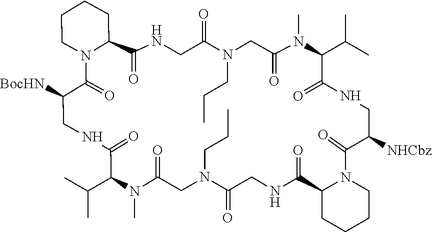 [SMILES: CCCN1CC(=O)N(C)[C@@H](C(C)C)C(=O)NC[C@@H](NC(=O)OC(C)(C)C)C(=O)N2CCCC[C@H]2C(=O)NCC(=O)N(CCC)CC(=O)N(C)[C@@H](C(C)C)C(=O)NC[C@@H](NC(=O)OCc2ccccc2)C(=O)N2CCCC[C@H]2C(=O)NCC1=O]

The title compound was synthesized as described in General Procedure 4, starting from Compound 415 (454 mg, 0.383 mmol). Purification according to General Procedure 12, eluting with 0 to 10% MeOH/CH₂Cl₂, afforded the title compound (378 mg, 0.324 mmol, 85% yield) as a white solid.

LC/MS: Calc'd m/z=1166.7 for C₅₇H₉₀N₁₂O₁₄, found [M−Boc+2H]²⁺=534.6.

### 2.170 (7R,11S,19aS,26R,30S,38aS)-7,26-diamino-11,30-diisopropyl-12,31-dimethyl-15,34-dipropyltetracosahydro-2H,6H-dipyrido[1,2-a:1′,2′-q][1,4,7,10,13,17,20,23,26,29]decaazacyclodotriacontine-6,10,13,16,19,25,29,32,35,38 (7H,19aH,26H)-decaone (Compound 417)

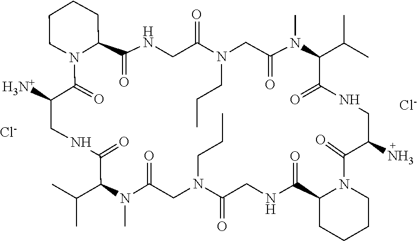 [SMILES: [Cl-] ; CCCN1CC(=O)N(C)[C@@H](C(C)C)C(=O)NC[C@@H]([NH3+])C(=O)N2CCCC[C@H]2C(=O)NCC(=O)N(CCC)CC(=O)N(C)[C@@H](C(C)C)C(=O)NC[C@@H](N)C(=O)N2CCCC[C@H]2C(=O)NCC1=O ; [Cl-]]

The title compound was prepared from Compound 416 (40 mg, 0.034 mmol) according to General Procedure 6 followed by General Procedure 5 and yielded the title compound (40 mg) which was used crude.

LC/MS: Calc'd m/z=932.6 for C₄₄H₇₆N₁₂O₁₀, found [M+2H]²⁺=467.4.

### 2.171 N,N′-((7R,11S,19aS,26R,30S,38aS)-11,30-diisopropyl-12,31-dimethyl-6,10,13,16,19,25,29,32,35,38-decaoxo-15,34-dipropylhexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 418)

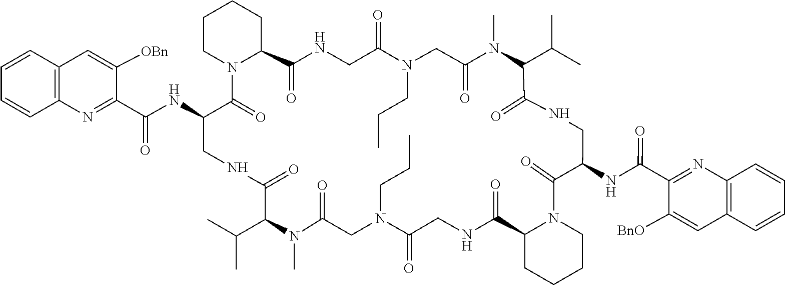

The title compound was prepared from Compound 417 (40 mg, 0.043 mmol) and Compound 4 (30 mg, 0.107 mmol) according to General Procedure 7. Purification as described in General Procedure 12 using a 10 g silica column and eluting with 0 to 10% MeOH/DCM afforded the title compound (12 mg, 0.008 mmol, 17%) as a faint yellow solid.

LC/MS: Calc'd m/z=1454.7 for C₇₈H₉₆N₁₄O₁₄, found [M+2H]²⁺=728.8.

### 2.172 N,N′-((7R,11S,19aS,26R,30S,38aS)-11,30-diisopropyl-12,31-dimethyl-6,10,13,16,19,25,29,32,35,38-decaoxo-15,34-dipropylhexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 419)

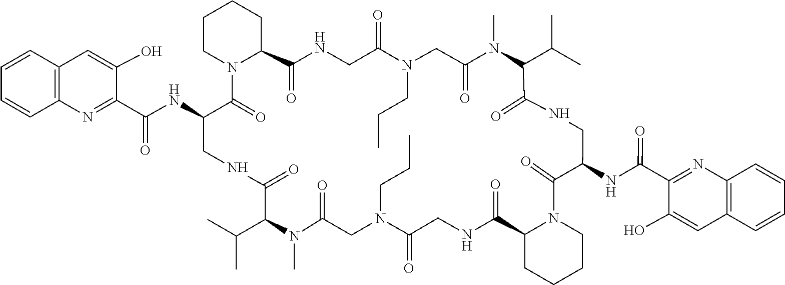

The title compound was synthesized from Compound 418 (12 mg, 0.008 mmol) as described in General Procedure 8. Preparative HPLC as described in General Procedure 12 eluting with a 40-70% gradient (CH₃CN/H₂O+0.1% TFA) afforded the title compound (3.8 mg, 36% yield) as a white solid post lyophilization.

LC/MS: Calc'd m/z=1274.6 for C₆₆H₈₆N₁₄O₁₄, found [M+H]⁺=1275.8, [M+2H]²⁺=638.6.

### 2.173 (S)-1-((9S,13R)-1-((S)-1-((R)-3-((S)-2-(2-(2-amino-N-isopropylacetamido)-N-methylacetamido)-3-methylbutanamido)-2-(((benzyloxy)carbonyl)amino)propanoyl) piperidin-2-yl)-13-((tert-butoxycarbonyl)amino)-5,9-diisopropyl-8-methyl-1,4,7,10-tetraoxo-2,5,8,11-tetraazatetradecan-14-oyl) piperidine-2-carboxylic acid (Compound 420)

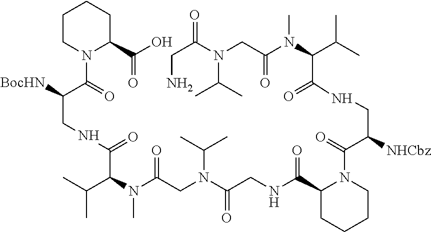 [SMILES: CC(C)[C@@H](C(=O)NC[C@@H](NC(=O)OCc1ccccc1)C(=O)N1CCCC[C@H]1C(=O)NCC(=O)N(CC(=O)N(C)[C@H](C(=O)NC[C@@H](NC(=O)OC(C)(C)C)C(=O)N1CCCC[C@H]1C(=O)O)C(C)C)C(C)C)N(C)C(=O)CN(C(=O)CN)C(C)C]

The title compound was synthesized on a 400 μM scale according to General Procedure 1 utilizing Fmoc-Pip-OH, Fmoc-N-methyl-Val-OH, Boc-D-Dap(Fmoc)-OH and Z-D-Dap (Fmoc)—OH, and Fmoc-Gly-OH, and Fmoc-N-isopropyl-Val-OH. The crude linear peptide (450 mg, 0.38 mmol), post ether precipitation, was used without further purification.

LC/MS: Calc'd m/z=1184.7 for C₅₇H₉₂N₁₂O₁₅, found [M−Boc+2H]²⁺=593.6.

### 2.174 Benzyl tert-butyl ((7R,11S,19aS,26R,30S,38aS)-11,15,30,34-tetraisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 421)

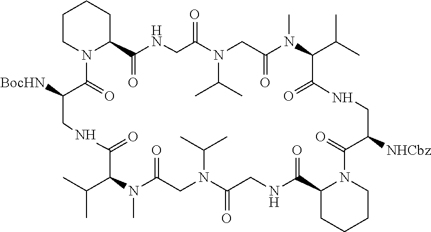 [SMILES: CC(C)[C@H]1C(=O)NC[C@@H](NC(=O)OC(C)(C)C)C(=O)N2CCCC[C@H]2C(=O)NCC(=O)N(C(C)C)CC(=O)N(C)[C@@H](C(C)C)C(=O)NC[C@@H](NC(=O)OCc2ccccc2)C(=O)N2CCCC[C@H]2C(=O)NCC(=O)N(C(C)C)CC(=O)N1C]

The title compound was synthesized as described in General Procedure 4 starting from Compound 420 (450 mg, 0.38 mmol). Purification according to General Procedure 12, eluting with 0 to 10% MeOH/CH₂Cl₂, afforded the title compound (415 mg, 0.355 mmol, 94% yield) as a white solid.

LC/MS: Calc'd m/z=1166.7 for C₅₇H₉₀N₁₂O₁₄, found [M−Boc+2H]²⁺=534.6.

### 2.175 (7R,11S,19aS,26R,30S,38aS)-7,26-diamino-11,15,30,34-tetraisopropyl-12,31-dimethyltetracosahydro-2H,6H-dipyrido[1,2-a:1′,2′-q][1,4,7,10,13,17,20,23,26,29]decaazacyclodotriacontine-6,10,13,16,19,25,29,32,35,38 (7H,19aH,26H)-decaone (Compound 422)

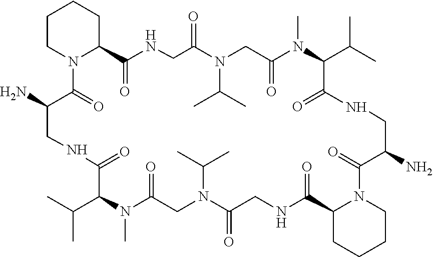 [SMILES: CC(C)[C@H]1C(=O)NC[C@@H](N)C(=O)N2CCCC[C@H]2C(=O)NCC(=O)N(C(C)C)CC(=O)N(C)[C@@H](C(C)C)C(=O)NC[C@@H](N)C(=O)N2CCCC[C@H]2C(=O)NCC(=O)N(C(C)C)CC(=O)N1C]

The title compound was prepared from Compound 421 (40 mg, 0.034 mmol) according to General Procedure 6 followed by General Procedure 5 yielded the title compound (40 mg) which was used crude.

LC/MS: Calc'd m/z=932.6 for C₄₄H₇₆N₁₂O₁₀, found [M+H]⁺=934.8, and [M+2H]²⁺=467.8.

### 2.178 N,N′-((7R,11S,19aS,26R,30S,38aS)-11,15,30,34-tetraisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-(benzyloxy)quinoline-2-carboxamide) (Compound 423)

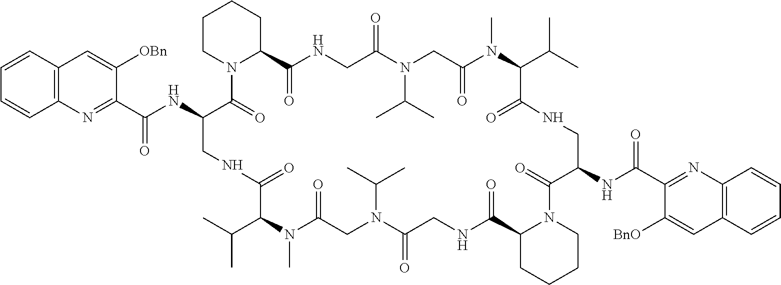 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C(C)C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C(C)C)CC(=O)N1C]

The title compound was prepared from Compound 422 (40 mg, 0.043 mmol) and Compound 4 (30 mg, 0.107 mmol) according to General Procedure 7. Purification as described in General Procedure 12 using a 10 g silica column and eluting with 0 to 10% MeOH/DCM afforded the title compound (47 mg, 0.033 mmol, 77%) as a faint yellow solid.

LC/MS: Calc'd m/z=1454.7 for C₇₈H₉₆N₁₄O₁₄, found [M+2H]²⁺=728.8.

### 2.179 N,N′-((7R,11S,19aS,26R,30S,38aS)-11,15,30,34-tetraisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)bis(3-hydroxyquinoline-2-carboxamide) (Compound 424)

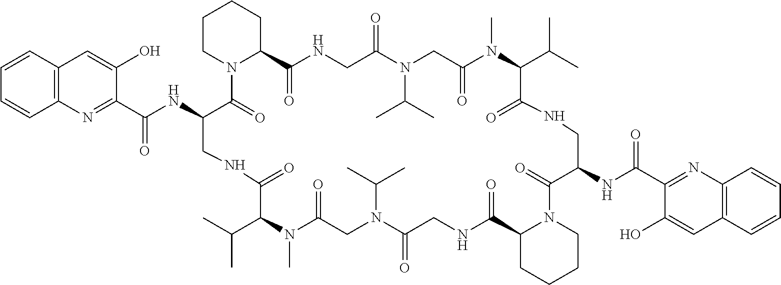 [SMILES: CC(C)[C@H]1C(=O)NC[C@@H](NC(=O)c2nc3ccccc3cc2O)C(=O)N2CCCC[C@H]2C(=O)NCC(=O)N(C(C)C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C(C)C)CC(=O)N1C]

The title compound was synthesized from Compound 423 (47 mg, 0.032 mmol) as described in General Procedure 8. Preparative HPLC as described in General Procedure 12 eluting with a 40-70% gradient (CH₃CN/H₂O+0.1% TFA) afforded the title compound (12.5 mg, 30% yield) as a white solid post lyophilization.

LC/MS: Calc'd m/z=1274.6 for C₆₆H₈₆N₁₄O₁₄, found [M+H]⁺=1275.8, [M+2H]²⁺=638.8.

### 2.180 2-methyl 1-(4-nitrobenzyl) (R)-aziridine-1,2-dicarboxylate (Compound 163)

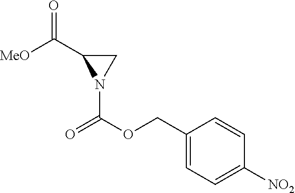 [SMILES: COC(=O)[C@H]1CN1C(=O)OCc1ccc([N+](=O)[O-])cc1]

The title compound was prepared according to Liu et al., 2007, Org. Lett., 9:4211-4214.

LC/MS: Calc'd m/z=280.0 for C₁₂H₁₂N₂O₆, found [M+H]⁺=281.2. ¹H NMR (300 MHZ, chloroform-d) δ 8.32-8.16 (m, 2H), 7.64-7.46 (m, 2H), 5.39-5.14 (m, 2H), 3.79 (s, 3H), 3.18 (dd, J=5.3, 3.2 Hz, 1H), 2.66 (dd, J=3.2, 1.3 Hz, 1H), 2.55 (dd, J=5.3, 1.3 Hz, 1H).

### 2.181 (S)—N-(1-hydroxy-3-methylbutan-2-yl)-2-nitrobenzenesulfonamide (Ns-valinol; Compound 164)

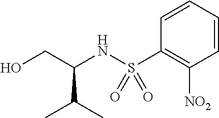 [SMILES: CC(C)[C@@H](CO)NS(=O)(=O)c1ccccc1[N+](=O)[O-]]

Methanol (24 mL) was cooled to 0° C. and AcCl (6 mL) was added slowly with vigorous stirring. To this solution was added Boc-valinol (3.9 g, 18.9 mmol, 1 equiv.) and stirring continued at rt for 1 h. Following complete Boc cleavage, the solvent was removed in vacuo, the residue brought up in EtOAc (50 mL) and evaporated to dryness. The crude valinol was dissolved in dioxane (50 mL) and H₂O (40 mL) followed by addition of K₂CO₃ (5.2 g, 37.8 mol, 2 equiv.) and 2-nitrobenzenesulfonamide (4.2 g, 18.9 mmol, 1 equiv.). This solution was stirred for 1 h, concentrated to 50% volume and partitioned between EtOAc (100 mL) and brine (50 mL). The layers were separated, the organic layer was washed with brine (2×20 mL) and dried over Na₂SO₄. Purification according to General Procedure 12, using a 50 g column and eluting with a linear gradient of 0 to 100% EtOAc/Hexanes, afforded the title compound (4.3 g, 14.9 mmol, 80% yield) as a white solid.

LC/MS: Calc'd m/z=288.1 for C₁₁H₁₆N₂O₅S, found [M+H]⁺=289.2. ¹H NMR (300 MHZ, chloroform-d) δ 8.24-8.09 (m, 1H), 7.96-7.83 (m, 1H), 7.83-7.68 (m, 2H), 5.50 (d, J=8.3 Hz, 1H), 3.70-3.57 (m, 2H), 3.33 (dtd, J=8.3, 5.9, 4.4 Hz, 1H), 1.91 (dq, J=13.5, 6.7 Hz, 1H), 1.68 (t, J=5.7 Hz, 1H), 0.91 (t, J=7.0 Hz, 6H).

### 2.182 (S)—N-(1-hydroxy-3-methylbutan-2-yl)-N-methyl-2-nitrobenzenesulfonamide (Compound 165)

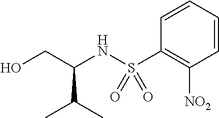 [SMILES: CC(C)[C@@H](CO)NS(=O)(=O)c1ccccc1[N+](=O)[O-]]

To a stirring solution of Compound 164 (4.3 g, 14.9 mmol, 1 equiv.) in DMF (75 mL, 0.2 M) at 0° C. was added NaH (60% wt, 700 mg, 17.9 mmol, 1.2 equiv.). This solution was stirred for 30 min then CH₃I (1.1 mL, 17.9 mmol, 1.2 equiv.) was added and the reaction was allowed to warm to rt while stirring for 2 h. Solvent was removed in vacuo. Purification according to General Procedure 12, using a 120 g C18 column and eluting with a linear gradient of 35 to 50% CH₃CN/H₂O, afforded the title compound (3.8 g, 12.5 mmol, 84% yield) as a white solid.

LC/MS: Calc'd m/z=302.1 for C₁₂H₁₈N₂O₅S, found [M+H]⁺=303.2. ¹H NMR (300 MHZ, Chloroform-d) δ 8.07-7.96 (m, 1H), 7.70-7.58 (m, 2H), 7.54 (dt, J=5.5, 3.8 Hz, 1H), 3.90-3.74 (m, 1H), 3.63-3.44 (m, 2H), 2.90 (s, 3H), 1.82-1.64 (m, 3H), 0.92 (d, J=6.6 Hz, 3H), 0.80 (d, J=6.7 Hz, 3H).

### 2.183 Methyl O—((S)-3-methyl-2-((N-methyl-2-nitrophenyl)sulfonamido)butyl)-N-(((4-nitrobenzyl)oxy)carbonyl)-D-serinate (Compound 166)

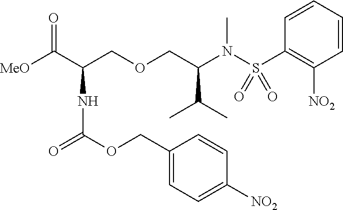 [SMILES: COC(=O)[C@@H](COC[C@H](C(C)C)N(C)S(=O)(=O)c1ccccc1[N+](=O)[O-])NC(=O)OCc1ccc([N+](=O)[O-])cc1]

The title compound was prepared following the protocol detailed in Liu et al., Org. Lett., 2007, 9, 4211-4214. To a stirring solution of Compound 163 (1 g, 3.57 mmol, 1 equiv.) and Compound 165 (2.1 g, 7.14 mmol, 2 equiv.) in toluene (35 mL, 0.1M) was added BF₃*OEt₂ (110 μL, 0.89 mmol, 0.25 equiv.) slowly via syringe. The flask was equipped with a reflux condenser and the reaction was heated at 110° C. for 1 h, cooled to rt, and evaporated to dryness. Purification according to General Procedure 12, using a 60 g C18 column and eluting with a linear gradient of 20 to 75% CH₃CN/H₂O, afforded the title compound (1.32 g, 2.27 mmol, 64% yield) as a white solid.

LC/MS: Calc'd m/z=582.6 for C₂₄H₃₀N₄O₁₁S, found [M+H]⁺=583.4. ¹H NMR (300 MHZ, chloroform-d) δ 8.25 (d, J=8.2 Hz, 2H), 8.07-7.93 (m, 1H), 7.75-7.63 (m, 2H), 7.63-7.47 (m, 3H), 5.81 (d, J=8.7 Hz, 1H), 5.24 (s, 2H), 4.47 (dt, J=8.7, 3.2 Hz, 1H), 3.88 (dd, J=9.4, 3.3 Hz, 1H), 3.76 (s, 3H), 3.73-3.56 (m, 2H), 3.62-3.55 (m, 2H), 2.86 (s, 3H), 1.92 (dd, J=10.2, 6.9 Hz, 1H), 0.97 (d, J=6.6 Hz, 3H), 0.81 (d, J=6.7 Hz, 3H).

### 2.184 O—((S)-3-methyl-2-((N-methyl-2-nitrophenyl)sulfonamido)butyl)-N-(((4-nitrobenzyl)oxy)carbonyl)-D-serine (Compound 167)

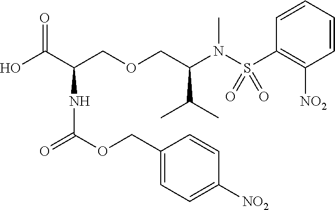 [SMILES: CC(C)[C@@H](COC[C@@H](NC(=O)OCc1ccc([N+](=O)[O-])cc1)C(=O)O)N(C)S(=O)(=O)c1ccccc1[N+](=O)[O-]]

To a stirring solution of Compound 166 (1 g, 1.72 mmol, 1 equiv.) in THF (30 mL), at 0° C., was added a 1 M aqueous solution of LiOH (3.4 mL, 3.4 mmol 2 equiv.). This solution was stirred at 0° C. for 30 min then quenched with a saturated aqueous solution of citric acid (3 mL) and warmed to rt. The aqueous layer was extracted with EtOAc (50 mL) then the organic layer was washed with brine (2×20 mL) and dried over Na₂SO₄. Purification according to General Procedure 12, using a 25 g column and eluting with a linear gradient of 0 to 10% MeOH/CH₂Cl₂, afforded the title compound (931 mg, 1.64 mmol, 95% yield) as a white solid.

LC/MS: Calc'd m/z=568.1 for C₂₃H₂₈N₄O₁₁S, found [M+H]⁺=569.2. ¹H NMR (300 MHz, chloroform-d) δ 8.24 (dq, J=8.9, 2.6 Hz, 2H), 8.07-7.95 (m, 1H), 7.74-7.62 (m, 2H), 7.62-7.50 (m, 3H), 6.27 (s, 2H), 5.98 (d, J=8.6 Hz, 1H), 5.26 (s, 2H), 4.50 (dt, J=8.8, 3.3 Hz, 1H), 3.97 (ddd, J=12.7, 8.5, 4.3 Hz, 1H), 3.82-3.49 (m, 3H), 2.85 (s, 3H), 1.87 (s, 1H), 0.98 (d, J=6.6 Hz, 3H), 0.81 (dd, J=9.6, 6.6 Hz, 3H).

### 2.185 (S)-1-(O—((S)-2-(2-(2-((S)-1-(O)—((S)-2-(2-(2-amino-N-methylacetamido)-N-methylacetamido)-3-methylbutyl)-N-(((4-nitrobenzyl)oxy)carbonyl)-D-seryl) piperidine-2-carboxamido)-N-methylacetamido)-N-methylacetamido)-3-methylbutyl)-N-(((4-nitrobenzyl)oxy)carbonyl)-D-seryl) piperidine-2-carboxylic acid (Compound 168)

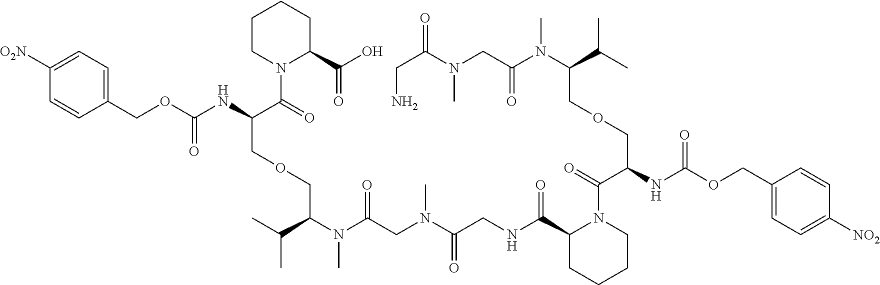 [SMILES: CC(C)[C@@H](COC[C@@H](NC(=O)OCc1ccc([N+](=O)[O-])cc1)C(=O)N1CCCC[C@H]1C(=O)NCC(=O)N(C)CC(=O)N(C)[C@H](COC[C@@H](NC(=O)OCc1ccc([N+](=O)[O-])cc1)C(=O)N1CCCC[C@H]1C(=O)O)C(C)C)N(C)C(=O)CN(C)C(=O)CN]

The title compound was synthesized according to General Procedure 1 utilizing Compound 167, Fmoc-Pip-OH, Fmoc-Sar-OH and Fmoc-Gly-OH, on a 0.075 mmol scale. The crude peptide was purified according to General Procedure 12, using a 12 g C18 column and eluting with a linear gradient of 10 to 100% CH₃CN/H₂O, to afford the title compound (32 mg, 0.026 mmol, 35% yield) as a white solid.

LC/MS: Calc'd m/z=1226.6 for C₅₆H₈₂N₁₂O₁₉, found [M+H]⁺=1228.2, [M+2H]²⁺=614.6.

### 2.186 Bis(4-nitrobenzyl) ((7R,11S,19aS,26R,30S,38aS)-11,30-diisopropyl-12,15,31,34-tetramethyl-6,13,16,19,25,32,35,38-octaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)dicarbamate (Compound 169)

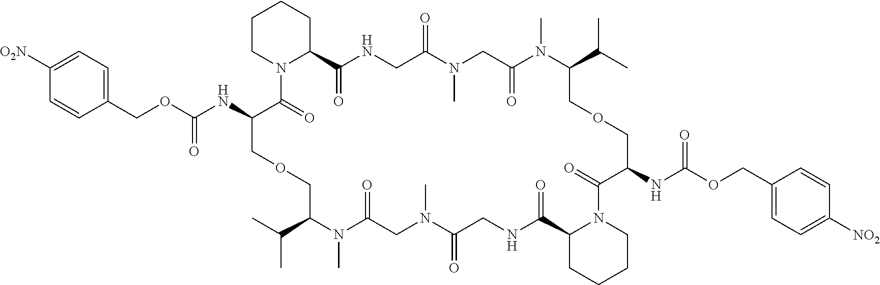 [SMILES: CC(C)[C@H]1COC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)COC[C@@H](NC(=O)OCc2ccc([N+](=O)[O-])cc2)C(=O)N2CCCC[C@H]2C(=O)NCC(=O)N(C)CC(=O)N1C]

The title compound was synthesized as described in General Procedure 4 starting from Compound 168 (18 mg, 0.015 mmol). Purification according to General Procedure 12, using a 10 g column and eluting with a linear gradient of 0 to 15% MeOH/CH₂Cl₂, afforded the title compound (12 mg, 0.01 mmol, 68% yield) as a white solid.

LC/MS: Calc'd m/z=1208.6 for C₅₆H₈₀N₁₂O₁₆, found [M+H]⁺=1210.0, [M+2H]²⁺=605.6.

### 2.187 (7R,11S,19aS,26R,30S,38aS)-7,26-diamino-11,30-diisopropyl-12,15,31,34-tetramethyl tetracosahydro-2H,6H,10H,25H-dipyrido[2,1-b₁: 2′,1′-l][1,17]dioxa[4,7,10,13,20,23,26,29]octaazacyclodotriacontin-6,13,16,19,25,32,35,38 (19aH,29H)-octaone (Compound 170)

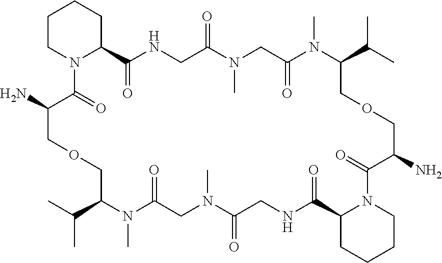 [SMILES: CC(C)[C@H]1COC[C@@H](N)C(=O)N2CCCC[C@H]2C(=O)NCC(=O)N(C)CC(=O)N(C)[C@@H](C(C)C)COC[C@@H](N)C(=O)N2CCCC[C@H]2C(=O)NCC(=O)N(C)CC(=O)N1C]

The title compound was synthesized as described in General Procedure 6 starting from Compound 169 (12 mg, 0.01 mmol), 10% Pd/C (10 mg) and MeOH (2 mL). Following removal of Pd/C, the title compound was isolated as a white solid.

LC/MS: Calc'd m/z=850.5 for C₄₀H₇₀N₁₀O₁₀, found [M+H]⁺=851.8, [M+2H]²⁺=426.4.

### 2.188 N,N′-((7R,11S,19aS,26R,30S,38aS)-11,30-diisopropyl-12,15,31,34-tetramethyl-6,13,16,19,25,32,35,38-octaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)bis(3-(benzyloxy)quinoline-2-carboxamide) (Compound 171)

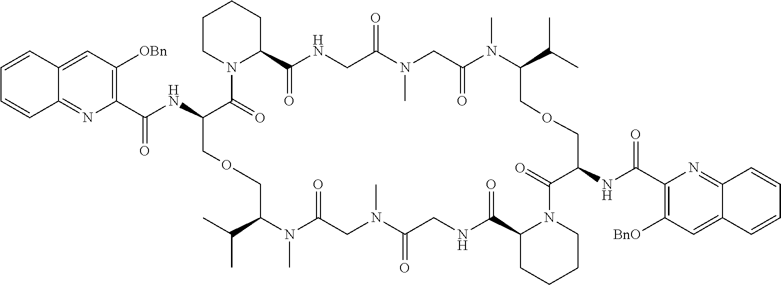 [SMILES: CC(C)[C@H]1COC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)COC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 170 (0.01 mmol). Purification according to General Procedure 12, using a 10 g column and eluting with a linear gradient of 0 to 20% MeOH/CH₂Cl₂, afforded the title compound (3.5 mg, 0.0025 mmol, 25% yield over 2 steps) as a white solid.

LC/MS: Calc'd m/z=1372.6 for C₇₄H₉₂N₁₂O₁₄, found [M+H]⁺=1373.6, [M+2H]²⁺=687.8.

### 2.189 N,N′-((7R,11S,19aS,26R,30S,38aS)-11,30-diisopropyl-12,15,31,34-tetramethyl-6,13,16,19,25,32,35,38-octaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)bis(3-hydroxyquinoline-2-carboxamide) (Compound 172; FD-17)

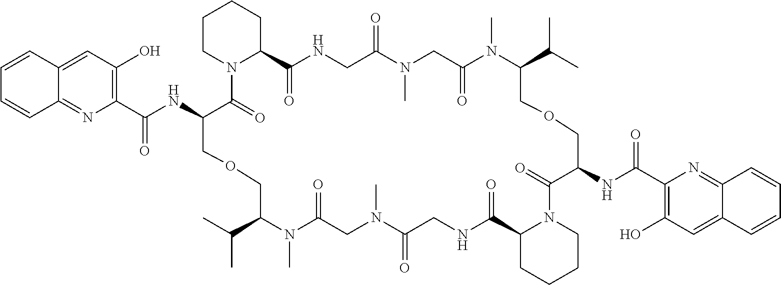 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)COC[C@@H](NC(=O)c2nc3ccccc3cc2O)C(=O)N2CCCC[C@H]2C(=O)NCC(=O)N(C)CC(=O)N1C]

Compound 172 was synthesized as described in General Procedure 6 starting from Compound 171 (3.5 mg, 0.0025 mmol), 10% Pd/C (10 mg) and MeOH (2 mL). Following removal of Pd/C, the title compound (2.6 mg, 0.022 mmol, 87% yield) was isolated as a vibrant yellow solid.

LC/MS: Calc'd m/z=1192.6 for C₆₀H₈₀N₁₂O₁₄, found [M+H]⁺=1193.8, [M+2H]²⁺=597.6.

### 2.190 (S)-1-(O—((S)-2-(2-(2-((S)-1-((R)-3-((S)-2-(2-(2-amino-N-methylacetamido)-N-methylacetamido)-3-methylbutanamido)-2-((tert-butoxycarbonyl)amino)propanoyl)piperidine-2-carboxamido)-N-methylacetamido)-N-methylacetamido)-3-methylbutyl)-N-(((4-nitrobenzyl)oxy)carbonyl)-D-seryl) piperidine-2-carboxylic acid (Compound 173)

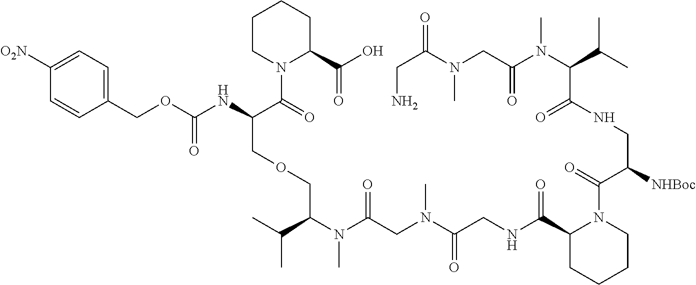 [SMILES: CC(C)[C@@H](COC[C@@H](NC(=O)OCc1ccc([N+](=O)[O-])cc1)C(=O)N1CCCC[C@H]1C(=O)O)N(C)C(=O)CN(C)C(=O)CNC(=O)[C@@H]1CCCCN1C(=O)[C@@H](CNC(=O)[C@H](C(C)C)N(C)C(=O)CN(C)C(=O)CN)NC(=O)OC(C)(C)C]

The title compound was synthesized according to General Procedure 1 utilizing Compound 167, Fmoc-Pip-OH, Boc-D-Dap(Fmoc)-OH, Fmoc-Sar-OH and Fmoc-Gly-OH, on a 0.075 mmol scale. The crude peptide was purified according to General Procedure 12, using a 12 g C18 column and eluting with 10 to 100% MeCN/H₂O, to afford the title compound (43 mg, 0.037 mmol, 49% yield) as a white solid.

LC/MS: Calc'd m/z=1160.6 for C₅₃H₈₄N₁₂O₁₇, found [M+H]⁺=1161.4, [M−Boc+2H]²⁺=531.6.

### 2.191 Tert-butyl (4-nitrobenzyl) ((7R,11S,19aS,26R,30S,38aS)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)dicarbamate (Compound 174)

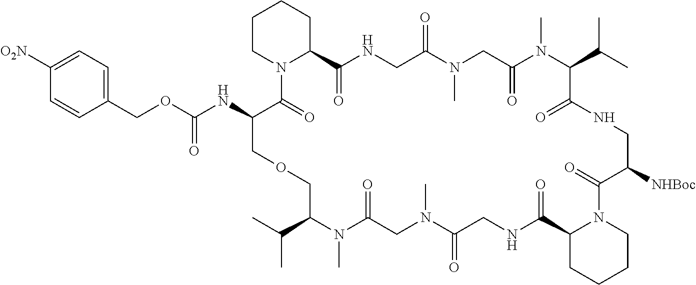 [SMILES: CC(C)[C@H]1COC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 173 (43 mg, 0.037 mmol). Purification according to General Procedure 12, using a 10 g column and eluting with 0 to 15% MeOH/CH₂Cl₂, afforded the title compound (12 mg, 0.01 mmol, 68% yield) as a white solid.

LC/MS: Calc'd m/z=1142.6 for C₅₃H₈₂N₁₂O₁₆, found [M+H]⁺=1144.0, [M−Boc+2H]²⁺=522.6.

### 2.192 Tert-butyl ((7R,11S,19aS,26R,30S,38aS)-7-amino-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamate (Compound 175)

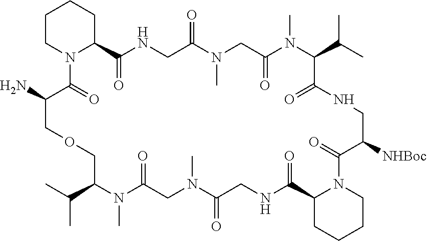 [SMILES: CC(C)[C@H]1COC[C@@H](N)C(=O)N2CCCC[C@H]2C(=O)NCC(=O)N(C)CC(=O)N(C)[C@@H](C(C)C)C(=O)NC[C@@H](NC(=O)OC(C)(C)C)C(=O)N2CCCC[C@H]2C(=O)NCC(=O)N(C)CC(=O)N1C]

The title compound was synthesized as described in General Procedure 6 starting from Compound 174 (12 mg, 0.01 mmol), 10% Pd/C (10 mg) and MeOH (2 mL). Following removal of Pd/C, the title compound was isolated as a white solid.

LC/MS: Calc'd m/z=963.6 for C₄₅H₇₇N₁₁O₁₂, found [M+H]⁺=964.8, [M−Boc+2H]²⁺=433.0.

### 2.193 (7R,11S,19aS,26R,30S,38aS)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diaminium chloride (Compound 176)

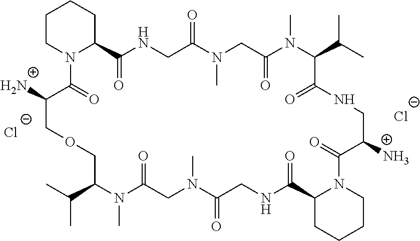 [SMILES: [Cl-] ; CC(C)[C@H]1COC[C@@H]([NH3+])C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 175 (0.01 mmol), and 2 mL of 4 M HCl/dioxane.

LC/MS: Calc'd m/z=864.5 for C₄₀H₆₉N₁₁O₁₀, found [M+H]⁺=864.8, [M+2H]²⁺=433.0.

### 2.194 N,N′-((7R,11S,19aS,26R,30S,38aS)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-(benzyloxy)quinoline-2-carboxamide) (Compound 177)

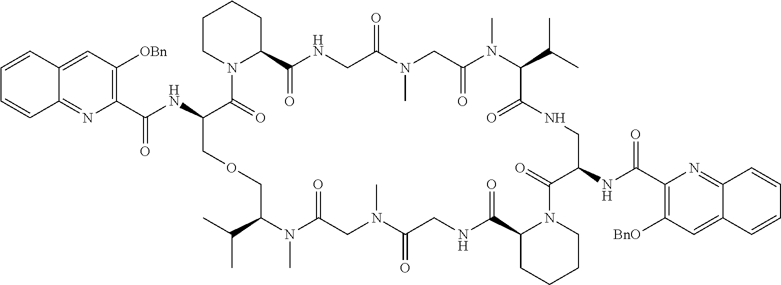 [SMILES: CC(C)[C@H]1COC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 176 (0.025 mmol). Purification according to General Procedure 12, using a 10 g column and eluting with a linear gradient of 0 to 20% MeOH/CH₂Cl₂, afforded the title compound (10 mg, 0.007 mmol, 29% yield over 2 steps) as a white solid.

LC/MS: Calc'd m/z=1385.7 for C₇₄H₉₁N₁₃O₁₄, found [M+H]⁺=1386.8, [M+2H]²⁺=694.4.

### 2.195 N,N′-((7R,11S,19aS,26R,30S,38aS)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-hydroxyquinoline-2-carboxamide) (Compound 178; FD-3)

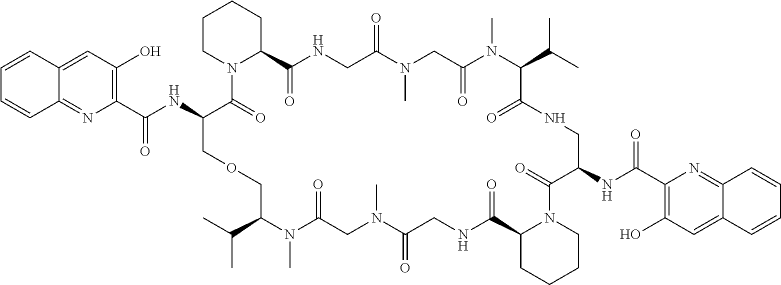 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

Compound 178 was synthesized as described in General Procedure 6 starting from Compound 177 (10 mg, 0.007 mmol), 10% Pd/C (10 mg) and MeOH (2 mL). Following removal of Pd/C, the title compound (7.2 mg, 0.006 mmol, 85% yield) was isolated as a vibrant yellow solid.

LC/MS: Calc'd m/z=1205.6 for C₆₀H₇₉N₁₃O₁₄, found [M+H]⁺=1207.0, [M−Boc+2H]²⁺=604.2.

### 2.196 (1-((2-nitrophenyl)sulfonyl)piperidin-2-yl) methanol (Compound 179)

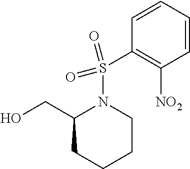 [SMILES: O=[N+]([O-])c1ccccc1S(=O)(=O)N1CCCC[C@H]1CO]

To a stirring solution of piperidin-2-ylmethanol (2.5 g, 21.7 mmol, 1 equiv.) in dioxane (50 mL) and H₂O (10 mL) was added NaHCO₃ (2.2 g, 26 mmol, 1.2 equiv.) and 2-nitrobenzenesulfonamide (4.8 g, 21.7 mmol, 1 equiv.). This solution was stirred for 1 h, concentrated to 50% volume and partitioned between EtOAc (100 mL) and brine (50 mL). The layers were separated, the organic layer was washed with brine (2×20 mL) and dried over Na₂SO₄. Purification according to General Procedure 12, using a 50 g column and eluting with 0 to 100% EtOAc/hexanes, afforded the title compound (3.3 g, 11 mmol, 52% yield) as a white solid.

LC/MS: Calc'd m/z=300.1 for C₁₂H₁₆N₂O₅S, found [M+H]⁺=301.2, ¹H NMR (300 MHZ, chloroform-d) δ 8.16-8.07 (m, 1H), 7.77-7.58 (m, 3H), 4.19-4.02 (m, 1H), 3.97-3.73 (m, 2H), 3.60 (dt, J=11.8, 6.1 Hz, 1H), 3.12 (ddd, J=14.1, 12.4, 2.8 Hz, 1H), 2.06 (d, J=7.2 Hz, 1H), 1.82-1.61 (m, 4H), 1.58-1.40 (m, 2H).

### 2.197 Methyl N-(((4-nitrobenzyl)oxy)carbonyl)-O-((1-((2-nitrophenyl)sulfonyl)piperidin-2-yl)methyl)-D-serinate (Compound 180)

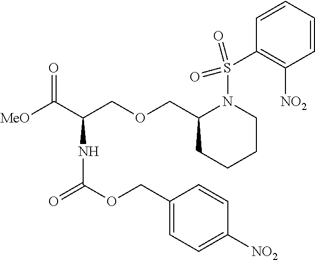 [SMILES: COC(=O)[C@@H](COC[C@@H]1CCCCN1S(=O)(=O)c1ccccc1[N+](=O)[O-])NC(=O)OCc1ccc([N+](=O)[O-])cc1]

The title compound was prepared following the protocol detailed in Liu et al., Org. Lett., 2007, 9:4211-4214. To a stirring solution of Compound 163 (0.83 g, 2.96 mmol, 1 equiv.) and Compound 179 (1.78 g, 5.93 mmol, 2 equiv.) in toluene (30 mL, 0.1M) was added BF₃*OEt₂ (91 μL, 0.74 mmol, 0.25 equiv.) slowly via syringe. The flask was equipped with a reflux condenser and the reaction was heated at 110° C. for 1 h, cooled to rt, and evaporated to dryness. Purification according to General Procedure 12, using a 60 g C18 column and eluting with a linear gradient of 10 to 75% CH₃CN/H₂O, afforded the title compound (1.16 g, 2.0 mmol, 68% yield) as a white solid.

LC/MS: Calc'd m/z=580.6 for C₂₄H₂₈N₄O₁₁S, found [M+H]⁺=581.4, ¹H NMR (300 MHZ, chloroform-d) δ 8.24 (d, J=8.2 Hz, 2H), 8.17-8.06 (m, 1H), 7.80-7.64 (m, 3H), 7.63-7.51 (m, 2H), 6.22 (d, J=8.9 Hz, 1H), 5.27 (s, 2H), 4.51 (dt, J=8.9, 3.1 Hz, 1H), 4.27 (m, 1H), 4.01 (dd, J=9.2, 3.1 Hz, 1H), 3.83 (s, 2H), 3.78 (s, 3H), 3.80-3.65 (m, 2H), 3.61-3.45 (m, 2H), 3.15-3.00 (m, 1H), 1.80-1.68 (m, 3H), 1.56-1.28 (m, 1H).

### 2.198 N-(((4-nitrobenzyl)oxy)carbonyl)-O-((1-((2-nitrophenyl)sulfonyl)piperidin-2-yl)methyl)-D-serine (Compound 181)

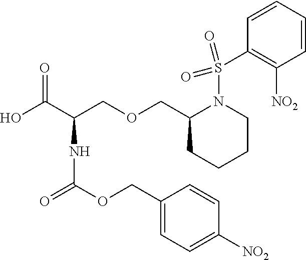 [SMILES: O=C(N[C@H](COC[C@@H]1CCCCN1S(=O)(=O)c1ccccc1[N+](=O)[O-])C(=O)O)OCc1ccc([N+](=O)[O-])cc1]

To a stirring solution of Compound 180 (1.16 g, 2 mmol, 1 equiv.) in THF (30 mL), at 0° C., was added a 1 M aqueous solution of LiOH (4 mL, 4 mmol 2 equiv.). This solution was stirred at 0° C. for 30 min then quenched with a saturated aqueous solution of citric acid (3 mL) and warmed to rt. The aqueous layer was extracted with EtOAc (50 mL) then the organic layer washed with brine (2×20 mL) and dried over Na₂SO₄. Purification according to General Procedure 12, using a 25 g column and eluting with a linear gradient of 0 to 10% MeOH/CH₂Cl₂, afforded the title compound (1 g, 1.76 mmol, 88% yield) as a white solid.

LC/MS: Calc'd m/z=568.1 for C₂₃H₂₆N₄O₁₁S, found [M+H]⁺=568.4. ¹H NMR (300 MHz, chloroform-d) δ 8.1 (d J=8.1 Hz, 2H), 8.14-7.97 (m, 1H), 7.69-7.54 (m, 3H), 7.53-7.41 (m, 2H), 6.18 (d, J=8.7 Hz, 1H), 5.80-5.34 (m, 3H), 5.20 (d, J=17.1 Hz, 2H), 4.19 (s, 1H), 4.03-3.84 (m, 1H), 3.74-3.58 (m, 2H), 3.47 (dd, J=9.9, 5.7 Hz, 1H), 3.39 (s, 1H), 2.98 (t, J=13.6 Hz, 1H), 1.72-1.47 (s, 3H), 1.29 (s, 2H).

### 2.199 (S)-1-(O—((S)-2-(2-(2-((S)-1-((R)-3-((S)-2-(2-(2-amino-N-methylacetamido)-N-methylacetamido)-3-methylbutanamido)-2-((tert-butoxycarbonyl)amino)propanoyl)piperidine-2-carboxamido)-N-methylacetamido)-N-methylacetamido)-3-methylbutyl)-N-(((4-nitrobenzyl)oxy)carbonyl)-D-seryl) piperidine-2-carboxylic acid (Compound 182)

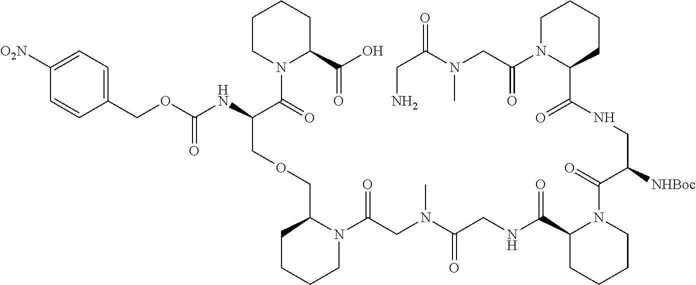 [SMILES: CN(CC(=O)N1CCCC[C@H]1COC[C@@H](NC(=O)OCc1ccc([N+](=O)[O-])cc1)C(=O)N1CCCC[C@H]1C(=O)O)C(=O)CNC(=O)[C@@H]1CCCCN1C(=O)[C@@H](CNC(=O)[C@@H]1CCCCN1C(=O)CN(C)C(=O)CN)NC(=O)OC(C)(C)C]

The title compound was synthesized according to General Procedure 1 utilizing Compound 181, Fmoc-Pip-OH, Boc-D-Dap(Fmoc)-OH, Fmoc-Sar-OH and Fmoc-Gly-OH, on a 0.1 mmol scale. The crude peptide (50 mg, 0.043 mmol, 43% yield) was isolated, post ether precipitation, as white powder and was used without further purification.

LC/MS: Calc'd m/z=1156.6 for C₅₃H₈₀N₁₂O₁₇, found [M+H]⁺=1157.6, [M−Boc+2H]²⁺=529.6.

### 2.200 Tert-butyl (4-nitrobenzyl) ((7R,10aS,22aS,29R,32aS,44aS)-18,40-dimethyl-6,16,19,22,28,32,38,41,44-nonaoxodotetracontahydro-6H-tetrapyrido[2,1-b₁: 2′,1′-c: 2″,1″-1:2′″,1′″-s][1]oxa[4,7,10,13,17,20,23,26,29]nonaazacyclodotriacontine-7,29-diyl)dicarbamate (Compound 183)

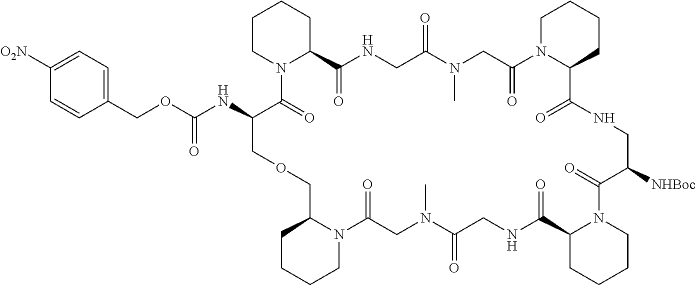 [SMILES: CN1CC(=O)N2CCCC[C@H]2COC[C@@H](NC(=O)OCc2ccc([N+](=O)[O-])cc2)C(=O)N2CCCC[C@H]2C(=O)NCC(=O)N(C)CC(=O)N2CCCC[C@H]2C(=O)NC[C@@H](NC(=O)OC(C)(C)C)C(=O)N2CCCC[C@H]2C(=O)NCC1=O]

The title compound was synthesized as described in General Procedure 4 starting from Compound 182 (50 mg, 0.043 mmol). Preparative HPLC purification according to General Procedure 12 and eluting with a linear gradient of 30 to 70% CH₃CN/H₂O, afforded the title compound (12 mg, 0.01 mmol, 23% yield) as a white solid.

LC/MS: Calc'd m/z=1138.6 for C₅₃H₇₈N₁₂O₁₆, found [M+H]⁺=1139.7, [M−Boc+2H]²⁺=520.4.

### 2.201 Tert-butyl ((7R,11S,19aS,26R,30S,38aS)-7-amino-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamate (Compound 184)

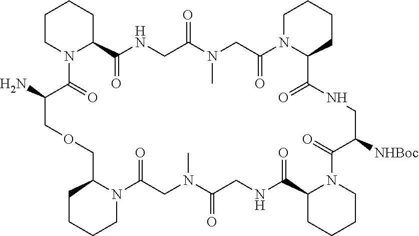 [SMILES: CN1CC(=O)N2CCCC[C@H]2COC[C@@H](N)C(=O)N2CCCC[C@H]2C(=O)NCC(=O)N(C)CC(=O)N2CCCC[C@H]2C(=O)NC[C@@H](NC(=O)OC(C)(C)C)C(=O)N2CCCC[C@H]2C(=O)NCC1=O]

The title compound was synthesized as described in General Procedure 6 starting from Compound 183 (12 mg, 0.01 mmol), 10% Pd/C (10 mg) and MeOH (2 mL). Following removal of Pd/C, the title compound was isolated as a white solid.

LC/MS: Calc'd m/z=959.5 for C₄₅H₇₃N₁₁O₁₂, found [M+H]⁺=960.8, [M−Boc+2H]²⁺=431.0.

### 2.202 (7R,10aS,22aS,29R,32aS,44aS)-18,40-dimethyl-6,16,19,22,28,32,38,41,44-nonaoxodo tetracontahydro-6H-tetrapyrido[2,1-b₁: 2′,1′-c: 2″,1″-1:2′″,1′″-s][1]oxa[4,7,10,13,17,20,23,26,29]nonaazacyclodotriacontine-7,29-diaminium chloride (Compound 185)

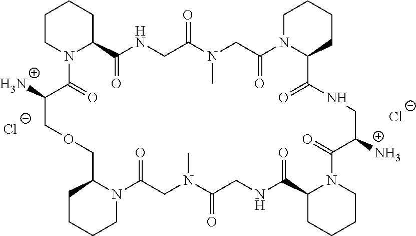 [SMILES: [Cl-] ; CN1CC(=O)N2CCCC[C@H]2COC[C@@H]([NH3+])C(=O)N2CCCC[C@H]2C(=O)NCC(=O)N(C)CC(=O)N2CCCC[C@H]2C(=O)NC[C@@H]([NH3+])C(=O)N2CCCC[C@H]2C(=O)NCC1=O ; [Cl-]]

The title compound was prepared according to General Procedure 5 starting from Compound 184 (0.01 mmol), and 2 mL of 4 M HCl/dioxane.

LC/MS: Calc'd m/z=859.5 for C₄₀H₆₅N₁₁O₁₀, found [M+H]⁺=860.8, [M+2H]²⁺=526.6.

### 2.203 N,N′-((7R,10aS,22aS,29R,32aS,44aS)-18,40-dimethyl-6,16,19,22,28,32,38,41,44-nonaoxodotetracontahydro-6H-tetrapyrido[2,1-b₁: 2′,1′-c: 2″,1″-1:2′″,1′″-s][1]oxa[4,7,10,13,17,20,23,26,29]nonaazacyclodotriacontine-7,29-diyl)bis(3-(benzyloxy)quinoline-2-carboxamide) (Compound 186)

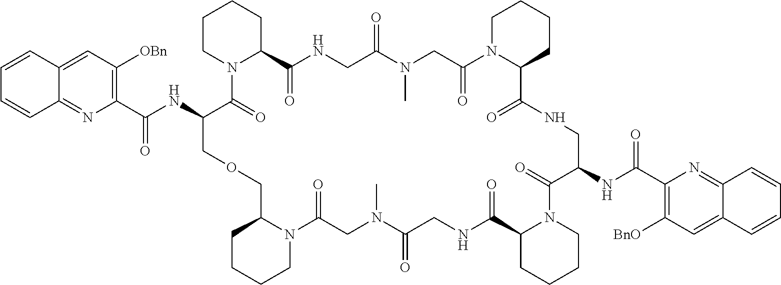 [SMILES: CN1CC(=O)N2CCCC[C@H]2COC[C@@H](NC(=O)c2nc3ccccc3cc2OCc2ccccc2)C(=O)N2CCCC[C@H]2C(=O)NCC(=O)N(C)CC(=O)N2CCCC[C@H]2C(=O)NC[C@@H](NC(=O)c2nc3ccccc3cc2OCc2ccccc2)C(=O)N2CCCC[C@H]2C(=O)NCC1=O]

The title compound was prepared according to General Procedure 7 starting from Compound 185 (0.01 mmol). Purification according to General Procedure 12, using a 10 g column and eluting with a linear gradient of 0 to 20% MeOH/CH₂Cl₂, afforded the title compound (11 mg, 0.008 mmol, 80% yield over 3 steps) as a white solid.

LC/MS: Calc'd m/z=1381.7 for C₇₄H₈₇N₁₃O₁₄, found [M+H]⁺=1383.0, [M+2H]²⁺=692.2.

### 2.204 N,N′-((7R,10aS,22aS,29R,32aS,44aS)-18,40-dimethyl-6,16,19,22,28,32,38,41,44-nonaoxodotetracontahydro-6H-tetrapyrido[2,1-b₁: 2′,1′-c: 2″,1″-1:2′″,1′″-s][1]oxa[4,7,10,13,17,20,23,26,29]nonaazacyclodotriacontine-7,29-diyl)bis(3-hydroxyquinoline-2-carboxamide) (Compound 187; FD-15)

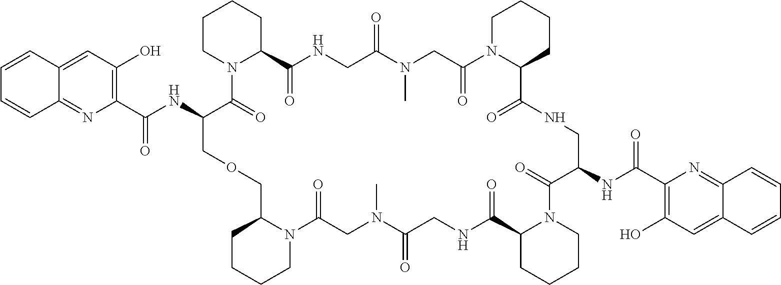 [SMILES: CN1CC(=O)N2CCCC[C@H]2COC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](NC(=O)c2nc3ccccc3cc2O)C(=O)N2CCCC[C@H]2C(=O)NCC1=O]

Compound 187 was synthesized as described in General Procedure 6 starting from Compound 186 (11 mg, 0.008 mmol), 10% Pd/C (10 mg) and MeOH (2 mL). Following removal of Pd/C, the title compound (8.0 mg, 0.0066 mmol, 83% yield) was isolated as a vibrant yellow solid.

LC/MS: Calc'd m/z=1201.6 for C₆₀H₇₅N₁₃O₁₄, found [M+H]⁺=1202.6, [M+2H]²⁺=602.0.

### 2.205 N-((2S,3S)-1-hydroxy-3-methylpentan-2-yl)-2-nitrobenzenesulfonamide (Compound 188)

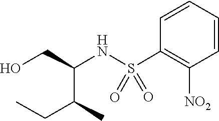 [SMILES: CC[C@H](C)[C@@H](CO)NS(=O)(=O)c1ccccc1[N+](=O)[O-]]

To a stirring solution of (2S,3S)-2-amino-3-methylpentan-1-ol (2 g, 17.1 mmol, 1 equiv.) in dioxane (50 mL) and H₂O (10 mL) was added NaHCO₃ (1.72 g, 20.5 mmol, 1.2 equiv.) and 2-nitrobenzenesulfonamide (3.8 g, 17.1 mmol, 1 equiv.). This solution was stirred for 1 h, concentrated to 50% volume and partitioned between EtOAc (100 mL) and brine (50 mL). The layers were separated, the organic layer was washed with brine (2×20 mL) and dried over Na₂SO₄. Purification according to General Procedure 12, using a 50 g column and eluting with a linear gradient of 0 to 100% EtOAc/hexanes, afforded the title compound (5.2 g) as a white solid.

LC/MS: calc'd m/z=302.1 for C₁₂H₁₈N₂O₅S, found [M+H]⁺=303.2.

### 2.206 N-((2S,3S)-1-hydroxy-3-methylpentan-2-yl)-N-methyl-2-nitrobenzenesulfonamide (Compound 189)

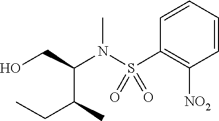 [SMILES: CC[C@H](C)[C@@H](CO)N(C)S(=O)(=O)c1ccccc1[N+](=O)[O-]]

To a stirring solution of Compound 188 (17.1 mmol, 1 equiv.) in DMF (170 mL, 0.1 M) at 0° C. was added NaH (60% wt, 820 mg, 20.5 mmol, 1.2 equiv.). This solution was stirred for 30 min, then CH₃I (1.3 mL, 20.5 mmol, 1.2 equiv.) was added and the reaction was allowed to warm to rt while stirring for 2 h. Solvent was removed in vacuo and purification according to General Procedure 12, using a 120 g C18 column and eluting with 35 to 50% CH₃CN/H₂O, afforded the title compound (2.1 g, 6.6 mmol, 39% yield, 2 steps) as a white solid.

LC/MS: Calc'd m/z=316.1 for C₁₂H₁₈N₂O₅S, found [M+H]⁺=317.2. ¹H NMR (300 MHZ, chloroform-d) δ 8.13-7.98 (m, 1H), 7.70 (dd, J=5.9, 3.4 Hz, 2H), 7.59 (dd, J=5.9, 3.4 Hz, 1H), 3.88 (dd, J=11.7, 3.8 Hz, 1H), 3.72 (td, J=9.4, 3.8 Hz, 1H), 3.58 (dd, J=11.7, 9.4 Hz, 1H), 2.95 (s, 3H), 2.32 (s, 2H), 1.66-1.46 (m, 1H), 1.08-0.80 (m, 6H).

### 2.207 Methyl O-((2S,3S)-3-methyl-2-((N-methyl-2-nitrophenyl)sulfonamido) pentyl)-N-(((4-nitrobenzyl)oxy)carbonyl)-D-serinate (Compound 190)

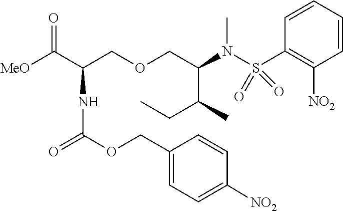 [SMILES: CC[C@H](C)[C@@H](COC[C@@H](NC(=O)OCc1ccc([N+](=O)[O-])cc1)C(=O)OC)N(C)S(=O)(=O)c1ccccc1[N+](=O)[O-]]

To a stirring solution of Compound 163 (0.97 g, 3.46 mmol, 1 equiv.) in toluene (35 mL), at rt, was added Compound 189 (2.19 g, 6.92 mmol, 2 equiv.) followed by BF₃*OEt₂ (0.107 mL, 0.865 mmol, 0.25 equiv.). This solution was heated at reflux for 2 h, cooled to rt, and the solvent removed. Purification according to General Procedure 12, using a 50 g column and eluting with 5 to 45% EtOAc/hexanes, afforded the title compound (0.86 g, 42%) as a colourless oil.

LC/MS: Calc'd m/z=596.2 for C₂₅H₃₂N₄O₁₁S found [M+H]⁺=597.4. ¹H NMR (300 MHz, chloroform-d) δ 8.27-8.19 (m, 2H), 8.00-7.93 (m, 1H), 7.71-7.62 (m, 2H), 7.62-7.47 (m, 3H), 5.79 (d, J=8.7 Hz, 1H), 5.22 (s, 2H), 4.44 (dt, J=8.8, 3.2 Hz, 1H), 3.84 (dd, J=9.4, 3.3 Hz, 1H), 3.78-3.67 (m, 4H), 3.61-3.50 (m, 3H), 2.82 (s, 3H), 1.79-1.58 (m, 1H), 1.46-1.32 (m, 1H), 0.89 (d, J=6.6 Hz, 3H), 0.81 (t, J=7.1 Hz, 3H).

### 2.208 O-((2S,3S)-3-methyl-2-((N-methyl-2-nitrophenyl)sulfonamido) pentyl)-N-(((4-nitrobenzyl)oxy)carbonyl)-D-serine (Compound 191)

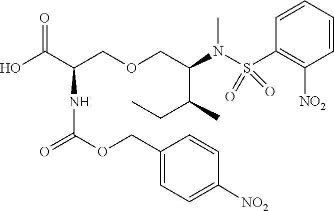 [SMILES: CC[C@H](C)[C@@H](COC[C@@H](NC(=O)OCc1ccc([N+](=O)[O-])cc1)C(=O)O)N(C)S(=O)(=O)c1ccccc1[N+](=O)[O-]]

To stirring solution of Compound 190 (390 mg, 0.654 mmol, 1 equiv.) in EtOAc (3.3 mL), in a 25 mL rb flask wrapped in aluminum foil, was added lithum iodide (0.437 g, 3.268 mmol, 5 equiv.) This solution was heated at reflux until full conversion, quenched with water (4 mL), acidified with aqueous 0.1 M HCl and extracted with ethyl acetate (3×15 mL). The organic phase was then washed with a 1 M aqueous solution of Na₂S₂O₃ (20 mL), water (20 mL) and brine (20 mL). The organic phase was dried over Na₂SO₄ and evaporated to dryness to afford the title compound (370 mg, 0.635 mmol, 97% yield) as a pale-yellow solid.

LC/MS: Calc'd m/z=582.2 for C₂₅H₃₂N₄O₁₁S, found [M+H]⁺=583.4, [M−H]=581.2. ¹H NMR (300 MHz, chloroform-d) δ 8.29-8.11 (m, 2H), 8.02-7.83 (m, 1H), 7.75-7.59 (m, 2H), 7.59-7.40 (m, 3H), 6.01 (s, 1H), 5.20 (s, 2H), 4.46-4.31 (m, 1H), 3.98-3.38 (m, 5H), 2.80 (s, 3H), 1.73-1.43 (m, 1H), 1.44-1.16 (m, 1H), 1.01-0.55 (m, 6H).

### 2.209 (S)-1-((R)-3-((S)-2-(2-(2-((S)-1-(O-((2R,3S)-2-(2-(2-amino-N-methylacetamido)-N-methylacetamido)-3-methylpentyl)-N-(((4-nitrobenzyl)oxy)carbonyl)-D-seryl) piperidine-2-carboxamido)-N-methylacetamido)-N-methylacetamido)-3-methylbutanamido)-2-((tert-butoxycarbonyl)amino)propanoyl)piperidine-2-carboxylic acid (Compound 192)

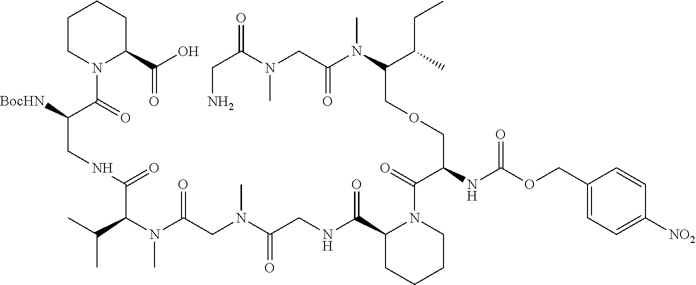

The title compound was synthesized according to General Procedure 1 utilizing Compound 191, Fmoc-Pip-OH, Boc-D-Dap(Fmoc)-OH, Fmoc-N-methyl-Val-OH, and Compound 9, on a 0.15 mmol scale. The crude linear peptide (152 mg, 0.13 mmol, 86% yield), post ether precipitation, was used without additional purification.

LC/MS: Calc'd m/z=1174.6 for C₅₄H₈₆N₁₂O₁₇, found [M+H]⁺=1175.8, [M−Boc+2H]²⁺=538.6.

### 2.210 Tert-butyl (4-nitrobenzyl) ((7R,11R,19aS,26R,30S,38aS)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)dicarbamate (Compound 193)

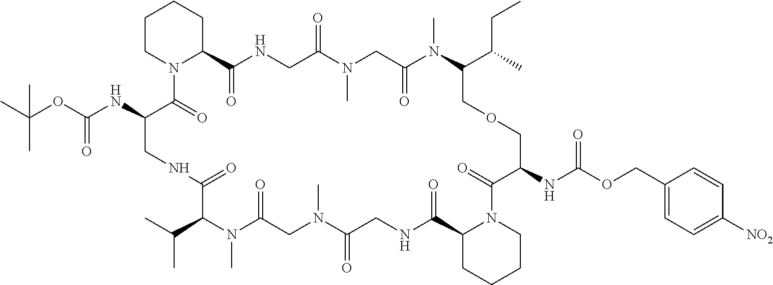

The title compound was synthesized as described in General Procedure 4, starting from Compound 192 (332 mg, 0.275 mmol). Purification according to General Procedure 12, eluting with 0 to 20% MeOH/CH₂Cl₂, afforded the title compound (237 mg, 0.199 mmol, 72% yield) as a white solid.

LC/MS: Calc'd m/z=1156.6 for C₅₄H₈₄N₁₂O₁₆, found [M+H]⁺=1157.8, [M−Boc+H]⁺=1057.8, [M−Boc+2H]²⁺=529.4.

### 2.211 Tert-butyl ((7R,11S,19aS,26R,30S,38aS)-7-amino-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamate (Compound 194)

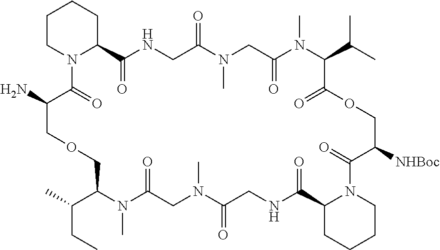 [SMILES: CC[C@H](C)[C@H]1COC[C@@H](N)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized as described in General Procedure 6 starting from Compound 193 (21 mg, 0.018 mmol), 10% Pd/C (10 mg) and MeOH (2 mL). Following removal of Pd/C, the title compound was isolated as a white solid.

LC/MS: Calc'd m/z=977.6 for C₄₆H₇₉N₁₁O₁₂, found [M+H]⁺=978.8, [M−Boc+2H]²⁺=439.8.

### 2.212 (7R,11S,19aS,26R,30S,38aS)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diaminium chloride (Compound 195)

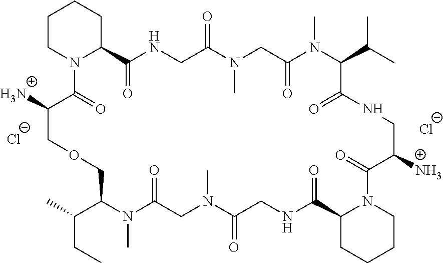 [SMILES: [Cl-] ; CC[C@H](C)[C@H]1COC[C@@H]([NH3+])C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 194 (0.018 mmol) and 2 mL of 4 M HCl/dioxane.

LC/MS: Calc'd m/z=877.5 for C₄₁H₇₁N₁₁O₁₀, found [M+H]⁺=878.5, [M+2H]²⁺=440.0.

### 2.213 N,N′-((7R,11S,19aS,26R,30S,38aS)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-(benzyloxy)quinoline-2-carboxamide) (Compound 196)

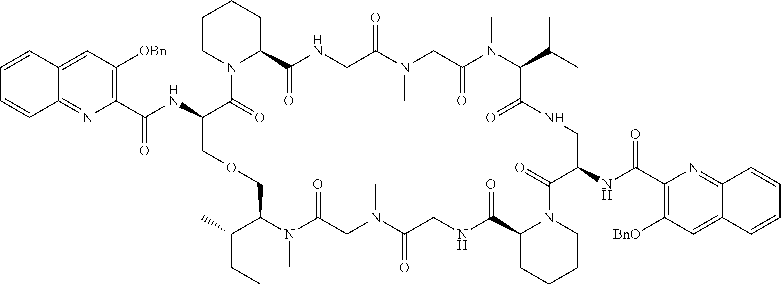 [SMILES: CC[C@H](C)[C@H]1COC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 195 (0.018 mmol). Purification according to General Procedure 12, using a 10 g column and eluting with 0 to 15% MeOH/CH₂Cl₂, afforded the title compound (22 mg, 0.016 mmol, 90% yield over 3 steps) as a white solid.

LC/MS: Calc'd m/z=1399.7 for C₇₅H₉₃N₁₃O₁₄, found [M+2H]²⁺=701.4.

### 2.214 N,N′-((7R,11S,19aS,26R,30S,38aS)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)bis(3-hydroxyquinoline-2-carboxamide) (Compound 197; FD-19)

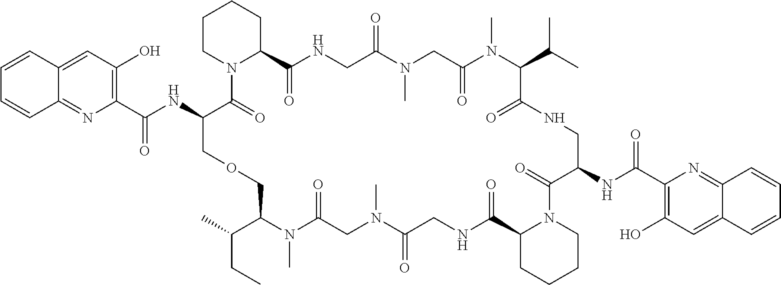 [SMILES: CC[C@H](C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

Compound 197 was synthesized as described in General Procedure 6 starting from Compound 196 (22 mg, 0.016 mmol), 10% Pd/C (4 mg) and MeOH (2 mL). Following removal of Pd/C, purification according to General Procedure 12, using a 12 g C18 column and eluting with a linear gradient of 30 to 90% CH₃CN/H₂O, afforded the title compound (8 mg, 0.007 mmol, 42% yield) as a pale-yellow solid.

LC/MS: Calc'd m/z=1219.6 for C₆₁H₈₁N₁₃O₁₄, found [M+H]⁺=1220.8, [M+2H]²⁺=611.2.

### 2.215 (S)-1-(O—((S)-2-(2-(2-((S)-1-(N-(tert-butoxycarbonyl)—O—(N—(N-glycyl-N-methylglycyl)-N-methyl-L-valyl)-D-seryl) piperidine-2-carboxamido)-N-methylacetamido)-N-methylacetamido)-3-methylbutyl)-N-(((4-nitrobenzyl)oxy)carbonyl)-D-seryl) piperidine-2-carboxylic acid (Compound 198)

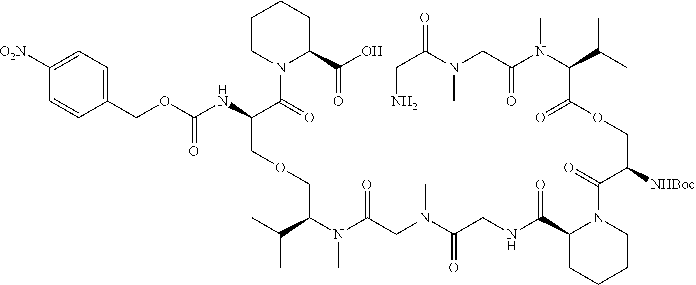 [SMILES: CC(C)[C@@H](COC[C@@H](NC(=O)OCc1ccc([N+](=O)[O-])cc1)C(=O)N1CCCC[C@H]1C(=O)O)N(C)C(=O)CN(C)C(=O)CNC(=O)[C@@H]1CCCCN1C(=O)[C@@H](COC(=O)[C@H](C(C)C)N(C)C(=O)CN(C)C(=O)CN)NC(=O)OC(C)(C)C]

The title compound was synthesized according to General Procedures 1 and 2 utilizing Fmoc-Pip-OH, Fmoc-Sar-OH, Fmoc-Gly-OH, Fmoc-Gly-Sar-OH, Compound 167 and Compound 8. The crude peptide (176 mg, 0.15 mmol, 38% yield) was isolated, post ether precipitation, as white powder and was used without additional purification.

LC/MS: Calc'd m/z=1161.6 for C₅₃H₈₃N₁₁O₁₈, found [M+H]⁺=1162.8, [M−Boc+2H]²⁺=532.0.

### 2.216 Tert-butyl (4-nitrobenzyl) ((7R,11S,19aS,26R,30S,38aS)-11,30-diisopropyl-12,15,31,34-tetramethyl-6,10,13,16,19,25,32,35,38-nonaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)dicarbamate (Compound 199)

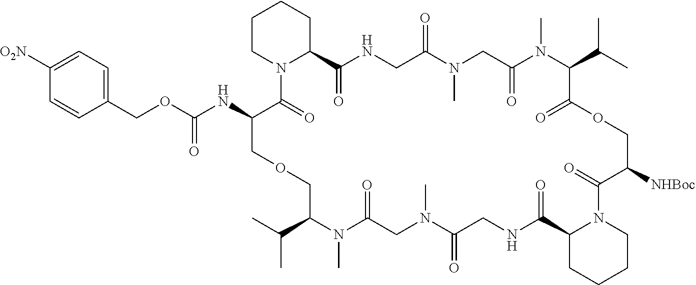 [SMILES: CC(C)[C@H]1COC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 198 (176 mg, 0.15 mmol). Purification according to General Procedure 12, using a 12 g C18 column, and eluting with 0 to 20% CH₂Cl₂/MeOH, afforded the title compound (86 mg, 0.075 mmol, 50% yield) as a white solid.

LC/MS: Calc'd m/z=1143.6 for C₅₃H₈₁N₁₁O₁₇, found [M+H]⁺=1144.9, [M−Boc+2H]²⁺=523.0.

### 2.217 Tert-butyl ((7R,11S,19aS,26R,30S,38aS)-26-amino-11,30-diisopropyl-12,15,31,34-tetramethyl-6,10,13,16,19,25,32,35,38-nonaoxodotriacontahydro-2H,6H,10H,25H-dipyrido[2,1-b₁: 2′,1′-l][1,17]dioxa[4,7,10,13,20,23,26,29]octaazacyclodotriacontin-7-yl)carbamate (Compound 200)

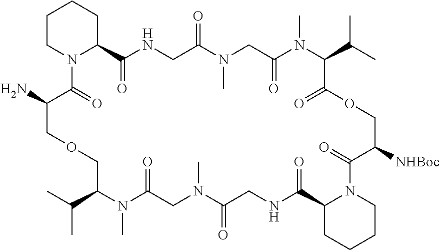 [SMILES: CC(C)[C@H]1COC[C@@H](N)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized as described in General Procedure 6 starting from Compound 199 (10 mg, 0.009 mmol), 10% Pd/C (2 mg) and MeOH (2 mL). Following removal of Pd/C, the title compound was isolated as a white solid.

LC/MS: Calc'd m/z=964.6 for C₄₅H₇₆N₁₀O₁₃, found [M+H]⁺=965.8, [M−Boc+2H]²⁺=433.4.

### 2.218 (7R,11S,19aS,26R,30S,38aS)-11,30-diisopropyl-12,15,31,34-tetramethyl-6,10,13,16,19,25,32,35,38-nonaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diaminium chloride (Compound 201)

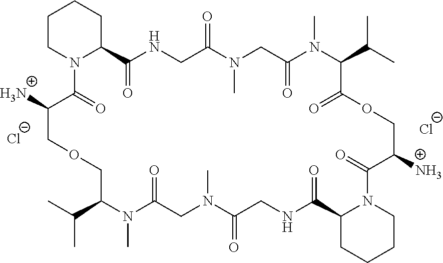 [SMILES: [Cl-] ; CC(C)[C@H]1COC[C@@H]([NH3+])C(=O)N2CCCC[C@H]2C(=O)NCC(=O)N(C)CC(=O)N(C)[C@@H](C(C)C)C(=O)OC[C@@H]([NH3+])C(=O)N2CCCC[C@H]2C(=O)NCC(=O)N(C)CC(=O)N1C ; [Cl-]]

The title compound was prepared according to General Procedure 5 starting from Compound 200 (0.009 mmol) and 2 mL of 4 M HCl/dioxane.

LC/MS: Calc'd m/z=864.5 for C₄₀H₆₈N₁₀O₁₁, found [M+H]⁺=865.6, [M+2H]²⁺=433.4.

### 2.219 N,N′-((7R,11S,19aS,26R,30S,38aS)-11,30-diisopropyl-12,15,31,34-tetramethyl-6,10,13,16,19,25,32,35,38-nonaoxodotriacontahydro-2H,6H,10H,25H-dipyrido[2,1-b₁: 2′,1′-l][1,17]dioxa[4,7,10,13,20,23,26,29]octaazacyclodotriacontine-7,26-diyl)bis(3-(benzyloxy)quinoline-2-carboxamide) (Compound 202)

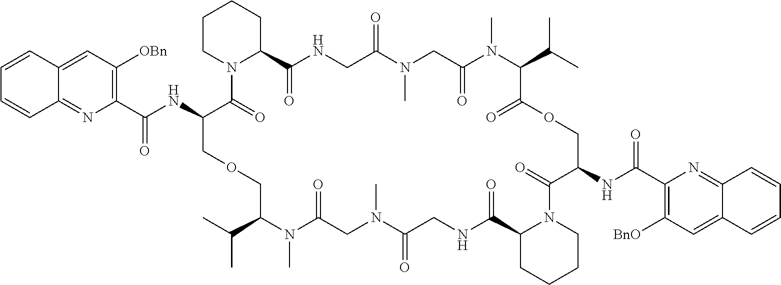 [SMILES: CC(C)[C@H]1COC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)OC[C@@H](NC(=O)c2nc3ccccc3cc2OCc2ccccc2)C(=O)N2CCCC[C@H]2C(=O)NCC(=O)N(C)CC(=O)N1C]

The title compound was prepared according to General Procedure 7 starting from Compound 201 (0.009 mmol). Purification according to General Procedure 12, using a 10 g column and eluting with 0 to 15% MeOH/CH₂Cl₂, afforded the title compound (6.4 mg, 0.0046 mmol, 51% yield over 3 steps) as a white solid.

LC/MS: Calc'd m/z=1386.7 for C₇₄H₉₀N₁₂O₁₅, found [M+H]⁺=1389.4, [M+2H]²⁺=694.8.

### 2.220 N,N′-((7R,11S,19aS,26R,30S,38aS)-11,30-diisopropyl-12,15,31,34-tetramethyl-6,10,13,16,19,25,32,35,38-nonaoxodotriacontahydro-2H,6H,10H,25H-dipyrido[2,1-b₁: 2′,1′-1][1,17]dioxa[4,7,10,13,20,23,26,29]octaazacyclodotriacontine-7,26-diyl)bis(3-hydroxyquinoline-2-carboxamide) (Compound 203; FD-18)

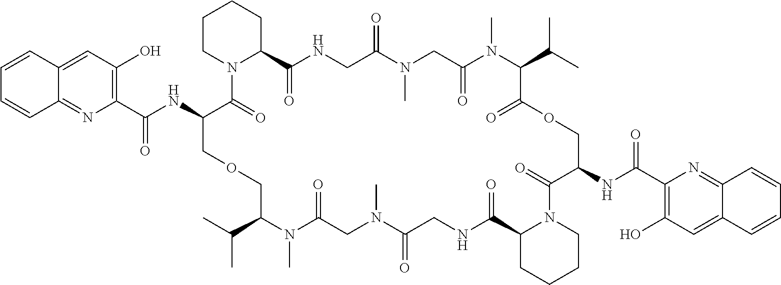 [SMILES: CC(C)[C@H]1COC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)c2nc3ccccc3cc2O)C(=O)N2CCCC[C@H]2C(=O)NCC(=O)N(C)CC(=O)N1C]

Compound 203 was synthesized as described in General Procedure 6 starting from Compound 202 (3.2 mg, 0.0023 mmol), 10% Pd/C (2 mg) and MeOH (2 mL). Following removal of Pd/C, the title compound (2.1 mg, 0.0017 mmol, 76% yield) was isolated as a vibrant yellow solid.

LC/MS: Calc'd m/z=1206.6 for C₆₀H₇₈N₁₂O₁₅, found [M+H]⁺=1207.8, [M+2H]²⁺=604.4.

### 2.221 (S)—N-ethyl-N-(1-hydroxy-3-methylbutan-2-yl)-2-nitrobenzenesulfonamide (Compound 204)

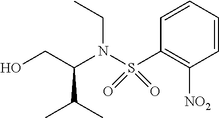 [SMILES: CCN([C@H](CO)C(C)C)S(=O)(=O)c1ccccc1[N+](=O)[O-]]

To a stirred solution of Compound 164 (520 mg, 1.80 mmol) in DMF (3 mL) at 0° C. was added sodium hydride (60%, 86 mg, 2.16 mmol). After 30 minutes stirring at 0° C., ethyl iodide (172 μL, 2.16 mmol) was added. The reaction mixture was stirred for 16 h at rt, then injected directly on a 60 g C18 column and eluted with a 20-60% CH₃CN/H₂O to afford the title compound (310 mg, 0.981 mmol, 54% yield) as white powder.

LC/MS: Calc'd 316.1 for C₁₃H₂₀N₂O₅S found [M+H]⁺=317.2. ¹H (300 MHz, acetone-d6) δ 8.22 (dd, J=7.7, 1.8 Hz, 1H), 7.88-7.74 (m, 3H), 3.79-3.74 (m, 2H), 3.65-3.57 (m, 1H), 3.57-3.50 (m, 1H), 3.44-3.35 (m, 1H), 2.02-1.94 (m, 1H), 1.25 (t, J=7.1 Hz, 3H), 0.99 (d, J=6.5 Hz, 3H), 0.86 (d, J=6.8 Hz, 3H).

### 2.222 N-ethyl-N-((2-nitrophenyl)sulfonyl)-L-valine (Compound 205)

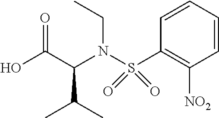 [SMILES: CCN([C@H](C(=O)O)C(C)C)S(=O)(=O)c1ccccc1[N+](=O)[O-]]

To a stirred solution of Compound 204 (310 mg, 0.981 mmol) in 5 mL acetonitrile was added 4 mL phosphate buffer (1M, pH 6.7). TEMPO (11 mg, 0.068 mmol) followed by bleach (25 mL in 0.5 mL H₂O). NaClO₂ (220 mg, 1.96 mmol, in 1 mL H₂O) was then added dropwise over 5 minutes. The reaction mixture was stirred for 16 h at rt. The reaction mixture was quenched with 10 mL 1M Na₂S₂O₃, adjusted to pH 2 with aqueous 1 M HCl, then extracted with EtOAc (3×50 mL). The combined organic extracts were dried with Na₂SO₄ then concentrated in vacuo and the crude product was used without purification.

LC/MS: Calc'd 330.1 for C₁₃H₁₈N₂O₆S found [M−H]=329.2. ¹H (300 MHz, acetone-d6) δ 8.11 (dd, J=7.6, 1.7 Hz, 1H), 7.92-7.77 (m, 3H), 4.19 (d, J=10 Hz, 1H), 3.56 (q, J=7.2 Hz, 2H), 2.30-2.17 (m, 1H), 1.26 (t, J=7.1 Hz, 3H), 1.02 (d, J=6.6 Hz, 1H), 0.99 (d, J=6.8 Hz, 3H).

### Example 3: Synthesis of Drug-Linkers Comprising Compounds of General Formula (I)

### 3.1 Tert-butyl ((S)-1-(((S)-1-((4-(hydroxymethyl)phenyl)amino)-1-oxo-5-ureidopentan-2-yl)amino)-3-methyl-1-oxobutan-2-yl)carbamate (Compound 206)

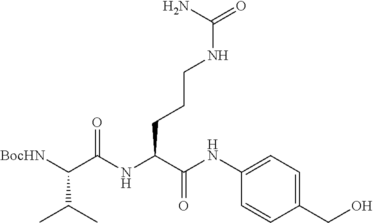 [SMILES: CC(C)[C@H](NC(=O)OC(C)(C)C)C(=O)N[C@@H](CCCNC(N)=O)C(=O)Nc1ccc(CO)cc1]

The title compound was prepared according to the protocol described in International Patent Publication No. WO 2005/112919.

LC/MS: Calc'd m/z=479.3 for C₂₃H₃₇N₅O₆, found [M+H]⁺=480.4, [M−Boc, +H]⁺=380.2.

### 3.2 Tert-butyl ((S)-1-(((S)-1-((4-(chloromethyl)phenyl)amino)-1-oxo-5-ureidopentan-2-yl)amino)-3-methyl-1-oxobutan-2-yl)carbamate (Compound 207)

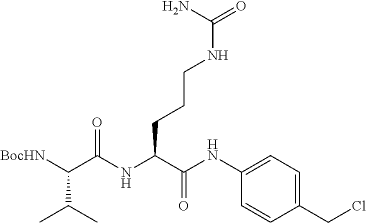 [SMILES: CC(C)[C@H](NC(=O)OC(C)(C)C)C(=O)N[C@@H](CCCNC(N)=O)C(=O)Nc1ccc(CCl)cc1]

A solution of Compound 206 (1.5 g, 3.13 mmol, 1 equiv.) and benzotriazole (0.45 g, 3.75 mmol, 1.2 equiv.) in THF (31 mL, 0.1M) was cooled to 0° C., and thionyl chloride (0.25 mL, 3.44 mmol, 1.1 equiv.) was added dropwise. The ice bath was removed, the solution was stirred, at rt for 1 h, then filtered through Celite, and concentrated. Purification according to General Procedure 12, using a 25 g column and eluting with 0 to 15% MeOH/DCM, afforded the title compound (1.15 g, 2.31 mmol, 74% yield) as a flaky white solid.

LC/MS: Calc'd m/z=497.2 for C₂₃H₃₆ClN₅O₅, found [M−Boc+H]⁺=398.2, [M−H]=496.2. ¹H NMR (300 MHz, methanol-d₄) δ 7.68-7.50 (m, 2H), 7.44-7.30 (m, 2H), 4.62 (s, 2H), 4.54 (dd, J=8.8, 5.2 Hz, 1H), 3.93 (d, J=6.7 Hz, 1H), 3.17 (ddt, J=26.8, 13.5, 6.7 Hz, 2H), 2.05 (dq, J=15.7, 8.3, 7.6 Hz, 1H), 1.92 (p, J=7.1 Hz, 1H), 1.78 (dtd, J=13.6, 9.0, 5.1 Hz, 1H), 1.70-1.51 (m, 2H), 1.46 (s, 9H), 1.07-0.86 (m, 6H).

### 3.3 Methyl 3-((4-((S)-2-((S)-2-((tert-butoxycarbonyl)amino)-3-methylbutanamido)-5-ureidopentanamido)benzyl)oxy)quinoline-2-carboxylate (Compound 208)

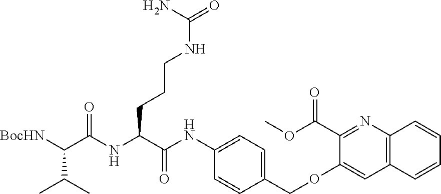 [SMILES: COC(=O)c1nc2ccccc2cc1OCc1ccc(NC(=O)[C@H](CCCNC(N)=O)NC(=O)[C@@H](NC(=O)OC(C)(C)C)C(C)C)cc1]

Compound 207 (1.15 g, 2.31 mmol, 1 equiv.), methyl 3-hydroxyquinoline-2-carboxylate (0.47 g, 2.31 mmol, 1 equiv.) and potassium carbonate (0.32 g, 2.31 mmol, 1 equiv.) were dissolved in DMF (23 mL, 0.1 M) in a 100 mL RB flask. This flask was fitted with a reflux condenser and heated at 70° C. for 2 h. The solution was cooled to rt, concentrated to an oil, and purified according to General Procedure 12, using a 25 g column and eluting with 0 to 15% MeOH/DCM to afford the title compound (1.3 g, 2.0 mmol, 85% yield) as a flaky off-white solid.

LC/MS: Calc'd m/z=664.3 for C₃₄H₄₄N₆O₈, found [M+H]⁺=665.6, [M−Boc+H]⁺=565.4, [M−H]⁻=663.4. ¹H NMR (300 MHz, methanol-d₄) δ 8.06-7.77 (m, 3H), 7.74-7.54 (m, 4H), 7.54-7.40 (m, 2H), 5.29 (s, 2H), 4.54 (dd, J=8.7, 5.2 Hz, 1H), 4.00 (s, 3H), 3.92 (d, J=6.6 Hz, 1H), 3.30-3.04 (m, 2H), 2.05 (dq, J=13.8, 7.0 Hz, 1H), 1.92 (p, J=7.3 Hz, 1H), 1.78 (dtd, J=13.7, 9.0, 5.1 Hz, 1H), 1.68-1.52 (m, 2H), 1.44 (s, 8H), 0.97 (dd, J=10.4, 6.8 Hz, 6H).

### 3.4 3-((4-((S)-2-((S)-2-((tert-butoxycarbonyl)amino)-3-methylbutanamido)-5-ureidopentanamido)benzyl)oxy)quinoline-2-carboxylic acid (Boc-VC-PAB-Chromophore-OH; Compound 209)

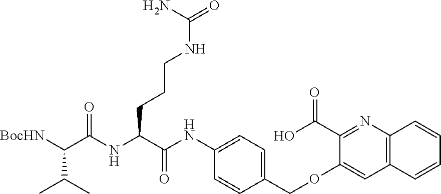 [SMILES: CC(C)[C@H](NC(=O)OC(C)(C)C)C(=O)N[C@@H](CCCNC(N)=O)C(=O)Nc1ccc(COc2cc3ccccc3nc2C(=O)O)cc1]

A solution of Compound 208 (1.3 g, 2.0 mmol, 1 equiv.) in THF (39 mL, 0.05 M) was cooled to 0° C. in an ice bath. Aqueous 1 M LiOH (4 mL, 3.9 mmol, 2 equiv.) was added slowly in a single portion, the ice bath removed, and the reaction stirred for 3 h. Excess LiOH was quenched with aqueous 1 M HCl added dropwise until a pH<5 was achieved. This cloudy solution was evaporated to dryness and purified according to General Procedure 12, using a 120 g C18 column and eluting with 10 to 60% CH₃CN/H₂O to afford the title compound (0.97 g, 1.5 mmol, 77% yield) as a faint yellow powder.

LC/MS: Calc'd m/z=650.3 for C₃₃H₄₂N₆O₈, found [M−H]⁻=649.4. ¹H NMR (300 MHZ, acetone-d₆) δ 9.57 (s, 1H), 8.03 (d, J=8.1 Hz, 2H), 7.91 (dd, J=7.8, 1.9 Hz, 1H), 7.81-7.56 (m, 5H), 7.52 (d, J=8.5 Hz, 2H), 6.20 (d, J=8.1 Hz, 1H), 5.34 (s, 2H), 4.81-4.61 (m, 1H), 4.07 (dd, J=8.2, 5.9 Hz, 1H), 3.45-3.08 (m, 2H), 2.17 (h, J=6.7 Hz, 1H), 1.98 (p, J=7.3, 6.4 Hz, 1H), 1.87-1.54 (m, 3H), 1.41 (s, 9H), 0.97 (dd, J=8.8, 6.8 Hz, 6H).

### 3.5 Tert-butyl ((S)-1-(((S)-1-((4-(hydroxymethyl)phenyl)amino)-1-oxopropan-2-yl)amino)-3-methyl-1-oxobutan-2-yl)carbamate (Boc-VA-PAB-OH; Compound 210)

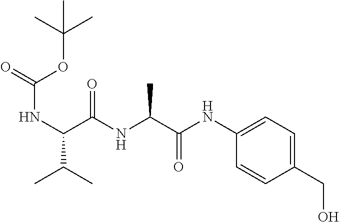 [SMILES: CC(C)[C@H](NC(=O)OC(C)(C)C)C(=O)N[C@@H](C)C(=O)Nc1ccc(CO)cc1]

The title compound was prepared according to the protocol described in International Patent Publication No. WO 2017/096311.

### 3.6 Tert-butyl ((S)-1-(((S)-1-((4-(chloromethyl)phenyl)amino)-1-oxopropan-2-yl)amino)-3-methyl-1-oxobutan-2-yl)carbamate (Boc-VA-PAB-CI; Compound 211)

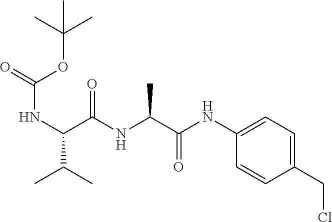 [SMILES: CC(C)[C@H](NC(=O)OC(C)(C)C)C(=O)N[C@@H](C)C(=O)Nc1ccc(CCl)cc1]

To a solution of Compound 210 (500 mg, 1.27 mmol) in anhydrous THF (10 mL) was added benzotriazole (181 mg, 1.52 mmol) and this solution cooled to 0° C. Thionyl chloride (101 μL, 1.40 mmol) was added dropwise, and the reaction mixture was stirred at 0° C. for 40 min. The reaction mixture was filtered, and the filtrate was partially concentrated in vacuo. The crude product was purified as described in General Procedure 12, using a 25 g column and eluting with 0 to 40% EtOAc/hexanes. The product was purified a second time, with the same conditions, to provide the title product (301 mg, 0.732 mmol, 58% yield).

LC/MS: Calc'd m/z=411.2 for C₂₀H₃₀ClN₃O₄ found [M−H]⁻=410.2.

### 3.7 Methyl 3-((4-((S)-2-((S)-2-((tert-butoxycarbonyl)amino)-3-methylbutanamido) propanamido)benzyl)oxy)quinoline-2-carboxylate (Compound 212)

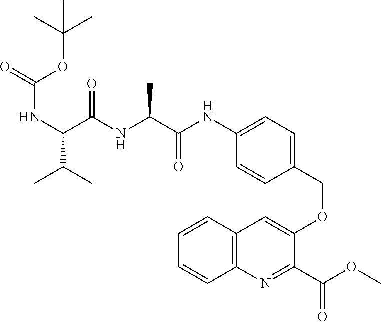 [SMILES: COC(=O)c1nc2ccccc2cc1OCc1ccc(NC(=O)[C@H](C)NC(=O)[C@@H](NC(=O)OC(C)(C)C)C(C)C)cc1]

To a stirred solution of methyl 3-hydroxyquinoline-2-carboxylate (200 mg, 0.632 mmol) in DMF (3 mL) was added potassium carbonate (87 mg, 0.632 mmol), followed by a solution of Compound 209 (260 mg, 0.632 mmol) in DMF (3 mL). The reaction mixture was heated at 40° C. for 45 minutes then partially concentrated in vacuo. The crude product was purified according to General Procedure 12, using a 25 g column and eluting with a 0 to 20% MeOH/CH₂Cl₂, to afford the title compound (158 mg, 0.273 mmol, 43%) as a light yellow solid.

LC/MS: Calc'd m/z=578.3 for C₃₁H₃₈N₄O₇, found [M+H]⁺=579.4.

### 3.8 3-((4-((S)-2-((S)-2-((tert-butoxycarbonyl)amino)-3-methylbutanamido) propanamido)benzyl)oxy)quinoline-2-carboxylic acid (Compound 213)

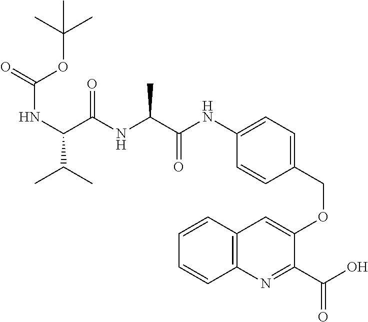 [SMILES: CC(C)[C@H](NC(=O)OC(C)(C)C)C(=O)N[C@@H](C)C(=O)Nc1ccc(COc2cc3ccccc3nc2C(=O)O)cc1]

To a stirred solution of Compound 212 (915 mg, 1.58 mmol) in THF (10 mL) was added aqueous 1 M LiOH (1.58 mL). After 5 h the solution was quenched by adjusting the pH to <5 with acetic acid then partially concentrated in vacuo. The crude product was purified according to General Procedure 12, using a 60 g C18 column and eluting with 30 to 60% CH₃CN/H₂O, to afford the title compound (418 mg, 0.741 mmol, 47%).

LC/MS: Calc'd m/z=564.3 for C₃₀H₃₆N₄O₇, found [M+Cl]⁻=599.4.

### 3.9 2,3,5,6-tetrafluorophenyl 3-(2-(2-(2-(2,5-dioxo-2,5-dihydro-1H-pyrrol-1-yl) ethoxy) ethoxy) ethoxy) propanoate (MT-OTfp; Compound 214)

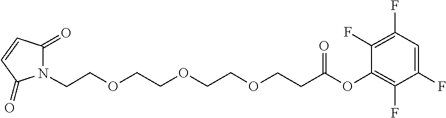 [SMILES: O=C(CCOCCOCCOCCN1C(=O)C=CC1=O)Oc1c(F)c(F)cc(F)c1F]

The title compound was prepared according to WO 2017/054080.

LC/MS: Calc'd m/z=449.4 for C₁₉H₁₉F₄O₇, found [M+H]⁺=450.4.

### 3.10 Benzyl ((7R,11S,19aS,26R,30S,38aS)-26-(3-(benzyloxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 215)

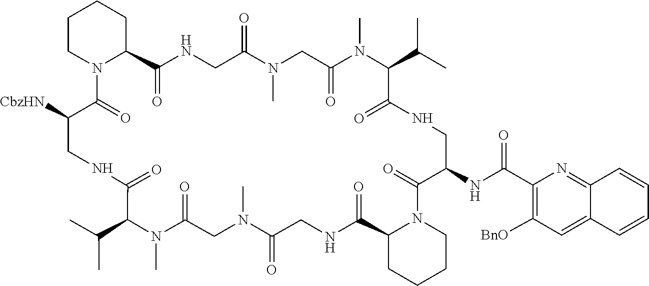 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](NC(=O)OCc2ccccc2)C(=O)N2CCCC[C@H]2C(=O)NCC(=O)N(C)CC(=O)N1C]

The title compound was synthesized from Compound 58 (292 mg, 279 μmol) according to General Procedure 7. Purification according to General Procedure 12, using a 10 g column and eluting 0 to 10% MeOH/CH₂Cl₂, afforded the title compound (158 mg, 45%) as a pale-yellow solid.

LC/MS: Calc'd m/z=1271.6 for C₆₅H₈₅N₁₃O₁₄ found [M+2H]²⁺=671.8.

### 3.11 N-((7R,11S,19aS,26R,30S,38aS)-26-amino-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)-3-hydroxyquinoline-2-carboxamide (Compound 216)

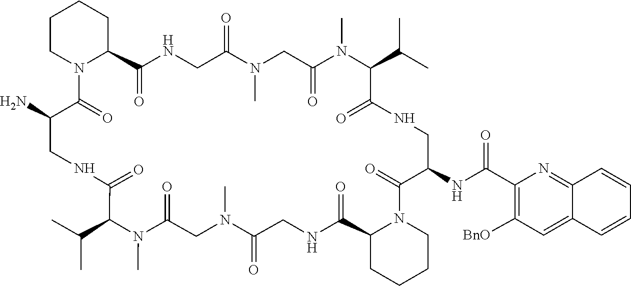 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)NC[C@@H](N)C(=O)N2CCCC[C@H]2C(=O)NCC(=O)N(C)CC(=O)N1C]

The title compound was synthesized from Compound 215 (24 mg, 19 μmol) according to General Procedure 6, and isolated as a bright yellow solid (12 mg, 61% yield).

LC/MS: Calc'd m/z=1047.6 for C₅₀H₇₃N₁₃O₁₂ found [M+2H]²⁺=525.0.

### 3.12 Tert-butyl ((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 217)

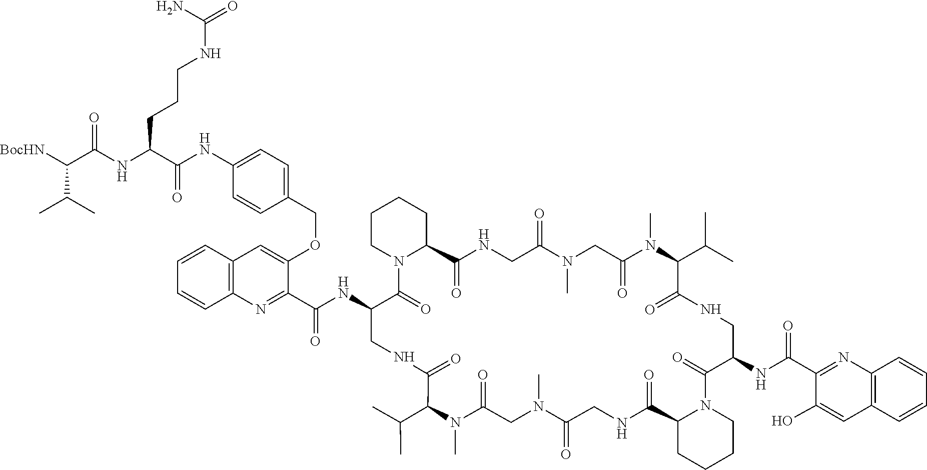 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 216 (12 mg, 11 μmol) according to General Procedure 9. Purification according to General Procedure 12, using a 12 g C18 column and eluting with 30 to 70% CH₃CN/H₂O, afforded the title compound (12.6 mg, 62% yield) as a yellow solid.

LC/MS: Calc'd m/z=1679.9 for C₈₃H₁₁₃N₁₉O₁₉ found [M+2H]²⁺=841.4, [M−Boc+3H]³⁺=527.8.

### 3.13 3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)-N-((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)quinoline-2-carboxamide (MT-VC-PAB-35; Compound 218; DL-1)

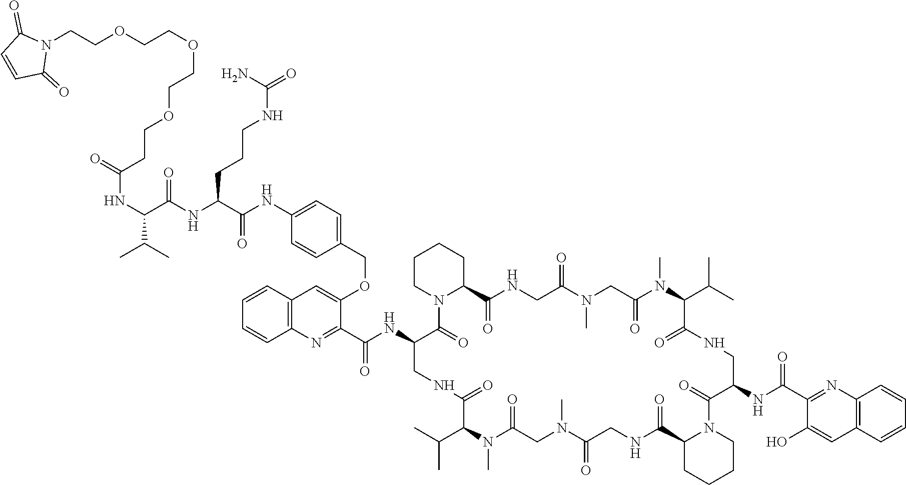 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

In 5 mL pear-shaped flask containing Compound 217 (12.6 mg, 7.1 μmol) was added a 4M solution of HCl in dioxane (0.71 mL, 0.01 M) at 0° C. This solution was stirred at 0° C. until completion. The solution was then evaporated to dryness, and the obtained residue was transformed according to General Procedure 11. Preparative HPLC purification according to General Procedure 12 afforded the title compound (7.7 mg, 58% yield) as pale-yellow solid.

LC/MS: Calc'd m/z=862.9 for C₉₁H₁₂₂N₂₀O₂₃, found [M+2H]²⁺=933.2, [M+3H]³⁺=622.4.

### 3.14 Tert-butyl ((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-yl)carbamate (Compound 219)

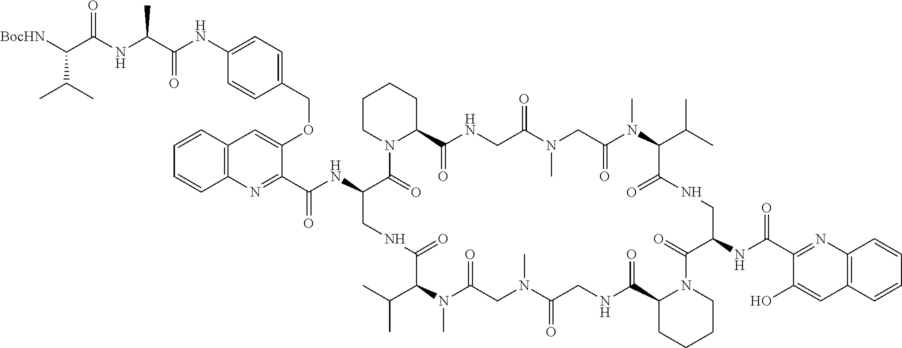 [SMILES: CC(C)[C@H](NC(=O)OC(C)(C)C)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 216 (120 mg, 144 μmol) according to General Procedure 9, and using Compound 213. Purification according to General Procedure 12, using a 30 g C18 column and eluting with 30 to 70% CH₃CN/H₂O, afforded the title compound as a yellow solid (112 mg, 61% yield).

LC/MS: Calc'd m/z=1593.8 for C₈₀H₁₀₇N₁₇O₁₈, found [M+2H]²⁺=798.4.

### 3.15 (S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-aminium chloride (Compound 220)

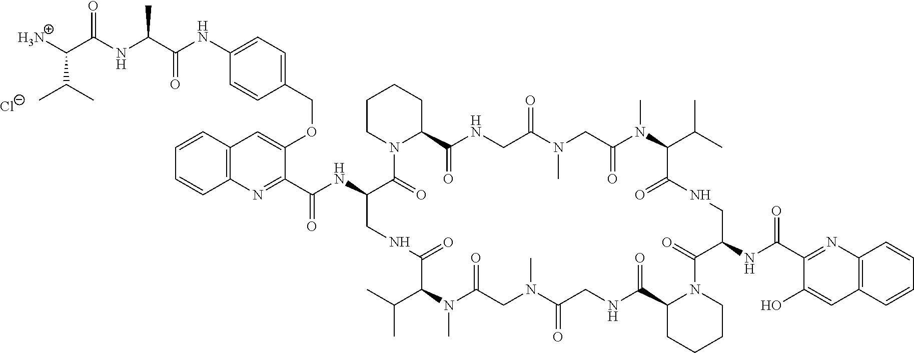 [SMILES: [Cl-] ; CC(C)[C@H]([NH3+])C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

To a 25 mL flask containing Compound 219 (0.112 g, 0.07 mmol, 1 equiv.), at 0° C., was added a 4 M solution of HCl in dioxane (7.0 mL, 0.01 M). The obtained suspension was then allowed to warm up to room temperature and was stirred for 2 h. After complete conversion, the reaction mixture was evaporated to dryness to afford the title product (0.1 g, 85% purity, 79% yield) as yellow solid. The 5% impurity was Compound 35, which was the result of acidic cleavage of the VA-PAB-ether.

LC/MS: Calc'd m/z=1493.8 for C₇₅H₉₉N₁₇O₁₆, found [M+2H]²⁺=748.4.

### 3.16 3-((4-((14S,17S)-1-(2,5-dioxo-2,5-dihydro-1H-pyrrol-1-yl)-14-isopropyl-17-methyl-12,15-dioxo-3,6,9-trioxa-13,16-diazaoctadecan-18-amido)benzyl)oxy)-N-((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)quinoline-2-carboxamide (MT-VA-PAB-35; Compound 221; DL-3)

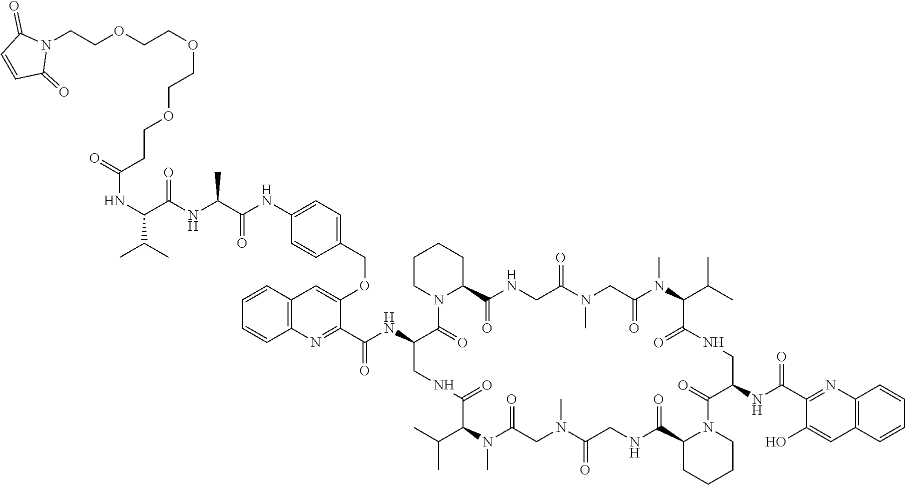 [SMILES: CC(C)[C@H](NC(=O)CCOCCOCCOCCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was prepared according to General Procedure 11 starting from Compound 220 (16 mg, 0.01 mmol, 1 equiv.), Compound 214 (5 mg, 0.01 mmol, 1 equiv.), NEt (i-Pr) 2 (3.7 μL, 0.02 mmol, 2 equiv.), and in DMF (500 μL). Preparative HPLC purification according to General Procedure 12, eluting with 40 to 55% CH₃CN/H₂O, afforded the title compound (12.4 mg, 0.007 mmol, 70% yield) as a white solid.

LC/MS: Calc'd m/z=1776.8 for C₈₈H₁₁₆N₁₈O₂₂, found [M+2H]²⁺=890.0, [M+3H]³⁺=593.8.

### 3.17 2-(1-(2,5-dioxo-2,5-dihydro-1H-pyrrol-1-yl)-3-oxo-7,10,13,16-tetraoxa-4-azanonadecan-19-amido)-N5-((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-yl)-N₁-(2,5,8,11,14,17,20,23,26,29,32,35,38,41,44,47,50,53,56,59,62,65,68,71-tetracosaoxatriheptacontan-73-yl) pentanediamide (ME-dPEG4-Glu-dPEG24-VA-PAB-35; Compound 222; DL-7)

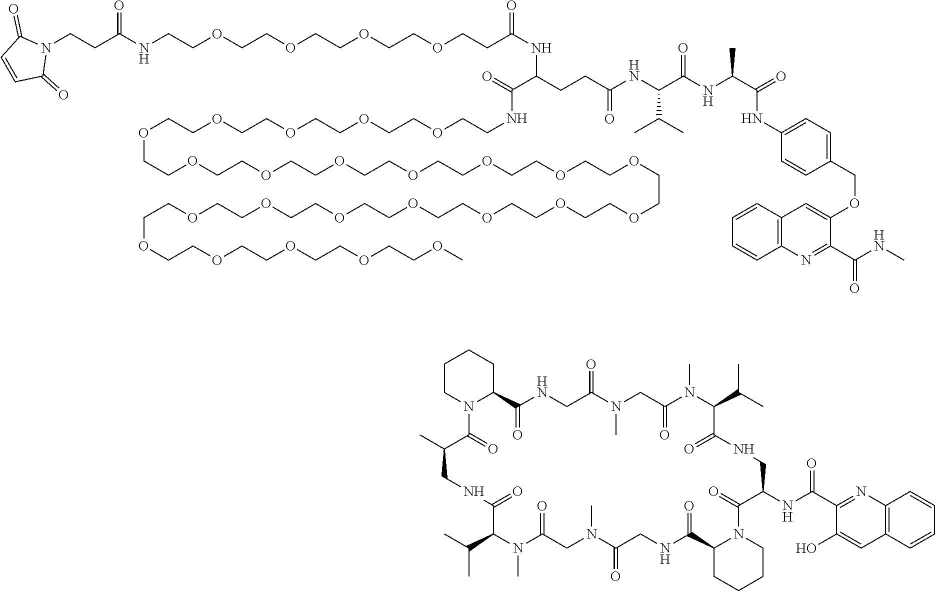

To a 5 mL pear-shaped flask containing Compound 220 (0.028 g, 0.018 mmol, 1 equiv.) was added a solution of ME-dPEG®₄-Glu(TFP ester)-NH-m-dPEG®₂₄ (19 mg, 0.01 mmol, 0.6 equiv.) and NEt(i-Pr)₂ (7 μL, 0.04 mmol, 2 equiv.) in DMF (1 mL). This solution was stirred at room temperature for 30 min then additional M-dPEG®₄-Glu(TFP ester)-NH-m-dPEG®₂₄ (14 mg, 0.008 mmol, 0.88 equiv.) was added along with additional NEt(i-Pr)₂ (3.36 μL, 0.019 mmol, 2.1 equiv.) and stirring was continued for 16 h. The reaction mixture was evaporated to dryness and preparative HPLC purification according to General Procedure 12 afforded the title compound (31 mg, 55% yield) as an oily yellowish residue.

LC/MS: Calc'd m/z=3090.6 for C₁₄₇H₂₃₁N₂₁O₅₀, found [M+3H]³⁺=1031.8.

### 3.18 (R)-2-((tert-butoxycarbonyl)amino)-3-((2,5-dioxopyrrolidin-1-yl)oxy)-3-oxopropane-1-sulfonic acid (Compound 223)

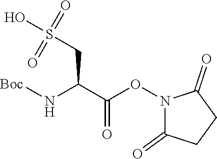 [SMILES: CC(C)(C)OC(=O)N[C@@H](CS(=O)(=O)O)C(=O)ON1C(=O)CCC1=O]

The title compound was prepared according to J. Bing et al., 2008, Bioconjugate Chemistry, 19:201-210.

LC/MS: Calc'd m/z=366.1 for C₁₂H₁₈N₂O₉S, found [M−H]⁻=365.2.

### 3.19 (R)-2-((tert-butoxycarbonyl)amino)-3-(((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-yl)amino)-3-oxopropane-1-sulfonic acid (Compound 224)

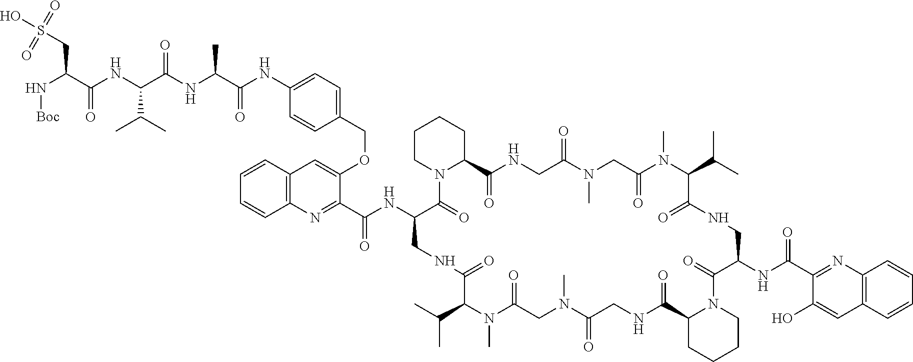 [SMILES: CC(C)[C@H](NC(=O)[C@H](CS(=O)(=O)O)NC(=O)OC(C)(C)C)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

To a solution of Compound 220 (19 mg, 0.0124 mmol) in DMF (500 μL) was added Compound 223 (7 mg, 0.0136 mmol) then NEt(i-Pr)₂ (6 μL, 0.0372 mmol). After 45 minutes the reaction mixture was purified by preparative HPLC eluting with 40 to 55% CH₃CN/H₂O to afford the title compound (14 mg, 0.0085 mmol, 68% yield) as a yellow solid.

LC/MS: Calc'd m/z=1744.8 for C₈₃H₁₁₂N₁₈O₂₂S, found [M+2H]²⁺=874.0.

### 3.20 (R)-2-amino-3-(((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-yl)amino)-3-oxopropane-1-sulfonic acid (Compound 225)

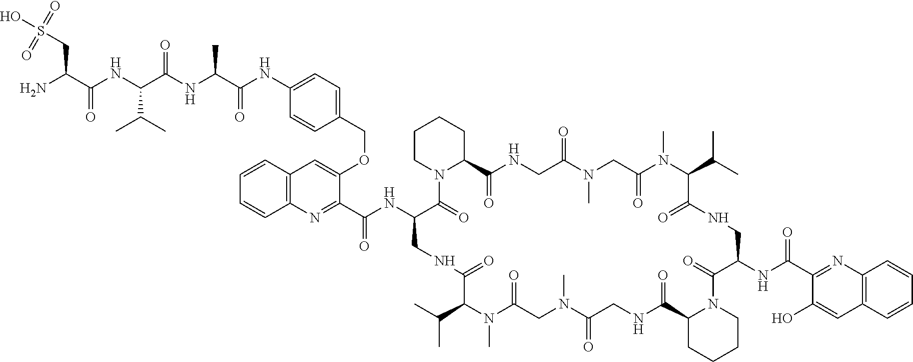 [SMILES: CC(C)[C@H](NC(=O)[C@@H](N)CS(=O)(=O)O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized according to General Procedure 10 starting from Compound 222 (14 mg, 0.0085 mmol).

LC/MS: Calc'd m/z=1644.7 for C₇₈H₁₀₄N₁₈O₂₀S, found [M+2H]²⁺=824.

### 3.21 (R)-1-(2,5-dioxo-2,5-dihydro-1H-pyrrol-1-yl)-14-(((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxopropan-2-yl)amino)-3-methyl-1-oxobutan-2-yl)carbamoyl)-12-oxo-3,6,9-trioxa-13-azapentadecane-15-sulfonic acid (MT-Cya-VA-PAB-35; Compound 226; DL-6)

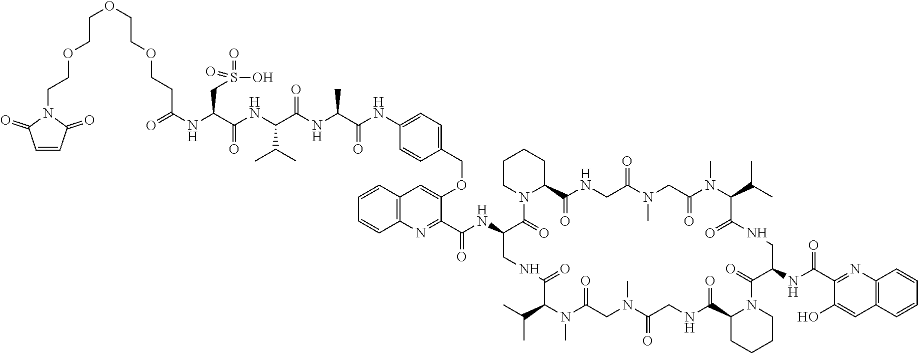 [SMILES: CC(C)[C@H](NC(=O)[C@H](CS(=O)(=O)O)NC(=O)CCOCCOCCOCCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized according to General Procedure 11, starting from Compound 225 (10 mg, 0.0061 mmol). Preparative HPLC purification according to General Procedure 12, eluting with 40 to 50% CH₃CN/H₂O, afforded the title compound (2.0 mg, 0.0010 mmol, 16% yield).

LC/MS: Calc'd m/z=1927.8 for C₉₁H₁₂₁N₁₉O₂₆S, found [M+2H]²⁺=965.6.

### 3.22 (R)-14-((tert-butoxycarbonyl)amino)-1-carboxy-13-oxo-3,6,9-trioxa-12-azapentadecane-15-sulfonate (Compound 227)

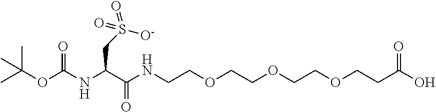 [SMILES: CC(C)(C)OC(=O)N[C@@H](CS(=O)(=O)[O-])C(=O)NCCOCCOCCOCCC(=O)O]

To a 25 mL flask was added 3-{2-[2-(2-aminoethoxy)ethoxy]ethoxy}propanoic acid (0.47 g, 2.14 mmol, 1 equiv.), DMF (4.3 mL, 0.5 M), NEt(i-Pr)₂ (1.12 mL, 6.42 mmol, 3 equiv.), and Compound 223 (1 g, 2.14 mmol, 1 equiv.). This solution was stirred at rt for 1 h, then stored at −20° C. overnight. Purification according to General Procedure 12, using a 60 g C18 column and eluting with 0 to 10% MeCN/H₂O (modified with 0.1% formic acid), afforded the NEt(i-Pr)₂ salt of the title compound (0.90 g, 70% yield) as a colourless oily residue.

LC/MS: Calc'd m/z=471.2 for C₁₇H₃₁N₂O₁₁S, found [M]⁻=471.2. ¹H NMR (300 MHZ, acetone-d₆) δ 8.66 (s, 1H), 7.62-7.33 (m, 1H), 6.65 (s, 1H), 4.40-4.23 (m, 1H), 3.89-3.75 (m, 2H), 3.72 (d, J=6.3 Hz, 2H), 3.64-3.55 (m, 8H), 3.53 (t, J=6.0 Hz, 2H), 3.42-3.33 (m, 2H), 3.33-3.22 (m, 2H), 3.10 (d, J=5.6 Hz, 2H), 2.55 (d, J=6.3 Hz, 2H), 1.50-1.38 (m, 24H).

### 3.23 (R)-1-carboxy-14-(3-(2,5-dioxo-2,5-dihydro-1H-pyrrol-1-yl) propanamido)-13-oxo-3,6,9-trioxa-12-azapentadecane-15-sulfonate (Compound 228)

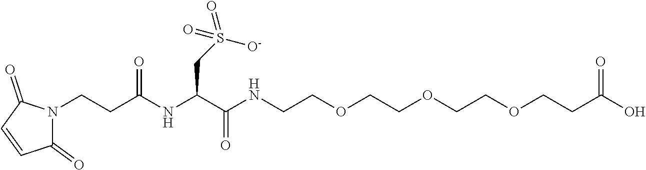 [SMILES: O=C(O)CCOCCOCCOCCNC(=O)[C@H](CS(=O)(=O)[O-])NC(=O)CCN1C(=O)C=CC1=O]

To a 50 mL flask containing Compound 227 (0.9 g, 1.50 mmol, 1 equiv.), at 0° C. was added a 4 M solution of HCl in dioxane (7.5 mL, 0.2 M). The obtained suspension was stirred for 1 h at 0° C. and then allowed to warm up to room temperature and stirred for an additional 3 h. The suspension was then evaporated to dryness and dried under high vacuum. One tenth of the obtained residue (0.15 mmol, 1 equiv.) was introduced into an 2 mL vial, followed by DMF (0.3 mL, 0.5 M), NEt(i-Pr)₂ (0.105 mL, 0.6 mmol, 4 equiv.), and 2,5-dioxopyrrolidin-1-yl 3-(2,5-dioxopyrrol-1-yl) propanoate (39.9 mg, 0.15 mmol, 1 equiv.). This solution was stirred at rt for 2 h, then evaporated to dryness. Purification according to General Procedure 12, using a 12 g C18 column and eluting with 0 to 6% CH₃CN/H₂O, afforded the title product (37.6 mg, 38% yield) as a colorless oily residue.

LC/MS: Calc'd m/z=471.2 for C₁₉H₂₈N₃O₁₂S⁻, found [M]⁻=522.2.

### 3.24 (2S,5S,21R)-21-(3-(2,5-dioxo-2,5-dihydro-1H-pyrrol-1-yl) propanamido)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-5-isopropyl-2-methyl-1,4,7,20-tetraoxo-10,13,16-trioxa-3,6,19-triazadocosane-22-sulfonate (ME-CyaT-VA-PAB-35; Compound 229; DL-5)

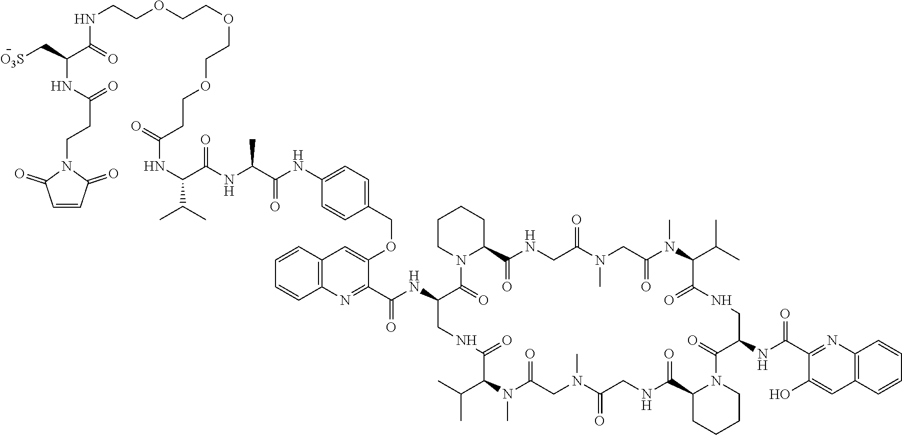

To a 2 mL vial containing Compound 220 (10 mg, 0.007 mmol, 1 equiv.) was added a solution of Compound 228 (4.7 mg, 0.007 mmol, 1.1 equiv.), NEt(i-Pr)₂ (4.57 μL, 0.026 mmol, 4 equiv.) and HATU (2.6 mg, 0.007 mmol, 1.05 equiv.) in DMF (0.327 mL, 0.02 M) at 0° C. This solution was stirred at 0° C. for 1 h, evaporated to dryness, and the residue was purified according to General Procedure 12, using a 12 g C18 column and eluting with 25 to 55% CH₃CN/H₂O, to afford the title compound (4.9 mg, 38% yield) as a yellow solid.

LC/MS: Calc'd m/z=1997.9 for C₉₄H₁₂₅N₂₀O₂₇S⁻, found [M+3H]²⁺=1000.8.

### 3.25 3-((4-((S)-2-((S)-2-(3-(2,5-dioxo-2,5-dihydro-1H-pyrrol-1-yl) propanamido)-3-methylbutanamido) propanamido)benzyl)oxy)-N-((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)quinoline-2-carboxamide (ME-VA-PAB-35; Compound 230; DL-4)

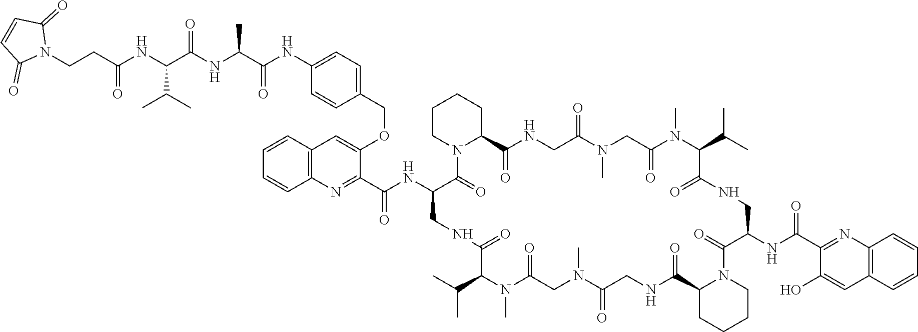 [SMILES: CC(C)[C@H](NC(=O)CCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized according to General Procedure 11, starting from Compound 220 (9 mg, 0.00588 mmol) and utilizing N-succinimidyl 3-maleimidopropionate. Purification according to General Procedure 12, eluting with 40 to 70% CH₃CN/H₂O, afforded the title compound (5 mg, 0.0030 mmol, 52% yield).

LC/MS: Calc'd m/z=1644.8 for C₈₂H₁₀₄N₁₈O₁₉, found [M+2H]²⁺=824.0.

### 3.26 Methyl 3-(((bis(benzyloxy) phosphoryl)oxy) methoxy)quinoline-2-carboxylate (Compound 231)

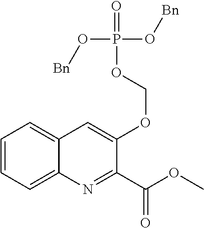 [SMILES: COC(=O)c1nc2ccccc2cc1OCOP(=O)(OCc1ccccc1)OCc1ccccc1]

To a stirred solution of methyl 3-hydroxyquinoline-2-carboxylate (1.00 g, 4.92 mmol) in DMF (10 mL) was added TBAI (181 mg, 0.492 mmol) followed by C₅₂CO₃ (2.40 g, 7.38 mmol). Dibenzyl (chloromethyl)phosphate (1.8 mL, 5.4 mmol) was added drop-wise, and the solution was allowed to stir for 16 h at rt. The reaction mixture was concentrated, then purified as described in General Procedure 12, using a 50 g column and eluting with 10 to 70% EtOAc/hexanes, to afford the title compound (981 mg, 1.99 mmol, 40%).

LC/MS: Calc'd m/z=493.1 for C₂₆H₂₄NO₇P, found [M−H]⁻=494.2

### 3.27 Methyl 3-((phosphonooxy) methoxy)quinoline-2-carboxylate (Compound 232)

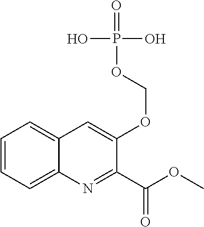 [SMILES: COC(=O)c1nc2ccccc2cc1OCOP(=O)(O)O]

The title compound was synthesized from Compound 231 (418 mg, 0.847 mmol) as described in General Procedure 8. The crude product was used without further purification.

LC/MS: Calc'd m/z=313.0 for C₁₂H₁₂NO₇P, found [M−H]⁻=312.0.

### 3.28 (9H-fluoren-9-yl)methyl (2-(phosphonooxy)ethyl)carbamate (Compound 233)

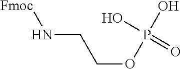

The title compound was prepared according to the procedure described in International Patent Publication No. WO 2011/031865.

LC/MS: Calc'd m/z=363.1 for C₁₇H₁₈NO₆P, found [M−H]⁻=362.2.

### 3.29 Methyl 3-((((((2-((((9H-fluoren-9-yl)methoxy)carbonyl)amino) ethoxy) (hydroxy)phosphoryl)oxy) (hydroxy)phosphoryl)oxy) methoxy)quinoline-2-carboxylate (Compound 234)

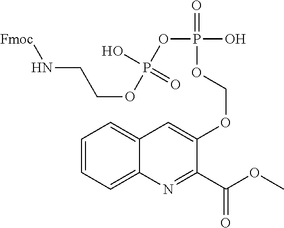

To a stirred solution of Compound 233 (150 mg, 0.413 mmol) was added CDI (160 mg, 0.99 mmol) followed by NEt₃ (0.057 mL, 0.413 mmol). After 30 minutes, a solution of Compound 232 (142 mg, 0.454 mmol) in DMF (2 mL) was added, followed by ZnCl₂ (224 mg, 1.65 mmol). This solution was allowed to stir at rt for 16 h. The reaction mixture was partially concentrated and purified as described in General Procedure 12, using a 30 g C18 column and eluting with 0 to 30% CH₃CN/H₂O+0.1% NH₄OH to provide the title compound (287 mg, 0.436 mmol, 96% yield).

LC/MS: Calc'd m/z=658.1 for C₂₉H₂₈N₂O₁₂P₂, found [M−H]⁻=657.2

### 3.30 3-((((((2-((((9H-fluoren-9-yl)methoxy)carbonyl)amino) ethoxy) (hydroxy)phosphoryl) oxy) (hydroxy)phosphoryl)oxy) methoxy)quinoline-2-carboxylic acid (Compound 235)

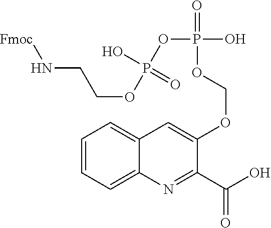

To a stirred solution of Compound 234 (320 mg, 0.486 mmol) in a 5:1 THF: H₂O (10 mL) at 0° C. was added aqueous 1 M LiOH (1.03 mL, 1.03 mmol). This solution was allowed to sit undisturbed in the fridge for 16 h. An additional aliquot of aqueous 1 M LiOH (0.486 mL, 0.486 mmol) was added, and the reaction mixture was kept in the fridge for an additional 4 h. The reaction mixture was quenched with aqueous 0.1% TFA (50 mL) then concentrated via lyophilization. The crude product was purified according to General Procedure 12, using a 30 g C18 column and eluting with 0 to 30% CH₃CN/H₂O+0.1% NH₄OH to provide the title compound (131 mg, 0.203 mmol, 42% yield).

LC/MS: Calc'd m/z=644.1 for C₂₈H₂₆N₂O₁₂P₂, found [M−H]⁻=643.2.

### 3.31 [2-aminoethoxy(hydroxy)phosphoryl]oxy([(2-{[(3R,7S,16S,23R,27S,36S)-23-(3-hydroxyquinoline-2-amido)-7,27-diisopropyl-8,11,28,31-tetramethyl-2,6,9,12,15,22,26,29,32,35-decaoxo-1,5,8,11,14,21,25,28,31,34-decaazatricyclo[34.4.0.0^{16,21}]tetracontan-3-yl]carbamoyl}quinolin-3-yl)oxy]methoxy)phosphinic acid (Compound 236)

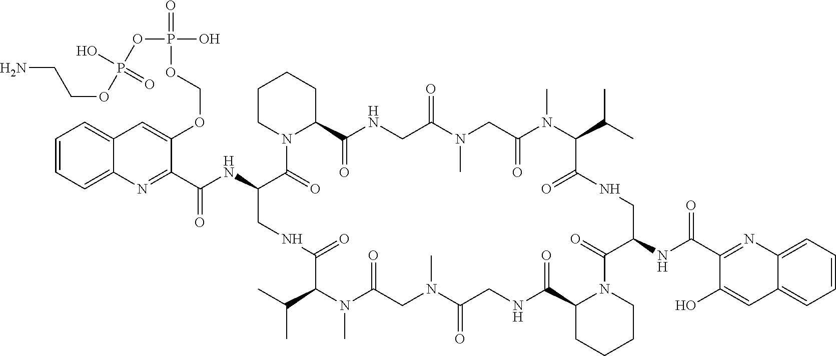 [SMILES: CC(C)[C@H]1C(=O)NC[C@@H](NC(=O)c2nc3ccccc3cc2OCOP(=O)(O)OP(=O)(O)OCCN)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

To a 25 mL rb flask containing Compound 235 (10 mg, 0.0155 mmol) was added a solution of 10% NEt(i-Pr)₂ in MeOH (3 mL). This solution was concentrated to dryness and then this process was repeated a second time. The resulting oil was taken up in DMF (0.5 mL) and to this stirred solution was added 4-(4,6-dimethoxy-1,3,5-triazin-2-yl)-4-methylmorpholinium chloride (4 mg, 0.015 mmol) then NEt(i-Pr)₂ (0.011 mL, 0.063 mmol). After 30 minutes, Compound 216 (16 mg, 0.015 mmol) was added and stirred for an additional 20 min before being quenched with a solution of 10% piperidine in DMF (0.1 mL). After 30 minutes, the reaction mixture was HPLC purified according to General Procedure 12, eluting with a 25 to 45% CH₃CN/H₂O to afford the title compound (9.0 mg, 0.0062 mmol, 40% yield) as a white solid.

LC/MS: Calc'd m/z=1451.6 for C₆₃H₈₇N₁₅O₂₁P₂, found [M+H]⁺=1452.8, [M+2H]²⁺=727.2.

### 3.32 {2-[3-(2-{2-[2-(2,5-dioxopyrrol-1-yl)ethoxy]ethoxy}ethoxy)propanamido]ethoxy (hydroxy)phosphoryl}oxy([(2-{[(3R,7S,16S,23R,27S,36S)-23-(3-hydroxyquinoline-2-amido)-7,27-diisopropyl-8,11,28,31-tetramethyl-2,6,9,12,15,22,26,29,32,35-decaoxo-1,5,8,11,14,21,25,28,31,34-decaazatricyclo[34.4.0.0^{16,21}]tetracontan-3-yl]carbamoyl}quinolin-3-yl)oxy]methoxy)phosphinic acid (MT-PP-35; Compound 237; DL-16)

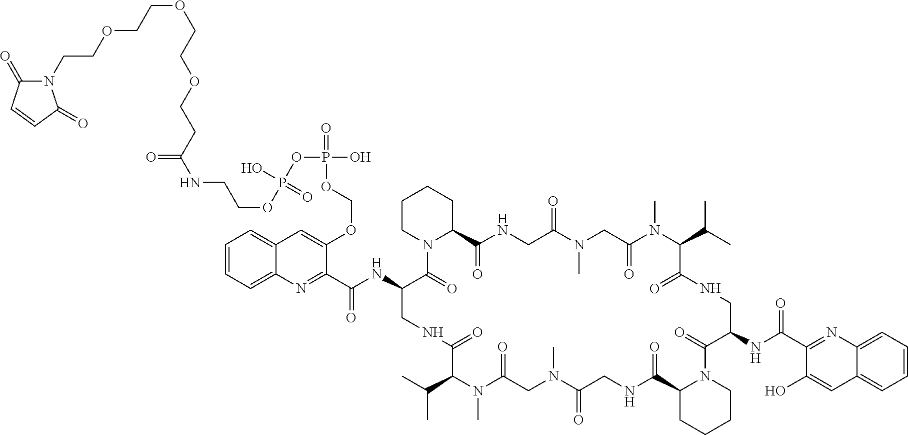 [SMILES: CC(C)[C@H]1C(=O)NC[C@@H](NC(=O)c2nc3ccccc3cc2OCOP(=O)(O)OP(=O)(O)OCCNC(=O)CCOCCOCCOCCN2C(=O)C=CC2=O)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

The title compound was synthesized according to General Procedure 11, starting from Compound 236 (9.0 mg, 0.0062 mmol). Purification according to General Procedure 12, eluting with 30 to 50% CH₃CN/H₂O, afforded the title compound (4.0 mg, 0.0023 mmol, 37% yield) as a white solid.

LC/MS: Calc'd m/z=1734.7 for C₇₆H₁₀₄N₁₆O₂₇P₂, found [M−2H]²⁻=866.8.

### 3.33 Benzyl ((7R,10aS,22aS,29R,32aS,44aS)-29-(3-(benzyloxy)quinoline-2-carboxamido)-18,40-dimethyl-6,10,16,19,22,28,32,38,41,44-decaoxotetratetracontahydrotetrapyrido[1,2-a:1′,2′-j:1″,2″-q:1′″,2′″-z][1,4,7,10,13,17,20,23,26,29]decaazacyclodotriacontin-7-yl)carbamate (Compound 238)

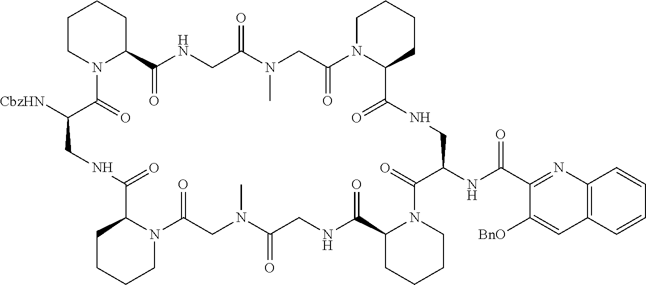 [SMILES: CN1CC(=O)N2CCCC[C@H]2C(=O)NC[C@@H](NC(=O)c2nc3ccccc3cc2OCc2ccccc2)C(=O)N2CCCC[C@H]2C(=O)NCC(=O)N(C)CC(=O)N2CCCC[C@H]2C(=O)NC[C@@H](NC(=O)OCc2ccccc2)C(=O)N2CCCC[C@H]2C(=O)NCC1=O]

A solution of Compound 4 (2.8 mg, 10.1 μmol, 1.5 equiv.), HATU (3.6 mg, 9.4 μmol, 1.4 equiv.), DIPEA (4.69 μL, 27.0 μmol, 4 equiv.) in DMF (447 μL, 0.015 M) was added to a 4 mL vial containing Compound 46 (7.0 mg, 6.7 μmol, 1 equiv.). This solution was stirred at rt for 1 h, then purified according to General Procedure 12, using a 10 g column and eluting with 0 to 10% MeOH/CH₂Cl₂, afforded the title compound (7.1 mg, 83%) as a white solid.

LC/MS: Calc'd m/z=1267.6 for C₆₅H₈₁N₁₃O₁₄, found [M+H]⁺=1268.8, [M+2H]²⁺=635.2.

### 3.34 N-((7R,10aS,22aS,29R,32aS,44aS)-29-amino-18,40-dimethyl-6,10,16,19,22,28,32,38,41,44-decaoxotetratetracontahydrotetrapyrido[1,2-a:1′,2′-j:1″,2″-q:1′″,2′″-z][1,4,7,10,13,17,20,23,26,29]decaazacyclodotriacontin-7-yl)-3-hydroxyquinoline-2-carboxamide (Compound 239)

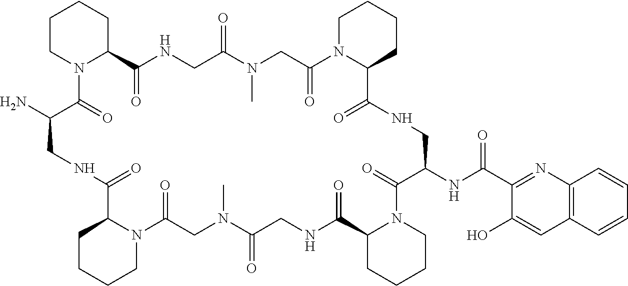 [SMILES: CN1CC(=O)N2CCCC[C@H]2C(=O)NC[C@@H](NC(=O)c2nc3ccccc3cc2O)C(=O)N2CCCC[C@H]2C(=O)NCC(=O)N(C)CC(=O)N2CCCC[C@H]2C(=O)NC[C@@H](N)C(=O)N2CCCC[C@H]2C(=O)NCC1=O]

The title compound was synthesized from Compound 238 (7.1 mg, 5.6 μmol) according to General Procedure 6, and isolated as a bright yellow solid (5.3 mg, 91% yield).

LC/MS: Calc'd m/z=1043.5 for C₅₀H₆₉N₁₃O₁₂ found [M+H]⁺=1044.8, [M+2H]²⁺=523.0.

### 3.35 Tert-butyl ((S)-1-(((S)-1-((4-(((2-(((7R,10aS,22aS,29R,32aS,44aS)-29-(3-hydroxyquinoline-2-carboxamido)-18,40-dimethyl-6,10,16,19,22,28,32,38,41,44-decaoxotetratetracontahydrotetrapyrido[1,2-a:1′,2′-j:1″,2″-q:1′″,2′″-z][1,4,7,10,13,17,20,23,26,29]decaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 240)

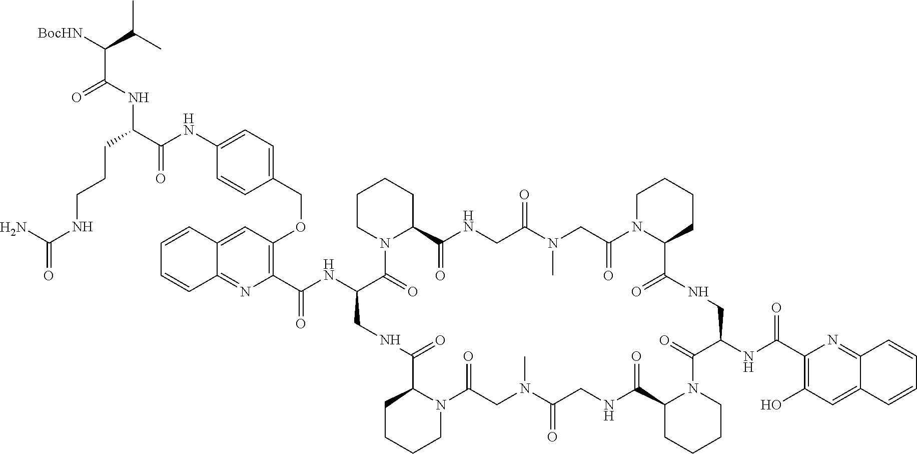 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2CNC(=O)[C@@H]3CCCCN3C(=O)CN(C)C(=O)CNC(=O)[C@@H]3CCCCN3C(=O)[C@H](NC(=O)c3nc4ccccc4cc3O)CNC(=O)[C@@H]3CCCCN3C(=O)CN(C)C(=O)CNC(=O)[C@@H]3CCCCN3C2=O)cc1]

The title compound was synthesized from Compound 239 (5.3 mg, 5.08 μmol, 1.0 equiv.). Purification according to General Procedure 12, using a 10 g column and eluting with 0 to 15% MeOH/CH₂Cl₂, afforded the title compound (9.2 mg) as a yellow solid.

LC/MS: Calc'd m/z=1675.8 for C₈₃H₁₀₉N₁₉O₁₉, found [M+2H]⁺=839.2, [M−Boc,+3H]³⁺=526.6.

### 3.36 3-((4-((S)-2-((S)-2-amino-3-methylbutanamido)-5-ureidopentanamido)benzyl)oxy)-N-((7R,10aS,22aS,29R,32aS,44aS)-29-(3-hydroxyquinoline-2-carboxamido)-18,40-dimethyl-6,10,16,19,22,28,32,38,41,44-decaoxotetratetracontahydrotetrapyrido[1,2-a:1′,2′-j:1″,2″-q:1′″,2′″-z][1,4,7,10,13,17,20,23,26,29]decaazacyclodotriacontin-7-yl)quinoline-2-carboxamide hydrochloride (Compound 241)

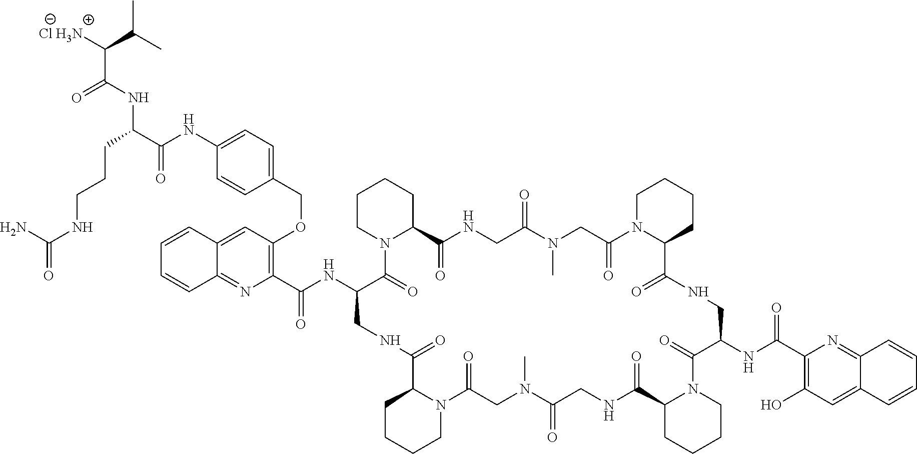

To a 4 mL vial containing Compound 240 (9.2 mg, 5.1 μmol, 1 equiv.), at 0° C. was added a 4M solution of HCl in dioxane (0.51 mL, 0.01 M). This solution was allowed to warm up to rt and was stirred for 1 h. The reaction mixture was evaporated to dryness to afford the title compound (7.9 mg, 96%) as a yellow solid.

LC/MS: Calc'd m/z=1575.8 for C₇₈H₁₀₁N₁₉O₁₇ found [M+2H]⁺=789.4, [M+3H]³⁺=526.6.

### 3.37 3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)-N-((7R,10aS,22aS,29R,32aS,44aS)-29-(3-hydroxyquinoline-2-carboxamido)-18,40-dimethyl-6,10,16,19,22,28,32,38,41,44-decaoxotetratetracontahydrotetrapyrido[1,2-a:1′,2′-j:1″,2″-q:1′″,2′″-z][1,4,7,10,13,17,20,23,26,29]decaazacyclodotriacontin-7-yl)quinoline-2-carboxamide (MT-VC-PAB-49; Compound 242; DL-24)

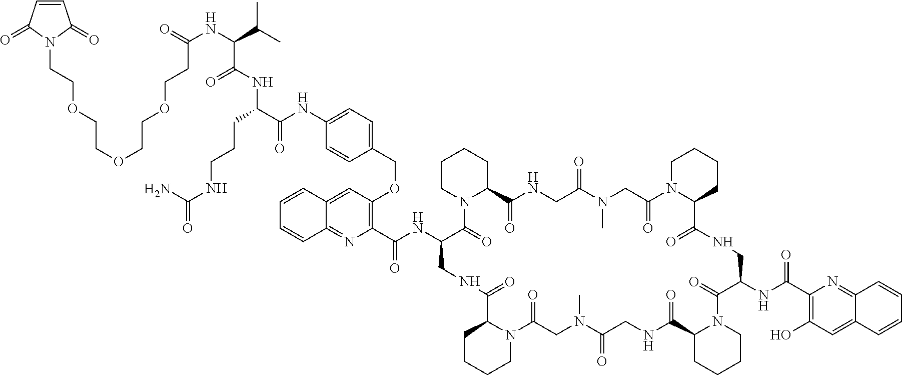 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@@H]3CCCCN3C(=O)CN(C)C(=O)CNC(=O)[C@@H]3CCCCN3C(=O)[C@H](NC(=O)c3nc4ccccc4cc3O)CNC(=O)[C@@H]3CCCCN3C(=O)CN(C)C(=O)CNC(=O)[C@@H]3CCCCN3C2=O)cc1]

The title compound was synthesized from Compound 241 (7.9 mg, 4.9 μmol) according to General Procedure 11. Preparative HPLC purification according to General Procedure 12 afforded the title compound as a bright yellow solid (2.1 mg, 23% yield).

LC/MS: Calc'd m/z=1858.9 for C₉₁H₁₁₈N₂₀O₂₃, found [M+2H]²⁺=931.0, [M+3H]³⁺=621.2.

### 3.38 (S)-1-(O—(N—(N—(((S)-1-((R)-3-((S)-2-(2-(2-amino-N-methylacetamido)-N-methylacetamido)-3-methylbutanamido)-2-(((benzyloxy)carbonyl) amino)propanoyl)piperidine-2-carbonyl)glycyl)-N-methylglycyl)-N-methyl-L-valyl)-N-(tert-butoxycarbonyl)-D-seryl) piperidine-2-carboxylic acid (Compound 243)

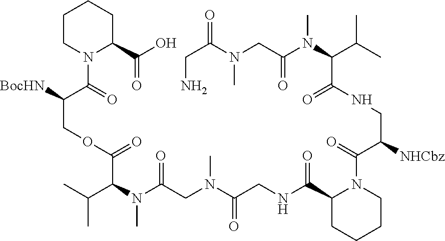 [SMILES: CC(C)[C@@H](C(=O)NC[C@@H](NC(=O)OCc1ccccc1)C(=O)N1CCCC[C@H]1C(=O)NCC(=O)N(C)CC(=O)N(C)[C@H](C(=O)OC[C@@H](NC(=O)OC(C)(C)C)C(=O)N1CCCC[C@H]1C(=O)O)C(C)C)N(C)C(=O)CN(C)C(=O)CN]

The title compound was synthesized according to General Procedures 1 and 2 utilizing Fmoc-Pip-OH, Cbz-D-Dap(Fmoc)-OH, Fmoc-N-methyl-Val-OH, Fmoc-Sar-OH and Fmoc-Gly-OH, Compound 9 and Compound 8, on a 0.4 mmol scale. The crude peptide (320 mg, 0.283 mmol, 71% yield) was isolated, post ether precipitation, as a white powder and was used without further purification.

LC/MS: Calc'd m/z=1129.6 for C₅₃H₈₃N₁₁O₁₆, found [M+H]⁺=1130.8, [M+2H]²⁺=516.0.

### 3.39 Benzyl tert-butyl ((7R,11S,19aS,26R,30S,38aS)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontine-7,26-diyl)dicarbamate (Compound 244)

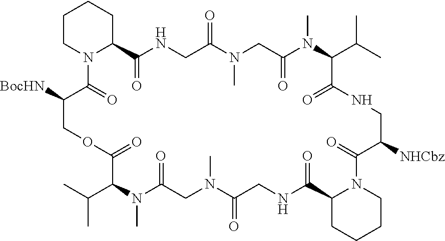 [SMILES: CC(C)[C@H]1C(=O)NC[C@@H](NC(=O)OCc2ccccc2)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized as described in General Procedure 4 starting from Compound 243 (320 mg, 0.283 mmol). Purification according to General Procedure 12, using a 10 g column and eluting with 0 to 20% MeOH/DCM, afforded the title compound (197 mg, 0.177 mmol, 63% yield) as a white solid.

LC/MS: Calc'd m/z=1111.6 for C₅₃H₈₁N₁₁O₁₅, found [M−Boc+2H]²⁺=507.0.

### 3.40 Tert-butyl ((7R,11S,19aS,26R,30S,38aS)-26-amino-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamate (Compound 245)

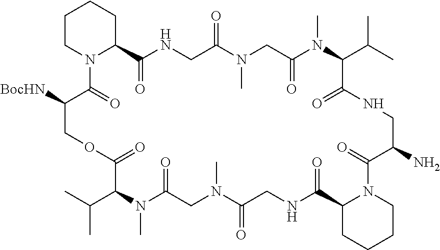 [SMILES: CC(C)[C@H]1C(=O)NC[C@@H](N)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized from Compound 244 (125 mg, 112 μmol) according to General Procedure 6. Following removal of Pd/C, the title compound was obtained as white solid that was used without purification.

LC/MS: Calc'd m/z=977.6 for C₄₅H₇₅N₁₁O₁₃ found [M+H]⁺=978.8, [M−Boc+2H]²⁺=440.0.

### 3.41 Tert-butyl ((7R,11S,19aS,26R,30S,38aS)-26-(3-(benzyloxy)quinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamate (Compound 246)

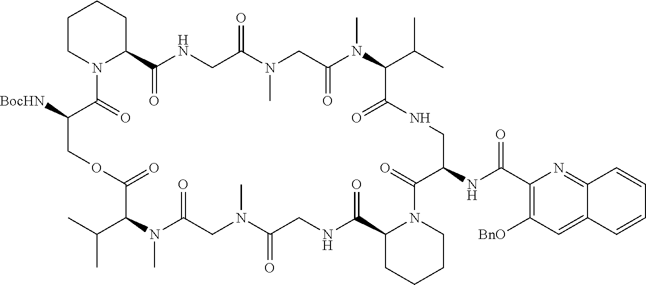 [SMILES: CC(C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was prepared according to General Procedure 7 starting from Compound 245 (0.112 mmol). Purification according to General Procedure 12, using a 10 g column and eluting with 0 to 20% MeOH/CH₂Cl₂, afforded the title compound (120 mg, 0.097 mmol, 87% yield over 2 steps) as a white solid.

LC/MS: Calc'd m/z=1238.6 for C₆₂H₈₆N₁₂O₁₅, found [M−Boc+2H]²⁺=570.6.

### 3.42 Tert-butyl ((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamate (Compound 247)

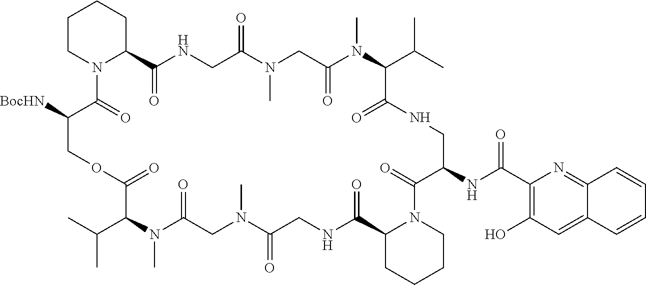 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H](NC(=O)OC(C)(C)C)C(=O)N2CCCC[C@H]2C(=O)NCC(=O)N(C)CC(=O)N1C]

The title compound was synthesized from Compound 246 (120 mg, 97 μmol) according to General Procedure 6. Following removal of Pd/C, the title compound was obtained as white solid that was used without purification.

LC/MS: Calc'd m/z=1148.6 for C₅₅H₈₀N₁₂O₁₅ found [M−Boc+2H]²⁺=525.6.

### 3.43 (7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-aminium chloride (Compound 248)

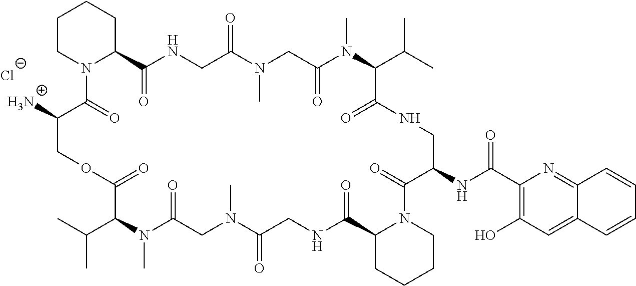 [SMILES: [Cl-] ; CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)OC[C@@H]([NH3+])C(=O)N2CCCC[C@H]2C(=O)NCC(=O)N(C)CC(=O)N1C]

The title compound was prepared according to General Procedure 5 starting from Compound 247 (0.097 mmol), and 2 mL of 4 M HCl/dioxane.

LC/MS: Calc'd m/z=1049.5 for C₅₀H₇₂N₁₂O₁₃, found [M+H]⁺=1050.6, [M+2H]²⁺=525.4.

### 3.44 Tert-butyl ((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 249)

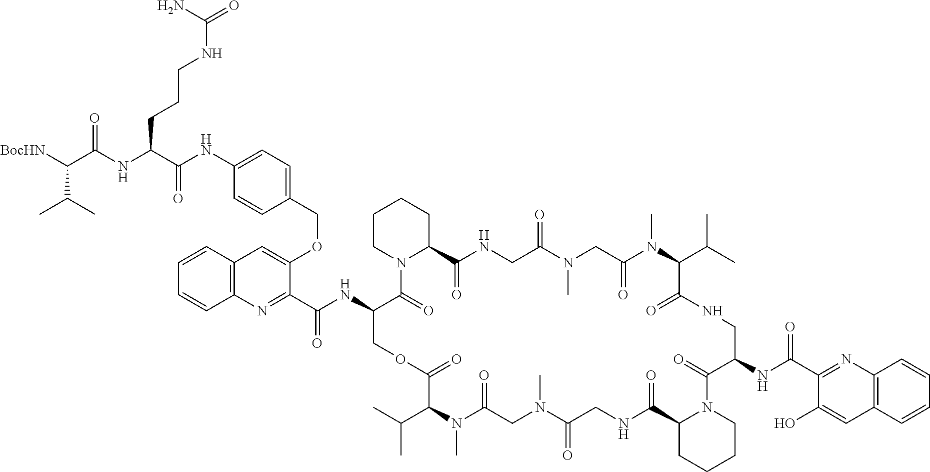 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 248 (0.097 mmol) as described in General Procedure 9. Purification according to General Procedure 12, using a 12 g C18 column and eluting with 40-60% CH₃CN/H₂O, afforded the title compound (120 mg, 0.071 mmol, 73% yield) as a white yellow solid.

LC/MS: Calc'd m/z=1680.8 for C₈₃H₁₁₂N₁₈O₂₀, found [M+2H]²⁺=842.0.

### 3.45 (S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-aminium chloride (Compound 250)

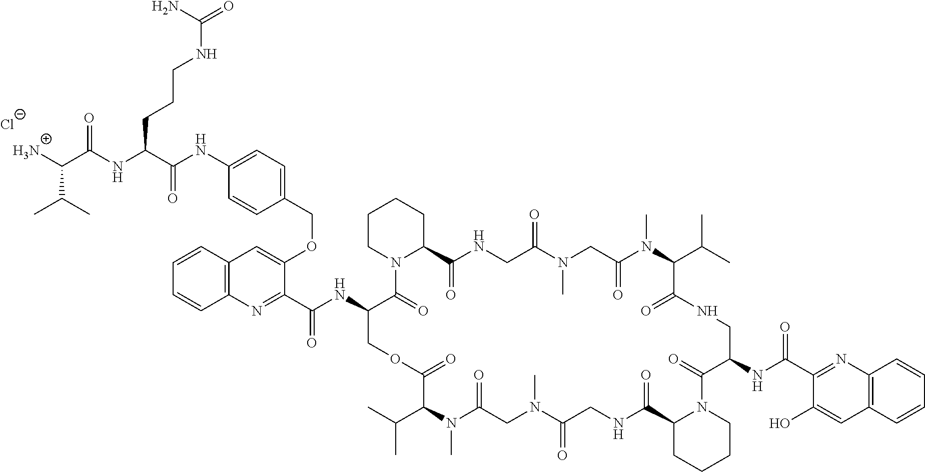 [SMILES: [Cl-] ; CC(C)[C@H]([NH3+])C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 249 (12 mg, 0.007 mmol) as described in General Procedure 10.

LC/MS: Calc. 1581.8 for C₇₈H₁₀₄N₁₈O₁₈, found [M+2H]²⁺=791.8.

### 3.46 3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)-N-((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-7-yl)quinoline-2-carboxamide (MT-VC-PAB-14; Compound 251; DL-12)

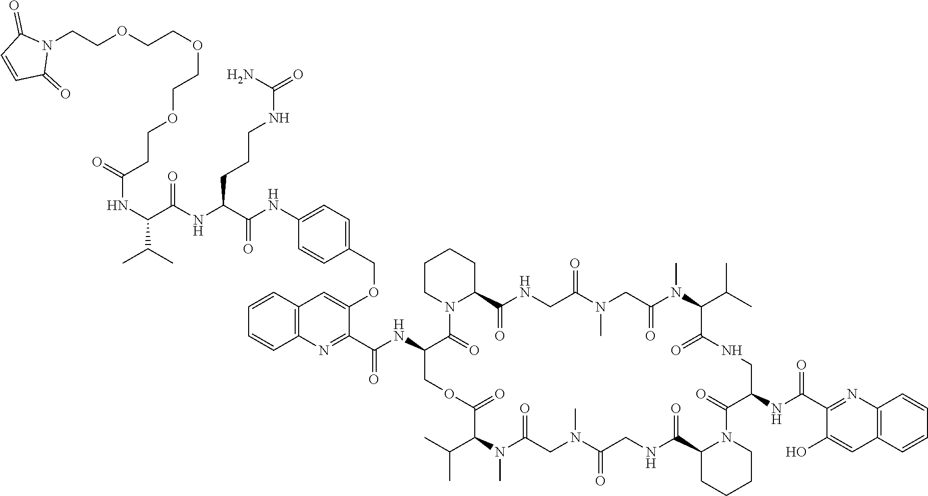 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from crude Compound 250 (0.007 mmol) as described in General Procedure 11. Preparative HPLC purification according to General Procedure 12, eluting with 35-55% CH₃CN/H₂O, afforded the title compound (4.2 mg, 0.002 mmol, 31% yield) as a white yellow solid.

LC/MS: Calc. 1863.9 for C₉₁H₁₂₃N₁₉O₂₃, found [M+H]²⁺=933.6, [M+3H]³⁺=622.8.

### 3.47 4-nitrobenzyl ((7R,11S,19aS,26R,30S,38aS)-26-amino-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamate (Compound 252)

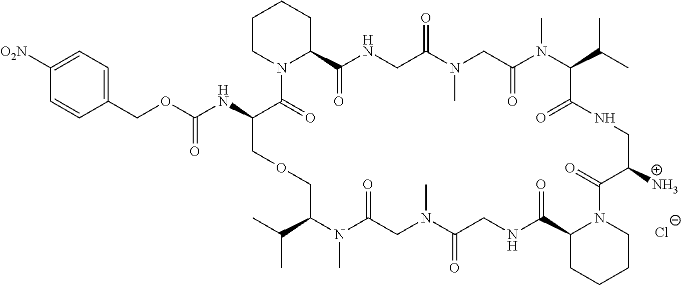 [SMILES: CC(C)[C@H]1COC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)C(=O)NC[C@@H]([NH3+])C(=O)N2CCCC[C@H]2C(=O)NCC(=O)N(C)CC(=O)N1C ; [Cl-]]

The title compound was synthesized as described in General Procedure 5, starting from Compound 174 (93 mg, 0.081 mmol).

LC/MS: Calc'd m/z=1042.5 for C₄₈H₇₄N₁₂O₁₄, found [M+H]⁺=1043.8, [M+2H]²⁺=522.6.

### 3.48 4-nitrobenzyl ((7R,11S,19aS,26R,30S,38aS)-26-(3-(benzyloxy)quinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamate (Compound 253)

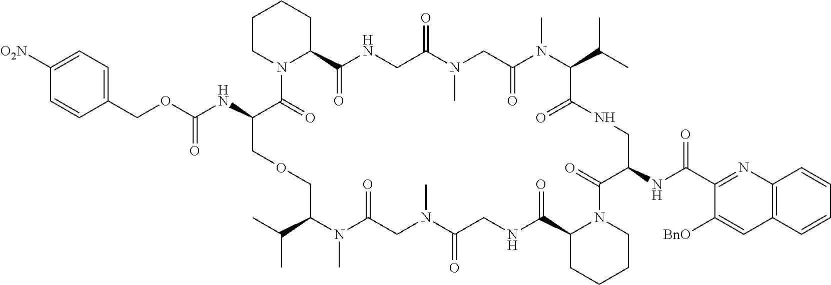 [SMILES: CC(C)[C@H]1COC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2OCc2ccccc2)C(=O)N2CCCC[C@H]2C(=O)NCC(=O)N(C)CC(=O)N1C]

The title compound was prepared from Compound 252 (0.081 mmol) according to General Procedure 7. Purification according to General Procedure 12, using a 10 g column and eluting with 0-20% MeOH/DCM gradient, afforded the title compound (112 mg, 0.086 mmol).

LC/MS: Calc'd m/z=1303.6 for C₆₅H₈₅N₁₃O₁₆, found [M+H]⁺=1304.8, [M+2H]²⁺=653.2.

### 3.49 N-((7R,11S,19aS,26R,30S,38aS)-7-amino-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)-3-hydroxyquinoline-2-carboxamide (Compound 254)

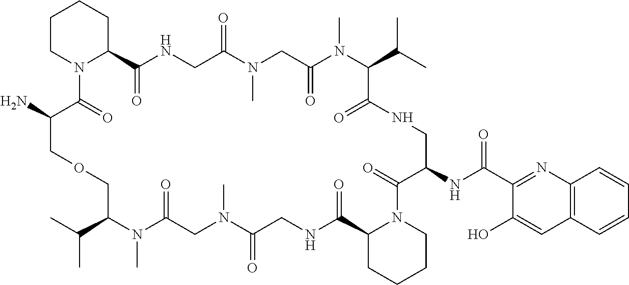 [SMILES: CC(C)[C@H]1COC[C@@H](N)C(=O)N2CCCC[C@H]2C(=O)NCC(=O)N(C)CC(=O)N(C)[C@@H](C(C)C)C(=O)NC[C@@H](NC(=O)c2nc3ccccc3cc2O)C(=O)N2CCCC[C@H]2C(=O)NCC(=O)N(C)CC(=O)N1C]

The title compound was prepared from crude Compound 253 (112 mg 0.086 mmol) according to General Procedure 8. Purification according to General Procedure 12, using a 12 g C18 column and eluting with 30-60% CH₃CN/H₂O, afforded the title compound (35 mg, 0.034 mmol, 39% yield) as a white solid.

LC/MS: Calc's m/z=1034.6 for C₅₀H₇₄N₁₂O₁₂, found [M+H]⁺=1035.8, [M+2H]²⁺=518.6.

### 3.50 Tert-butyl ((S)-1-(((S)-1-((4-(((2-(((7R,11S,19aS,26R,30S,38aS)-26-(3-hydroxyquinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 255)

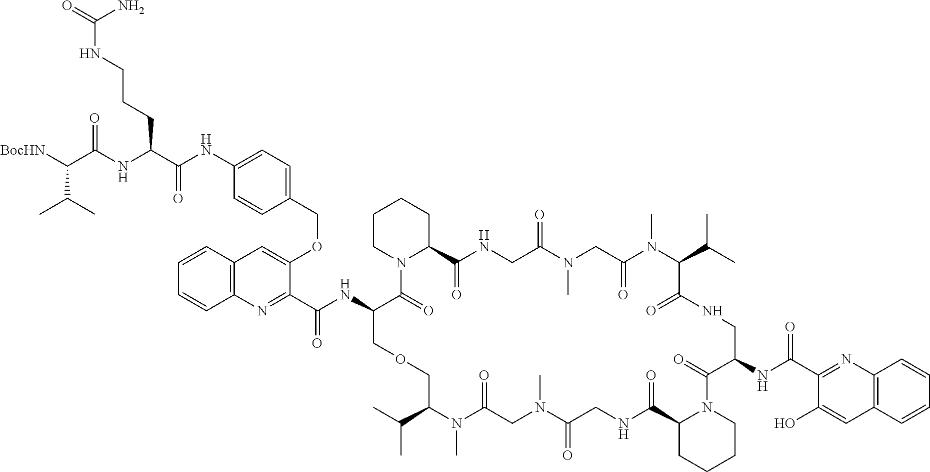 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 254 (27 mg, 0.026 mmol) as described in General Procedure 9. Purification according to General Procedure 12, using a 12 g C18 column and eluting with 40-60% CH₃CN/H₂O, afforded the title compound (20 mg, 0.012 mmol, 46% yield) as a white yellow solid.

LC/MS: Calc'd m/z=1666.8 for C₈₃H₁₁₄N₁₈O₁₉, found [M+2H]²⁺=835.0.

### 3.51 3-((4-((S)-2-((S)-2-amino-3-methylbutanamido)-5-ureidopentanamido)benzyl)oxy)-N-((7R,11S,19aS,26R,30S,38aS)-26-(3-hydroxyquinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-7-yl)quinoline-2-carboxamide (Compound 256)

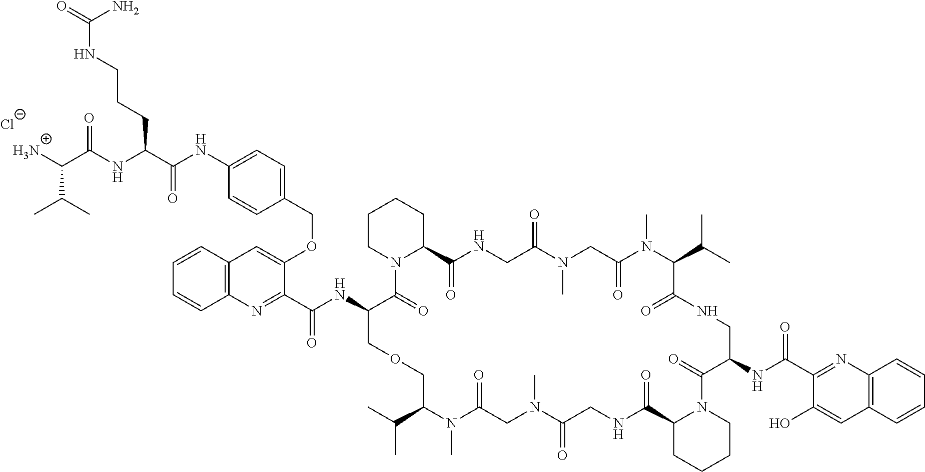 [SMILES: [Cl-] ; CC(C)[C@H]([NH3+])C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from Compound 255 (20 mg, 0.012 mmol) as described in General Procedure 10.

LC/MS: Calc. 1566.8 for C₇₈H₁₀₆N₁₈O₁₇, found [M+2H]²⁺=785.0.

### 3.52 3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)-N-((7R,11S,19aS,26R,30S,38aS)-26-(3-hydroxyquinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-7-yl)quinoline-2-carboxamide (MT-VC-PAB-178; Compound 257; DL-17)

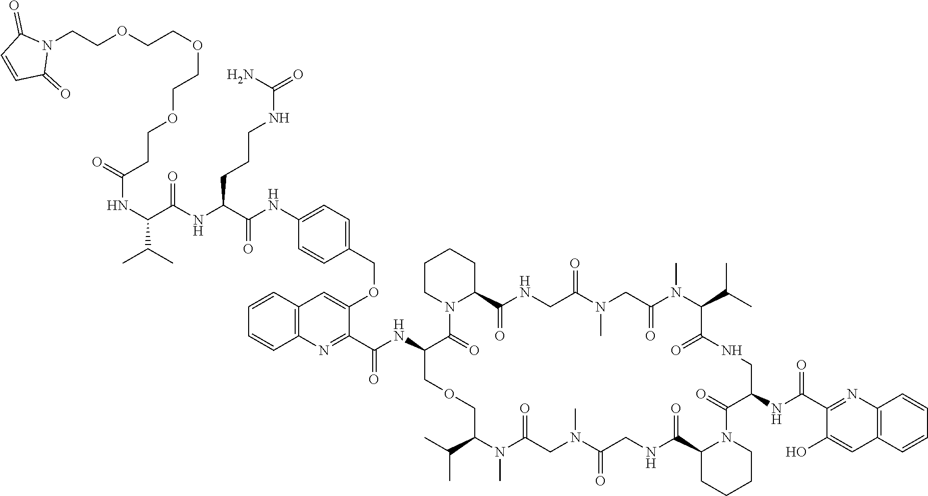 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O)CNC(=O)[C@H](C(C)C)N(C)C(=O)CN(C)C(=O)CNC(=O)[C@@H]3CCCCN3C2=O)cc1]

The title compound was synthesized from crude Compound 256 (0.012 mmol) as described in General Procedure 11. Purification according to General Procedure 12, eluting with 35-55% CH₃CN/H₂O, afforded the title compound (16.5 mg, 0.0089 mmol, 74% yield) as a white yellow solid.

LC/MS: Calc. 1849.9 for C₉₁H₁₂₃N₁₉O₂₃, found [M+H]²⁺=926.6.

### 3.53 Benzyl ((7R,11S,19aS,26R,30S,38aS)-26-amino-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 258)

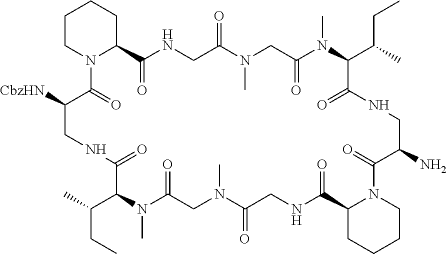 [SMILES: CC[C@H](C)[C@H]1C(=O)NC[C@@H](NC(=O)OCc2ccccc2)C(=O)N2CCCC[C@H]2C(=O)NCC(=O)N(C)CC(=O)N(C)[C@@H]([C@@H](C)CC)C(=O)NC[C@@H](N)C(=O)N2CCCC[C@H]2C(=O)NCC(=O)N(C)CC(=O)N1C]

The title compound was synthesized as described in General Procedure 5, starting from Compound 64 (220 mg, 0.193 mmol).

LC/MS: Calc'd m/z=1038.6 for C₅₀H₇₈N₁₂O₁₂, found [M+H]⁺=1039.8, [M+2H]²⁺=520.6.

### 3.54 Benzyl ((7R,11S,19aS,26R,30S,38aS)-26-(3-(benzyloxy)quinoline-2-carboxamido)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 259)

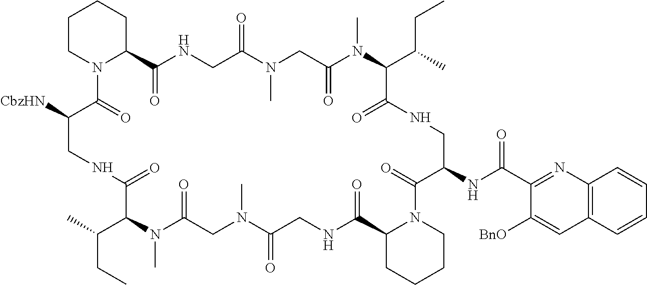 [SMILES: CC[C@H](C)[C@H]1C(=O)NC[C@@H](NC(=O)c2nc3ccccc3cc2OCc2ccccc2)C(=O)N2CCCC[C@H]2C(=O)NCC(=O)N(C)CC(=O)N(C)[C@@H]([C@@H](C)CC)C(=O)NC[C@@H](NC(=O)OCc2ccccc2)C(=O)N2CCCC[C@H]2C(=O)NCC(=O)N(C)CC(=O)N1C]

The title compound was prepared from Compound 258 (28 mg, 0.027 mmol) according to General Procedure 7. Purification according to General Procedure 12, using a 10 g column and eluting with 30-60% CH₃CN/H₂O, afforded the title compound (20 mg, 0.015 mmol, 57%).

LC/MS: Calc'd m/z=1299.67 for C₆₇H₈₉N₁₃O₁₄, found [M+H]⁺=1300.8, [M+2H]²⁺=651.2.

### 3.55 N-((7R,11S,19aS,26R,30S,38aS)-26-amino-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)-3-hydroxyquinoline-2-carboxamide (Compound 260)

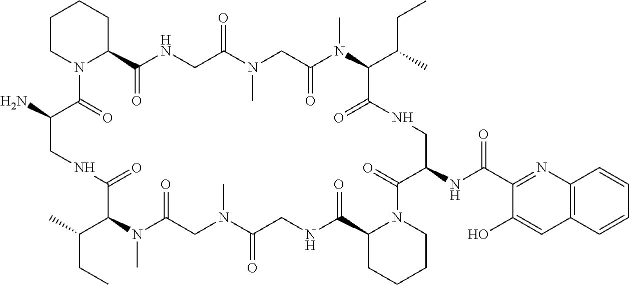 [SMILES: CC[C@H](C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H]([C@@H](C)CC)C(=O)NC[C@@H](N)C(=O)N2CCCC[C@H]2C(=O)NCC(=O)N(C)CC(=O)N1C]

The title compound was prepared from crude Compound 259 (20 mg, 0.015 mmol) according to General Procedure 8.

LC/MS: Calc'd m/z=1075.6 for C₅₂H₇₇N₁₃O₁₂, found [M+H]⁺=1076.8, [M+2H]²⁺=539.0.

### 3.56 Tert-butyl ((S)-1-(((S)-1-((4-(((2-(((7R,11S,19aS,26R,30S,38aS)-11,30-di((S)-sec-butyl)-26-(3-hydroxyquinoline-2-carboxamido)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 261)

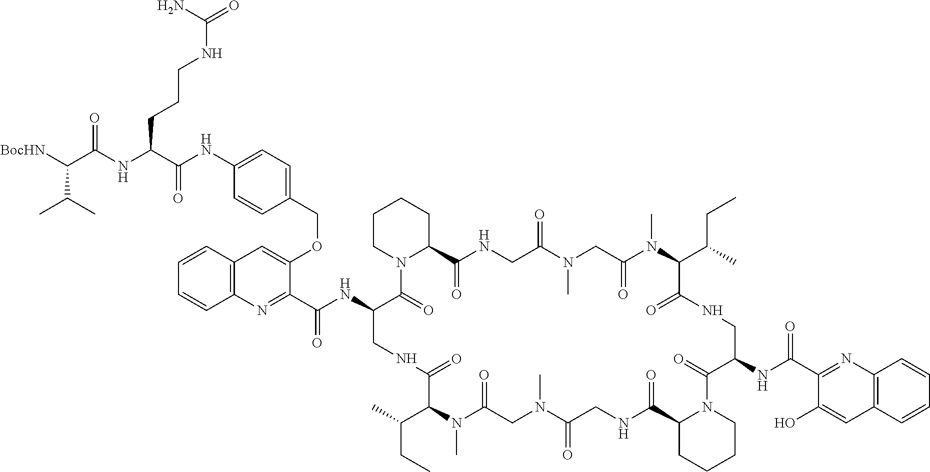 [SMILES: CC[C@H](C)[C@H]1C(=O)NC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Compound 260 (0.0154 mmol) as described in General Procedure 9. Purification according to General Procedure 12, using a 12 g C18 column and eluting with 35-60% CH₃CN/H₂O, afforded the title compound (8 mg, 0.0047 mmol, 30% yield) as a white yellow solid.

LC/MS: Calc'd m/z=1707.9 for C₈₅H₁₁₇N₁₉O₁₉, found [M+2H]²⁺=855.8.

### 3.57 (S)-1-(((S)-1-((4-(((2-(((7R,11S,19aS,26R,30S,38aS)-11,30-di((S)-sec-butyl)-26-(3-hydroxyquinoline-2-carboxamido)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-aminium chloride (Compound 262)

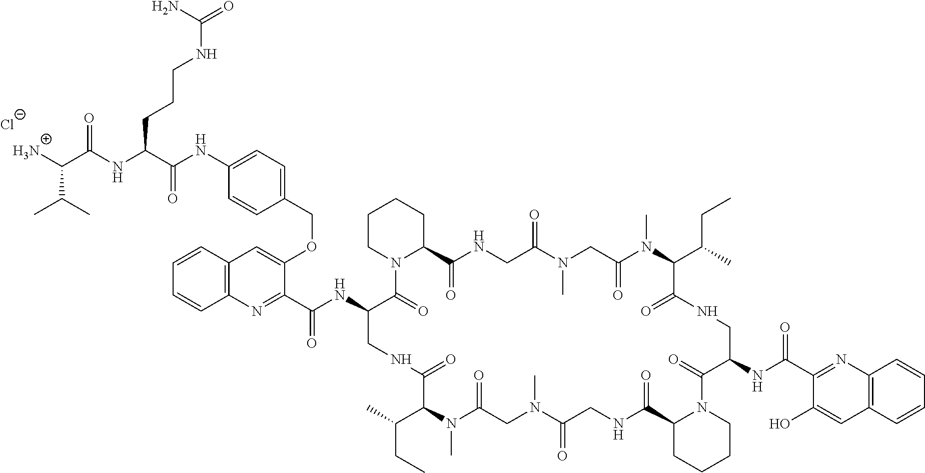 [SMILES: [Cl-] ; CC[C@H](C)[C@H]1C(=O)NC[C@@H](NC(=O)c2nc3ccccc3cc2OCc2ccc(NC(=O)[C@H](CCCNC(N)=O)NC(=O)[C@@H]([NH3+])C(C)C)cc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2O)C(=O)N2CCCC[C@H]2C(=O)NCC(=O)N(C)CC(=O)N1C]

The title compound was synthesized from Compound 261 (8 mg 0.0047 mmol) as described in General Procedure 10.

LC/MS: Calc'd m/z=1607.8 for C₈₀H₁₀₉N₁₉O₁₇, found [M+2H]²⁺=805.4.

### 3.58 N-((7R,11S,19aS,26R,30S,38aS)-11,30-di((S)-sec-butyl)-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)-3-hydroxyquinoline-2-carboxamide (MT-VC-PAB-67; Compound 263; DL-18)

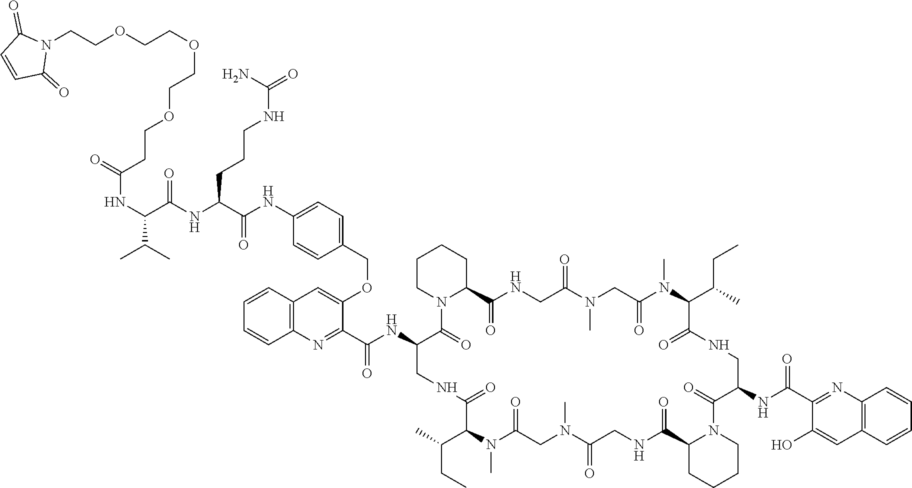 [SMILES: CC[C@H](C)[C@H]1C(=O)NC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2O)C(=O)N2CCCC[C@H]2C(=O)NCC(=O)N(C)CC(=O)N1C]

The title compound was synthesized from crude Compound 262 (0.0047 mmol) as described in General Procedure 11. Purification according to General Procedure 12, eluting with 35-60% CH₃CN/H₂O, afforded the title compound (5.9 mg, 0.0031 mmol, 66% yield) as a white-solid.

LC/MS: Calc'd m/z=1890.9 for C₉₃H₁₂₆N₂₀O₂₃, found [M+2H]²⁺=947.0, [M+3H]³⁺=631.8.

### Example 4: Preparation of Masked Drug-Linkers Comprising Compounds of General Formula (I)

### 4.1 Benzyl 3-hydroxyquinoline-2-carboxylate (Compound 300)

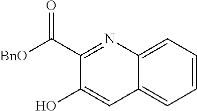 [SMILES: O=C(OCc1ccccc1)c1nc2ccccc2cc1O]

To a solution of methyl 3-hydroxyquinoline-2-carboxylate (430 mg, 2.12 mmol) in benzyl alcohol (5 mL) was added iPr₂NEt (50 μL) and this solution was heated at 70° C. for 1 h. After cooling to RT, the solution was loaded onto a 60 g C18 flash column and eluted with 30 to 60% CH₃CN/H₂O to give the title compound (337 mg, 57% yield) as yellow solid after lyophilization.

LC/MS: Calc'd m/z=279.1 for C₁₇H₁₃NO₃ found [M+H]⁺=280.0.

### 4.2 (2S,3R,4S,5S,6S)-2-((2-((benzyloxy)carbonyl)quinolin-3-yl)oxy)-6-(methoxycarbonyl)tetrahydro-2H-pyran-3,4,5-triyl triacetate (Compound 301)

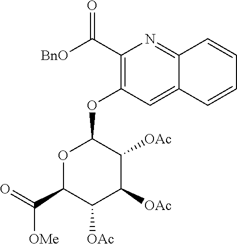 [SMILES: COC(=O)[C@H]1O[C@@H](Oc2cc3ccccc3nc2C(=O)OCc2ccccc2)[C@H](OC(C)=O)[C@@H](OC(C)=O)[C@@H]1OC(C)=O]

To a solution of Compound 300 (1.58 g, 5.66 mmol) in 20 mL dry acetonitrile was added 80 mL of toluene. This solution was concentrated to dryness, then 80 mL dry acetonitrile was added followed by 4 Å molecular sieves. Acetobromo-α-D-glucuronic acid methyl ester (4.5 g, 11 mmol, 2.0 eq.) was added followed by silver (I) oxide (5.2 g, 22.6 mmol, 4.0 eq.), then the flask was covered in aluminum foil and allowed to stir at RT for 16 h. The reaction mixture was filtered over Celite, washed with 80 mL acetonitrile then concentrated to dryness. The crude product was purified using a 30 g C18 flash column and eluting with 30 to 60% CH₃CN/H₂O to provide the title compound (1.20 g, 36%) as a white solid post lyophilization.

LC/MS: Calc'd m/z=595.2 for C₃₀H₂₉NO₁₂ found [M+H]⁺=596.0.

¹H NMR (300 MHz, acetone-d6) δ 8.24 (s, 1H), 8.08-7.99 (m, 1H), 7.96-7.81 (m, 1H), 7.80-7.62 (m, 2H), 7.62-7.54 (m, 2H), 7.51-7.33 (m, 3H), 5.77 (d, J=7.6 Hz, 1H), 5.56-5.51 (m, 1H), 5.48 (d, J=2.9 Hz, 1H), 5.46-5.31 (m, 2H), 5.29 (t, J=9.6 Hz, 1H), 4.73 (d, J=9.8 Hz, 1H), 3.72 (s, 3H), 2.04 (s, 3H), 2.03 (s, 3H).

### 4.3 3-(((2S,3R,4S,5S,6S)-3,4,5-triacetoxy-6-(methoxycarbonyl)tetrahydro-2H-pyran-2-yl)oxy)quinoline-2-carboxylic acid (Compound 302)

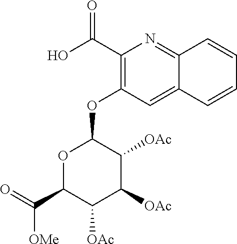 [SMILES: COC(=O)[C@H]1O[C@@H](Oc2cc3ccccc3nc2C(=O)O)[C@H](OC(C)=O)[C@@H](OC(C)=O)[C@@H]1OC(C)=O]

A solution of Compound 301 (450 mg, 0.756 mmol) in 25 mL MeOH was purged with N₂, then 10% Pd/C (75 mg) was added. The flask was purged with H₂, then allowed to stir under 1 atm H₂ for 1.5 h. The reaction mixture was filtered through Celite, washed with 25 mL MeOH then concentrated to dryness. Purification using a 12 g silica column and eluting with 20 to 60% CH₃CN/H₂O provided the title compound (179 mg, 47% yield) as a white solid post lyophilization (47% yield).

LC/MS: Calc'd m/z=505.1 for C₂₃H₂₃NO₁₂ found [M+H]⁺=506.20.

### 4.4 (S)-1-((9S,13R)-1-((S)-1-((R)-3-((S)-2-(2-(2-amino-N-methylacetamido)-N-methylacetamido)-3-methylbutanamido)-2-(((benzyloxy)carbonyl)amino)propanoyl) piperidin-2-yl)-13-((tert-butoxycarbonyl)amino)-9-isopropyl-5,8-dimethyl-1,4,7,10-tetraoxo-2,5,8,11-tetraazatetradecan-14-oyl) piperidine-2-carboxylic acid (Compound 303)

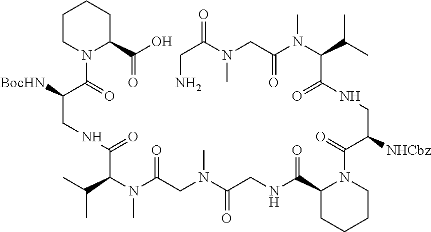 [SMILES: CC(C)[C@@H](C(=O)NC[C@@H](NC(=O)OCc1ccccc1)C(=O)N1CCCC[C@H]1C(=O)NCC(=O)N(C)CC(=O)N(C)[C@H](C(=O)NC[C@@H](NC(=O)OC(C)(C)C)C(=O)N1CCCC[C@H]1C(=O)O)C(C)C)N(C)C(=O)CN(C)C(=O)CN]

The title compound was synthesized according to General Procedure 1 utilizing commercial Fmoc-Pip-OH, Boc-D-Dap(Fmoc)-OH, Z-D-Dap(Fmoc)-OH, Fmoc-NMethyl-Val-OH, Fmoc-Sar-OH and Fmoc Gly-OH, on 1 mM scale. The crude linear peptide (1 g, 80%), post ether precipitation, was used without additional purification.

LC/MS: Calc'd m/z=1128.6 for C₅₃H₈₄N₁₂O₁₅ found [M+H]⁺=1129.8.

### 4.5 Benzyl tert-butyl ((7R,11S,19aS,26R,30S,38aS)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontine-7,26-diyl)dicarbamate (Compound 304)

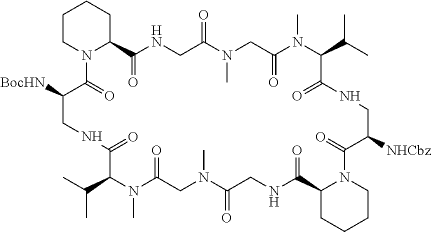 [SMILES: CC(C)[C@H]1C(=O)NC[C@@H](NC(=O)OC(C)(C)C)C(=O)N2CCCC[C@H]2C(=O)NCC(=O)N(C)CC(=O)N(C)[C@@H](C(C)C)C(=O)NC[C@@H](NC(=O)OCc2ccccc2)C(=O)N2CCCC[C@H]2C(=O)NCC(=O)N(C)CC(=O)N1C]

The title compound was synthesized according to General Procedure 4, starting from Compound 303 (950 mg, 841 μmol). Purification was accomplished according to General Procedure 12, using a 10 g silica column and eluting with 0 to 20% MeOH/CH₂Cl₂ to afford the title compound (625 mg, 69%) as a white solid.

LC/MS: Calc'd m/z=1110.6 for C₅₃H₈₂N₁₂O₁₄ found [M+H]⁺=1111.8, [M−Boc+H]⁺=1011.8, [M−Boc+2H]²⁺=505.6.

### 4.6 (2S,3R,4S,5S,6S)-2-((2-(((7R,11S,19aS,26R,30S,38aS)-26-(((benzyloxy)carbonyl)amino)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 305)

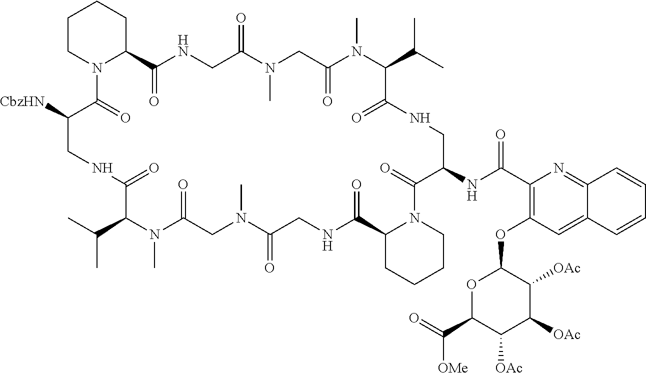 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)OCc3ccccc3)CNC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

Starting from Compound 304 (150 mg, 0.135 mmol), the Boc group was removed according to General Procedure 5. Next Compound 302 (94 mg, 0.186 mmol) was coupled according to General Procedure 9. Purification using a 30 g C18 flash column and eluting with 40 to 55% CH₃CN/H₂O to give the title compound (179 mg, 0.134 mmol, 89% yield) as an off white solid after lyophilization.

LC/MS: Calc'd m/z=1497.6 for C₇₁H₉₅N₁₃O₂₃ found [M+2H]²⁺=750.2.

### 4.7 (2S,3R,4S,5S,6S)-2-((2-(((7R,11S,19aS,26R,30S,38aS)-26-amino-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 306)

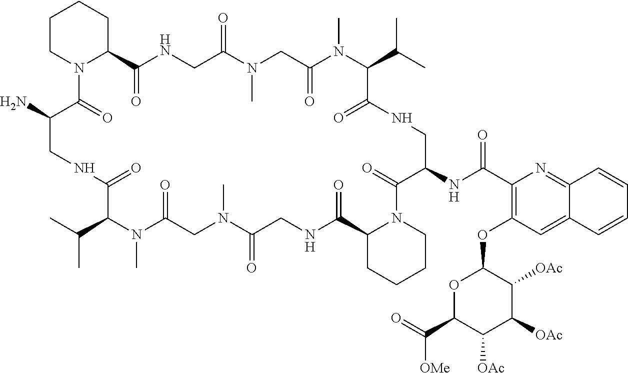 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](N)CNC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

Prepared starting from Compound 305 (179 mg, 0.134 mmol) following General Procedure 6. The crude product (162 mg, 0.119 mmol, 89% yield) was used without additional purification.

LC/MS: Calc'd m/z=1363.6 for C₃₆H₈₉N₁₃O₂₁ found [M+H]⁺=1365.8 and [M+2H]²⁺=683.2.

### 4.8 (2S,3R,4S,5S,6S)-2-((2-(((7R,11S,19aS,26R,30S,38aS)-26-(3-((4-((S)-2-((S)-2-((tert-butoxycarbonyl)amino)-3-methylbutanamido) propanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 307)

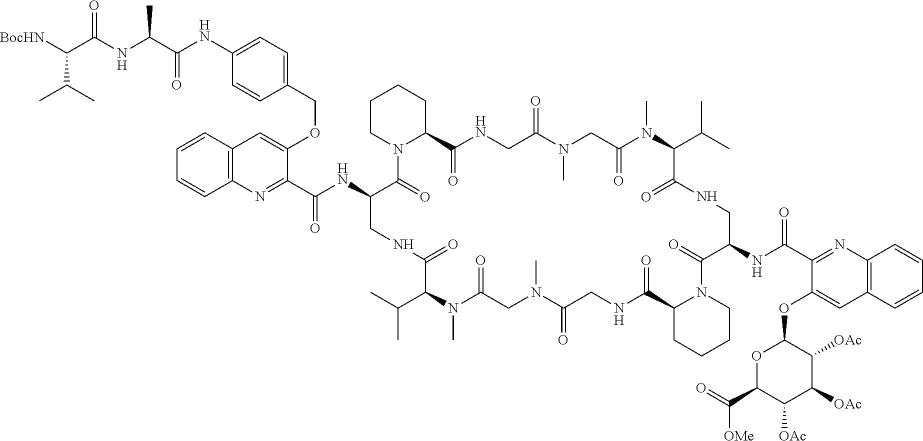 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](C)NC(=O)[C@@H](NC(=O)OC(C)(C)C)C(C)C)cc3)CNC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was synthesized from Compound 306 (27 mg, 0.02 mmol) and Compound 212 following General Procedure 9. Purification according to General Procedure 12, using a 12 g C18 column and eluting with 35-65% CH₃CN/H₂O, afforded the title compound (23 mg, 0.012 mmol, 61% yield) as a pale-yellow solid.

LC/MS: Calc'd m/z=1909.9 for C₉₃H₁₂₃N₁₇O₂₇ found [M+2H]²⁺=956.4.

### 4.9 (2S,3S,4S,5R,6S)-6-((2-(((7R,11S,19aS,26R,30S,38aS)-26-(3-((4-((S)-2-((S)-2-((tert-butoxycarbonyl)amino)-3-methylbutanamido) propanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 308)

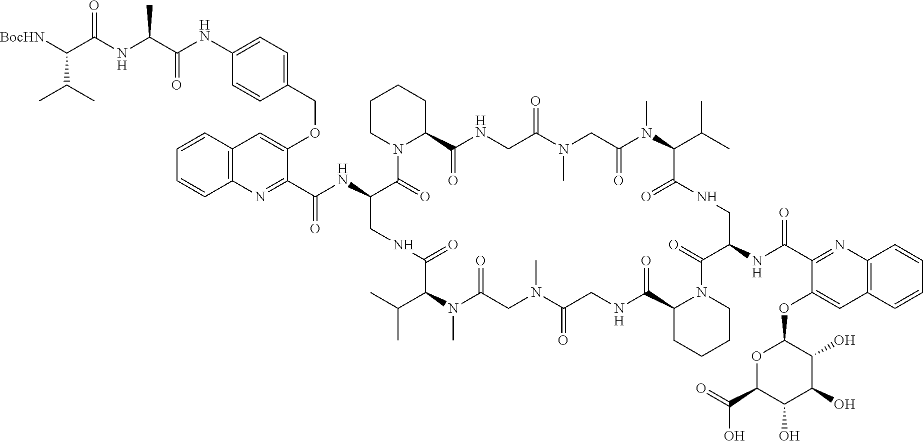 [SMILES: CC(C)[C@H](NC(=O)OC(C)(C)C)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

To a 10 mL round bottomed flask containing a solution of Compound 307 (23 mg, 0.012 mmol) in THF (3 mL) at 0° C. was added a 1M aqueous solution of LiOH (0.144 mL, 0.144 mmol, 12 eq.). The ice bath was removed, and the solution stirred at RT for 3 h then quenched by the addition of a 1M aqueous solution of citric acid (1.15 mL). This mixture was extracted with EtOAc, the organic layer was washed with brine, dried over MgSO₄, and evaporated to dryness to give the title compound (21 mg, 0.012 mmol, 98% yield) as a faint yellow solid that was used with additional purification.

LC/MS: Calc'd m/z=1769.8 for C₈₆H₁₁₅N₁₇O₂₄ found [M+2H]²⁺=886.2.

### 4.10 (2S,3S,4S,5R,6S)-6-((2-(((7R,11S,19aS,26R,30S,38aS)-26-(3-((4-((S)-2-((S)-2-amino-3-methylbutanamido) propanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 309)

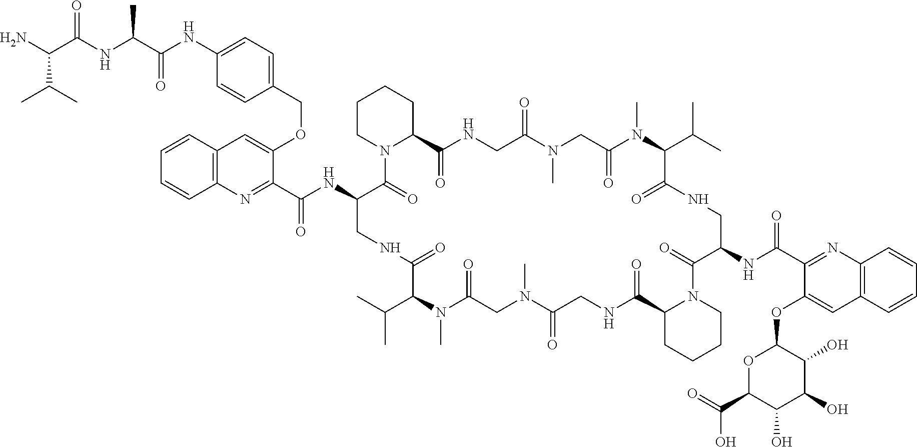 [SMILES: CC(C)[C@H](N)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was synthesized from Compound 308 (21 mg, 0.012 mmol) as described in General Procedure 10.

LC/MS: Calc'd m/z=1669.8 for C₈₁H₁₀₇N₁₇O₂₂ found [M+2H]²⁺=836.4 and [M+3H]³⁺=558.0.

### 4.11 (2S,3S,4S,5R,6S)-6-((2-(((7R,11S,19aS,26R,30S,38aS)-26-(3-((4-((S)-2-((S)-2-(3-(2,5-dioxo-2,5-dihydro-1H-pyrrol-1-yl) propanamido)-3-methylbutanamido) propanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (ME-VA-PAB-35-Glucoronide; Compound 310; DL-9)

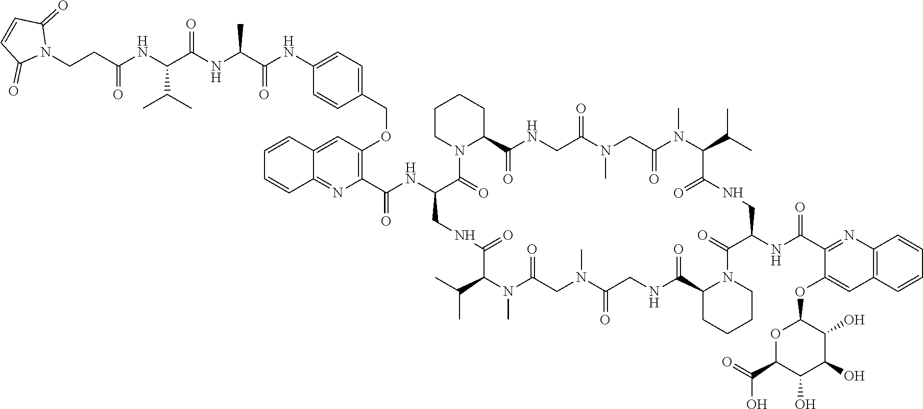 [SMILES: CC(C)[C@H](NC(=O)CCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was synthesized from crude Compound 309 (0.012 mmol) as described in General Procedure 11. Preparative HPLC purification according to General Procedure 12, eluting with 30-55% CH₃CN/H₂O, afforded the title compound (12.5 mg, 0.007 mmol, 56% yield) as a white yellow solid.

LC/MS: Calc'd m/z=1821.9 for C₈₈H₁₁₂N₁₈O₂₅ found [M+2H]²⁺=912.0 and [M+3H]³⁺=608.4.

### 4.12 (2S,3S,4S,5R,6S)-6-((2-(((7R,11S,19aS,26R,30S,38aS)-26-(3-((4-((14S,17S)-1-(2,5-dioxo-2,5-dihydro-1H-pyrrol-1-yl)-14-isopropyl-17-methyl-12,15-dioxo-3,6,9-trioxa-13,16-diazaoctadecan-18-amido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (MT-VA-PAB-35-Glucoronide; Compound 311; DL-8)

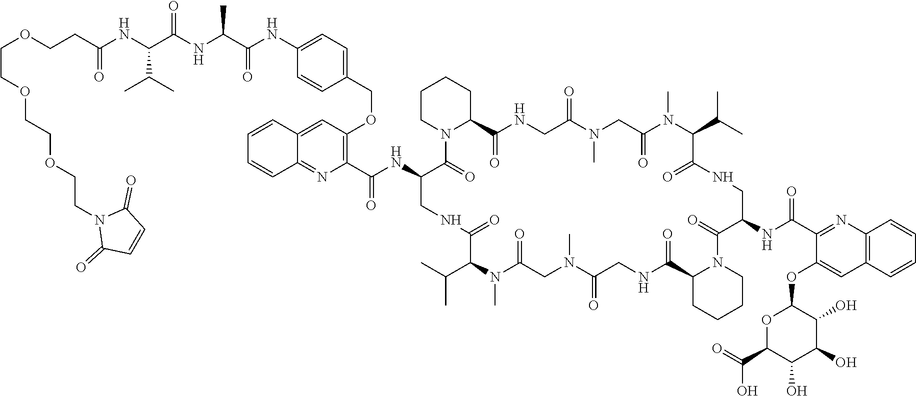 [SMILES: CC(C)[C@H](NC(=O)CCOCCOCCOCCN1C(=O)C=CC1=O)C(=O)N[C@@H](C)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was prepared as described in General Procedure 11. Preparative HPLC purification as described in General Procedure 12, eluting with 30 to 50% CH₃CN/H₂O, afforded the title compound (4.1 mg, 0.002 mmol) as a white solid.

LC/MS: Calc'd m/z=1952.9 for C₉₄H₁₂₄N₁₈O₂₈ found [M+2H]²⁺=978.0 and [M+3H]³⁺=652.4.

### 4.13 (2S,3R,4S,5S,6S)-2-(4-formylphenoxy)-6-(methoxycarbonyl)tetrahydro-2H-pyran-3,4,5-triyl triacetate (Compound 312)

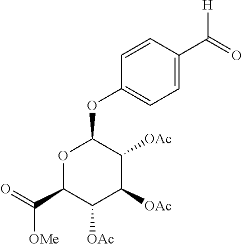 [SMILES: COC(=O)[C@H]1O[C@@H](Oc2ccc(C=O)cc2)[C@H](OC(C)=O)[C@@H](OC(C)=O)[C@@H]1OC(C)=O]

To a solution of 4-hydroxybenzaldehyde (1.54 g, 12.6 mmol, 1.0 eq.) in 100 mL dry acetonitrile was added acetobromo-α-D-glucuronic acid methyl ester (5.0 g, 12.6 mmol, 1.0 eq.) followed by silver (I) oxide (3.58 g, 15.5 mmol, 1.2 eq). The flask was covered in aluminum foil and allowed to stir at RT for 16 h. The reaction mixture was filtered through Celite, washed with 80 mL acetonitrile then the filtrate was concentrated to dryness. Purification using a 50 g silica column and eluting with 20 to 100% hexanes/ethyl acetate provided the title compound (2.85 g, 52% yield) as a white solid after evaporation of solvents and drying under high vacuum.

LC/MS: Calc'd m/z=438.1 for C₂₀H₂₂O₁₁ found [M+Na]⁺=461.2.

¹H NMR (300 MHZ, acetone-d₆) δ 9.97 (s, 1H), 7.93 (d, J=8.8 Hz, 2H), 7.28 (d, J=8.7 Hz, 2H), 5.77 (d, J=7.7 Hz, 1H), 5.56-5.43 (m, 1H), 5.38-5.15 (m, 2H), 4.69 (d, J=9.8 Hz, 1H), 3.70 (s, 3H), 2.04 (s, 3H), 2.03 (s, 3H), 2.02 (s, 3H).

### 4.14 (2S,3R,4S,5S,6S)-2-(4-(bromomethyl) phenoxy)-6-(methoxycarbonyl)tetrahydro-2H-pyran-3,4,5-triyl triacetate (Compound 313)

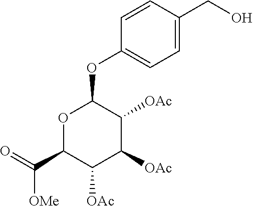 [SMILES: COC(=O)[C@H]1O[C@@H](Oc2ccc(CO)cc2)[C@H](OC(C)=O)[C@@H](OC(C)=O)[C@@H]1OC(C)=O]

A solution of Compound 312 (510 mg, 1.16 mmol, 1 eq) in a 1:1 solution of MeOH/DCM was cooled to 0° C. NaBH₄ (50 mg, 1.26 mmol, 1.1 eq) was added in a single portion and the solution was stirred for 30 min at 0° C., then quenched with saturated aqueous ammonium chloride solution (30 mL). This solution was partially concentrated and extracted with ethyl acetate (2×50 mL). The combined organic layers were dried with sodium sulfate and concentrated to dryness. The crude product was purified using a 10 g silica column eluting with 20 to 100% hexanes/ethyl acetate to give the title compound (291 mg, 57% yield) as a white solid after evaporation of solvents and drying under high vacuum.

LC/MS: Calc'd m/z=440.1 for C₂₀H₂₄O₁₁ found [M+Na]⁺=463.2.

¹H NMR (300 MHZ, acetone-d₆) δ 7.32 (d, J=8.6 Hz, 2H), 7.04 (d, J=8.7 Hz, 2H), 5.54 (d, J=7.8 Hz, 1H), 5.45 (d, J=9.5 Hz, 1H), 5.29-5.17 (m, 2H), 4.58 (s, 2H), 3.71 (s, 3H), 2.04 (s, 3H), 2.01 (s, 3H), 2.01 (s, 3H).

### 4.15 2S,3R,4S,5S,6S)-2-(4-(bromomethyl) phenoxy)-6-(methoxycarbonyl)tetrahydro-2H-pyran-3,4,5-triyl triacetate (SI-19) (Compound 314)

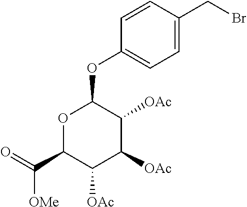 [SMILES: COC(=O)[C@H]1O[C@@H](Oc2ccc(CBr)cc2)[C@H](OC(C)=O)[C@@H](OC(C)=O)[C@@H]1OC(C)=O]

To a solution of Compound 313 (271 mg, 0.615 mmol, 1 eq.) in THF (10 mL) was added triphenyl phosphine (242 mg, 0.923 mmol, 1.5 eq.) followed by NBS (164 mg, 0.923 mmol, 1.5 eq.). This solution was stirred for 20 min at RT, then concentrated to dryness and purified using a 10 g silica column and eluting with 20 to 100% hexanes/ethyl acetate to give the title compound (230 mg, 74% yield) as a white solid post drying on high vacuum.

LC/MS: Calc'd m/z=502.0 for C₂₀H₂₃BrO₁₀ found [M+Na]⁺=525.2.

### 4.16 tert-butyl ((7R,11S,19aS,26R,30S,38aS)-26-(3-hydroxyquinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 315)

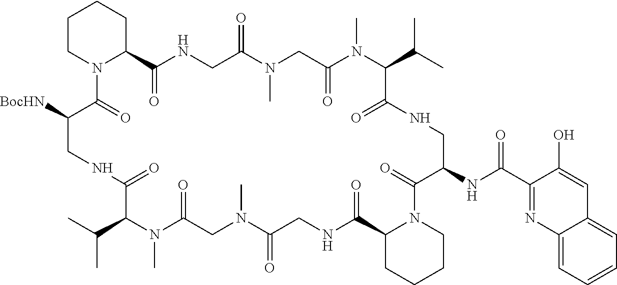 [SMILES: CC(C)[C@H]1C(=O)NC[C@@H](NC(=O)c2nc3ccccc3cc2O)C(=O)N2CCCC[C@H]2C(=O)NCC(=O)N(C)CC(=O)N(C)[C@@H](C(C)C)C(=O)NC[C@@H](NC(=O)OC(C)(C)C)C(=O)N2CCCC[C@H]2C(=O)NCC(=O)N(C)CC(=O)N1C]

The title compound was prepard in 3 steps starting with Compound 304 (100 mg, 0.09 mmol). Cleavage of Cbz protecting group according to General Procedure 6 provided a free amine which was coupled to Compound 4 as decribed in General Procedure 7, then purified as decribed in General Procedure 12 using a 12 g C18 flash column and eluting with 10 to 75% CH₃CN/H₂O. After lyophilization, the yellow solid was taken up in MeOH and the benzyl group removed according to General Procedure 8 to give title compound (80 mg, 77% yield).

LC/MS: Calc'd m/z=1147.6 for C₅₅H₈₁N₁₃O₁₄ found [M+H]⁺=1148.9.

### 4.17 2S,3R,4S,5S,6S)-2-(4-(((2-(((7R,11S,19aS,26R,30S,38aS)-26-((tert-butoxycarbonyl)amino)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl) phenoxy)-6-(methoxycarbonyl)tetrahydro-2H-pyran-3,4,5-triyl (Compound 316)triacetate

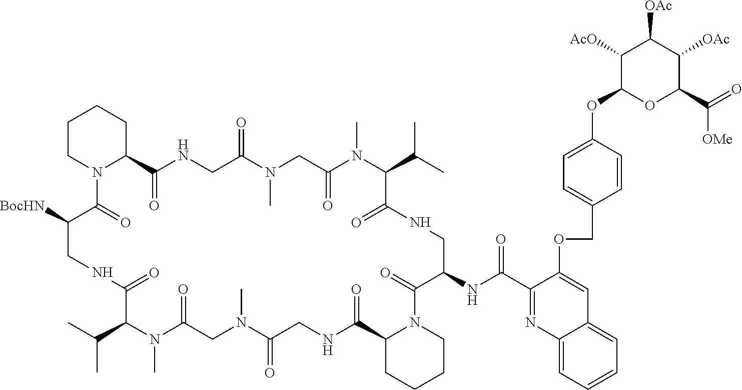 [SMILES: COC(=O)[C@H]1O[C@@H](Oc2ccc(COc3cc4ccccc4nc3C(=O)N[C@@H]3CNC(=O)[C@H](C(C)C)N(C)C(=O)CN(C)C(=O)CNC(=O)[C@@H]4CCCCN4C(=O)[C@H](NC(=O)OC(C)(C)C)CNC(=O)[C@H](C(C)C)N(C)C(=O)CN(C)C(=O)CNC(=O)[C@@H]4CCCCN4C3=O)cc2)[C@H](OC(C)=O)[C@@H](OC(C)=O)[C@@H]1OC(C)=O]

To a solution of Compound 315 (50 mg, 0.0435 mmol) in dry acetonitrile (2 mL) was added cesium carbonate (21 mg, 0.065 mmol), followed by Compound 314 (33 mg, 0.065 mmol). This solution was stirred for 6 h at RT, then the reaction was quenched with an aqueous pH 5 phosphate buffer (1 mL). Preparative HPLC as decribed in General Procedure 12 eluting with a 10-70% gradient (CH₃CN/H₂O+0.1% TFA) afforded the title compound (33 mg, 48% yield) as a white solid post lyophilization.

LC/MS: Calc'd m/z=1569.7 for C₇₅H₁₀₃N₁₃O₂₄ found [M−Boc, +2H]²⁺=736.2.

### 4.18 (2S,3R,4S,5S,6S)-2-(4-(((2-(((7R,11S,19aS,26R,30S,38aS)-26-(3-((4-((S)-2-((S)-2-((tert- butoxycarbonyl)amino)-3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl) phenoxy)-6-(methoxycarbonyl)tetrahydro-2H-pyran-3,4,5-triyl triacetate (Compound 317)

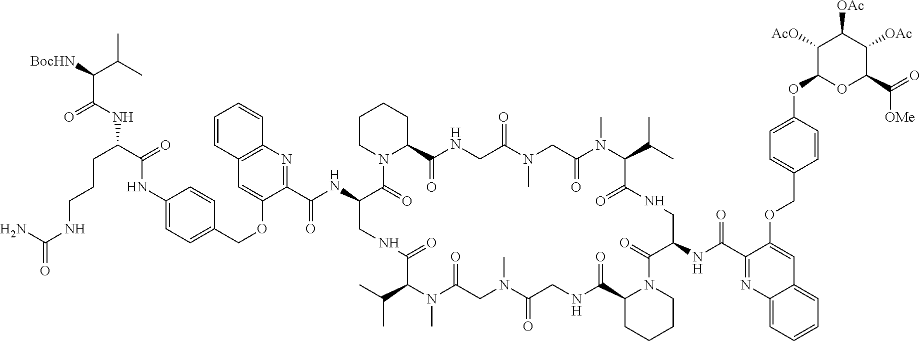 [SMILES: COC(=O)[C@H]1O[C@@H](Oc2ccc(COc3cc4ccccc4nc3C(=O)N[C@@H]3CNC(=O)[C@H](C(C)C)N(C)C(=O)CN(C)C(=O)CNC(=O)[C@@H]4CCCCN4C(=O)[C@H](NC(=O)c4nc5ccccc5cc4OCc4ccc(NC(=O)[C@H](CCCNC(N)=O)NC(=O)[C@@H](NC(=O)OC(C)(C)C)C(C)C)cc4)CNC(=O)[C@H](C(C)C)N(C)C(=O)CN(C)C(=O)CNC(=O)[C@@H]4CCCCN4C3=O)cc2)[C@H](OC(C)=O)[C@@H](OC(C)=O)[C@@H]1OC(C)=O]

A solution of Compound 316 (32 mg, 0.0203 mmol) in DCM (2 mL) was cooled to 0° C., TFA (0.4 mL) was added, and this solution was stirred at 0° C. for 2 h. After concentrating to dryness, the residue was taken up in toluene and evaporated to dryness once again. The resulting solid was taken up in DMF (1 mL) and Compound 209 (16 mg, 0.024 mmol) was coupled as described in General Procedure 9. Preparative HPLC purification eluting with 20 to 70% CH₃CN/H₂O afforded the title compound (21 mg, 49%) as a white solid post lyophilization.

LC/MS: Calc'd m/z=2101.97 for C₁₀₃H₁₃₅N₁₉O₂₉ found [M+2H]²⁺=1052.1.

### 4.19 2S,3S,4S,5R,6S)-6-(4-(((2-(((7R,11S,19aS,26R,30S,38aS)-26-(3-((4-((S)-2-((S)-2-((tert- butoxycarbonyl)amino)-3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl) phenoxy)-3,4,5-trihydroxytetrahydro-2H-pyran-2-carboxylic acid (Compound 318)

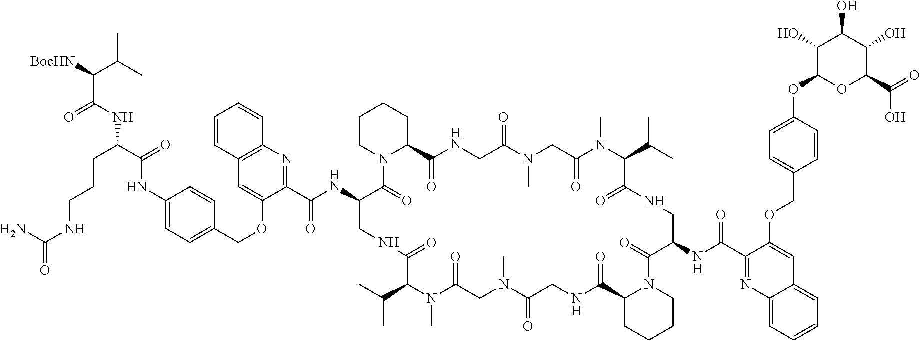 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(O[C@@H]4O[C@H](C(=O)O)[C@@H](O)[C@H](O)[C@H]4O)cc3)CNC(=O)[C@H](C(C)C)N(C)C(=O)CN(C)C(=O)CNC(=O)[C@@H]3CCCCN3C2=O)cc1]

To a solution of Compound 317 (20 mg, 0.0095 mmol) in THF (1 mL) was added an aqueous 1 M solution of LiOH (57 μL, 6 eq.). This solution was stirred for 4 hours, quenched by the addition of an aqueous 0.1% v/v solution of TFA (3 mL), then purified using a 12 g C18 flash column and eluting with a 20 to 60% CH₃CN/H₂O to give the title compound (13 mg, 70% yield) as a white solid post lyophilization.

LC/MS: Calc'd m/z=1961.9 for C₉₆H₁₂₇N₁₉O₂₆ found [M−Boc, +2H]²⁺=932.2.

### 4.20 (2S,3S,4S,5R,6S)-6-(4-(((2-(((7R,11S,19aS,26R,30S,38aS)-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl) phenoxy)-3,4,5-trihydroxytetrahydro-2H-pyran-2-carboxylic acid (MT-VC-PAB-35-Glucoronide; Compound 319; DL-10)

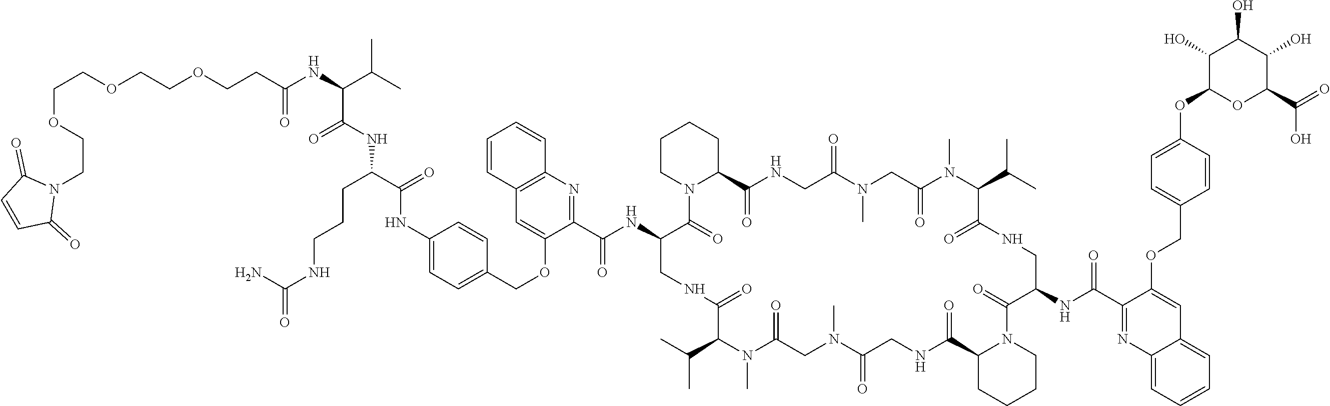 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(O[C@@H]4O[C@H](C(=O)O)[C@@H](O)[C@H](O)[C@H]4O)cc3)CNC(=O)[C@H](C(C)C)N(C)C(=O)CN(C)C(=O)CNC(=O)[C@@H]3CCCCN3C2=O)cc1]

A solution of Compound 318 (13 mg, 0.0066 mmol) in DCM (1 mL) was cooled to 0° C., TFA (0.2 mL) was added, and this solution was stirred at 0° C. for 2 h. After concentrating to dryness, the residue was taken up in toluene and evaporated to dryness once again. The solid was taken up in DMF (0.5 mL) and CH₃CN (0.5 mL) then MT-OTFP (4 mg, 0.008 mmol) was added followed by iPr₂NEt (0.004 mL). This solution was stirred at RT for 3 h, then purified by preparative HPLC and eluting with 10 to 60% CH₃CN/H₂O to give the title compound (8.2 mg, 66% yield) as a white solid post lyophilization.

HRMS: Calc'd m/z=2144.9731 for C₁₀₄H₁₃₆N₂₀O₃₀ found [M+H]⁺=2145.9845.

### 4.21 Benzyl ((7R,11S,19aS,26R,30S,38aS)-26-(3-((((((2-((((9H-fluoren-9-yl)methoxy)carbonyl)amino) ethoxy) (hydroxy)phosphoryl)oxy) (hydroxy)phosphoryl)oxy) methoxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 320)

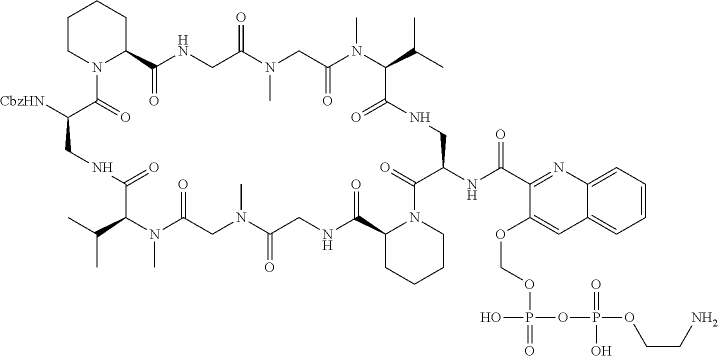 [SMILES: CC(C)[C@H]1C(=O)NC[C@@H](NC(=O)c2nc3ccccc3cc2OCOP(=O)(O)OP(=O)(O)OCCN)C(=O)N2CCCC[C@H]2C(=O)NCC(=O)N(C)CC(=O)N(C)[C@@H](C(C)C)C(=O)NC[C@@H](NC(=O)OCc2ccccc2)C(=O)N2CCCC[C@H]2C(=O)NCC(=O)N(C)CC(=O)N1C]

Compound 235 (38 mg, 0.059 mmol) was taken up in MeOH (5 mL) and iPr₂NEt (0.5 mL), then evaporated to dryness. The solid was dissolved in DMF (1 mL), and iPr₂NEt (0.035 mL, 0.198 mmol) was added followed by DMTMM (15 mg, 0.054 mmol). The solution was stirred at RT for 30 min, then added to a stirring solution of Compound 212 (50 mg, 0.049 mmol) in DMF (0.5 mL) and the combined solution was stirred at RT for 2 h. A solution of 10% piperidine in DMF (1 mL) was added, stirred for 30 min, then purified by preparative HPLC eluting with 25 to 45% CH₃CN/H₂O to give the title compound (28 mg, 0.02 mmol, 40% yield) as a yellowing solid post lyophilization.

LC/MS: Calc'd m/z=1414.6 for C₆₁H₈₈N₁₄O₂₁P₂ found [M+2H]²⁺=708.6.

### 4.22 Benzyl ((7R,11S,19aS,26R,30S,38aS)-26-(3-((((((2-acetamidoethoxy)(hydroxy)phosphoryl)oxy)(hydroxy)phosphoryl)oxy) methoxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamate (Compound 321)

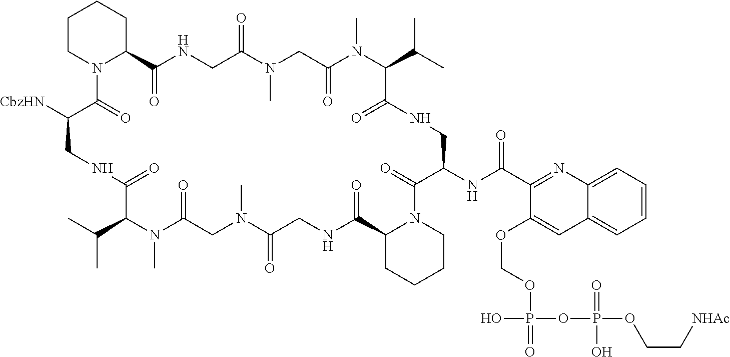 [SMILES: CC(=O)NCCOP(=O)(O)OP(=O)(O)OCOc1cc2ccccc2nc1C(=O)N[C@@H]1CNC(=O)[C@H](C(C)C)N(C)C(=O)CN(C)C(=O)CNC(=O)[C@@H]2CCCCN2C(=O)[C@H](NC(=O)OCc2ccccc2)CNC(=O)[C@H](C(C)C)N(C)C(=O)CN(C)C(=O)CNC(=O)[C@@H]2CCCCN2C1=O]

To a stirring solution of Compound 320 (28 mg, 0.02 mmol) in DMF (0.5 mL) was added iPr₂NEt (0.01 mL, 0.059 mmol), DMAP (0.2 mg, ˜0.1 eq.) and acetic anhydride (0.002 ml, 0.02 mmol). This solution was stirred at RT for 30 min, then purified by preparative HPLC eluting with 20 to 45% CH₃CN/H₂O to give the title compound (24 mg, 0.016 mmol, 83% yield) as a yellow solid post lyophilization.

LC/MS: Calc'd m/z=1455.6 for C₆₃H₉₀N₁₄O₂₂P₂ found [M+2H]²⁺=727.8.

### 4.23 Tert-butyl ((2S)-1-(((2S)-1-((4-(((2-(((7R,11S,19aS,26R,30S,38aS)-26-(3-((((((2-acetamidoethoxy) (hydroxy)phosphoryl)oxy) (hydroxy)phosphoryl)oxy) methoxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)methyl)phenyl)amino)-1-oxo-5-ureidopentan-2-yl)amino)-3-methyl-1-oxobutan-2-yl)carbamate (Compound 322)

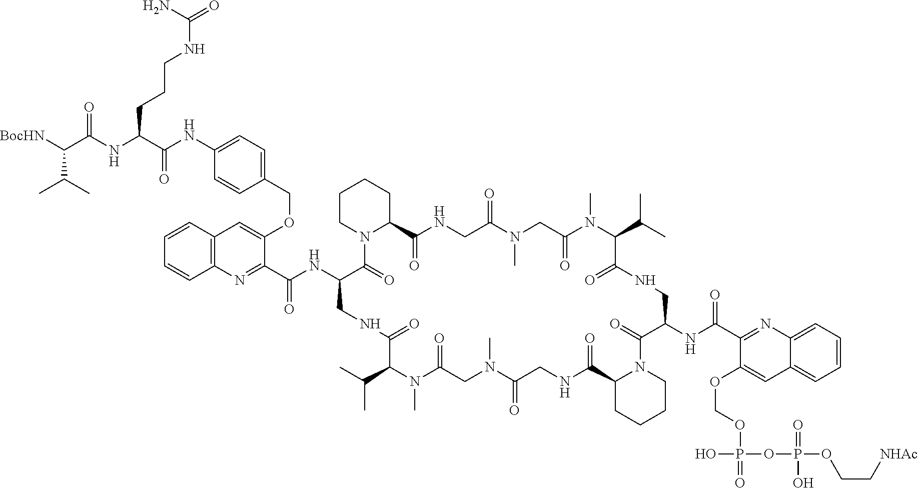 [SMILES: CC(=O)NCCOP(=O)(O)OP(=O)(O)OCOc1cc2ccccc2nc1C(=O)N[C@@H]1CNC(=O)[C@H](C(C)C)N(C)C(=O)CN(C)C(=O)CNC(=O)[C@@H]2CCCCN2C(=O)[C@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)CNC(=O)[C@H](C(C)C)N(C)C(=O)CN(C)C(=O)CNC(=O)[C@@H]2CCCCN2C1=O]

Starting from Compound 321 (24 mg, 0.016 mmol), the Cbz protecting group was removed as described in General Procedure 6 with the addition of NaHCO₃ (4 mg) to the reaction solution. The obtained white solid was dissolved in DMF (1 mL) and Compound 209 (13 mg, 0.021 mmol) was coupled as described in General Procedure 9. Preparative HPLC purification eluting with 20 to 47% CH₃CN/H₂O provided the title compound (11 mg, 0.006 mmol, 35% yield) as a white solid post lyophilization.

LC/MS: Calc'd m/z=1954.8 for C₈₈H₁₂₄N₂₀O₂₇P₂ found [M−2H]²⁻=976.8.

### 4.23 [(2-{[(3R,7S,16S,23R,27S,36S)-23-[3-({4-[(2S)-5-(carbamoylamino)-2-[(2S)-2-[3-(2-{2- [2-(2,5-dioxopyrrol-1-yl)ethoxy]ethoxy}ethoxy)propanamido]-3-methylbutanamido]pentanamido]phenyl}methoxy)quinoline-2-amido]-7,27-diisopropyl-8,11,28,31-tetramethyl-2,6,9,12,15,22,26,29,32,35-decaoxo-1,5,8,11,14,21,25,28,31,34-decaazatricyclo[34.4.0.0^{16,21}]tetracontan-3-yl]carbamoyl}quinolin-3-yl)oxy]methoxy ([2-acetamidoethoxy(hydroxy)phosphoryl]oxy)phosphinic acid (MT-VC-PAB-35-pyrophosphate; Compound 323; DL-11)

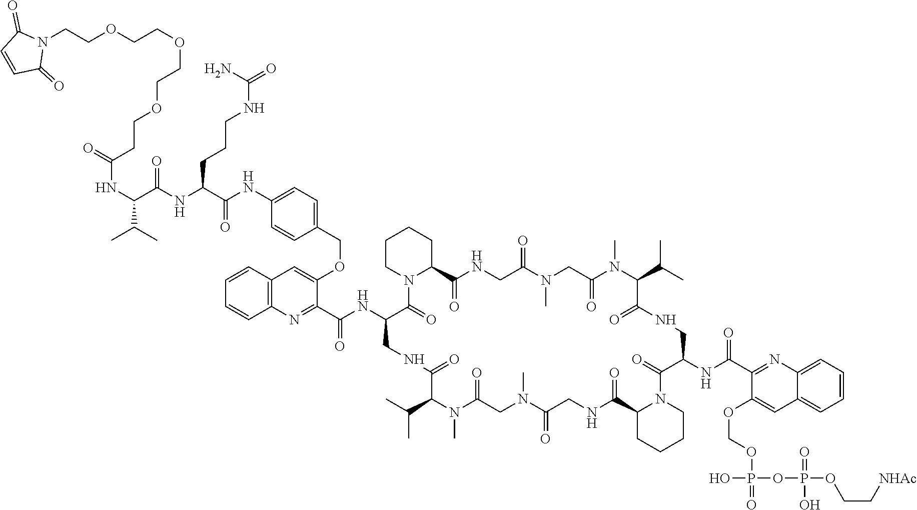 [SMILES: CC(=O)NCCOP(=O)(O)OP(=O)(O)OCOc1cc2ccccc2nc1C(=O)N[C@@H]1CNC(=O)[C@H](C(C)C)N(C)C(=O)CN(C)C(=O)CNC(=O)[C@@H]2CCCCN2C(=O)[C@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)CNC(=O)[C@H](C(C)C)N(C)C(=O)CN(C)C(=O)CNC(=O)[C@@H]2CCCCN2C1=O]

A solution of Compound 322 (11 mg, 0.006 mmol) in DCM (1 mL) was cooled to 0° C., TFA (0.2 mL) was added, stirred at 0° C. for 2 h, then evaporated to dryness. The obtained solid was taken up in DMF (0.5 mL), and iPr₂NEt (0.004 mL, 0.028 mmol) and MT-OTfp (3 mg, 0.007 mmol) were added, then this solution was stirred at RT for 30 min. Preparative HPLC purification eluting with 20 to 50% CH₃CN/H₂O provided the title compound (5.9 mg, 0.003 mmol, 49% yield) as a white solid post lyophilization.

HRMS: Calc'd m/z=2137.8952 for C₉₆H₁₃₃N₂₁O₃₁P₂ found [M+2H]²⁺=1069.9521.

### 4.24 (2S,3R,4S,5S,6S)-2-((2-(((7R,11S,19aS,26R,30S,38aS)-7-((tert-butoxycarbonyl)amino)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 324)

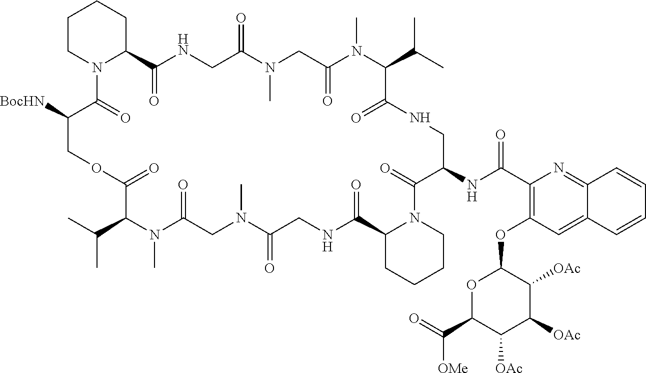 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)OC(C)(C)C)COC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared starting from Compound 244 (130 mg, 0.117 mmol). The Cbz protecting group was removed as described in General Procedure 6, then Compound 302 (75 mg, 0.149 mmol) was coupled as decribed in General Procedure 9. Purification as described in General Procedure 12 using a 10 g silica column and eluting with 0 to 15% MeOH/DCM afforded the title compound (83 mg, 0.057 mmol, 48% yield) as a white solid.

LC/MS: Calc'd m/z=1464.7 for C₆₈H₉₆N₁₂O₂₄ found [M+H+Na]²⁺=743.6 and [M−Boc+2H]²⁺=683.2.

### 4.25 (2S,3R,4S,5S,6S)-2-((2-(((7R,11S,19aS,26R,30S,38aS)-7-(3-((4-((S)-2-((S)-2-((tert-butoxycarbonyl)amino)-3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 325)

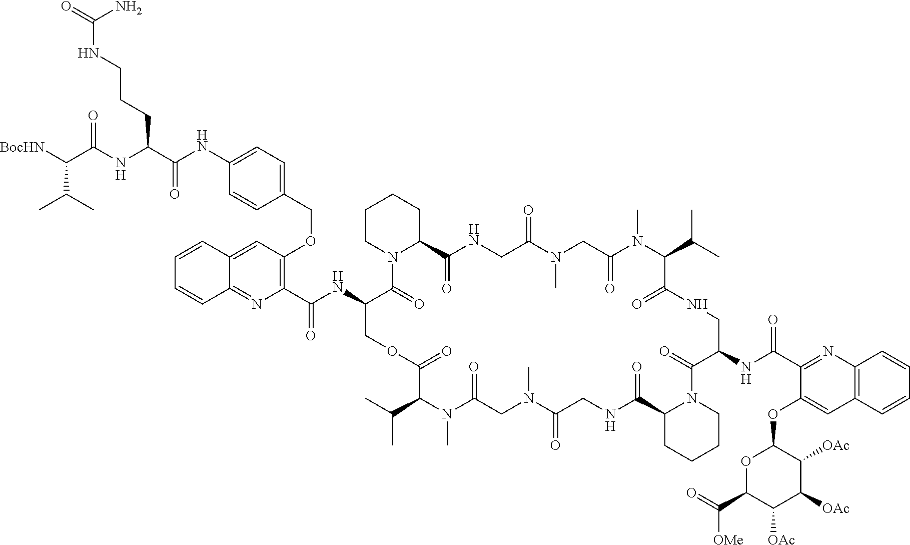 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](CCCNC(N)=O)NC(=O)[C@@H](NC(=O)OC(C)(C)C)C(C)C)cc3)COC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared starting from Compound 324 (83 mg, 0.057 mmol). The Boc protecting group was removed as described in General Procedure 10, then Compound 212 (44.5 mg, 0.068 mmol) was coupled according to General Procedure 9. Preparative HPLC purification as described in General Procedure 12 eluting with 40 to 55% CH₃CN/H₂O afforded the title compound (51 mg, 0.026 mmol, 45% yield) as a white solid.

LC/MS: Calc'd m/z=1996.9 for C₉₆H₁₂₈N₁₈O₂₉ found [M+2H]²⁺=1000.2 and [M−Boc, +3H]³⁺=633.8.

### 4.26 (2S,3S,4S,5R,6S)-6-((2-(((7R,11S,19aS,26R,30S,38aS)-7-(3-((4-((S)-2-((S)-2-amino-3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (Compound 326)

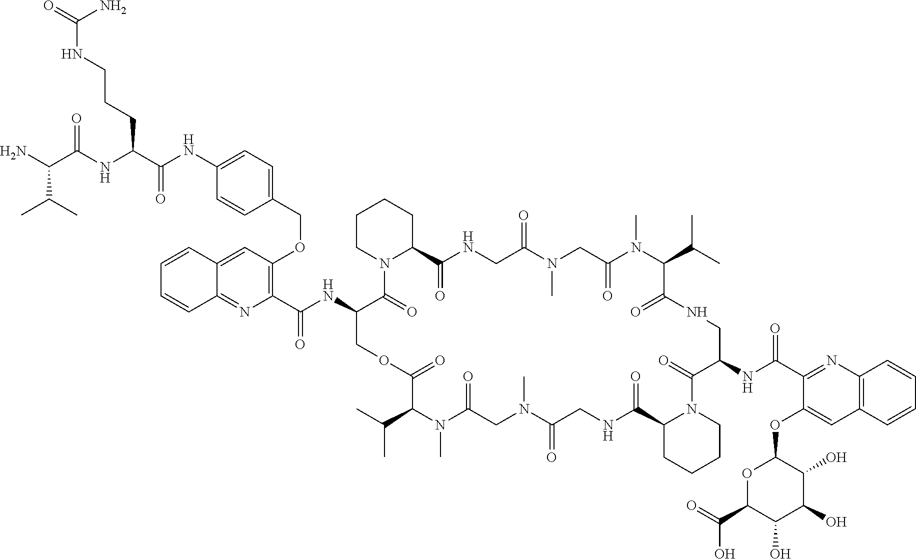 [SMILES: CC(C)[C@H](N)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

A solution of Compound 325 (25 mg, 0.0125 mmol) in THF (1.25 mL) was cooled to 0° C. and an aqueous 1 M solution of LiOH (56 μL, 4.5 eq.) was added. The flask was placed in the fridge overnight. The solution was evaporated to dryness, then the Boc protecting group was removed according to General Procedure 10. Purification as described in General Procedure 12 using a 12 g C18 flash column and eluting with 20 to 60% CH₃CN/H₂O afforded the title compound (11 mg, 0.006 mmol, 50% yield).

LC/MS: Calc'd m/z=1756.8 for C₈₄H₁₁₂N₁₈O₂₄ found [M+2H]²⁺=880.0 and [M+3H]³⁺=587.0.

### 4.27 (2S,3S,4S,5R,6S)-6-((2-(((7R,11S,19aS,26R,30S,38aS)-7-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-11,30-diisopropyl-12,15,31,34-tetramethyl-6,10,13,16,19,25,29,32,35,38-decaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (MT-VC-PAB-14-Glucoronide; Compound 327; DL-13)

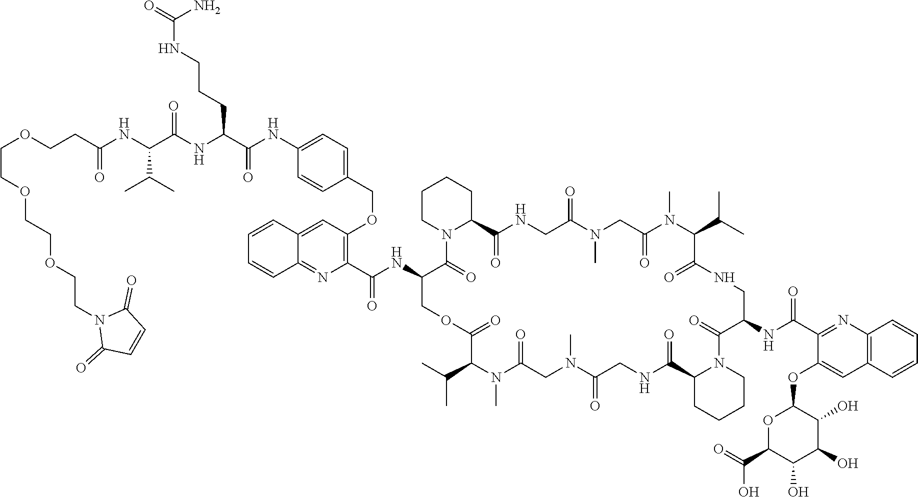 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was synthesized from Compound 326 (11 mg, 0.0062 mmol) as described in General Procedure 11. Preparative HPLC purification according to General Procedure 12, eluting with 30-60% CH₃CN/H₂O, afforded the title compound (5.3 mg, 0.0026 mmol, 42% yield) as a white solid.

LC/MS: Calc'd m/z=2039.9 for C₉₇H₁₂₉N₁₉O₃₀ found [M+3H]³⁺=681.4.

### 4.28 (2S,3R,4S,5S,6S)-2-((2-(((7R,11S,19aS,26R,30S,38aS)-11,30-diisopropyl-12,15,31,34-tetramethyl-7-((((4-nitrobenzyl)oxy)carbonyl)amino)-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 328)

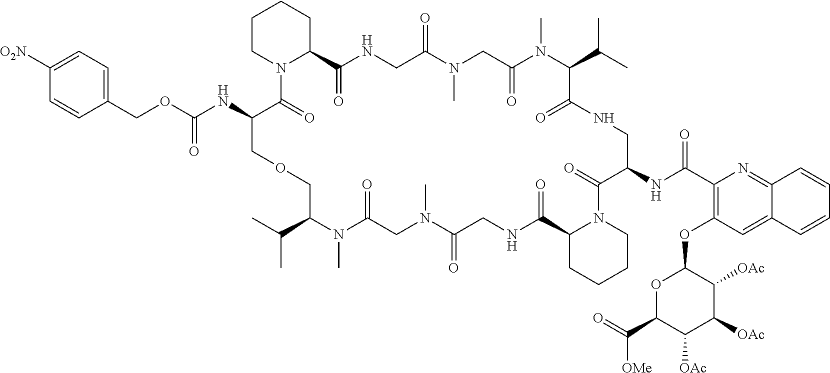 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)OCc3ccc([N+](=O)[O-])cc3)COC[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 174 (83 mg, 0.0726 mmol). The Boc protecting group was cleaved according to General Procedure 5, then Compound 302 (48 mg, 0.094 mmol) was coupled as decribed in General Procedure 9. Purification as described in General Procedure 12 using a 10 g silica column and eluting with 0 to 10% MeOH/DCM afforded the title compound (101 mg, 0.066 mmol, 90% yield) as a white solid.

LC/MS: Calc'd m/z=1529.6 for C₇₁H₉₅N₁₃O₂₅ found [M+2H]²⁺=766.4.

### 4.29 (2S,3R,4S,5S,6S)-2-((2-(((7R,11S,19aS,26R,30S,38aS)-7-(3-((4-((S)-2-((S)-2-((tert-butoxycarbonyl)amino)-3-methylbutanamido)-5-ureidopentanamido)benzyl)oxy)quinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 329)

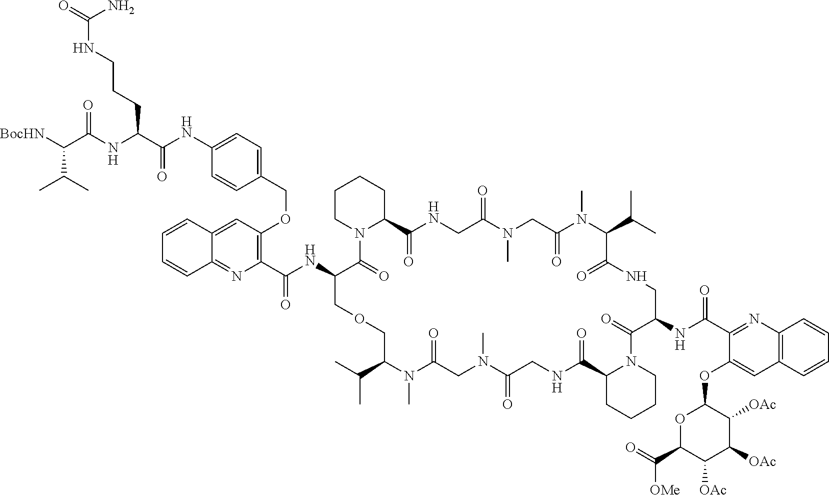 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](CCCNC(N)=O)NC(=O)[C@@H](NC(=O)OC(C)(C)C)C(C)C)cc3)COC[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 328 (101 mg, 0.066 mmol). The 4-NO₂—Cbz protecting group was removed according to General Procedure 6 to provide 40 mg of free amine. Compound 209 (23 mg, 0.030 mmol) was coupled according to General Procedure 9. Prepartive HPLC purification as described in General Procedure 12 and eluting with 20 to 60% CH₃CN/H₂O afforded the title compound (32 mg, 0.016 mmol, 25% yield) as a white solid.

LC/MS: Calc'd m/z=1982.9 for C₉₆H₁₃₀N₁₈O₂₈ found [M+2H]²⁺=993.2 and [M−Boc, +3H]³⁺=629.2.

### 4.30 (2S,3S,4S,5R,6S)-6-((2-(((7R,11S,19aS,26R,30S,38aS)-7-(3-((4-((S)-2-((S)-2-((tert-butoxycarbonyl)amino)-3-methylbutanamido)-5-ureidopentanamido)benzyl)oxy)quinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (Compound 330)

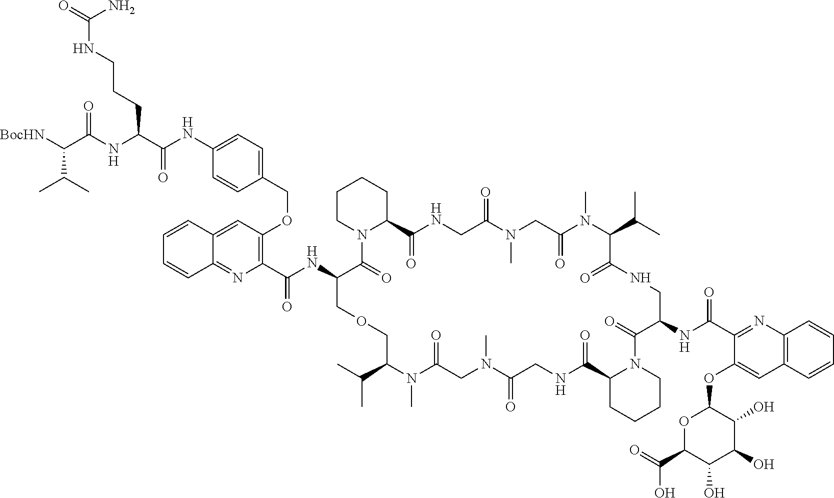 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

To a 10 mL round bottomed flask containing a solution of Compound 329 (32 mg, 0.016 mmol) in THF (2 mL) at 0° C. was added a 1M aqueous solution of LiOH (0.97 mL, 0.097 mmol, 6 eq.). The ice bath was removed, and the solution was stirred at RT for 3 h, then quenched by the addition of a 1M aqueous solution of citric acid (1.15 mL). The mixture was extracted with EtOAc, and the organic layer washed with brine, dried over MgSO₄, and evaporated to dryness. Purification according to General Procedure 12 using a 12 g C18 flash column and eluting with 20 to 70% CH₃CH/H₂O gave the title compound (10 mg, 0.0054 mmol, 62% yield) as a faint yellow solid.

LC/MS: Calc'd m/z=1842.9 for C₈₉H₁₂₂N₁₈O₂₅ found [M+2H]²⁺=923.2 and [M−Boc, +3H]³⁺=582.2.

### 4.31 (2S,3S,4S,5R,6S)-6-((2-(((7R,11S,19aS,26R,30S,38aS)-7-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-11,30-diisopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (MT-VC-PAB-178-Glucoronide; Compound 331; DL-14)

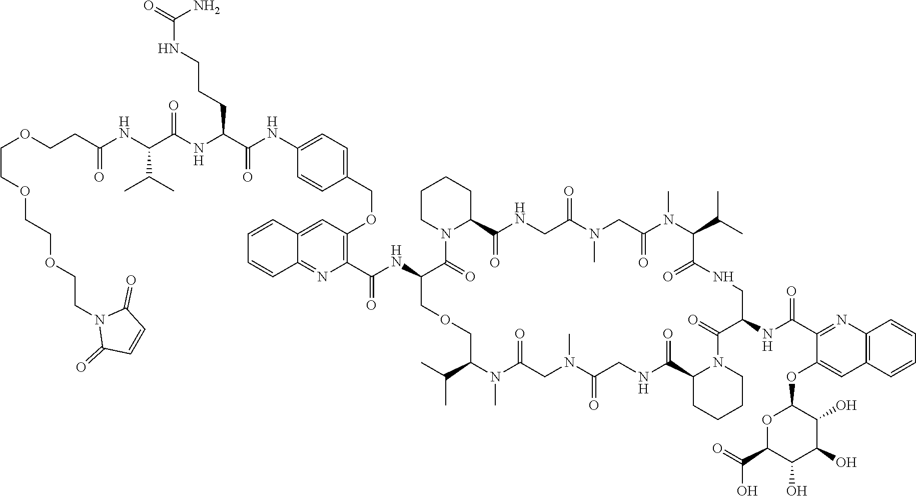 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2COC[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was prepared from Compound 330 (10 mg, 0.0054 mmol) according to General Procedure 10 followed by General Procedure 11. Preparative HPLC purification according to General Procedure 12 and eluting with 40 to 55% CH₃CN/H₂O afforded the title compound (5.1 mg, 0.0025 mmol, 47% yield) as a yellowish solid.

LC/MS: Calc'd m/z=2025.9 for C₉₇H₁₃₁N₁₉O₂₉ found [M+3H]³⁺=676.8.

### 4.32 (2S,3R,4S,5S,6S)-2-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 332)

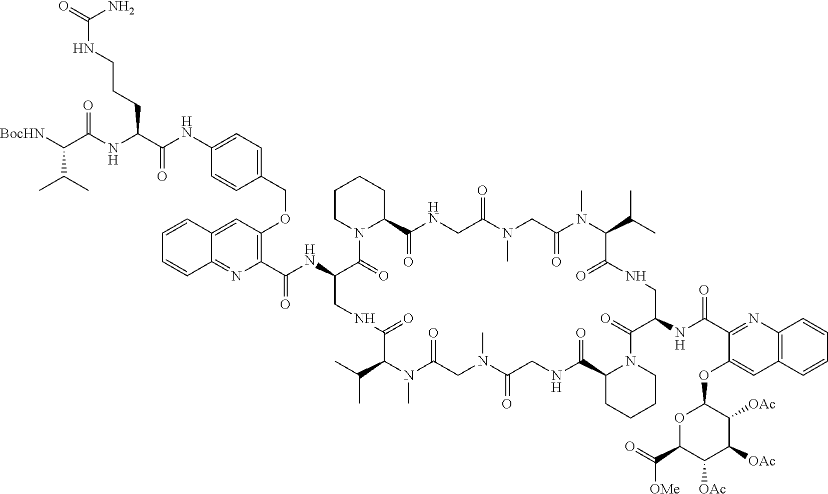 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](CCCNC(N)=O)NC(=O)[C@@H](NC(=O)OC(C)(C)C)C(C)C)cc3)CNC(=O)[C@H](C(C)C)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 306 (57 mg, 0.042 mmol) and Compound 209 (32.6 mg, 0.05 mmol) according to General Procedure 9. Purification according to General Procedure 12 using a 12 g C18 flash column and eluting with 35 to 65% CH₃CN/H₂O afforded the title compound (47 mg, 0.024 mmol, 56% yield) as a white solid.

LC/MS: Calc'd m/z=1995.9 for C₉₆H₁₂₉N₁₉O₂₈ found [M+2H]²⁺=999.6 and [M−Boc, +3H]³⁺=633.4.

### 4.33 (2S,3S,4S,5R,6S)-6-((2-(((7R,11S,19aS,26R,30S,38aS)-26-(3-((4-((S)-2-((S)-2-amino-3-methylbutanamido)-5-ureidopentanamido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 333)

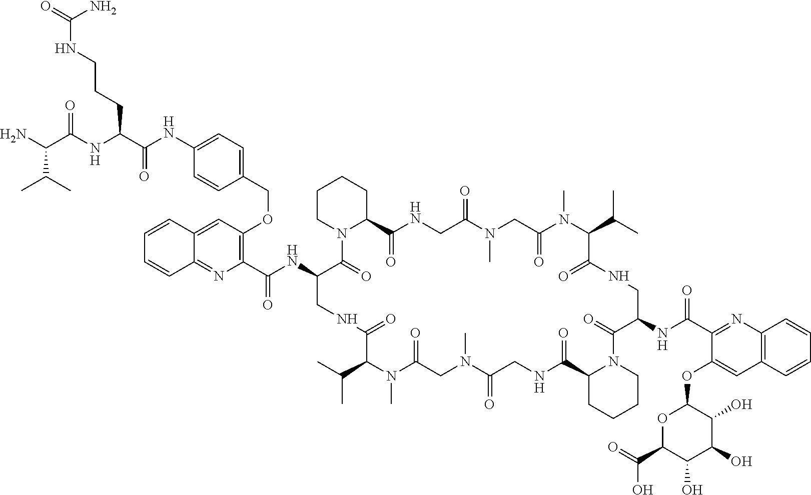 [SMILES: CC(C)[C@H](N)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

To a solution of Compound 323 (23.5 mg, 0.012 mmol) in THF (3 mL) at 0° C. was added a 1M aqueous solution of LiOH (0.141 mL, 0.141 mmol, 12 eq.). The ice bath was removed, and the solution was stirred at RT for 3 h, then quenched by the addition of a 1M aqueous solution of NaHSO₄ (3 mL). The mixture was extracted with EtOAc, amd the organic layer washed with brine, dried over MgSO₄, and evaporated to dryness. Purification was acheived according to General Procedure 12 using a 12 g C18 flash column and eluting with 25 to 50% CH₃CH/H₂O. Solvent was removed by lyophilization then the Boc protecting group was cleaved according to General Procedure 10 to give the title compound (11.2 mg, 0.007 mmol, 58% yield).

LC/MS: Calc'd m/z=1755.8 for C₈₄H₁₁₃N₁₉O₂₃ found [M+2H]²⁺=879.4 and [M+3H]³⁺=586.0.

### 4.34 (2S,3S,4S,5R,6S)-6-((2-(((7R,11S,19aS,26R,30S,38aS)-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-11,30-diisopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (MT-VC-PAB-35-Glucoronide; Compound 334; DL-15)

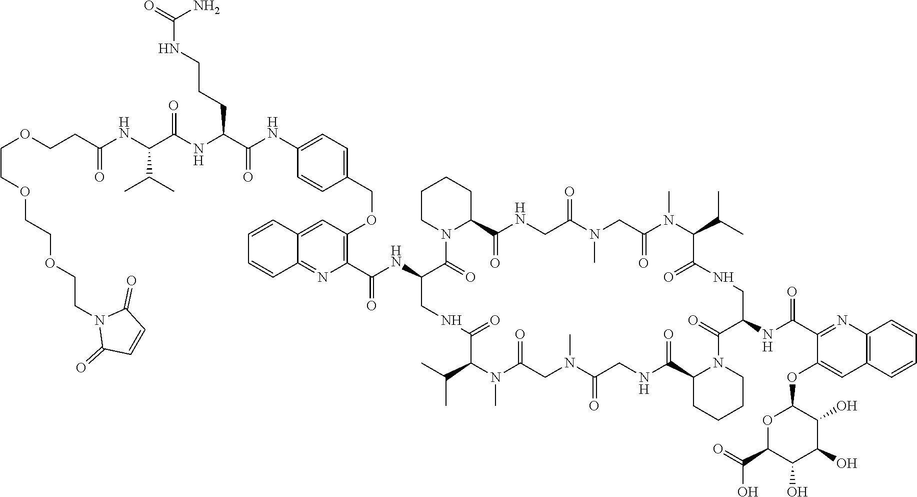 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was prepared from Compound 333 (11.2 mg, 0.006 mmol) according to General Procedure 11. Purification according to General Procedure 12 using a 12 g C₁₈ flash column and eluting with 25 to 45% CH₃CN/H₂O afforded the title compound (4.7 mg, 0.002 mmol, 40% yield) as a faint yellow solid.

LC/MS: Calc'd m/z=2038.9 for C₉₇H₁₃₀N₂₀O₂₉ found [M+2H]²⁺=1021.6 and [M+3H]³⁺=681.4.

### 4.35 (2S,3R,4S,5S,6S)-2-((2-(((7R,11S,19aS,26R,30S,38aS)-26-(((benzyloxy)carbonyl)amino)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 335)

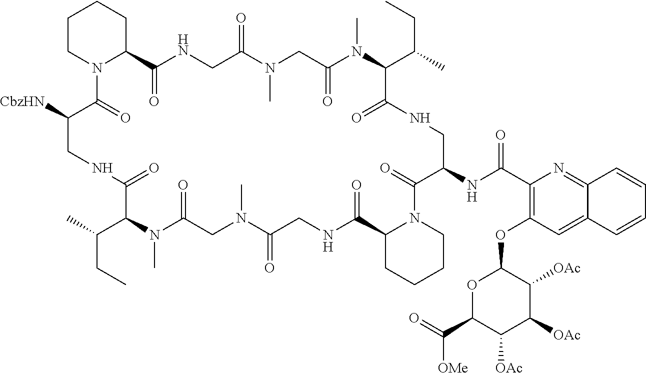 [SMILES: CC[C@H](C)[C@H]1C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(C)CC(=O)N(C)[C@@H]([C@@H](C)CC)C(=O)NC[C@@H](NC(=O)OCc2ccccc2)C(=O)N2CCCC[C@H]2C(=O)NCC(=O)N(C)CC(=O)N1C]

The title compound was prepared from Compound 64 (30 mg, 0.029 mmol) and Compound 302 (16 mg, 0.032 mmol) according to General Procedure 9. Purification according to General Procedure 12 using a 12 g C18 flash column and eluting with 30 to 80% CH₃CN/H₂O afforded the title compound (16 mg, 0.01 mmol, 36% yield).

LC/MS: Calc'd m/z=1525.7 for C₇₃H₉₉N₁₃O₂₃ found [M+2H]²⁺=764.2.

### 4.36 (2S,3R,4S,5S,6S)-2-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 336)

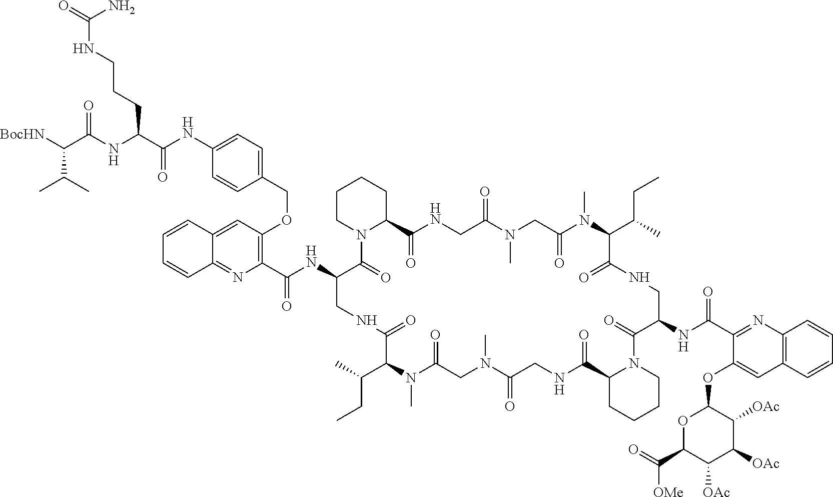 [SMILES: CC[C@H](C)[C@H]1C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC(=O)N(C)CC(=O)N1C]

The title compound was prepared from Compound 335 (16 mg, 0.01 mmol). The Cbz protecting group was cleaved according to General Procedure 6, then Compound 209 was coupled as described in General Procedure 9. Purification according to General Procedure 12 using a 12 g C18 flash column and eluting with 30 to 80% CH₃CN/H₂O afforded the title compound (11.3 mg, 0.0056 mmol, 56% yield) as a faint yellow solid.

LC/MS: Calc'd m/z=2023.9 for C₉₈H₁₃₃N₁₉O₂₈ found [M+2H]²⁺=1013.6 and [M−Boc, +3H]³⁺=642.8.

### 4.37 (2S,3S,4S,5R,6S)-6-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-11,30-di((S)-sec-butyl)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 337)

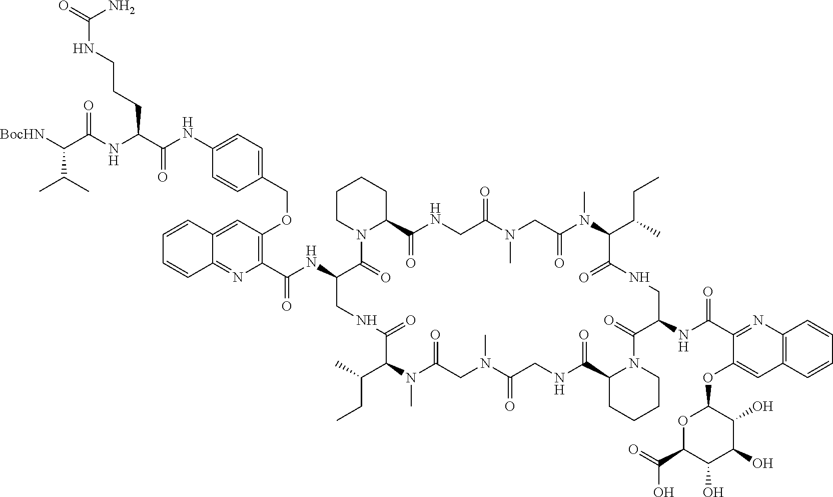 [SMILES: CC[C@H](C)[C@H]1C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC(=O)N(C)CC(=O)N1C]

To a solution of Compound 336 (11.3 mg, 0.0056 mmol) in THF (2 mL) at 0° C. was added a 1M aqueous solution of LiOH (0.033 mL, 0.033 mmol, 6 eq.). The ice bath was removed, and the solution was stirred at RT for 3 h, then quenched by the addition of a 1M aqueous solution of citric acid (3 mL). This mixture was extracted with EtOAc, and the organic layer washed with brine, dried over MgSO₄, and evaporated to dryness. Purification according to General Procedure 12 using a 12 g C18 flash column and eluting with 20 to 75% CH₃CH/H₂O afforded the title compound (6 mg, 0.0032 mmol, 57% yield) as a white solid.

LC/MS: Calc'd m/z=1883.9 for C₉₁H₁₂₅N₁₉O₂₅ found [M+2H]²⁺=943.6 and [M−Boc, +3H]₃₊=596.0.

### 4.38 (2S,3S,4S,5R,6S)-6-((2-(((7R,11S,19aS,26R,30S,38aS)-11,30-di((S)-sec-butyl)-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (MT-VC-PAB-67-Glucoronide; Compound 338; DL-19)

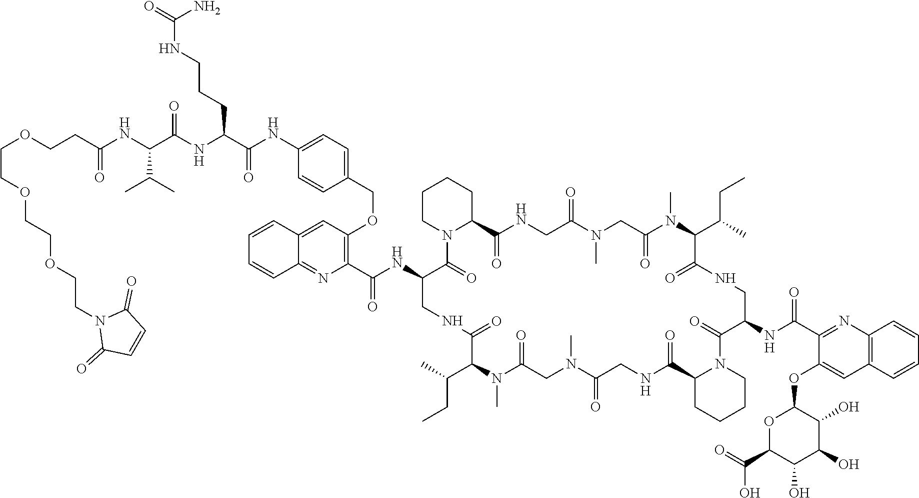 [SMILES: CC[C@H](C)[C@H]1C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(C)CC(=O)N(C)[C@@H]([C@@H](C)CC)C(=O)NC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(C)CC(=O)N1C]

The title compound was prepared from Compound 337 (6 mg, 0.0032 mmol) according to General Procedure 10 followed by General Procedure 11. Preparative HPLC purification according to General Procedure 12 and eluting with 30 to 60% CH₃CN/H₂O afforded the title compound (3.6 mg, 0.0017 mmol, 54% yield) as a faint yellow solid.

LC/MS: Calc'd m/z=2066.9 for C₉₉H₁₃₄N₂₀O₂₉ found [M+2H]²⁺=1035.2 and [M+3H]³⁺=690.4.

4.39 (2S,3R,4S,5S,6S)-2-((2-(((5aS,9R,15aS,24S,28R,34aS)-9-(((benzyloxy)carbonyl)amino)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontin-28-yl)carbamoyl)quinolin-3-yl)oxy)-6-(methoxycarbonyl)tetrahydro-2H-pyran-3,4,5-triyl triacetate (Compound 339)

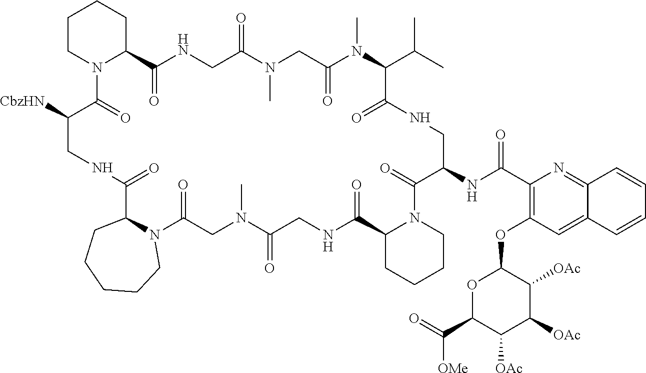 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)OCc3ccccc3)CNC(=O)[C@@H]3CCCCCN3C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 84 (50 mg, 0.044 mmol). The Boc protecting group was cleaved according to General Procedure 5, followed by coupling of Compound 302 (33 mg, 0.066 mmol). Purification according to General Procedure 12 using a 12 g C18 flash column and eluting with 38 to 55% CH₃CN/H₂O afforded the title compound (31.4 mg, 0.021 mmol, 47% yield) as a white solid.

LC/MS: Calc'd m/z=1509.6 for C₇₂H₉₅N₁₃O₂₃ found [M+2H]²⁺=756.1.

### 4.40 (2S,3R,4S,5S,6S)-2-((2-(((5aS,9R,15aS,24S,28R,34aS)-9-(3-((4-((S)-2-((S)-2-((tert-butoxycarbonyl)amino)-3-methylbutanamido)-5-ureidopentanamido)benzyl)oxy)quinoline-2-carboxamido)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontin-28-yl)carbamoyl)quinolin-3-yl)oxy)-6-(methoxycarbonyl)tetrahydro-2H-pyran-3,4,5-triyl triacetate (Compound 340)

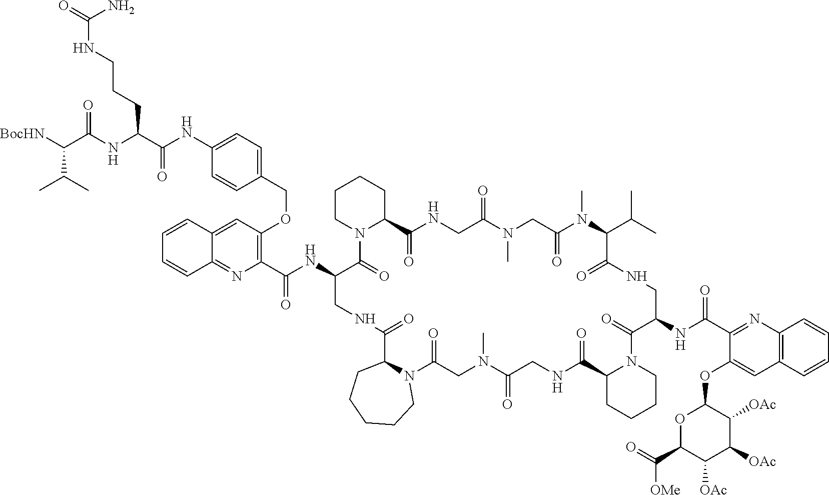 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](CCCNC(N)=O)NC(=O)[C@@H](NC(=O)OC(C)(C)C)C(C)C)cc3)CNC(=O)[C@@H]3CCCCCN3C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 339 (31.4 mg, 0.021 mmol). The Cbz protecting group was removed according to General Procedure 6, followed by coupling of Compound 209 (17 mg, 0.026 mmol) as described in General Procedure 9. Purification as described in General Procedure 12 using a 12 g C18 flash column and eluting with 25 to 45% CH₃CN/H₂O afforded the title compound (34 mg, 0.017 mmol, 81% yield) as a white solid.

LC/MS: Calc'd m/z=2007.9 for C₉₇H₁₂₉N₁₉O₂₈ found [M+2H]²⁺=1005.8 and [M−Boc, +3H]³⁺=637.4.

### 4.41 (2S,3S,4S,5R,6S)-6-((2-(((5aS,9R,15aS,24S,28R,34aS)-9-(3-((4-((S)-2-((S)-2-((tert-butoxycarbonyl)amino)-3-methylbutanamido)-5-ureidopentanamido)benzyl)oxy)quinoline-2-carboxamido)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontin-28-yl)carbamoyl)quinolin-3-yl)oxy)-3,4,5-trihydroxytetrahydro-2H-pyran-2-carboxylic acid (Compound 341)

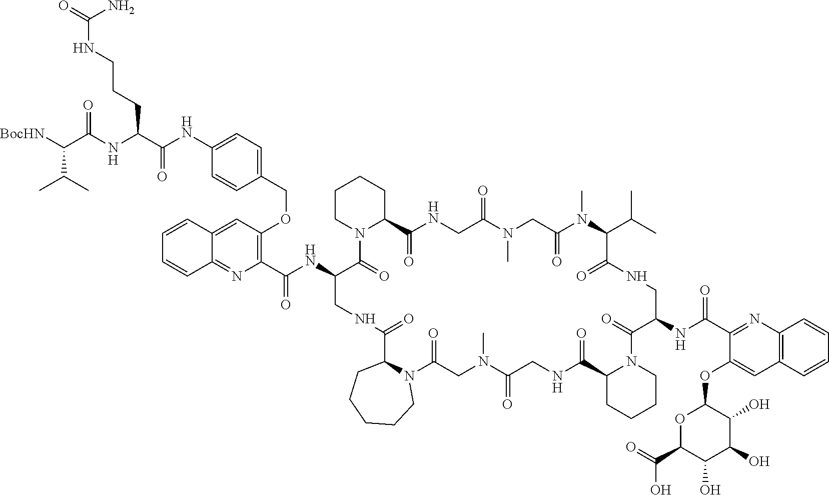 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2CNC(=O)[C@@H]3CCCCCN3C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

To a solution of Compound 340 (34 mg, 0.017 mmol) in THF (4.5 mL) at 0° C. was added a 1M aqueous solution of LiOH (0.203 mL, 0.203 mmol, 12 eq.). The ice bath was removed, and the solution was stirred at RT for 3 h, then quenched by the addition of a 1M aqueous solution of NaHSO₄ (5 mL). The mixture was extracted with EtOAc, and the organic layer washed with brine, dried over MgSO₄ and evaporated to dryness. Purification according to General Procedure 12 using a 12 g C18 flash column and eluting with 20 to 43% CH₃CH/H₂O afforded the title compound (14 mg, 0.0075 mmol, 44% yield) as a yellow solid.

LC/MS: Calc'd m/z=1867.9 for C₉₀H₁₂₁N₁₉O₂₅ found [M+2H]²⁺=935.6 and [M−Boc, +3H]³⁺=591.0.

### 4.42 (2S,3S,4S,5R,6S)-6-((2-(((5aS,9R,15aS,24S,28R,34aS)-9-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-24-isopropyl-20,23,39-trimethyl-6,10,16,19,22,25,29,35,38,41-decaoxotetracontahydro-1H,12H-azepino[1,2-j]dipyrido[1,2-a:1′,2′-q][1,4,7,10,13,17,20,23,26,29]decaazacyclodotriacontin-28-yl)carbamoyl)quinolin-3-yl)oxy)-3,4,5-trihydroxytetrahydro-2H-pyran-2-carboxylic acid (MT-VC-PAB-88-Glucoronide; Compound 342; DL-20)

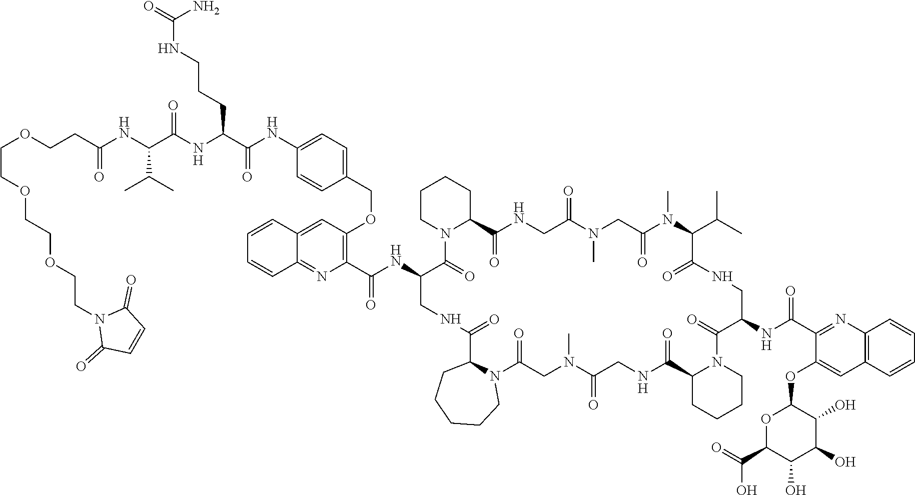 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@@H]3CCCCCN3C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was prepared from Compound 341 (14 mg, 0.0075 mmol) according to General Procedure 10 followed by General Procedure 11. Preparative HPLC purification according to General Procedure 12 and eluting with 35 to 50% CH₃CN/H₂O afforded the title compound (6.1 mg, 0.004 mmol, 58% yield) as a faint yellow solid.

LC/MS: Calc'd m/z=2050.9 for C₉₈H₁₃₀N₂₀O₂₉ found [M+2H]²⁺=1027.2 and [M+3H]³⁺=685.2.

### 4.43 (2S,3R,4S,5S,6S)-2-((2-(((7R,11S,19aS,26R,30S,38aS)-26-((tert-butoxycarbonyl)amino)-30-cyclohexyl-11-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 343)

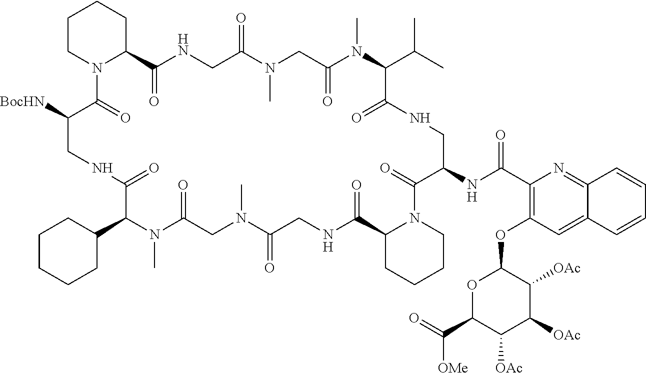 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)OC(C)(C)C)CNC(=O)[C@H](C3CCCCC3)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 94 (37 mg, 0.0364 mmol) and Compound 302 (27 mg, 0.054 mmol) according to General Procedure 9. Preparative HPLC purfification according to General Procedure 12 and eluting with 30 to 70% CH₃CN/H₂O afforded the title compound (25 mg, 0.017 mmol, 46% yield) as a yellow solid.

LC/MS: Calc'd m/z=2050.9 for C₉₈H₁₃₀N₂₀O₂₉ found [M+2H]²⁺=703.2.

### 4.44 (2S,3R,4S,5S,6S)-2-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-30-cyclohexyl-11-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 344)

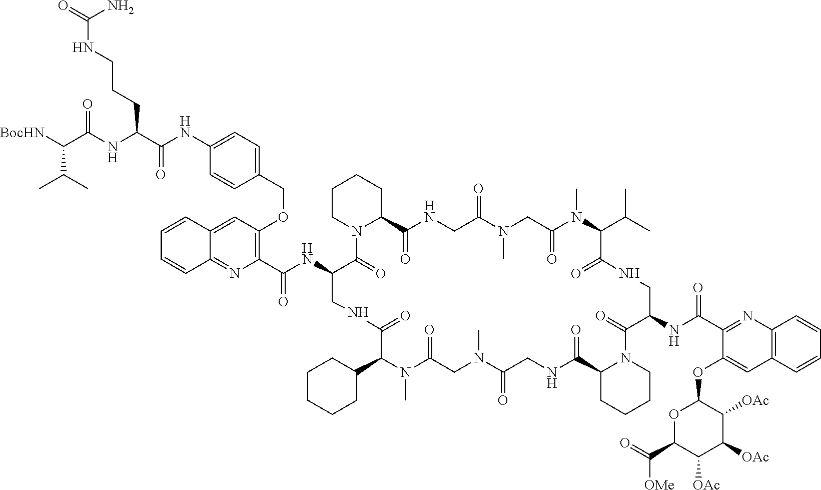 [SMILES: COC(=O)[C@H]1O[C@@H](Oc2cc3ccccc3nc2C(=O)N[C@@H]2CNC(=O)[C@H](C(C)C)N(C)C(=O)CN(C)C(=O)CNC(=O)[C@@H]3CCCCN3C(=O)[C@H](NC(=O)c3nc4ccccc4cc3OCc3ccc(NC(=O)[C@H](CCCNC(N)=O)NC(=O)[C@@H](NC(=O)OC(C)(C)C)C(C)C)cc3)CNC(=O)[C@H](C3CCCCC3)N(C)C(=O)CN(C)C(=O)CNC(=O)[C@@H]3CCCCN3C2=O)[C@H](OC(C)=O)[C@@H](OC(C)=O)[C@@H]1OC(C)=O]

The title compound was prepared from Compound 344 (25 mg, 0.017 mmol). The Boc protecting group was removed according to General Procedure 10, followed by coupling of Compound 209 (13 mg, 0.02 mmol) as described in General Procedure 9. Preparative HPLC purification according to General Procedure 12 and eluting with 35 to 70% CH₃CN/H₂O afforded the title compound (28 mg, 0.014 mmol, 80% yield) as a faint yellow powder.

LC/MS: Calc'd m/z=2035.9 for C₉₉H₁₃₃N₁₉O₂₈ found [M+2H]²⁺=1019.6 and [M−Boc, +3H]³⁺=646.8.

### 4.45 (2S,3S,4S,5R,6S)-6-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-30-cyclohexyl-11-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 345)

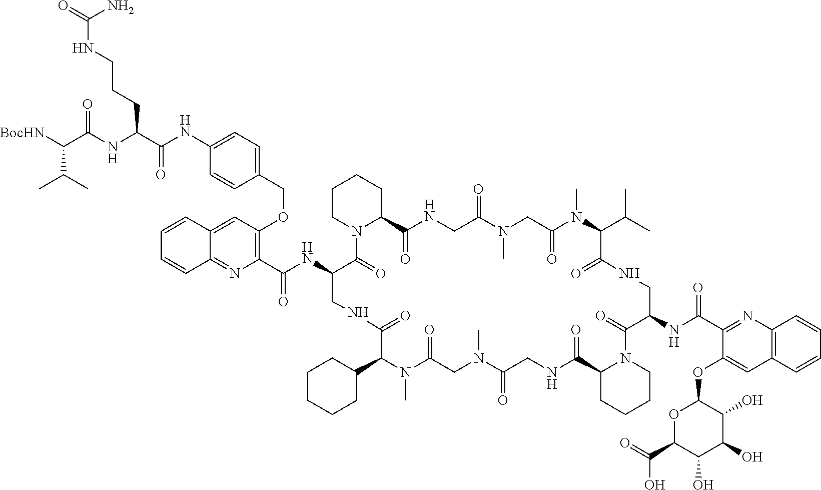 [SMILES: CC(C)[C@H](NC(=O)OC(C)(C)C)C(=O)N[C@@H](CCCNC(N)=O)C(=O)Nc1ccc(COc2cc3ccccc3nc2C(=O)N[C@@H]2CNC(=O)[C@H](C3CCCCC3)N(C)C(=O)CN(C)C(=O)CNC(=O)[C@@H]3CCCCN3C(=O)[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

To a solution of Compound 344 (28 mg, 0.014 mmol) in THF (1 mL) at 0° C. was added a 1M aqueous solution of LiOH (0.082 mL, 0.082 mmol, 6 eq.). The ice bath was removed, and the solution was stirred at RT for 3 h, quenched by the addition of 0.1% TFA/H₂O (2 mL) and then loaded directly onto a 12 g C18 flash column. Purification according to General Procedure 12 eluting with 20 to 50% CH₃CH/H₂O afforded the title compound (18 mg, 0.0095 mmol, 68% yield) as a yellow solid.

LC/MS: Calc'd m/z=1895.9 for C₉₂H₁₂₅N₁₉O₂₅ found [M+2H]²⁺=959.6 and [M−Boc, +3H]³⁺=600.0.

### 4.46 (2S,3S,4S,5R,6S)-6-((2-(((7R,11S,19aS,26R,30S,38aS)-30-cyclohexyl-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-11-isopropyl-12,15,31,34-tetramethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (MT-VC-PAB-82-Glucoronide; Compound 346; DL-21)

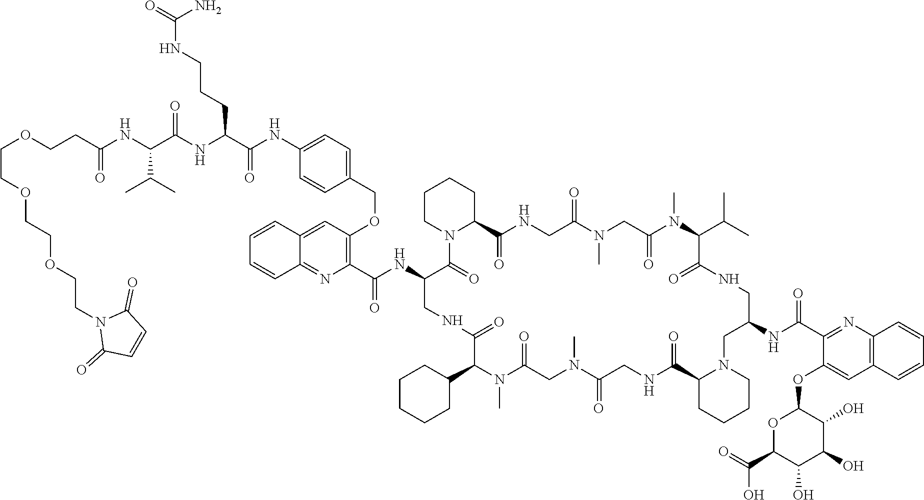 [SMILES: CC(C)[C@H](NC(=O)CCOCCOCCOCCN1C(=O)C=CC1=O)C(=O)N[C@@H](CCCNC(N)=O)C(=O)Nc1ccc(COc2cc3ccccc3nc2C(=O)N[C@@H]2CNC(=O)[C@H](C3CCCCC3)N(C)C(=O)CN(C)C(=O)CNC(=O)[C@@H]3CCCCN3C[C@H](NC(=O)c3nc4ccccc4cc3O[C@@H]3O[C@H](C(=O)O)[C@@H](O)[C@H](O)[C@H]3O)CNC(=O)[C@H](C(C)C)N(C)C(=O)CN(C)C(=O)CNC(=O)[C@@H]3CCCCN3C2=O)cc1]

The title compound was prepared from Compound 345 (18 mg, 0.0095 mmol) according to General Procedure 10 followed by General Procedure 11. Preparative HPLC purification as described in General Procedure 12 and eluting with 30 to 55% CH₃CN/H₂O afforded the title compound (11.9 mg, 0.0057 mmol, 60% yield) as a faint yellow solid.

LC/MS: Calc'd m/z=2078.9 for C₁₀₀H₁₃₄N₂₀O₂₉ found [M+2H]²⁺=1041.0 and [M+3H]₃₊=694.6.

### 4.47 (2S,3R,4S,5S,6S)-2-((2-(((7R,11S,19aS,26R,30S,38aS)-26-(((benzyloxy)carbonyl)amino)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 347)

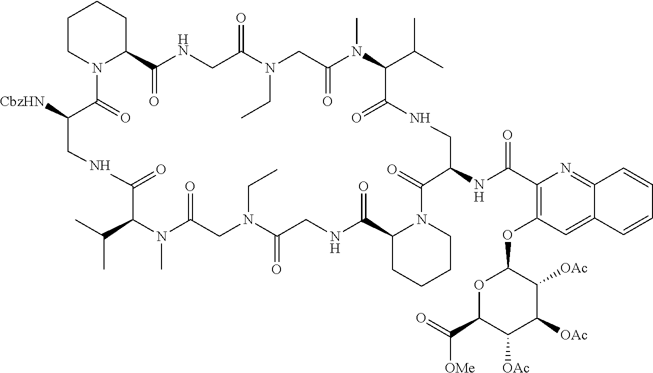 [SMILES: CCN1CC(=O)N(C)[C@@H](C(C)C)C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(CC)CC(=O)N(C)[C@@H](C(C)C)C(=O)NC[C@@H](NC(=O)OCc2ccccc2)C(=O)N2CCCC[C@H]2C(=O)NCC1=O]

The title compound was prepared from Compound 129 (45 mg, 0.04 mmol). The Boc protecting group was removed according to General Procedure 5 followed by coupling of Compound 302 (24 mg, 0.047 mmol) as described in General Procedure 9. Purification according to General Procedure 12 using a 12 g C18 flash column and eluting with 10 to 90% CH₃CN/H₂O afforded the title compound (58 mg, 0.038 mmol, 95% yield) as a yellow solid.

LC/MS: Calc'd m/z=1525.7 for C₇₃H₉₉N₁₃O₂₃ found [M+2H]²⁺=764.2.

### 4.48 (2S,3R,4S,5S,6S)-2-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-6-(methoxycarbonyl)tetrahydro-2H-pyran-3,4,5-triyl triacetate (Compound 348)

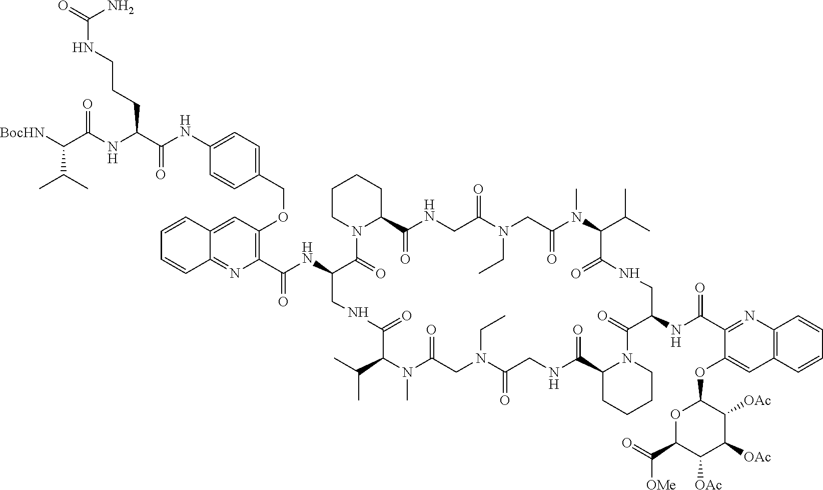 [SMILES: CCN1CC(=O)N(C)[C@@H](C(C)C)C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(CC)CC(=O)N(C)[C@@H](C(C)C)C(=O)NC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC1=O]

The title compound was prepared from Compound 347 (54 mg, 0.035 mmol). The Cbz protecting group was removed as described in General Procedure 6 followed by coupling of Compound 209 (25 mg, 0.039 mmol) as described in General Procedure 9. Purification as described in General Procedure 12 using a 12 g C18 flash column and eluting with 10 to 90% CH₃CN/H₂O afforded the title compound (43 mg, 0.02 mmol, 61% yield) as a faint yellow solid.

LC/MS: Calc'd m/z=2023.9 for C₉₈H₁₃₃N₁₉O₂₈ found [M+2H]²⁺=1013.6 and [M−Boc, +3H]³⁺=642.8.

### 4.49 (2S,3S,4S,5R,6S)-6-((2-(((7R,11S,19aS,26R,30S,38aS)-26-(3-((4-((S)-2-((S)-2-((tert-butoxycarbonyl)amino)- 3-methylbutanamido)-5-ureidopentanamido)benzyl)oxy)quinoline-2-carboxamido)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (Compound 349)

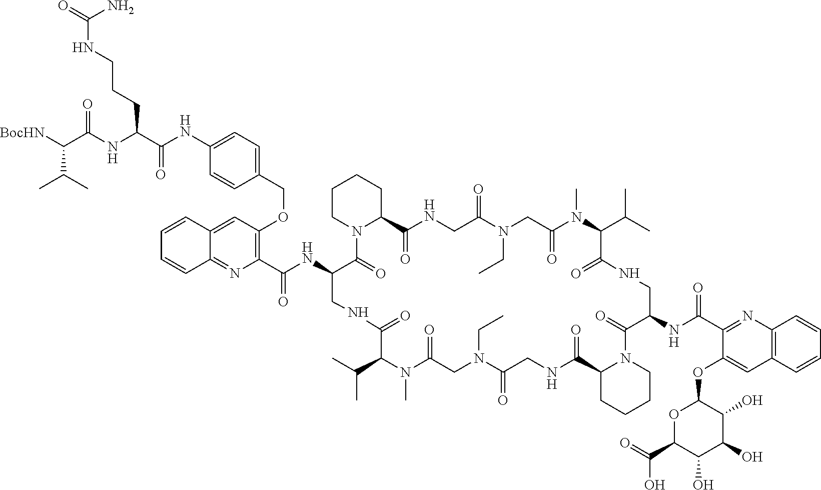 [SMILES: CCN1CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(CC)CC(=O)N(C)[C@@H](C(C)C)C(=O)NC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC1=O]

To a solution of Compound 348 (43 mg, 0.021 mmol) in THF (1 mL) at 0° C. was added a 1M aqueous solution of LiOH (0.127 mL, 0.127 mmol, 6 eq.). The ice bath was removed, and the solution was stirred at RT for 3 h, quenched by the addition of 0.1% TFA/H₂O (2 mL) and loaded directly on to a 12 g C18 flash column. Purification according to General Procedure 12 eluting with 10 to 100% CH₃CH/H₂O afforded the title compound (21 mg, 0.011 mmol, 52% yield) as a yellow solid.

LC/MS: Calc'd m/z=1883.9 for C₉₁H₁₂₅N₁₉O₂₅ found [M+2H]²⁺=943.6 and [M−Boc, +3H]³⁺=592.6.

### 4.50 (2S,3S,4S,5R,6S)-6-((2-(((7R,11S,19aS,26R,30S,38aS)-26-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-15,34-diethyl-11,30-diisopropyl-12,31-dimethyl-6,10,13,16,19,25,29,32,35,38-decaoxohexatriacontahydro-2H,6H-dipyrido[1,2-a:1′,2′-q][1,4,7,10,13,17,20,23,26,29]decaazacyclodotriacontin-7-yl)carbamoyl)quinolin-3-yl)oxy)-3,4,5-trihydroxytetrahydro-2H-pyran-2-carboxylic acid (MT-VC-PAB-132-Glucoronide; Compound 350; DL-22)

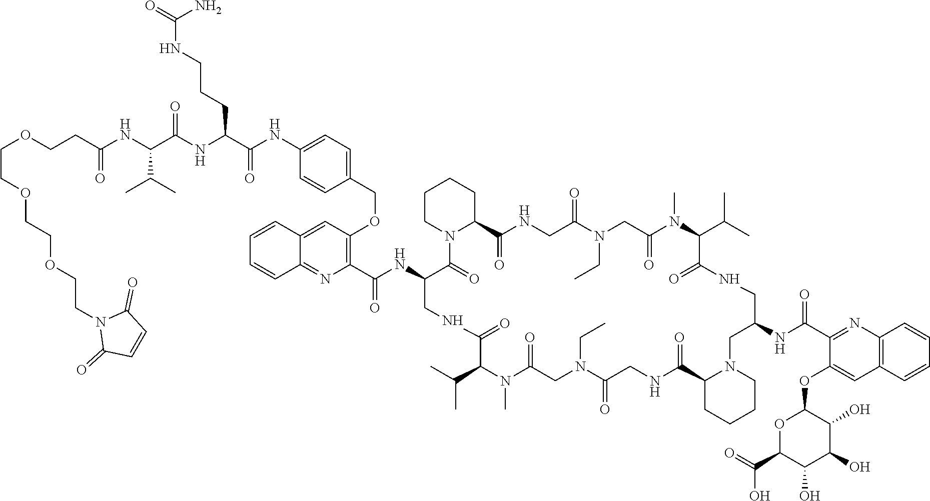 [SMILES: CCN1CC(=O)N(C)[C@@H](C(C)C)C(=O)NC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(CC)CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)CN2CCCC[C@H]2C(=O)NCC1=O]

The title compound was prepared from Compound 349 (21 mg, 0.011 mmol) according to General Procedure 10 followed by General Procedure 11. Preparative HPLC purification as described in General Procedure 12 eluting with 30 to 55% CH₃CN/H₂O afforded the title compound (8.6 mmol, 0.004 mmol, 37% yield) as a white solid.

LC/MS: Calc'd m/z=2066.9 for C₉₉H₁₃₄N₂₀O₂₉ found [M+2H]²⁺=1035.0 and [M+3H]³⁺=690.6.

### 4.51 (2S,3R,4S,5S,6S)-2-((2-(((7R,11S,19aS,26R,30S,38aS)-11-((S)-sec-butyl)-30-isopropyl-12,15,31,34-tetramethyl-7-((((4-nitrobenzyl)oxy)carbonyl)amino)-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 351)

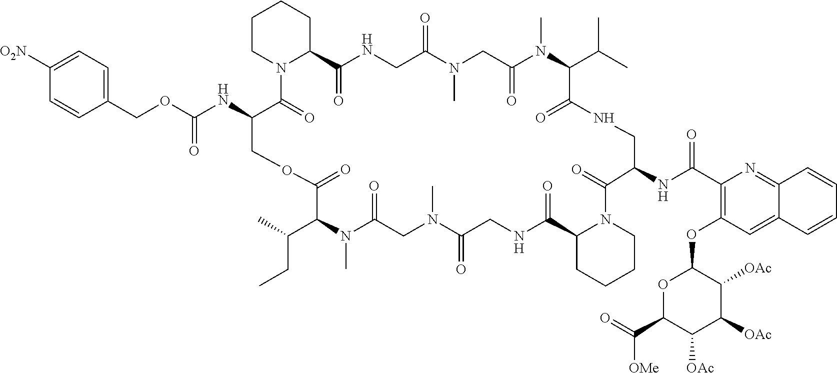 [SMILES: CC[C@H](C)[C@H]1C(=O)OC[C@@H](NC(=O)OCc2ccc([N+](=O)[O-])cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(C)CC(=O)N1C]

The title compound was prepared from Compound 193 (51 mg, 0.044 mmol). The Boc protecting group was removed as described in General Procedure 5 followed by coupling of Compound 302 (26.6 mg, 0.053 mmol) as described in General Procedure 9. Purification according to General Procedure 12 using a 12 g C18 flash column and eluting with 10 to 90% CH₃CN/H₂O afforded the title compound (61 mg, 0.039 mmol, 90% yield) as a faint yellow solid.

LC/MS: Calc'd m/z=1543.7 for C₇₂H₉₇N₁₃O₂₅ found [M+2H]²⁺=773.2.

### 4.52 (2S,3R,4S,5S,6S)-2-((2-(((7R,11S,19aS,26R,30S,38aS)-7-(3-((4-((S)-2-((S)-2-((tert-butoxycarbonyl)amino)-3-methylbutanamido)-5-ureidopentanamido)benzyl)oxy)quinoline-2-carboxamido)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-6-(methoxycarbonyl)tetrahydro-2H-pyran-3,4,5-triyl triacetate (Compound 352)

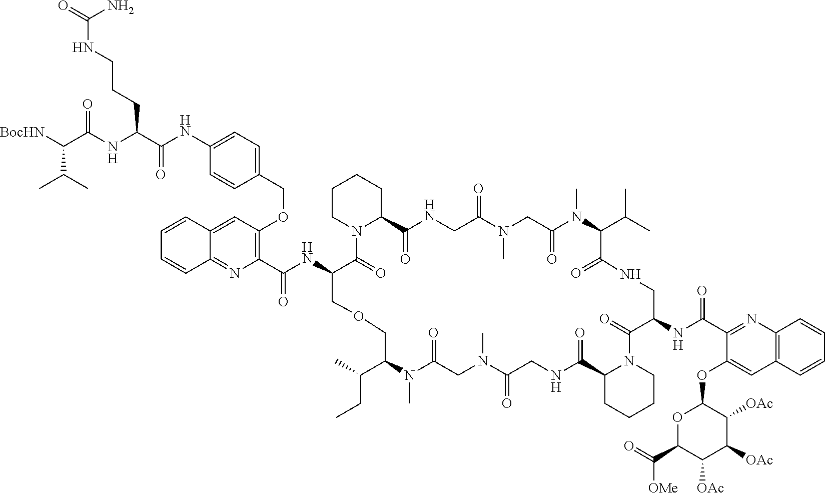 [SMILES: CC[C@H](C)[C@H]1COC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2O[C@@H]2O[C@H](C(=O)OC)[C@@H](OC(C)=O)[C@H](OC(C)=O)[C@H]2OC(C)=O)C(=O)N2CCCC[C@H]2C(=O)NCC(=O)N(C)CC(=O)N1C]

The title compound was prepared from Compound 351 (54 mg, 0.035 mmol). The 4-NO₂—Cbz protecting group was cleaved as described in General Procedure 6 followed by coupling of Compound 209 (25 mg, 0.039 mmol) as described in General Procedure 9. Purification according to General Procedure 12 using a 12 g C18 flash column and eluting with 30 to 90% CH₃CN/H₂O afforded the title compound (35 mg, 0.018 mmol, 50% yield) as a faint yellow solid.

LC/MS: Calc'd m/z=1996.9 for C₉₇H₁₃₂N₁₈O₂₈ found [M+2H]²⁺=1000.2 and [M−Boc, +3H]³⁺=633.8.

### 4.53 (2S,3S,4S,5R,6S)-6-((2-(((7R,11S,19aS,26R,30S,38aS)-7-(3-((4-((S)-2-((S)-2-((tert-butoxycarbonyl)amino)-3-methylbutanamido)-5-ureidopentanamido)benzyl)oxy)quinoline-2-carboxamido)-11-((S)-sec-butyl)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (Compound 353)

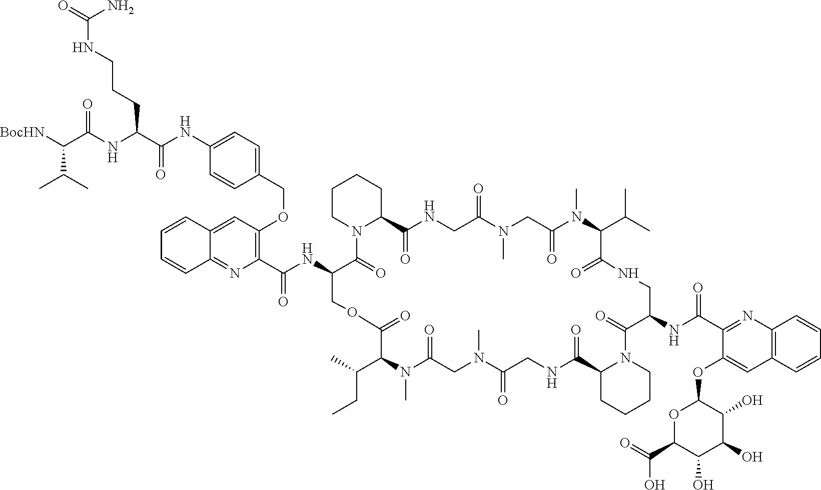 [SMILES: CC[C@H](C)[C@H]1C(=O)OC[C@@H](NC(=O)c2nc3ccccc3cc2OCc2ccc(NC(=O)[C@H](CCCNC(N)=O)NC(=O)[C@@H](NC(=O)OC(C)(C)C)C(C)C)cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)C(=O)N2CCCC[C@H]2C(=O)NCC(=O)N(C)CC(=O)N1C]

To a solution of Compound 352 (30 mg, 0.015 mmol) in THF (1 mL) at 0° C. was added a 1M aqueous solution of LiOH (0.075 mL, 0.075 mmol, 5 eq.). The ice bath was removed, and the solution was stirred at RT for 3 h, quenched by the addition of 0.1% TFA/H₂O (2 mL) and loaded directly on to a 12 g C18 flash column. Purification according to General Procedure 12 eluting with 25 to 60% CH₃CH/H₂O afforded the title compound (25 mg, 0.013 mmol, 90% yield) as a yellow solid.

LC/MS: Calc'd m/z=1856.9 for C₉₀H₁₂₄N₁₈O₂₅ found [M+2H]²⁺=930.0 and [M−Boc, +3H]³⁺=587.0.

### 4.54 (2S,3S,4S,5R,6S)-6-((2-(((7R,11S,19aS,26R,30S,38aS)-11-((S)-sec-butyl)-7-(3-((4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl)oxy)quinoline-2-carboxamido)-30-isopropyl-12,15,31,34-tetramethyl-6,13,16,19,25,29,32,35,38-nonaoxotetratriacontahydro-2H,6H,10H-dipyrido[2,1-b₁: 2′,1′-l][1]oxa[4,7,10,13,17,20,23,26,29]nonaazacyclodotriacontin-26-yl)carbamoyl)quinolin-3-yl)oxy)-3,4,5-trihydroxytetrahydro-2H-pyran-2-carboxylic acid (MT-VC-PAB-197-Glucoronide; Compound 354; DL-23)

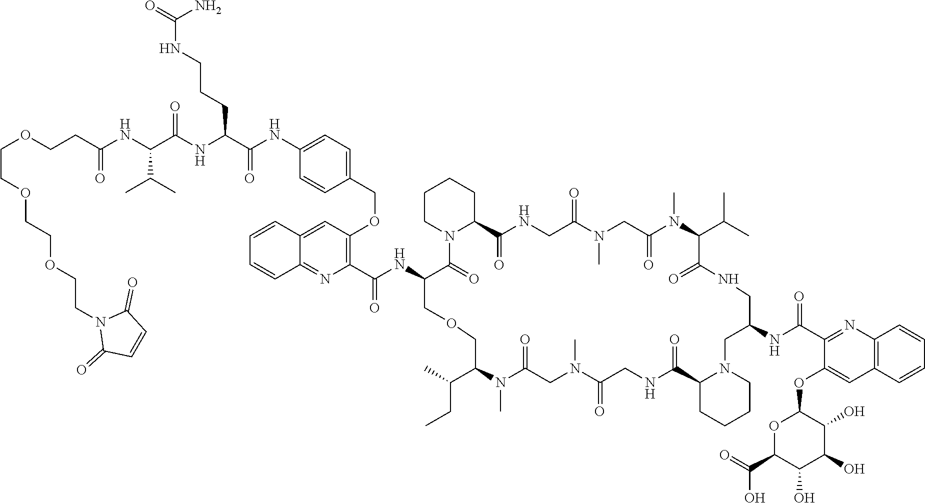 [SMILES: CC[C@H](C)[C@H]1COC[C@@H](NC(=O)c2nc3ccccc3cc2OCc2ccc(NC(=O)[C@H](CCCNC(N)=O)NC(=O)[C@@H](NC(=O)CCOCCOCCOCCN3C(=O)C=CC3=O)C(C)C)cc2)C(=O)N2CCCC[C@H]2C(=O)NCC(=O)N(C)CC(=O)N(C)[C@@H](C(C)C)C(=O)NC[C@@H](NC(=O)c2nc3ccccc3cc2O[C@@H]2O[C@H](C(=O)O)[C@@H](O)[C@H](O)[C@H]2O)CN2CCCC[C@H]2C(=O)NCC(=O)N(C)CC(=O)N1C]

The title compound was prepared from Compound 353 (10 mg, 0.0054 mmol) according to General Procedure 10 followed by General Procedure 11. Preparative HPLC purification as described in General Procedure 12 eluting with 35 to 50% CH₃CN/H₂O afforded the title compound (3.2 mmol, 0.0016 mmol, 30% yield) as a white solid.

LC/MS: Calc'd m/z=2039.9 for C₉₈H₁₃₃N₁₉O₂₉ found [M+2H]²⁺=1021.6 and [M+3H]³⁺=681.4.

### Example 5: In Vitro Cytotoxicity of Compounds of Formula I #1

The ability of representative compounds of Formula I to inhibit proliferation of tumour cells in vitro was evaluated in at least 4 of the following cell lines: OVCAR-3 (ovarian, ATCC), SKOV-3 (ovarian, ATCC), IGROV-1 (ovarian, national research council of Canada (NRC)), OV-90 (ovarian, ATCC), OVKATE (Ovarian, Japanese Collection of Research Bioresources (JCRB) (JCRB1044)), JIMT-1 (breast, NRC), SK-BR-3 (breast, ATCC), T-47D (breast, ATCC), ZR-75-1 (breast, ATCC), MDA-MB-468 (breast, AddexBio), BT-20 (breast, ATCC), BT-474 (breast, NRC), MDA-MB-175 (breast, AddexBio), A549 (lung, collaborator), NCI-H226 (lung, NRC), NCI-H661 (lung, NRC), NCI-H1573 (lung, ATCC). NCI-H1563 (lung, ATCC), H358 (lung, collaborator), EBC-1 (lung, Xenotech), H1299 (lung, NRC), NCI-H292 (lung, ATCC), HCC817 (lung, ATCC), NCI-H441 (lung, ATCC), MIA-PaCa-2 (pancreatic, ATCC), BxPC-3 (pancreatic, ATCC), Panc-1 (pancreatic, ATCC), AsPC-1 (pancreatic, ATCC), JEG-3 (placenta, AddexBio), HT-29 (colon, ATCC).

Cells were cultured per vendor instructions and in log-phase growth were detached by brief incubation in 0.05% trypsin, then resuspended in respective culturing media at 20,000 cells/mL (except for ZR-75 cells, which were resuspended at 10,000 cells/mL). 50 μL/well (˜1000 cells/well) were transferred to sterile tissue culture (TC)-treated 384-well plates (ThermoFisher Scientific, Catalogue #164610) and incubated overnight at 37° C. with 5% CO₂ to allow the cells to adhere to the plate surface. The next day, in a sterile, V-bottom, 96-well plate, test articles were titrated 1:3 in complete growth medium, creating a 10-point dose response titration with compounds at 3.5-times the desired final concentration. 20 μL from each dilution were added to the 384-well plate containing the seeded cells and these plates were incubated at 37° C. with 5% CO₂ for 5 nights. Cell viability was quantified by first incubating with 15 μL/well of CellTiter-Glo® (Promega, Catalogue #G7572) for 30 minutes, then measuring luminescence with a microplate luminometer (BioTek® Synergy™ H1 Microplate Reader). IC₅₀ values were determined by GraphPad Prism (GraphPad Software, San Diego, CA).

### Results

The results are presented in Table 7.1 and discussed in Example 7. Table 7.1 presents the results as average pIC₅₀ values. pIC₅₀=−log (IC₅₀), thus, for example, pIC₅₀=9 is equivalent to IC₅₀=1 nM, and pIC₅₀=6 is equivalent to IC₅₀=1 μM.

### Example 6: DNA-Binding by Compounds of Formula I

Representative compounds prepared as described in Example 2 were assessed in two complementary DNA-binding assays. Increased melting temperature of herring sperm DNA was measured using differential scanning calorimetry (DSC) and binding kinetics were determined by surface plasmon resonance (SPR) using a short DNA hairpin sequence. The assays are described below and the results are shown in Table 7.1.

### Differential Scanning Calorimetry (DSC)

DSC experiments were conducted as described in Leng, 2003, Nucleic Acids Res., 31 (21): 6191-6097, using a Nano DSC (TA Instruments, New Castle, DE) and analyzed using the NanoAnalyze™ software package. Herring sperm DNA solution (Invitrogen 15634017, 10 mg/mL) was diluted to 1 mg/mL (1.4 mM base pair) in BPE buffer (6 mM Na₂HPO₄, 2 mM NaH₂PO₄, 1 mM Na₄EDTA, pH 7.0). Test articles (20 μL, 10 mM in DMSO) were added and allowed reach equilibrium binding (>3 h), any insoluble material was removed by low-speed centrifugation, then samples were heated from 25 to 110° C. at a scan rate of 1° C./min. Melting curves were plotted and melting temperature was determined using NanoAnalyze™.

### Surface Plasmon Resonance (SPR) Assay

SPR experiments were conducted at 25° C. using a Biacore™ T200 instrument and SA-Biotin chip. PBST Buffer: Gibco PBS, pH 7.4 was modified with Tween®20 (0.01%) and DMSO (2%). Immobilization: a short biotinylated hairpin DNA sequence 5′-GCATGCTTTTGCATGC (5′ biotin) (SEQ ID NO: 1) that contains two known sandramycin binding segments (in bold) was heated to 90° C., cooled to RT then immobilized to 700 RU. Bis-intercalating peptides at concentrations of 5, 2.5, 1.25, 0.625, and 0.312 μM in PBST were flowed over the chip at 20 μL/min with an association time of 600 s followed by dissociation for 600 s. Reference cell values were subtracted and binding constants kₐ, k_{d}, and K_{D} were determined using Biacore™ T200 Evaluation Software, version 3.0.

### Results

Compound 137 (FD-24), in which the sarcosine (N-methyl Gly) residue of sandramycin was replaced with Gly, did not demonstrate measurable DNA binding via SPR and was inactive in cell assays (see Table 7.1). Conversely, increasing the size of the N-substitution from methyl to ethyl (Compound 132 (FD-23)) increased DNA binding affinity. The same trend was observed for Compounds 147 and 152 (FD-25 and FD-26, respectively), where the N-methyl Val was replaced with either Val (resulting in near complete loss of binding) or N-ethyl Val (which increased DNA affinity and in vitro cytotoxicity). Compounds 404 and 409 (FD-27 and FD-28, respectively) in which the Gly-Sar dipeptide unit was replaced with a much more flexible amino-PEG1-OH did not demonstrate measurable DNA binding.

The results also showed that replacement of the N-methyl Val residues of Compound 35 (FD-1) with pipecolic acid (Pip) to give des-methyl quinaldopeptin (Compound 49 (FD-14)) or quinaldopeptin produced two of the highest DNA binding affinity compounds tested. High affinity was also observed for Compound 187 (FD-15), which contains four Pip residues and an ether linkage in place of one of the native amides of quinaldopeptin. Transposing the N-methyl Val and Pip residues (Compound 414 (FD-29)) resulted in a non-binding and inactive compound. Finally, a series of asymmetric compounds (Compounds 77, 82 and 88 (FD-20, FD-21 and FD-22, respectively) illustrated that a 6-membered ring (pipecolic) appeared to be optimal for DNA binding, with both the 5-membered ring (proline) and 7-membered ring (azepane) showing slightly lower binding affinity. For non-cyclic amino acids at this position, increasing the side chain steric bulk of the amino acids also generated compounds with increased DNA binding affinity, with the Ile and cyclohexyl glycine (Chg) side chains (Compounds 67 and 162 (FD-9 and FD-13, respectively)) being particularly effective.

The correlation between DNA binding and cytotoxic potency is discussed in Example 7 below.

### Example 7: Lipophilicity of Compounds of Formula I

The ability to kill neighboring cells (i.e. bystander effect) is an important property for ADC payloads. For a payload comprising a compound of Formula I to be capable of bystander killing, membrane permeability (or transport) is required and the ability of compounds of Formula I to cross cell membranes and exert a cytotoxic effect was expected to be heavily influenced by lipophilicity. The lipophilicity of representative compounds prepared as described in Example 2 was assessed by calculated log D values (Instant JChem; Chemaxon Ltd., Budapest, Hungary) and experimental HPLC retention times determined as described below. The results are shown in Table 7.1.

Compounds were analyzed using an Agilent 1260 HPLC (Agilent Technologies, Inc., Santa Clara, CA) equipped with a Diode array detector and using a Phenomenex Kinetex EVO C18 150×4.6, 2.6 μm column (Phenomenex, Torrance, CA), eluting with 10 to 90% CH₃CN/H₂O with 0.1% FA over 25 min.

### Results

In order to assess the impact of lipophilicity on cytotoxic potency, calculated log D values (Instant JChem) and experimental HPLC retention times were correlated with the average pIC₅₀ on all cell lines tested (see Table 7.1). Across the compounds evaluated, both measures of lipophilicity demonstrate a stronger correlation with cytotoxic potency than DNA binding. For example, lipophilic compounds that demonstrate tight DNA binding (e.g. sandramycin, Compound 14 (FD-2) and Compound 197 (FD-19)) are expected to be highly potent. However, even compounds that show reduced DNA affinity can also demonstrate high potency due to their relatively high lipophilicity (e.g. Compound 97 (FD-11) and Compound 162 (FD-13)). Conversely, compounds such as Compound 49 (FD-14) and quinaldopeptin are significantly less potent than their high DNA affinity would predict due to their relatively low lipophilicity. While lipophilicity appears to play a larger a role in determining the potency of the compounds of Formula (I) than DNA binding, only compounds that stabilized DNA melting above 85° C. (in the DSC assay) showed good cytotoxicity.

TABLE 7.1
In vitro Cytotoxicity and Biophysical Characterization
Data for Compounds of Formula I

| | | | | DNA | |
|---|---|---|---|---|---|
| | pIC₅₀ | | HPLC | melt | K_{D} |
| Compound | (average) | cLogD | (min) | (° C.)¹ | (mM) |
| Sandramycin | 9.0 | 0.6 | 21.6 | 102 | 0.36 |
| (Compound 5) | | | | | |
| Quinaldopeptin | 8.0 | −0.6 | 16.7 | ND | 0.009 |
| Compound 35 (FD-1) | 7.3 | −0.9 | 17.1 | 88 | 3.3 |
| Compound 14 (FD-2) | 8.5 | −0.2 | 19.6 | 97 | 0.08 |
| Compound 178 (FD-3) | 8.1 | 0.0 | 18.4 | 92 | 0.3 |
| Compound 42 (FD-4) | 7.3 | −0.5 | 17.7 | 88 | ND² |
| Compound 22 (FD-6) | 7.6 | 1.4 | 21.6 | 85 | ND |
| Compound 54 (FD-7) | 8.1 | −0.7 | 18.4 | 88 | ND |
| Compound 55 (FD-8) | inactive | −2.4 | 10.6 | 86 | ND |
| Compound 67 (FD-9) | 8.6 | −0.0 | 18.8 | 91 | 0.4 |
| Compound 72 (FD-10) | 7.6 | 0.5 | 18.0 | 89 | 0.2 |
| Compound 97 (FD-11) | 8.4 | −0.0 | 18.9 | 86 | 0.4 |
| Compound 103 (FD-12) | 7.3 | −0.4 | 17.2 | ND | 1 |
| Compound 162 (FD-13) | 9.1 | 2.3 | 20.7 | ND | 0.3 |
| Compound 49 (FD-14) | 7.2 | −1.7 | 15.7 | 97 | 0.5 |
| Compound 187 (FD-15) | 8.0 | −0.8 | 16.6 | 94 | 0.95 |
| Compound 157 (FD-16) | 7.1 | −0.8 | 15.8 | ND | 0.77 |
| Compound 172 (FD-17) | 7.9 | 0.9 | 18.2 | 91 | ND |
| Compound 203 (FD-18) | 8.5 | 0.7 | 20.2 | 96 | 0.3 |
| Compound 197 (FD-19) | 9.4 | 0.5 | 19.4 | ND | 0.05 |
| Compound 77 (FD-20) | 6.9 | −1.7 | 15.0 | 88 | 1.1 |
| Compound 82 (FD-21) | 8.0 | −1.3 | 16.4 | 92 | 0.57 |
| Compound 88 (FD-22) | 8.5 | −0.9 | 16.4 | 88 | 0.7 |
| Compound 132 (FD-23) | 8.6 | −0.9 | 18.5 | ND | 0.3 |
| Compound 137 (FD-24) | Inactive | 0.1 | 15.1 | ND | none |
| Compound 147 (FD-25) | 8.5 | −0.2 | 17.9 | ND | 0.5 |
| Compound 152 (FD-26) | 6.9 | −1.3 | 14.6 | ND | 9 |
| Compound 404 (FD-27) | Inactive | 0.8 | 16.0 | 68¹ | ND |
| Compound 409 (FD-28) | inactive | −0.0 | 16.1 | 68¹ | ND |
| Compound 414 (FD-29) | inactive | −0.9 | 16.8 | ND | none |
| Compound 30 (FD-30) | 8.3 | 1.1 | 20.9 | 85 | ND |
| Compound 127 (FD-31) | 8.7 | 1.5 | 21.0 | ND | ND |
| Compound 109 (FD-32) | 7.77 | −1.66 | 15.4 | ND | 0.7 |
| Compound 115 (FD-33) | Inactive | −1.35 | 15.8 | ND | 0.7 |
| Compound 121 (FD-34) | 8.18 | −1.21 | 17.0 | ND | 0.3 |

¹DNA melting temperature = 68° C.
²ND = not determined

### Example 8: In Vitro Cytotoxicity of Compounds of Formula I #2

The ability of Compound 419 and Compound 424 to inhibit proliferation of tumour cells in vitro was measured by a cell proliferation assay using the following tumour cell lines: IGROV-1 (Ovarian Adenocarcinoma, NCI), JEG-3 (Placental Choriocarcinoma, AddexBio (C0030003)) and OVKATE (Ovarian Adenocarcinoma, Japanese Collection of Research Bioresources (JCRB) (JCRB1044)).

Cell lines were cultured as per vendor instructions prior to running the cell proliferation assay. Briefly, on the day prior to adding compound, cells (50 μL/well, 1000 cells/well) were added to sterile, tissue culture (TC)-treated, 384-well plates (ThermoFisher Scientific, Catalogue #164610) and incubated overnight at 37° C./5% CO₂ to allow the cells to adhere to the plate surface. The next day, in a sterile, V-bottom, 96-well plate, compounds were titrated 1:3 in complete growth medium, creating a 10-point dose response titration with compounds at 3.5-times the desired final concentration. Control wells with no compound (growth medium only) were included for each compound titration in 96-well dilution plates. 20 uL from the 10-point dose response titration were added to the 384-well plate containing the seeded cells, in duplicate. Plates were incubated at 37° C./5% CO₂ for 5 nights. After 5-night incubation, cell viability was quantified by the addition of 15 uL/well of CellTiter-Glo® (Promega, Catalogue #G7572) and incubation at room temperature for 30 minutes. After 30-minute incubation, luminescence was measured using a microplate luminometer (BioTek® Synergy™ H1 Microplate Reader). The resulting relative light units (RLU values) were converted to % cytotoxicity using the growth medium only control wells mentioned above:

$$\% Cytotoxicity = 1 - [ Well RLU / average medium only control well RLU ] .$$

Data was fit into 4-parameter logistic to curves using non-linear regression methods available with GraphPad Prism software (GraphPad Software, Inc.). The results are shown in Table 8.1.

TABLE 8.1
In vitro Cytotoxicity of Compounds of Formula I

| | Cell line EC₅₀ (nM)* | | |
|---|---|---|---|
| Compound | IGROV-1 | JEG-3 | OVKATE |
| Sandramycin | 1.6 | 3.3 | 0.8 |
| (Compound 5) | | | |
| Quinaldopeptin | 6.5 | 9.8 | 14.8 |
| Compound 419 | 11.5 | 6.9 | 24.1 |
| Compound 424 | 54.0 | 55.7 | n/a^{§} |

*Values reflect single assay results or in the case of multiple repeated assays, the average value of EC₅₀'s obtained by these methods.
^{§}“n/a” indicates a level of killing for which an EC₅₀ could not accurately be determined.

### Example 9: Plasma Stability of Compounds of Formula I

The stability of exemplary compounds of general Formula I in mouse plasma was evaluated as follows.

Compounds were incubated at 1 μM in duplicate in female mouse plasma, 250 μL final volume, at 37° C. for 24 h. Aliquots were sampled at 0, 15, 30, 60, 120, 240, 480 and 1440 min. Proteins were precipitated by addition of 5 volumes of cold methanol. After centrifugation 10000×g for 10 min, the residual concentration of compound in the supernatant was measured using an appropriate LC-MS/MS method. The measured concentrations were expressed as a function of time through a one-phase decay exponential regression calculated using GraphPad Prism software (GraphPad Software, Inc.) to generate the plasma half-life for each compound.

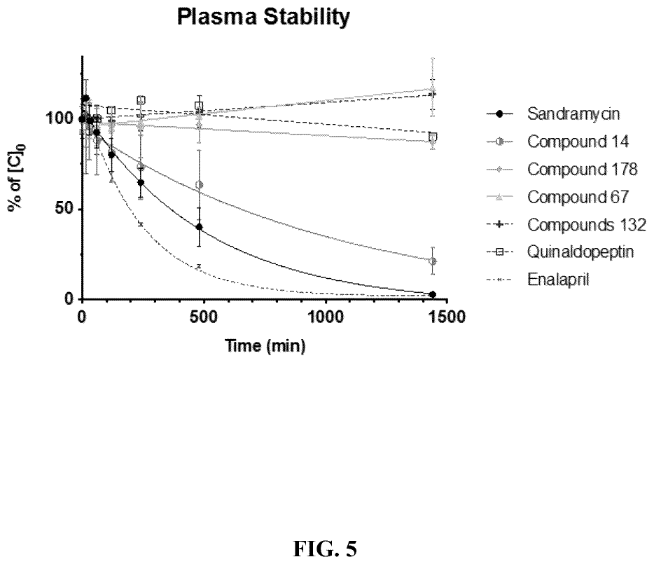

The results are shown in FIG. 5 (data for quinaldopeptin was collected as a singlet only). Enalapril, used as positive control for esterase activity, demonstrated a half-life of 2.7 h (163 min) in this assay. The plasma half-life determined for sandramycin was 5.9 h (352 min). Compound 14 (FD-2), Compound 67 (FD-9), Compound 132 (FD-23), Compound 178 (FD-3) and quinaldopeptin all showed improved half-lives compared to sandramycin, with Compound 14 (FD-2) having a half-life of 12.3 h (737 min), and quinaldopeptin, Compound 67 (FD-9), Compound 132 (FD-23) and Compound 178 (FD-3) demonstrating stability over the course of the experiment.

### Example 10: Preparation of Stochastically Conjugated Dar4 Antibody-Drug Conjugates

The antibody-drug conjugates (ADCs) comprising trastuzumab (anti-HER2), telisotuzumab (anti-cMet) or mirvetuximab (anti-FRα) as listed in Table 10.1 were prepared as follows.

To a solution of antibody (1-21 mg/mL) in PBS, 1 mM DTPA (pH 7.4), was added 2.0-3.0 molar equivalents of TCEP (1-10 mM dissolved in dH₂O). The solution was mixed thoroughly and incubated at 37° C. for 90-120 minutes. To the reduced antibody solution was added the maleimide-functionalized toxin (10-15 molar equivalents) from a 20 mM DMSO stock solution. The conjugation reaction was immediately mixed thoroughly by inversion and conjugation was allowed to proceed on ice for 1-4 hours. The resulting conjugate was then purified by passage over Zeba™ Spin Desalting Columns (40 kDa MWCO; Pierce) pre-equilibrated with PBS buffer, pH 7.4. The eluate was pooled, filter sterilized (Costar® Spin-X®, Corning) and stored at 4° C. The purified ADCs were analyzed for total protein content (bicinchonic acid assay, Pierce Catalogue #23225).

The average degree of conjugation of toxin-linker to antibody (DAR) was assessed by hydrophobic interaction chromatography (HIC). These techniques are described in Antibody Drug Conjugates, Methods in Molecular Biology, 2013, vol. 1045, pp. 275-284. L. Ducry, Ed. The DAR for these ADCs is 4±0.5 drugs.

Briefly, ADCs were subjected to HIC on a TSKgel® Butyl-NPR column (Tosoh Bioscience; 4.6 mm×3 mm i.d.; 2.5 m particle size) connected to an Agilent 1100 series HPLC. Samples were injected (5 μL) at or above 4 mg/mL. Where necessary, ADCs were concentrated prior to injection using Pall Nanosep® Omega™ centrifugal concentration devices (part #ODOC34). A linear gradient elution was employed starting at 95% mobile phase A/5% mobile phase B, transitioning to 5% mobile phase A/95% mobile phase B over a period of 12 min (mobile phase A: 1.5 M ammonium sulfate+25 mM sodium phosphate, pH 6.95; mobile phase B: 25% isopropanol, 75% 25 mM sodium phosphate, pH 6.95). Injection of unmodified antibody provided a means of identifying the peak with DAR=0. Antibodies were detected on the basis of absorbance at 280 nm.

The hydrophilicity of conjugates was evaluated by determining the HIC relative retention time (RRT) of the DAR4 species compared to the DAR0 species. For example, RRT DAR4=(RT DAR4/RT DAR0).

The amount of high molecular weight species (HMWS) present following conjugation was assessed by size exclusion chromatography (SEC). These techniques are described in Antibody Drug Conjugates, Methods in Molecular Biology, 2013, vol. 1045, pp. 275-284. L. Ducry, Ed.

Briefly, SEC was performed using an Agilent Infinity II 1260 HPLC (Agilent Technologies, Santa Clara, CA) with Advance Bio SEC column (300 Å, 2.7 μm, 7.8×150 mm) equilibrated with 5 column volumes of buffer (150 mM Na₂PO₄, pH 6.95) at room temperature. In general, 20-30 μg of sample at 2-3 mg/mL concentration was eluted isostatically for 7 min at 1 mL/min with absorbance monitoring at 280 nm. Chromatograms were integrated to provide complete, baseline-to-baseline integration of each peak, with reasonably placed separation between partially resolved peaks. The peak corresponding to the major component for IgG (approximate retention time 3.3 min) was reported as the monomer based on the SEC profile of the unmodified antibody. Any peak occurring prior to 3.3 min was designated as HMWS (high molecular weight species), and any peak occurring after 3.3 min was designated as LMWS (low molecular weight species), excluding solvent peaks (over 5.2 min).

TABLE 10.1
DAR4 Stochastic Antibody-Drug Conjugates

| | | | | RRT |
|---|---|---|---|---|
| ADC | Antibody¹ | Drug-Linker | % HMWS | DAR4 |
| Tr-1 | Trastuzumab | MT-VC-PAB-35 (DL-1) | 9.5 | 2.05 |
| ADC1 | | | | |
| Tr-2 | Trastuzumab | MT-VA-PAB-35 (DL-3) | 10 | 2.00 |
| ADC3 | | | | |
| Tr-3 | Trastuzumab | ME-dPEG4-Glu-dPEG24-VA-PAB-35 | 1.5 | 1.98 |
| ADC7 | | (DL-7) | | |
| Tr-4 | Trastuzumab | MT-Cya-VA-PAB-35 (DL-6) | 4 | 2.00 |
| ADC6 | | | | |
| Tr-5 | Trastuzumab | ME-CyaT-VA-PAB-35 (DL-5) | 9 | 2.04 |
| ADC5 | | | | |
| Tr-6 | Trastuzumab | ME-VA-PAB-35 (DL-4) | 8.4 | 1.91 |
| ADC4 | | | | |
| Tr-7 | Trastuzumab | MT-VA-PAB-35-Glucoronide | <1 | 1.46 |
| ADC8 | | (DL-8) | | |
| Tr-8 | Trastuzumab | ME-VA-PAB-35-Glucoronide | ND | 1.41 |
| ADC9 | | (DL-9) | | |
| Tr-9 | Trastuzumab | MT-VC-PAB-14 (DL-12) | ND | 2.11 |
| ADC12 | | | | |
| Tr-10 | Trastuzumab | MT-VC-PAB-14-Glucoronide | <1 | 1.50 |
| ADC13 | | (DL-13) | | |
| Tr-11 | Trastuzumab | MT-VC-PAB-178-Glucoronide | <1 | 1.42 |
| ADC14 | | (DL-14) | | |
| Te-1 | Telisotuzumab | ME-VA-PAB-35 (DL-4) | 9 | 2.04 |
| ADC15 | | | | |
| Te-2 | Telisotuzumab | MT-VA-PAB-35-Glucoronide | 1 | 1.50 |
| ADC16 | | (DL-8) | | |
| Te-3 | Telisotuzumab | MT-VC-PAB-14 (DL-13) | <1 | 1.50 |
| ADC19 | | | | |
| Te-4 | Telisotuzumab | MT-VC-PAB-178-Glucoronide | <1 | 1.41 |
| ADC20 | | (DL-14) | | |
| Mi-1 | Mivetuximab | MT-VC-PAB-35 (DL-1) | 4.6 | 2.01 |
| ADC21 | | | | |
| Mi-2 | Mivetuximab | MT-VC-PAB-35-Glucoronide | 2.4 | 1.48 |
| ADC22 | | (DL-15) | | |
| Mi-3 | Mivetuximab | MT-VC-PAB-35-Glucoronide | 4.5 | 1.49 |
| ADC10 | | (DL-10) | | |
| Mi-4 | Mivetuximab | MT-VC-PAB-35-pyrophosphate | 1.3 | 1.44 |
| ADC11 | | (DL-11) | | |
| Mi-5 | Mivetuximab | MT-PP-35 (DL-16) | 1.3 | 1.66 |
| ADC23 | | | | |
| Mi-6 | Mivetuximab | MT-VC-PAB-14 (DL-12) | 17 | 2.07 |
| ADC24 | | | | |
| Mi-7 | Mivetuximab | MT-VC-PAB-14-Glucoronide | 4.0 | 1.51 |
| ADC25 | | (DL-13) | | |
| Mi-8 | Mivetuximab | MT-VC-PAB-178 (DL-17) | 5.5 | 1.89 |
| ADC26 | | | | |
| Mi-9 | Mivetuximab | MT-VC-PAB-178-Glucoronide | 3.9 | 1.51 |
| ADC27 | | (DL-14) | | |
| Mi-10 | Mivetuximab | MT-VC-PAB-67 (DL-18) | 28 | 2.24 |
| ADC28 | | | | |
| Mi-11 | Mivetuximab | MT-VC-PAB-67-Glucoronide | 3.9 | 1.62 |
| ADC29 | | (DL-19) | | |
| Mi-12 | Mivetuximab | MT-VC-PAB-88-Glucoronide | ND | 1.43 |
| ADC30 | | (DL-20) | | |
| Mi-13 | Mivetuximab | MT-VC-PAB-82-Glucoronide | 4.6 | 1.63 |
| ADC31 | | (DL-21) | | |
| Mi-14 | Mivetuximab | MT-VC-PAB-132-Glucoronide | ND | 1.52 |
| ADC32 | | (DL-22) | | |
| Mi-15 | Mivetuximab | MT-VC-PAB-197-Glucoronide | ND | 1.48 |
| ADC33 | | (DL-23) | | |

¹ADCs listed as comprising telisotuzumab or mirvetuximab comprised an antibody having the Fab regions from telisotuzumab or mirvetuimab, respectively, fused to a heterodimeric IgG1 Fc comprising the following mutations: Chain A: T350V_L351Y_F405A_Y407V; Chain B: T350V_T366L_K392L_T394W.

### Example 11: Preparation of Site-Specific Antibody-Drug Conjugates

The site-specifically conjugated ADCs listed in Table 11.1 were prepared as described below using an antibody comprising the Fab regions from telisotuzumab fused to a heterodimeric IgG1 Fc comprising the following mutations: Chain A: T350V_L351Y_F405A_Y407V; Chain B: T350V_T366L_K392L_T394W. The antibody further comprised two cysteine substitutions (A114C) for site specific conjugation of drug-linker to yield a conjugate with an average DAR of approximately 2.0.

To a solution of antibody (1-10 mg/mL) in DPBS, 1 mM DTPA (pH 7.4), was added 20 molar equivalents of TCEP (1-10 mM dissolved in dH₂O). The solution was mixed thoroughly and incubated at 37° C. for 180-240 minutes. The reduced antibody solution was then passaged over a Zeba™ Spin Desalting Column (40 kDa MWCO; ThermoFisher Scientific, Waltham, MA) pre-equilibrated with PBS, pH 7.4. The purified antibody was mixed with 25 molar equivalents of DHAA (25 mM dissolved in H₂O) and incubated for 16-24 hours at 4° C. Following incubation, the antibody solution was incubated at room temperature for 60 minutes. To the antibody solution (1-5 mg/mL) was added the drug-linker (10-15 molar equivalents) from a 20 mM DMSO stock solution, and propylene glycol (5% v/v, final). The mixture was incubated at room temperature on a rotator for a period of 2-4 hours. The resulting conjugate was purified using a centrifugal filter device (30 kDa MWCO, Pall) using PBS, pH 7.4. The purified retentate was filter sterilized and stored at −80° C. The purified ADCs were analyzed for total protein content (bicinchonic acid assay; ThermoFisher Scientific, Waltham, MA; Cat #23225). % HMWS was determined by SEC as described in Example 10.

TABLE 11.1
DAR2 Site-Specific Antibody-Drug Conjugates

| ADC | Antibody | Drug-Linker | % HMWS |
|---|---|---|---|
| Te-5 | Telisotuzumab | MT-VC-PAB-35 (DL-1) | 1.1 |
| ADC11 | | | |
| Te-6 | Telisotuzumab | MT-VC-PAB-35-Glucoronide | 1.8 |
| ADC18 | | (DL-15) | |

### Example 12: In Vitro Activity of Antibody-Drug Conjugates

The ability of the antibody-drug conjugates Tr-1 to Tr-11, Te-1 to Te-6, Mi-1 to Mi-15 (see Examples 10 and 11) to inhibit proliferation of tumour cells in vitro was measured by cell proliferation assay as described in Example 5 using the following tumour cell lines:

For trastuzumab ADCs: SK-BR-3 (breast epithelial adenocarcinoma; ATCC), JIMT-1 (breast epithelial carcinoma; AddexBio Technologies, San Diego, CA (C0006005)) and MDA-MB-468 (breast epithelial adenocarcinoma; AddexBio Technologies, San Diego, CA).

For telisotuzumab ADCs: EBC-1 (squamous cell carcinoma; XenoTech, Kansas City, KS (JCRB0820)), HT-29 (colorectal epithelial adenocarcinoma; ATCC (HTB-38)) and HCC827 (lung epithelial adenocarcinoma; ATCC (CRL-2868)).

For mirvetuximab ADCs: IGROV-1 (ovarian carcinoma; National Research Council of Canada (NRC)), OVKATE (ovarian adenocarcinoma, Japanese Collection of Research Bioresources (JCRB) (JCRB1044)) and OVCAR-3 (ovarian epithelial adenocarcinoma, ATCC).

The results are shown in Tables 12.1, 12.2 and 12.3 below. In the high antigen expressing cell lines (SK-BR-3: HER2 3+, EBC-1: cMET 3+, IGROV-1: FRα 3+), all ADCs showed potent activity (low pM) with minimal differentiation regardless of payload, linker trigger, or presence of a hydrophilic mask. Moderate and low antigen expressing cell lines were less sensitive to glucuronide masked ADCs compared to unmasked ADCs with the same payload. In JIMT-1 (HER2 2+), the ADCs Tr-8 and Tr-10 with payloads FD-1 and FD-2, respectively, did not show killing at the highest assay concentration (16 nM), while the payload matched unmasked ADCs had single digit nM activity. In OVKATE (FRα 2+) cells the ADCs Mi-2, Mi-7, Mi-9, Mi-11, Mi-12, Mi-13, Mi-14 and Mi-15 showed potent IC₅₀ values but low percent maximum (% max) cytotoxicity, while in OVCAR-3 (FRα 1+) cells, these ADCs achieved high % max killing but at low potency. Additionally, Mi-2, Mi-7 and Mi-9 were 10× less potent than the payload matched non-masked ADCs. Incorporation of a PAB spacer between the payload and glucuronide mask (Mi-3) partially recovered activity. Finally, ADCs containing a cleavable pyrophosphate, with a self-immolative methylene spacer, either as the cleavage sequence (Mi-5) or as a mask (Mi-4), displayed similar potency and max killing compared to non-masked ADCs.

TABLE 12.1
Inhibition of Tumour Cell Proliferation
in vitro by Trastuzumab ADCs

| | IC₅₀ (nM) | | |
|---|---|---|---|
| | SK-BR-3 | JIMT-1 | MDA-MB-468 |
| ADC | (HER2 High) | (HER2 Mid) | (HER2 Negative) |
| Tr-1 | 0.02 | 1.9 | >10 |
| Tr-2¹ | 0.01 | 1.1 | >10 |
| Tr-3 | 0.01 | 3.3 | >10 |
| Tr-4 | 0.01 | 1.8 | >10 |
| Tr-5 | 0.01 | 1.4 | >10 |
| Tr-6 | 0.02 | 3.0 | >10 |
| Tr-7 | 0.02 | >10 | >10 |
| Tr-8 | 0.02 | >10 | >10 |
| Tr-9 | ND | 1.35 | >10 |
| Tr-10 | 0.04 | >10 | >10 |
| Tr-11 | 0.03 | >10 | >10 |

¹An analogous ADC prepared with a drug-linker comprising 8 PEG units instead of 3 (DL-2 in Table 3) showed similar activity, but was slightly more hydrophobic.

TABLE 12.2
Inhibition of Tumour Cell Proliferation
in vitro by Telisotuzumab ADCs

| | IC₅₀ (nM) | | |
|---|---|---|---|
| | EBC-1 | HCC827 | HT-29 |
| ADC | (cMET High) | (cMET Mid) | (cMET Negative) |
| Te-1 | 0.01 | 0.05 | 0.15 |
| Te-2 | 0.02 | 0.07 | 0.23 |
| Te-3 | 0.02 | 0.07 | 0.46 |
| Te-4 | 0.02 | 0.20 | 5.56 |
| Te-5 | 0.14 | ND | ND |
| Te-6 | 0.15 | ND | ND |

TABLE 12.3
Inhibition of Tumour Cell Proliferation
in vitro by Mirvetuximab ADCs

| | IC₅₀ (nM) | | |
|---|---|---|---|
| | IGROV-1 | OVKATE | OVCAR-3 |
| ADC | (FRα High) | (FRα Mid) | (FRα Negative) |
| Mi-1 | 0.01 | 0.15 | 1.77 |
| Mi-2 | 0.02 | 0.18 | 31.3 |
| Mi-3 | 0.01 | 0.11 | 20.0 |
| Mi-4 | 0.01 | 0.15 | 0.75 |
| Mi-5 | 0.01 | 1.14 | 0.66 |
| Mi-6 | 0.01 | 0.03 | 0.01 |
| Mi-7 | 0.02 | 0.05 | 0.78 |
| Mi-8 | 0.01 | 0.03 | 2.55 |
| Mi-9 | 0.02 | ND | 36.3 |
| Mi-10 | 0.01 | 0.18 | 1.71 |
| Mi-11 | 0.01 | 1.43 | 40.9 |
| Mi-12 | 0.02 | 0.23 | 36.5 |
| Mi-13 | 0.02 | 0.46 | 41.4 |
| Mi-14 | 0.08 | 1.54 | 160 |
| Mi-15 | 0.02 | 0.43 | 40.6 |

### Example 13: In Vivo Activity of Stochastic Dar4 Antibody-Drug Conjugates

The ability of the mirvetuximab ADCs, Mi-1 and Mi-2 (see Example 10), to inhibit in vivo growth of OVCAR-3 ovarian cancer cells in a xenograft model was assessed as follows.

Female Balb/c nude mice (AK, Beijing, China) at 6-8 weeks of age were subcutaneously implanted in the right flank with 3×10⁶ OVCAR-3 tumour cells (n=6 per group). Once tumours reached approximately 150 mm³ in size, animals were assigned to treatment groups and were dosed by intravenous single injection with vehicle, Mi-1 (0.1, 1, and 3 mg/kg), Mi-2 (0.3, 1, and 3 mg/kg). Tumour measurements were performed with a caliper biweekly in two dimensions. Mice were ethically sacrificed when tumours reached a size of 2000 mm³. Tumour volumes are reported as the mean±SEM for each group.

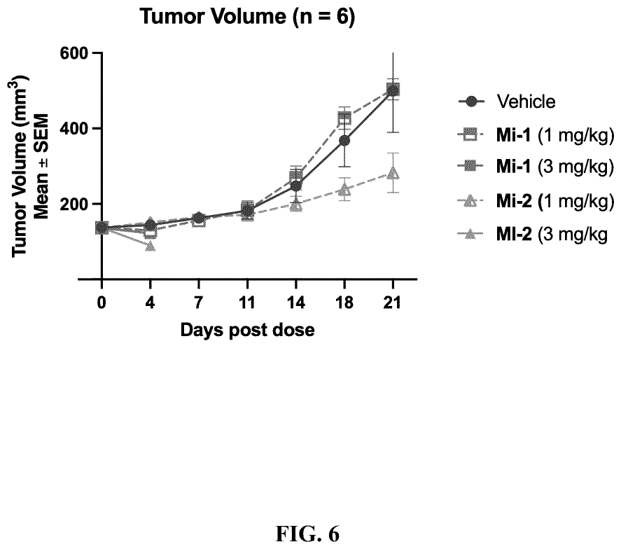

Differentiation between the glucuronide masked ADC, Mi-2, and the unmasked ADC, Mi-1, was seen only at the 1 mg/kg dose level (FIG. 6). Mice treated with the more hydrophilic ADC, Mi-2, showed minimal (2.5%) body weight loss and modest tumour growth inhibition. Conversely, mice treated with Mi-1 experienced >5% body weight loss, including the death of a single animal, and no tumour growth inhibition was observed. Mice treated with 3 mg/kg of either ADC suffered severe body weight loss and mortality by day 4, while mice treated at 0.3 mg/kg with either ADC did not show tumour volume reduction relative to vehicle.

### Example 14: In Vivo Activity of Site-Specific Antibody-Drug Conjugates

The ability of the telisotuzumab site-specific ADCs, Te-5 and Te-6 (see Example 11), to inhibit in vivo growth of EBC-1 lung cancer cells in a xenograft model was assessed as follows.

Female Balb/c nude mice (AK, Beijing, China) at 6-8 weeks of age were subcutaneously implanted in the right flank with 3×10⁶ EBC-1 tumour cells (n=6 per group). Once tumours reached approximately 125 to 200 mm³ in size, animals were assigned to treatment groups and were dosed by intravenous single injection with vehicle, Te-5 (5 mg/kg) or Te-6 (5 mg/kg). Tumour measurements were performed with a caliper biweekly in two dimensions. Mice were ethically sacrificed when tumours reached a size of 2000 mm³. Tumour volumes are reported as the mean±SEM for each group.

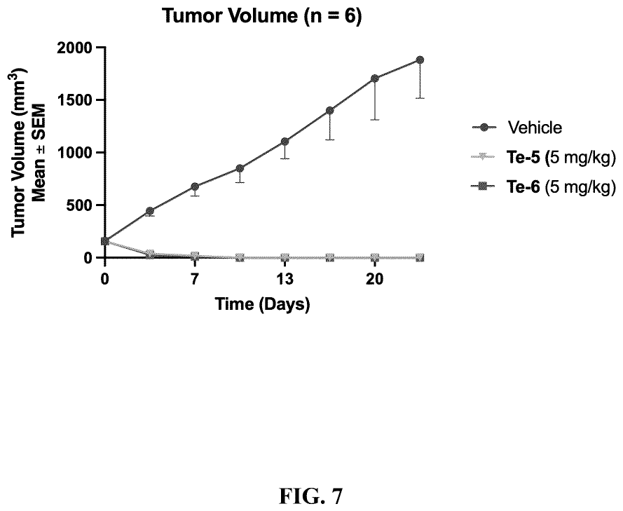

Both ADCs achieved complete tumour regression at 5 mg/kg with minimal body weight loss (FIG. 7). It is noted that the payload dose delivered by a 5 mg/kg dose of these site-specific DAR2 ADCs corresponds to the approximate payload dose that would be delivered by a 2.5 mg/kg dose of a stochastically conjugated DAR4 ADC.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

1. A compound having Formula I: 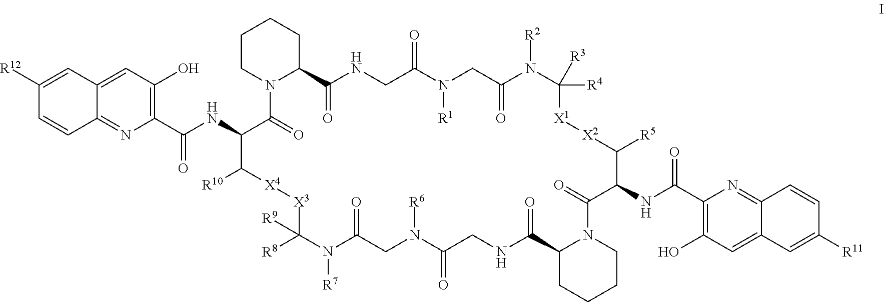
or a pharmaceutically acceptable salt thereof,
wherein:
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—, and
X² and X⁴ are each independently —NR¹⁴— or —O—, and R¹⁴ is H or C₁-C₂ alkyl, with the proviso that the compound is other than:

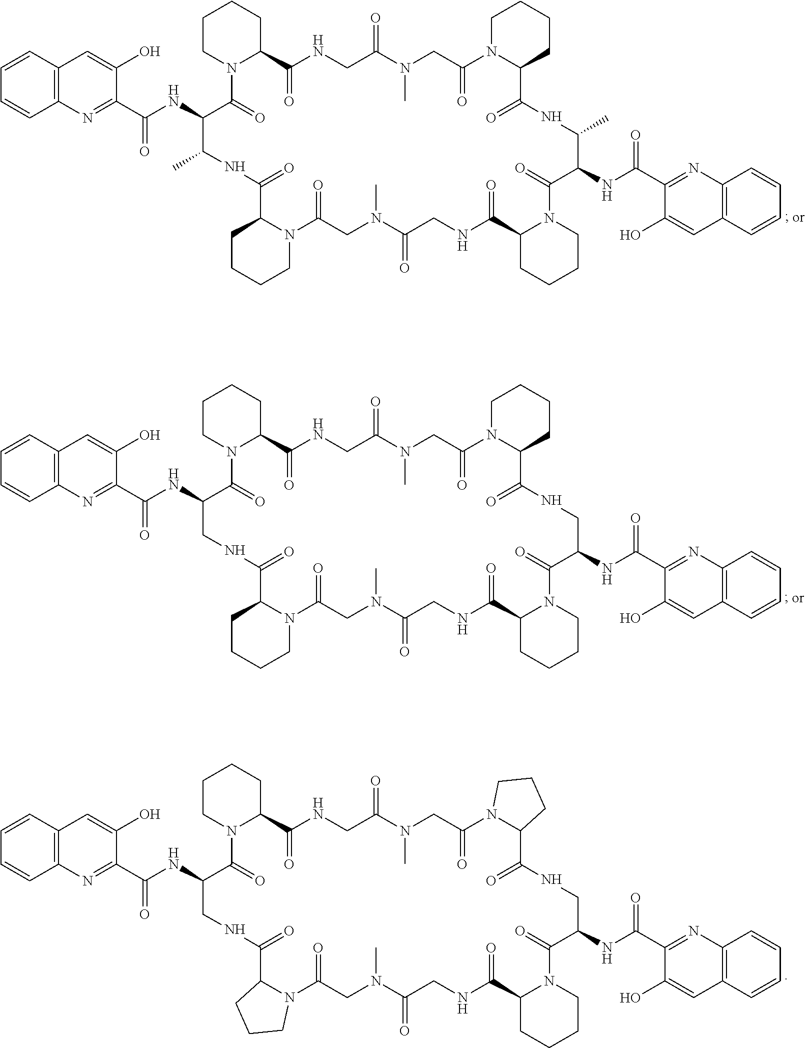

2. The compound according to claim 1, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; or R² and R³ together with the N and C atoms to which they are bonded form a 7-membered heterocycloalkyl, and R⁴ is H, and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl;
or R⁷ and R⁸ together with the N and C atoms to which they are bonded form a 7-membered heterocycloalkyl, and R⁹ is H, and/or
(c) R¹¹ and R¹² are each H, and/or
(d) R⁵ and R¹⁰ are each H.

3. The compound according to claim 1, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl, and/or
(c) R¹¹ and R¹² are each H, and/or
(d) R⁵ and R¹⁰ are each H.

4. (canceled)

5. The compound according to claim 1, wherein:
(a) X¹ is —CH₂— and X² is —O—, and/or X³ is —CH₂— and X⁴ is —O—, or
(b) X¹ is —C(O)— and X² is —NR¹⁴— or —O—, and/or X³ is —C(O)— and X⁴ is —NR¹⁴— or —O—.

6.-8. (canceled)

9. The compound according to claim 1, wherein X¹ and X³ are each —C(O)—, and X² and X⁴ are each —NR¹⁴—.

10. (canceled)

11. The compound according to claim 1, wherein the compound is selected from the compounds set forth in Tables 1 and 2.

12. A composition comprising the compound according to claim 1 and a pharmaceutically acceptable carrier, diluent or excipient.

13. A method of inhibiting the proliferation of tumour cells, microbial cells or viral cells comprising contacting the cells with the compound according to claim 1.

14. A method of treating a microbial infection or a cancer in a subject in need thereof, comprising administering to the subject an effective amount of the compound according to claim 1.

15.-21. (canceled)

22. A conjugate having Formula XIII: 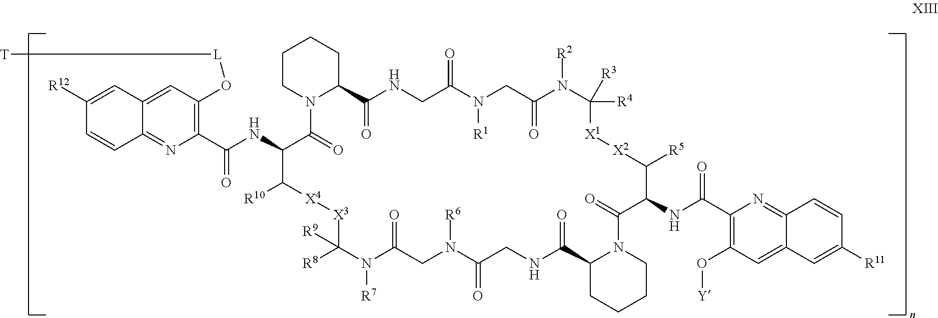
wherein:
T is a targeting moiety;
L is a linker;
n is between 1 and about 12;
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—;
X² and X⁴ are each independently —NR¹⁴— or —O—, and R¹⁴ is H or C₁-C₂ alkyl, and
Y′ is H or (L^{A})ₚ-HM, where L^{A} is a self-immolative moiety, p is 0, 1 or 2, and HM is a hydrophilic moiety.

23. The conjugate according to claim 22, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; or R² and R³ together with the N and C atoms to which they are bonded form a 7-membered heterocycloalkyl, and R⁴ is H, and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl; or R⁷ and R⁸ together with the N and C atoms to which they are bonded form a 7-membered heterocycloalkyl, and R⁹ is H, and/or
(c) R¹¹ and R¹² are each H, and/or
(d) R⁵ and R¹⁰ are each H.

24. The conjugate according to claim 22, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl, and/or
(c) R¹¹ and R¹² are each H, and/or
(d) R⁵ and R¹⁰ are each H.

25. (canceled)

26. The conjugate according to claim 22, wherein:
(a) X¹ is —CH₂— and X² is —O—, and/or X³ is —CH₂— and X⁴ is —O—, or
(b) X¹ is —C(O)— and X² is —NR¹⁴— or —O—, and/or X³ is —C(O)— and X⁴ is —NR¹⁴— or —O—.

27.-29. (canceled)

30. The conjugate according to claim 22, wherein X¹ and X³ are each —C(O)—, and X² and X⁴ are each —NR¹⁴—.

31.-32. (canceled)

33. The conjugate according to claim 22, wherein Y′ is (L^{A})ₚ-HM.

34. The conjugate according to claim 33, wherein HM is an enzymatically cleavable hydrophilic moiety and p is 0 or 1.

35. (canceled)

36. The conjugate according to claim 22, wherein the conjugate comprises a drug-linker selected from the drug-linkers set forth in Table 3.

37. The conjugate according to claim 22, wherein T is an antibody or antigen-binding antibody fragment.

38. The conjugate according to claim 37, wherein the antibody or antigen-binding antibody fragment binds to a tumour-associated antigen.

39. A composition comprising the conjugate according to claim 22 and a pharmaceutically acceptable carrier, diluent or excipient.

40. A method of inhibiting the proliferation of tumour cells, microbial cells or viral cells comprising contacting the cells with the conjugate according to claim 22.

41. A method of treating a microbial infection or a cancer in a subject in need thereof, comprising administering to the subject an effective amount of the conjugate according to claim 22.

42. (canceled)

43. A drug-linker having Formula XI: 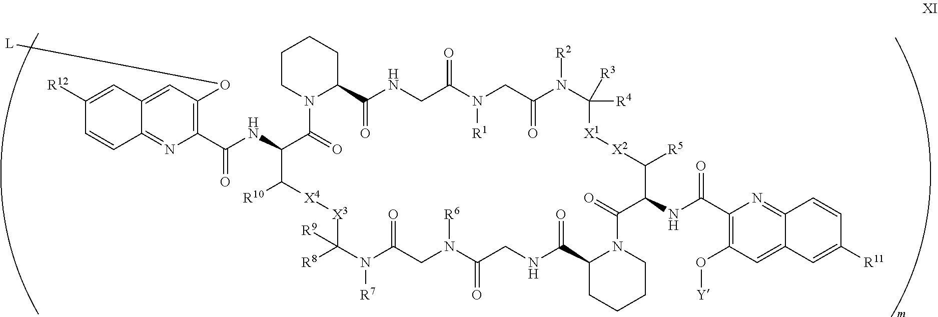
wherein:
L is a linker;
m is between 1 and about 10;
R¹ and R⁶ are each independently C₁-C₄ alkyl;
R² is H or C₁-C₄ alkyl, R³ is C₃-C₆ cycloalkyl, phenyl or C₁-C₄ alkyl optionally substituted with C₁-C₄ alkoxy, and R⁴ is H; or
R² is H or C₁-C₄ alkyl, and R³ and R⁴ together with the C atom to which they are bonded form a 3- or 4-membered cycloalkyl; or
R² and R³ together with the N and C atoms to which they are bonded form a 5-, 6- or 7-membered heterocycloalkyl, and R⁴ is H;
R⁵ and R¹⁰ are each independently H or C₁-C₂ alkyl;
R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or
R⁷ is H or C₁-C₄ alkyl, and R⁸ and R⁹ together with the C atom to which they are bonded form a 3- or 4-membered cycloalkyl; or
R⁷ and R⁸ together with the N and C atoms to which they are bonded form a 5-, 6- or 7-membered heterocycloalkyl, and R⁹ is H;
R¹¹ and R¹² are each independently H or halo;
X¹ and X³ are each independently —CH₂— or —C(O)—;
X² and X⁴ are each independently —NR¹⁴— or —O—, and R¹⁴ is H or C₁-C₂ alkyl, and
Y′ is H or (L^{A})ₚ-HM, where L^{A} is a self-immolative moiety, p is 0, 1 or 2, and HM is a hydrophilic moiety.

44. The drug-linker according to claim 43, wherein m is between 1 and 3.

45. The drug-linker according to claim 43, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; or R² and R³ together with the N and C atoms to which they are bonded form a 7-membered heterocycloalkyl, and R⁴ is H, and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl; or R⁷ and R⁸ together with the N and C atoms to which they are bonded form a 7-membered heterocycloalkyl, and R⁹ is H, and/or
(c) R¹¹ and R¹² are each H, and/or
(d) R⁵ and R¹⁰ are each H.

46. The drug-linker according to claim 43, wherein:
(a) R² is H or C₁-C₄ alkyl, R³ is C₃-C₆ cycloalkyl, phenyl or C₁-C₄ alkyl optionally substituted with C₁-C₄ alkoxy, and R⁴ is H; or R² is H or C₁-C₄ alkyl, and R³ and R⁴ together with the C atom to which they are bonded form a 3- or 4-membered cycloalkyl; and/or
(b) R⁷ is H or C₁-C₄ alkyl, R⁸ is C₃-C₆ cycloalkyl, phenyl or C₁-C₄ alkyl optionally substituted with C₁-C₄ alkoxy, and R⁹ is H; or R⁷ is H or C₁-C₄ alkyl, and R⁸ and R⁹ together with the C atom to which they are bonded form a 3- or 4-membered cycloalkyl, and/or
(c) R¹¹ and R¹² are each H, and/or
(d) R⁵ and R¹⁰ are each H.

47. (canceled)

48. The drug-linker according to claim 43, wherein;
(a) X¹ is —CH₂— and X² is —O—, and/or X³ is —CH₂— and X⁴ is —O—, or
(b) X¹ is —C(O)— and X² is —NR¹⁴— or —O—, and/or X³ is —C(O)— and X⁴ is —NR¹⁴— or —O—.

49.-51. (canceled)

52. The drug-linker according to claim 43, wherein X¹ and X³ are each —C(O)—, and X² and X⁴ are each —NR¹⁴—.

53.-54. (canceled)

55. The drug-linker according to claim 43, wherein Y′ is (L^{A})ₚ-HM.

56. The drug-linker according to claim 55, wherein HM is an enzymatically cleavable hydrophilic moiety and p is 0 or 1.

57. (canceled)

58. The drug-linker according to claim 43, wherein the drug-linker is selected from the drug-linkers set forth in Table 3.
